SEQUENCE LISTING (11 sequences)

SEQ ID NO: 1 - DNA, 3429 bp, Homo sapiens
atgggggacaaggatatgcctactgctgggatgccgagtcttctccagagttcctctgagagtcctcagagttgtcctgagggggaggactcccagtctcctctccagattccccagagttctcctgagagcgacgacaccctgtatcctctccagagtcctcagagtcgttctgagggggaggactcctcggatcctctccagagacctcctgaggggaaggactcccagtctcctctccagattccccagagttctcctgagggcgacgacacccagtctcctctccagaattctcagagttctcctgaggggaaggactccctgtctcctctagagatttctcagagccctcctgagggtgaggatgtccagtctcctctgcagaatcctgcgagttccttcttctcctctgctttattgagtattttccagagttcccctgagagtactcaaagtccttttgagggttttccccagtctgttctccagattcctgtgagcgccgcctcctcctccactttagtgagtattttccagagttcccctgagagtactcaaagtccttttgagggttttccccagtctccactccagattcctgtgagccgctccttctcctccactttattgagtattttccagagttcccctgagagaactcagagtacttttgagggttttgcccagtctcctctccagattcctgtgagcccctcctcctcctccactttactgagtcttttccagagtttctctgagagaactcagagtacttttgagggttttgcccagtcttctctccagattcctgtgagcccctccttctcctccactttagtgagtcttttccagagttcccctgagagaactcagagtacttttgagggttttccccagtctcctctccagattcctgtgagctcctcctcctcctccactttattgagtcttttccagagttcccctgagagaactcacagtacttttgagggttttccccagtctcttctccagattcctatgacctcctccttctcctctactttattgagtattttccagagttctcctgagagtgctcaaagtacttttgagggttttccccagtctcctctccagattcctgggagcccctccttctcctccactttactgagtcttttccagagttcccctgagagaactcacagtacttttgagggttttccccagtctcctctccagattcctatgacctcctccttctcctctactttattgagtattttacagagttctcctgagagtgctcaaagtgcttttgagggttttccccagtctcctctccagattcctgtgagctcctctttctcctacactttattgagtcttttccagagttcccctgagagaactcacagtacttttgagggttttccccagtctcctctccagattcctgtgagctcctcctcctcctcctccactttattgagtcttttccagagttcccctgagtgtactcaaagtacttttgagggttttccccagtctcctctccagattcctcagagtcctcctgaaggggagaatacccattctcctctccagattgttccaagtcttcctgagtgggaggactccctgtctcctcactactttcctcagagccctcctcagggggaggactccctatctcctcactactttcctcagagccctcctcagggggaggactccctgtctcctcactactttcctcagagccctcagggggaggactccctgtctcctcactactttcctcagagccctcctcagggggaggactccatgtctcctctctactttcctcagagtcctcttcagggggaggaattccagtcttctctccagagccctgtgagcatctgctcctcctccactccatccagtcttccccagagtttccctgagagttctcagagtcctcctgaggggcctgtccagtctcctctccatagtcctcagagccctcctgaggggatgcactcccaatctcctctccagagtcctgagagtgctcctgagggggaggattccctgtctcctctccaaattcctcagagtcctcttgagggagaggactccctgtcttctctccattttcctcagagtcctcctgagtgggaggactccctctctcctctccactttcctcagtttcctcctcagggggaggacttccagtcttctctccagagtcctgtgagtatctgctcctcctccacttctttgagtcttccccagagtttccctgagagtcctcagagtcctcctgaggggcctgctcagtctcctctccagagacctgtcagctccttcttctcctacactttagcgagtcttctccaaagttcccatgagagtcctcagagtcctcctgaggggcctgcccagtctcctctccagagtcctgtgagctccttcccctcctccacttcatcgagtctttcccagagttctcctgtgagctccttcccctcctccacttcatcgagtctttccaagagttcccctgagagtcctctccagagtcctgtgatctccttctcctcctccacttcattgagcccattcagtgaagagtccagcagcccagtagatgaatatacaagttcctcagacaccttgctagagagtgattccttgacagacagcgagtccttgatagagagcgagcccttgttcacttatacactggatgaaaaggtggacgagttggcgcggtttcttctcctcaaatatcaagtgaagcagcctatcacaaaggcagagatgctgacgaatgtcatcagcaggtacacgggctactttcctgtgatcttcaggaaagcccgtgagttcatagagatactttttggcatttccctgagagaagtggaccctgatgactcctatgtctttgtaaacacattagacctcacctctgaggggtgtctgagtgatgagcagggcatgtcccagaaccgcctcctgattcttattctgagtatcatcttcataaagggcacctatgcctctgaggaggtcatctgggatgtgctgagtggaataggggtgcgtgctgggagggagcactttgcctttggggagcccagggagctcctcactaaagtttgggtgcaggaacattacctagagtaccgggaggtgcccaactcttctcctcctcgttacgaattcctgtggggtccaagagctcattcagaagtcattaagaggaaagtagtagagtttttggccatgctaaagaataccgtccctattacctttccatcctcttacaaggatgctttgaaagatgtggaagagagagcccaggccataattgacaccacagatgattcgactgccacagaaagtgcaagctccagtgtcatgtcccccagcttctcttctgagtga

SEQ ID NO: 2 - DNA, 3429 bp, Homo sapiens
atgggcgacaaggacatgcccaccgccgggatgccgagcctgctccagtccagctccgagagcccccagtcctgccccgagggcgaggacagccagtcccccctgcagatcccgcagagctcccccgagagcgacgacaccctgtaccccctccagtccccgcagagccggtccgagggggaggacagctccgacccgctgcagcgcccccccgagggcaaggacagccagtccccgctgcagatcccgcagagctcccccgagggggacgacacgcagagccccctccagaacagccagtccagccccgagggcaaggactccctgagcccgctggagatctcccagagcccccccgagggcgaggacgtgcagtccccgctccagaacccggccagctccttcttcagctccgcgctgctgagcatcttccagtccagccccgagtccacccagagccccttcgaggggttcccccagtccgtcctccagatcccggtgagcgccgcctccagcagcaccctggtgtccatcttccagagctcccccgagagcacccagtcccccttcgagggcttcccccagagcccgctgcagatccccgtgtcccggagcttctccagcacgctcctgtccatcttccagagctcccccgagcgcacccagagcaccttcgaggggttcgcccagtccccgctgcagatccccgtgagcccctccagcagctccaccctcctgagcctgttccagtccttcagcgagcggacgcagtccaccttcgagggcttcgcccagagctccctccagatccccgtgagcccgtccttcagctccaccctggtcagcctgttccagtccagccccgagcgcacccagtccacgttcgaggggttcccccagagccccctccagatcccggtgtccagctccagcagctccaccctgctgagcctcttccagtccagccccgagcggacccactccaccttcgagggcttcccccagagcctgctgcagatccccatgacgtccagcttctccagcaccctcctgtccatcttccagagctccccggagagcgcgcagtccaccttcgagggcttcccccagagccccctgcagatccccgggtccccgagcttctccagcaccctcctgagcctgttccagtccagccccgagcgcacgcactccaccttcgagggcttcccccagagccccctccagatcccgatgacctccagcttctccagcaccctgctgtccatcctccagagctcccccgagagcgcccagtccgccttcgaggggttcccccagagccccctgcagatcccggtgtccagctccttcagctacacgctgctctccctgttccagagcagccccgagcggacccactccaccttcgagggcttcccccagagcccgctgcagatccccgtgtccagctccagctccagctccaccctcctgagcctgttccagtccagccccgagtgcacgcagtccaccttcgagggcttcccccagagcccgctgcagatcccccagtccccccccgagggggagaacacccacagcccgctccagatcgtgccctccctgcccgagtgggaggacagcctgtccccgcactacttcccgcagagccccccgcagggcgaggacagcctctccccccactacttcccgcagagcccgccccagggggaggactccctgagcccccactacttcccgcagtccccccagggcgaggacagcctgtccccgcactacttcccccagagcccgccccagggggaggactccatgagccccctctacttcccccagtccccgctgcagggcgaggagttccagagctccctgcagagccccgtgtccatctgcagctccagcaccccctccagcctcccgcagagcttccccgagtccagccagtccccccccgagggcccggtccagagccccctgcactccccgcagagccccccggaggggatgcactcccagagccccctgcagtcccccgagagcgcccccgagggcgaggactccctcagcccgctgcagatcccccagtccccgctggagggggaggacagcctctccagcctgcacttcccccagtccccgcccgagtgggaggacagcctgagccccctccacttcccccagttcccgccccagggcgaggacttccagtccagcctgcagtcccccgtgagcatctgctccagctccacgagcctgtccctcccccagagcttcccggagtccccccagagcccgcccgaggggccggcgcagtcccccctgcagcgccccgtgagctccttcttcagctacaccctggcctccctcctgcagagctcccacgagagcccgcagagcccgcccgagggccccgcccagtccccgctgcagagccccgtgtccagcttcccctccagcacctccagctccctcagccagtccagccccgtgtccagcttcccgtccagcacctccagctccctgagcaagagctcccccgagagccccctgcagtcccccgtgatcagcttctccagctccacgagcctctccccgttcagcgaggagtccagctcccccgtcgacgagtacaccagctccagcgacaccctgctggagtccgacagcctcaccgactccgagagcctgatcgagagcgagcccctgttcacctacacgctcgacgagaaggtggacgagctggcccggttcctgctcctgaagtaccaggtgaagcagcccatcaccaaggccgagatgctgaccaacgtcatctcccgctacaccggctacttcccggtgatcttccggaaggcgcgcgagttcatcgagatcctcttcgggatcagcctgcgggaggtggaccccgacgactcctacgtcttcgtgaacacgctggacctcaccagcgagggctgcctgtccgacgagcaggggatgagccagaaccgcctgctcatcctgatcctgtccatcatcttcatcaagggcacctacgccagcgaggaggtcatctgggacgtgctctccgggatcggcgtgcgggccggccgcgagcacttcgccttcggggagccccgggagctgctgaccaaggtctgggtgcaggagcactacctcgagtaccgcgaggtgcccaacagctccccgccccggtacgagttcctgtggggcccccgcgcccacagcgaggtcatcaagcggaaggtggtggagttcctggcgatgctcaagaacacggtccccatcaccttcccgtccagctacaaggacgccctgaaggacgtggaggagcgggcccaggccatcatcgacaccaccgacgactccacggccaccgagagcgcgtccagctccgtgatgagccccagcttctccagcgagtga

SEQ ID NO: 3 - DNA, 1596 bp, Homo sapiens
atgcagtccccgctgcagggcgaggagttccagagctccctgcagagccccgtgtccatctgcagctccagcaccccctccagcctcccgcagagcttccccgagtccagccagtccccccccgagggcccggtccagagccccctgcactccccgcagagccccccggaggggatgcactcccagagccccctgcagtcccccgagagcgcccccgagggcgaggactccctcagcccgctgcagatcccccagtccccgctggagggggaggacagcctctccagcctgcacttcccccagtccccgcccgagtgggaggacagcctgagccccctccacttcccccagttcccgccccagggcgaggacttccagtccagcctgcagtcccccgtgagcatctgctccagctccacgagcctgtccctcccccagagcttcccggagtccccccagagcccgcccgaggggccggcgcagtcccccctgcagcgccccgtgagctccttcttcagctacaccctggcctccctcctgcagagctcccacgagagcccgcagagcccgcccgagggccccgcccagtccccgctgcagagccccgtgtccagcttcccctccagcacctccagctccctcagccagtccagccccgtgtccagcttcccgtccagcacctccagctccctgagcaagagctcccccgagagccccctgcagtcccccgtgatcagcttctccagctccacgagcctctccccgttcagcgaggagtccagctcccccgtcgacgagtacaccagctccagcgacaccctgctggagtccgacagcctcaccgactccgagagcctgatcgagagcgagcccctgttcacctacacgctcgacgagaaggtggacgagctggcccggttcctgctcctgaagtaccaggtgaagcagcccatcaccaaggccgagatgctgaccaacgtcatctcccgctacaccggctacttcccggtgatcttccggaaggcgcgcgagttcatcgagatcctcttcgggatcagcctgcgggaggtggaccccgacgactcctacgtcttcgtgaacacgctggacctcaccagcgagggctgcctgtccgacgagcaggggatgagccagaaccgcctgctcatcctgatcctgtccatcatcttcatcaagggcacctacgccagcgaggaggtcatctgggacgtgctctccgggatcggcgtgcgggccggccgcgagcacttcgccttcggggagccccgggagctgctgaccaaggtctgggtgcaggagcactacctcgagtaccgcgaggtgcccaacagctccccgccccggtacgagttcctgtggggcccccgcgcccacagcgaggtcatcaagcggaaggtggtggagttcctggcgatgctcaagaacacggtccccatcaccttcccgtccagctacaaggacgccctgaaggacgtggaggagcgggcccaggccatcatcgacaccaccgacgactccacggccaccgagagcgcgtccagctccgtgatgagccccagcttctccagcgagtga

SEQ ID NO: 4 - DNA, 1122 bp, Homo sapiens
atgcctcccgttccaggcgttccattccgcaacgttgacaacgactccccgacctcagttgagttagaagactgggtagatgcacagcatcccacagatgaggaagaggaggaagcctcctccgcctcttccactttgtacttagtattttccccctcttctttctccacatcctcttctctgattcttggtggtcctgaggaggaggaggtgccctctggtgtgataccaaatcttaccgagagcattcccagtagtcctccacagggtcctccacagggtccttcccagagtcctctgagctcctgctgctcctctttttcatggagctcattcagtgaggagtccagcagccagaaaggggaggatacaggcacctgtcagggcctgccagacagtgagtcctctttcacatatacactagatgaaaaggtggccgagttagtggagttcctgctcctcaaatacgaagcagaggagcctgtaacagaggcagagatgctgatgattgtcatcaagtacaaagattactttcctgtgatactcaagagagcccgtgagttcatggagcttctttttggccttgccctgatagaagtgggccctgaccacttctgtgtgtttgcaaacacagtaggcctcaccgatgagggtagtgatgatgagggcatgcccgagaacagcctcctgattattattctgagtgtgatcttcataaagggcaactgtgcctctgaggaggtcatctgggaagtgctgaatgcagtaggggtatatgctgggagggagcacttcgtctatggggagcctagggagctcctcactaaagtttgggtgcagggacattacctggagtatcgggaggtgccccacagttctcctccatattatgaattcctgtggggtccaagagcccattcagaaagcatcaagaagaaagtactagagtttttagccaagctgaacaacactgttcctagttcctttccatcctggtacaaggatgctttgaaagatgtggaagagagagtccaggccacaattgataccgcagatgatgccactgtcatggccagtgaaagcctcagtgtcatgtccagcaacgtctccttttctgagtga

SEQ ID NO: 5 - DNA, 1122 bp, Homo sapiens
atgcccccggtgcccggcgtccccttccggaacgtggacaacgacagccccacctccgtggagctggaggactgggtcgacgcccagcacccgaccgacgaggaggaggaggaggccagctccgcgagctccacgctctacctggtgttcagcccctccagcttctccaccagctccagcctgatcctcgggggccccgaggaggaggaggtgccctccggggtcatcccgaacctgaccgagagcatcccctccagccccccgcagggcccgccccaggggccctcccagagccccctgtccagctgctgcagctccttcagctggtccagcttctccgaggagagctccagccagaagggcgaggacaccggcacgtgccaggggctcccggactccgagagctccttcacctacaccctggacgagaaggtggccgagctggtggagttcctcctgctgaagtacgaggccgaggagcccgtcaccgaggccgagatgctcatgatcgtgatcaagtacaaggactacttccccgtgatcctgaagcgcgcccgggagttcatggagctgctcttcggcctggcgctgatcgaggtcgggcccgaccacttctgcgtgttcgccaacacggtgggcctcaccgacgaggggagcgacgacgagggcatgccggagaactccctgctgatcatcatcctcagcgtcatcttcatcaagggcaactgcgcctccgaggaggtgatctgggaggtgctgaacgccgtcggggtgtacgcgggccgcgagcacttcgtgtacggggagccccgggagctgctcaccaaggtctgggtgcagggccactacctggagtaccgcgaggtgccgcacagctcccccccgtactacgagttcctgtggggcccccgggcccacagcgagtccatcaagaagaaggtcctcgagttcctggccaagctgaacaacaccgtgcccagcagcttcccctcctggtacaaggacgccctcaaggacgtcgaggagcgcgtgcaggccacgatcgacaccgcggacgacgccaccgtgatggccagcgagtccctgagcgtcatgtccagcaacgtgtccttcagcgagtga

SEQ ID NO: 6 - DNA, 543 bp, Homo sapiens
atgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaa

SEQ ID NO: 7 - DNA, 543 bp, Homo sapiens
atgcaggccgagggccgcggcaccggcggctcgaccggcgacgccgacgggcccggcggcccgggcatcccggacggcccgggcgggaacgcgggcggcccgggcgaggccggcgccaccggcgggcggggcccgcggggcgccggcgccgcccgggcgagcggccccggcgggggcgccccgcggggcccgcacggcggcgccgccagcggcctgaacgggtgctgccggtgcggcgcccgcggcccggagagccggctcctggagttctacctggccatgccgttcgcgaccccgatggaggccgagctggcccggcggagcctggcccaggacgccccgccgctgcccgtgccgggcgtgctcctgaaggagttcacggtgagcggcaacatcctgaccatccggctgaccgccgcggaccaccggcagctgcagctgtcgatcagcagctgcctccagcagctgagcctgctgatgtggatcacccagtgcttcctgccggtgttcctggcccagccgcccagcggccagcgccggtga

SEQ ID NO: 8 - DNA, 429 bp, Homo sapiens
atgggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcgaagaaagtgcgccgtgccatcgagcagctggctgccatggattga

SEQ ID NO: 9 - DNA, 429 bp, Homo sapiens
atgggcgcccccaccctgccgccggcctggcagccgttcctcaaggaccaccgcatctcgaccttcaagaactggccgttcctggagggctgcgcgtgcaccccggagcggatggccgaggccggcttcatccactgccccaccgagaacgagccggacctggcccagtgcttcttctgcttcaaggagctggagggctgggagccggacgacgacccgatcgaggagcacaagaagcacagcagcggctgcgccttcctgagcgtgaagaagcagttcgaggagctgacgctcggggagttcctgaagctggaccgggagcgggccaagaacaagatcgcgaaggagaccaacaacaagaagaaggagttcgaggagaccgccaagaaggtgcggcgggccatcgagcagctggccgccatggactga

SEQ ID NO: 10 - DNA, 1263 bp, Homo sapiens
atgcctggggggtgctcccggggccccgccgccggggacgggcgtctgcggctggcgcgactagcgctggtactcctgggctgggtctcctcgtcttctcccacctcctcggcatcctccttctcctcctcggcgccgttcctggcttccgccgtgtccgcccagcccccgctgccggaccagtgccccgcgctgtgcgagtgctccgaggcagcgcgcacagtcaagtgcgttaaccgcaatctgaccgaggtgcccacggacctgcccgcctacgtgcgcaacctcttccttaccggcaaccagctggccgtgctccctgccggcgccttcgcccgccggccgccgctggcggagctggccgcgctcaacctcagcggcagccgcctggacgaggtgcgcgcgggcgccttcgagcatctgcccagcctgcgccagctcgacctcagccacaacccactggccgacctcagtcccttcgctttctcgggcagcaatgccagcgtctcggcccccagtccccttgtggaactgatcctgaaccacatcgtgccccctgaagatgagcggcagaaccggagcttcgagggcatggtggtggcggccctgctggcgggccgtgcactgcaggggctccgccgcttggagctggccagcaaccacttcctttacctgccgcgggatgtgctggcccaactgcccagcctcaggcacctggacttaagtaataattcgctggtgagcctgacctacgtgtccttccgcaacctgacacatctagaaagcctccacctggaggacaatgccctcaaggtccttcacaatggcaccctggctgagttgcaaggtctaccccacattagggttttcctggacaacaatccctgggtctgcgactgccacatggcagacatggtgacctggctcaaggaaacagaggtagtgcagggcaaagaccggctcacctgtgcatatccggaaaaaatgaggaatcgggtcctcttggaactcaacagtgctgacctggactgtgacccgattcttcccccatccctgcaaacctcttatgtcttcctgggtattgttttagccctgataggcgctattttcctcctggttttgtatttgaaccgcaaggggataaaaaagtggatgcataacatcagagatgcctgcagggatcacatggaagggtatcattacagatatgaaatcaatgcggaccccagattaacgaacctcagttctaactcggatgtctga

SEQ ID NO: 11 - DNA, 1263 bp, Homo sapiens
atgcccggcgggtgcagccggggcccggccgccggggacggccgcctgcggctcgcgcgcctggccctggtgctcctggggtgggtctccagctccagccccacctccagcgcctccagcttctccagctccgcccccttcctggccagcgcggtgtccgcccagcccccgctccccgaccagtgccccgccctgtgcgagtgcagcgaggccgcgcggaccgtgaagtgcgtcaaccgcaacctgacggaggtgcccaccgacctcccggcctacgtgcggaacctgttcctgaccggcaaccagctcgccgtcctgcccgccggcgccttcgcgcgccggccgcccctggccgagctcgccgccctgaacctgtccgggagccgcctcgacgaggtgcgggccggcgcgttcgagcacctgccgtccctgcgccagctcgacctgagccacaaccccctggccgacctctcccccttcgccttcagcgggagcaacgcctccgtgagcgccccctccccgctggtcgagctgatcctcaaccacatcgtgccccccgaggacgagcggcagaaccgcagcttcgagggcatggtggtcgcggccctgctggccgggcgggccctccagggcctgcgccggctggagctcgcctccaaccacttcctgtacctgccccgcgacgtgctcgcgcagctgccgagcctgcggcacctcgacctgtccaacaacagcctggtgtccctcacctacgtcagcttccgcaacctgacgcacctggagtccctccacctggaggacaacgccctgaaggtgctgcacaacggcaccctcgccgagctgcaggggctgccccacatccgggtgttcctcgacaacaacccctgggtctgcgactgccacatggccgacatggtgacctggctgaaggagaccgaggtggtccagggcaaggaccgcctgacgtgcgcgtaccccgagaagatgcggaaccgggtgctcctggagctgaacagcgccgacctcgactgcgacccgatcctgcccccctccctgcagaccagctacgtgttcctcgggatcgtcctggccctgatcggcgccatcttcctcctggtgctgtacctcaaccgcaagggcatcaagaagtggatgcacaacatccgggacgcctgccgcgaccacatggaggggtaccactaccggtacgagatcaacgcggacccccgcctgaccaacctgtccagcaactccgacgtctga

(19) United States
(12) Patent Application Publication
(10) Pub. No.: US 2019/0194760 A1
(43) Pub. Date: Jun. 27, 2019

(54) NOVEL BIOMARKERS

(71) Applicant: CureVac AG, Tübingen (DE)

(72) Inventors: Sven KOCH, München (DE); Henoch HONG, Heidelberg (DE); Mariola FOTIN-MLECZEK, Sindelfingen (DE)

(21) Appl. No.: 16/304,381

(22) PCT Filed: May 24, 2017
(86) PCT No.: PCT/EP2017/062694

§ 371 Date: Nov. 26, 2018

(30) Foreign Application Priority Data
May 25, 2016 (EP) PCT/EP2016/061829

(51) Int. Cl. C12Q 1/6886

(52) U.S. Cl. CPC C12Q 1/6886; C12Q 2600/106; C12Q 2600/158; C12Q 2600/118

(57) ABSTRACT

The present invention relates to methods of diagnosing, monitoring of a subject or determining the prognosis of a subject. In particular, the invention relates to a method of determining the prognosis of a cancer patient comprising the steps (a) determining the expression level of at least one marker gene selected from the group consisting of the marker genes as described herein in a sample of the cancer patient to obtain a gene expression profile; (b) determining the prognosis of the cancer patient based on the gene expression profile obtained in step (a). In addition, the present invention refers to kits, diagnostic compositions devices and microarrays for determining at least one marker gene and uses thereof.

### FIELD OF THE INVENTION

The present invention relates to methods of diagnosing, monitoring of a subject or determining the prognosis of a subject. In particular, the invention relates to a method of determining the prognosis of a cancer patient comprising the steps (a) determining the expression level of at least one marker gene selected from the group consisting of the marker genes as described herein in a sample of the cancer patient to obtain a gene expression profile; (b) determining the prognosis of the cancer patient based on the gene expression profile obtained in step (a). In addition, the present invention refers to kits, diagnostic compositions devices and microarrays for determining at least one marker gene and uses thereof.

### BACKGROUND OF THE INVENTION

Technological advances have greatly increased our understanding of the molecular basis of diseases. In the past, biomarkers have been developed that can be used for a range of applications including predicting disease risk, diagnosis, predicting prognosis, identifying appropriate therapy for an individual, monitoring disease or for return of a disease, predicting the survival of a patient and other applications.

However, new diagnostic, prognostic and predictive biomarkers are necessary to improve disease prognosis and prediction.

In particular, in the treatment of cancer, for example lung cancer or prostate cancer, improved biomarkers are necessary. Lung cancer is the leading cause of cancer-related deaths world-wide and 85-90% of those malignancies are classified as non-small cell lung cancer (NSCLC). For decades, clinical outcomes of patients with advanced NSCLC were poor with 5-year survival rates of less than 5%. However, immunotherapeutic approaches, in particular checkpoint inhibitors, have recently dramatically impacted the field of cancer therapy. The aim of immunotherapy is to induce and activate host immune responses against the tumor. Antibodies blocking the interaction of inhibitory T cell receptors CTLA-4 or PD-1 with their ligands were demonstrated to improve survival rates in patients with metastasized melanoma and NSCLC. In addition, encouraging long lasting responses have been observed in a variety of solid tumors and hematologic malignancies after treatment with antibodies blocking PD-1 or PD-L1. Whereas these clinical results are highly encouraging, a substantial portion of patients fail to benefit from checkpoint inhibition treatment. Furthermore, immune-mediated adverse effects occur even in patients who do not respond to treatment.

Thus, there is a need for new biomarkers for prognosis and prediction in diseases, in particular in cancer, such as lung cancer or prostate cancer.

### SUMMARY OF THE INVENTION

To address these need, the present invention relates to a method of diagnosing, monitoring of a subject or determining the prognosis of a subject comprising the steps of (a) determining the expression level of at least one marker gene selected from the group consisting of the genes set out in table 1 in a sample of the subject to obtain a gene expression profile; (b) diagnosing, monitoring of a subject or determining the prognosis of the subject based on the gene expression profile obtained in step (a).

In particular, the invention relates to a method of determining the prognosis of a cancer patient comprising the steps of

(a) determining the expression level of at least one marker gene selected from the group consisting of the marker genes set out in table 1 in a sample of the cancer patient to obtain a gene expression profile;
(b) determining the prognosis of the cancer patient based on the gene expression profile obtained in step (a).

In specific embodiments, the at least one marker gene is selected from the group consisting of genes set out in table 2. In preferred embodiments, the at least one marker gene is selected from the group consisting of marker genes set out in table 3. In more preferred embodiments, the at least one marker gene is selected from group consisting of marker genes set out in table 4. In even more preferred embodiments, the at least one marker gene is selected from group consisting of marker genes set out in table 5.

In specific embodiments, the at least one marker gene is selected from the group consisting of genes set out in table 2B. In preferred embodiments, the at least one marker gene is selected from the group consisting of marker genes set out in table 3B. In more preferred embodiments, the at least one marker gene is selected from group consisting of marker genes set out in table 4B. In even more preferred embodiments, the at least one marker gene is selected from group consisting of marker genes set out in table 5B.

In some embodiments at least 10 marker genes are selected from table 1. Preferably at least 30 marker genes, more preferably at least 50 marker genes, most preferably at least 100 marker genes are selected from table 1. In some embodiments at least 10 marker genes are selected from table 2; preferably at least 30 marker genes, more preferably at least 50 marker genes, most preferably at least 100 marker genes are selected from table 2. In other embodiments at least 10 genes are selected from table 3; preferably at least 30 marker genes, more preferably at least 50 marker genes are selected from table 3. In further embodiments at least 10 genes are selected from table 4. Preferably at least 20 marker genes, more preferably at least 30 marker genes are selected from table 4. In some embodiments at least 10 marker genes are selected from table 5.

In some embodiments at least 10 marker genes are selected from table 2B; preferably at least 30 marker genes, more preferably at least 50 marker genes, most preferably at least 90 marker genes are selected from table 2B. In other embodiments at least 10 genes are selected from table 3B; preferably at least 30 marker genes, more preferably at least 40 marker genes are selected from table 3B. In further embodiments at least 10 genes are selected from table 4B. Preferably at least 20 marker genes, more preferably at least 25 marker genes are selected from table 4B. In some embodiments at least 8 marker genes are selected from table 5B.

In specific embodiments, the gene expression profile of step (a) is obtained by determining the difference of the expression level of the at least one marker gene measured before administration of the therapeutic agent and after administration of at least one dose of the therapeutic agent.

Typically, the therapeutic agent is any agent used for therapy of a disease, preferably of cancer or tumor diseases. Preferably the therapeutic agent is an immunostimulatory composition and/or a vaccine and/or an immunotherapeutic agent.

In a preferred embodiment, the immunostimulatory composition and/or vaccine comprises at least one antigen selected from the group consisting of MAGE-C1, MAGE-C2, NY-ESO-1, Survivin and 5T4. Preferably, the immunostimulatory composition and/or vaccine comprises the antigens MAGE-C1, MAGE-C2, NY-ESO-1, Survivin and 5T4 or fragments or variants thereof.

Typically, the antigen(s) are present as peptides or proteins and/or are encoded by at least one nucleotide sequence. In preferred embodiments, the antigen(s) are encoded by at least one mRNA molecule.

The invention refers particularly to the prognosis of lung cancer, more particularly to non-small cell lung cancer (NSCLC).

Typically, the sample of the patient comprises peripheral blood mono-nuclear cells (PBMCs).

In some embodiments, in step (b) a hierarchical clustering algorithm is applied.

A further aspect of the invention relates to a kit, diagnostic composition or device for the analysis of at least one marker gene set out in table 1 comprising at least one primer and/or probe selective for determining the expression level of at least one marker gene set out in table 1. The kit, diagnostic composition or device may further comprise an enzyme for primer elongation, nucleotides and/or labeling agents. Also contemplated is the use of said kit, diagnostic composition or device for determining the prognosis of a cancer patient.

A further aspect of the invention relates to a microarray, comprising at least one probe selective for determining the expression level of at least one marker gene set out in table 1 and the use of said microarray for determining the prognosis of a cancer patient.

More specifically, the invention refers to a method of determining the prognosis of a patient comprising the steps of
(a) determining the expression level of at least 30 marker genes selected from the group consisting of the marker genes set out in table 4 in a sample of the patient to obtain a gene expression profile;
(b) determining the prognosis of the patient based on the gene expression profile obtained in step (a).

The inventor found out that by the 30 marker genes of table 4 is possible to classify cancer patients (FIG. 3 C). In a specific embodiment, the patient is a lung cancer patient, preferably a NSCLC patient.

In specific embodiments further marker genes such as least 10 additional marker genes, preferably at least 20 additional marker genes, more preferably at least 70 additional marker genes are selected from table 1, table 2 or table 3 are employed for the determining the prognosis of the patient.

The invention further refers to a method of determining the prognosis of a patient comprising the steps of
(a) determining the expression level of at least one marker gene selected from the group consisting of the marker genes set out in table 11 in a sample of the patient to obtain a gene expression profile;
(b) determining the prognosis of the patient based on the gene expression profile obtained in step (a).

The inventors found that the marker genes set out in tables 11 to 15 are particular suitable to classify both lung cancer and prostate cancer patients. Therefore, the patient may be cancer or tumor patient, in particular a lung cancer or prostate cancer patient. In a specific embodiment, the cancer patient is a NSCLC cancer or prostate cancer patient.

In specific embodiments the at least one marker genes is selected from the group consisting of marker genes set out in table 12, table 13, table 14 or table 15.

In specific embodiments least 10 marker genes, preferably at least 30 marker genes, more preferably at least 50 marker genes, most preferably at least 100 marker genes are selected from table 11 or table 12. In a further embodiment, at least 10 marker genes, preferably at least 30 marker genes, more preferably at least 50 marker genes are selected from table 13. In another embodiment at least 10 marker genes, preferably at least 20 marker genes, more preferably at least 30 marker genes are selected from the group consisting of from table 14.

In some embodiments, the expression level of at least one marker gene is measured before administration and/or after administration of at least one dose of a therapeutic agent. Typically, the therapeutic agent is an immunostimulatory composition and/or a vaccine and/or an immunotherapeutic agent. In one embodiment, the immunostimulatory composition and/or vaccine comprises at least one antigen selected from the group consisting of MAGE-C1, MAGE-C2, NY-ESO-1, Survivin, 5T4, PSA, PSMA, PSCA, STEAP, PAP and MUC1 or fragments or variants thereof. In a specific embodiment, the immunostimulatory composition and/or vaccine comprises the antigens (i) MAGE-C1, MAGE-C2, NY-ESO-1, Survivin and 5T4 or fragments or variants thereof; or (ii) PSA, PSMA, PSCA, STEAP, PAP and MUC1 or fragments or variants thereof.

Accordingly, a further aspect of the invention refers to a kit, diagnostic composition or device for the analysis of at least one marker gene set out in table 11 comprising at least one primer and/or probe selective for determining the expression level of at least one marker gene set out in table 11.

Another aspect refers to a microarray, comprising at least one probe selective for determining the expression level of at least one marker gene set out in table 11.

Another aspect of the invention refers to a method of determining the prognosis of a patient comprising the steps of
(a) determining the expression level of at least one marker gene selected from the group consisting of the marker genes set out in table 6 in a sample of the patient to obtain a gene expression profile;
(b) determining the prognosis of the patient based on the gene expression profile obtained in step (a).

The inventors found that the marker genes set out in tables 6 to 10 are particularly suitable to classify prostate cancer patients. Accordingly, the patient may be a cancer or tumor patient, in particular a prostate cancer patient. Therefore, in one embodiment, the immunostimulatory composition and/or vaccine comprises at least one antigen selected from the group consisting of PSA, PSMA, PSCA, STEAP, PAP and MUC1 or fragments or variants thereof.

Accordingly, another aspect of the invention refers to a kit, diagnostic composition or device for the analysis of at least one marker gene set out in table 6 comprising at least one primer and/or probe selective for determining the expression level of at least one marker gene set out in table 6.

An additional aspect refers to a microarray, comprising at least one probe selective for determining the expression level of at least one marker gene set out in table 6.

### BRIEF DESCRIPTION OF THE FIGURES

FIG. 1: Transcriptional changes post treatment cluster patients into distinct groups. Week 5 to 0 BTM activity score differences were calculated and unsupervised hierarchical clustering was performed.

FIG. 2: Patients with NK and T cell BTM enrichment post CV9201 treatment are associated with a prolonged survival. Kaplan-Meier curves for overall survival (A) and progression-free survival (C) are shown for patients belonging to cluster 1, 2a and 2b. Overall survival (B) and progression-free survival rates (D) are shown for patients belonging to cluster 1 compared to patients belonging to cluster 2. Log-rank test was performed to calculate the hazard ratio and p value.

FIG. 3. Identification of short-term survivors using hierarchical clustering on gene expression differences between post-vaccine time point week 5 and baseline week 0. Genes were ranked based on their ability to differentiate between patients derived from cluster 1 and patients derived from cluster 2. Top 100 (A), top 50 (B), or top 30 (C) genes were selected, and gene expression differences between week 5 and 0 were calculated followed by average-linkage hierarchical clustering. Clustering trees of patients are shown and short-term survivors are highlighted.

FIG. 4: Transcriptional modules consistent with a T and NK cell profile are enriched at week 6 post initiation of CV9104 treatment. Results from gene set enrichment analyses contrasting post treatment week 6 samples to baseline samples are shown for cohorts A, B, C and A&B combined. The top 10 most enriched modules are presented.

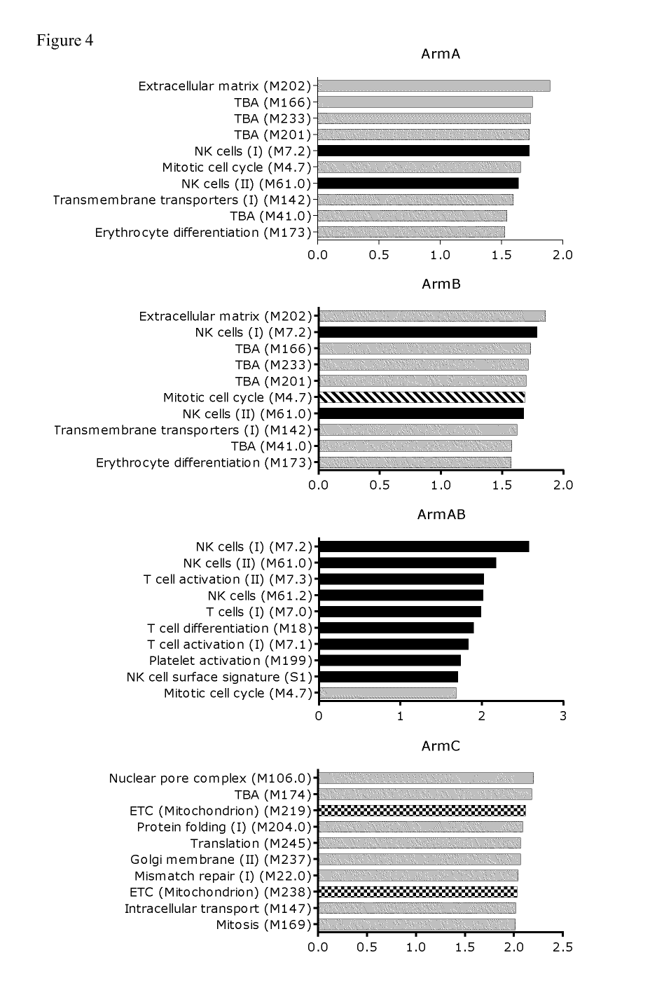

### DEFINITIONS

For the sake of clarity and readability, the following definitions are provided. Any technical feature mentioned for these definitions may be read on each and every embodiment of the invention. Additional definitions and explanations may be specifically provided in the context of these embodiments. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization are those well known and commonly employed in the art. Standard techniques are used for molecular biotechnology assays. The techniques and procedures are generally performed according to conventional methods in the art and various general references (e.g., Sambrook et al., 1989, Molecular Cloning: A Laboratory Manual, 2d ed. Cold Spring Harbor Laboratory Press, Cold Spring Harbor, N.Y.), which are provided throughout this document.

Marker, marker gene: The terms “marker” or “maker gene”, can be used interchangeably and relate to a gene, genetic unit or sequence (a nucleotide sequence or amino acid or protein sequence) as defined herein, the term also refers to any expression product of said genetic unit or sequence, in particular mRNA transcript, a polypeptide or protein encoded by the transcript or fragments thereof, as well as homologous derivatives thereof as described herein above. In particular, the term marker gene refers to a gene set out in table 1. Preferably, the marker gene is selected from the group consisting of genes set out in table 2, more preferably from the group consisting of genes set out in table 3, even more preferably from group consisting of genes set out in table 4, most preferably at least from group consisting of genes set out in table 5.

Subject, individual, patient: A subject, individual or patient according to the present invention is an animal, preferably a mammal, more preferably a human being. A subject, individual or patient according to the present invention is an animal, preferably a mammal, more preferably a human being. The method also relates to healthy subjects and to patients. In particular, the method of the invention refers to patients who are cancer or tumor patients. A cancer or tumor patient is a subject or individual who is diagnosed as having cancer or tumor.

non-small-cell lung cancer (NSCLC): non-small-cell lung cancer as used herein refers to the three main sub-types of non-small-cell lung cancer including, without being restricted thereto, squamous cell lung carcinoma, adenocarcinoma and large cell lung carcinoma.

determining the prognosis of a patient: The term “determining the prognosis of a patient” (and also indication specific terms such as “determining the prognosis of a cancer or tumor patient”) as used herein refers to the prediction of the course or outcome of a diagnosed or detected disease, e.g. during a certain period of time, before a treatment, during a treatment or after a treatment. The term also refers to a determination of chance of survival or recovery from the disease, as well as to a prediction of the expected survival time of a subject. A prognosis may, specifically, involve establishing the likelihood for survival of a subject during a period of time into the future, such as 6 months, 1 year, 2 years, 3 years, 5 years, 10 years or any other period of time. In a specific embodiment the prognosis may involve predicting the survival of a patient, i.e. whether the survival of a subject is shorter than or equal to a certain period of time, such as 6 months, 12 months or 15 months or any other period of time, or longer than a certain period of time, such as 6 months, 12 months, 15 months or 30 months or any other period of time. For example, the prognosis may involve a prediction whether the survival time of a cancer patient is equal or shorter than 15 months or whether the survival time of a cancer patient is longer than 15 months.

Obtain a gene expression profile: The gene expression profile is obtained by relating the expression level of the at least one marker gene to a reference value. The reference value may be for example the expression level of a control gene measured in the same sample of the individual. Alternatively, the reference value may be a control expression level derived from a healthy control sample or a sample from a pathological sample (of the disease of interest). The pathological sample may be for example from a patient with tumor or cancer (e.g. progressive tumor or non-progressive tumor). Alternatively, the reference value may be the expression level measured at a different time point in the same patient. The different time point may be a different treatment stage, e.g. before treatment, during treatment, after treatment, after the administration of a certain amount of treatment doses. The different time point may be any type of periodical time segment, such as one week, 2 weeks one months, 2, 3, 4, 5, 6, 7, 8, 10, 11 or 12 months, 1.5 years, 2, 3, 4, 5, 6, 7, 8, 9, 10 years ago. In a preferred embodiment the gene expression profile is obtained by relation of the expression level of the at least one marker gene after administration of at least one dose of therapeutic agent to the expression level of the at least one marker gene before administration of the therapeutic agent.

Monitoring: The term “monitoring” as used herein relates to the accompaniment of a diagnosed or detected disease or disorder, e.g. during a treatment procedure or during a certain period of time, typically during 2 months, 3 months, 4 months, 6 months, 1 year, 2 years, 3 years, 5 years, 10 years, or any other period of time, changes of these sates of disease may be detected by comparing the expression. The term “accompaniment” means that states of disease as defined herein above and, in particular level of the marker genes of the present invention in a sample are measured and a gene expression profile is obtained by comparison to a reference value in any type of periodical time segment, e.g. every week, every 2 weeks, every month, every 2, 3, 4, 5, 6, 7, 8, 9, 10, 11 or 12 month, every 1.5 year, every 2, 3, 4, 5, 6, 7, 8, 9 or 10 years, during any period of time, e.g. during 2 weeks, 3 weeks, 1, 2, 3, 4, 5, 6, 7, 8, 9, 10, 11, 12 months, 1.5, 2, 3, 4, 5, 6, 7, 8, 9, 10, 15 or 20 years, respectively.

Diagnosing: The term “diagnosing” as used herein means that a patient or subject may be considered to be suffering from a disease. In particular, term “diagnosing” as used herein means that a patient or subject may be considered to be suffering from a disease based on the marker gen expression profile of the present invention. The term “diagnosing” also refers to the conclusion reached through that comparison process.

Reference gene: The term “reference gene” or “control gene” as used herein refers to any suitable gene, e.g. to any steadily expressed and continuously detectable gene, gene product, expression product, protein or protein variant in the organism of choice. The term also includes gene products such as expressed proteins, peptides, polypeptides, as well as modified variants thereof. The invention hence also includes reference proteins derived from a reference gene. Also encompassed are all kinds of transcripts derivable from the reference gene as well as modifications thereof or secondary parameters linked thereto. Alternatively, or additionally, other reference parameters may also be used for reference purposes, e.g. metabolic concentrations, cell sizes etc.

Microarray: A (DNA) microarray (also commonly known as DNA chip or biochip) is a collection of microscopic DNA spots attached to a solid surface. DNA microarrays are used to measure the expression levels of large numbers of genes simultaneously or to genotype multiple regions of a genome. Each DNA spot contains picomoles (10-12 moles) of a specific DNA sequence, known as probes (or reporters or oligos). These can be a short section of a gene or other DNA element that are used to hybridize a nucleic acid sample (called target) under high-stringency conditions. Probe-target hybridization is usually detected and quantified by detection of fluorophore-, silver-, or chemiluminescence-labeled targets to determine relative abundance of nucleic acid sequences in the target. A “microarray” is a linear or two-dimensional array of discrete regions, each having a defined area, formed on the surface of a generally solid support such as, but not limited to, glass, plastic, or synthetic membrane. The density of the discrete regions on a microarray is determined by the total numbers of immobilized oligonucleotides to be detected on the surface of a single solid phase support, such as at least about 50/cm2, at least about 100/cm2, at least about 500/cm2, but below about 1,000/cm2 in some embodiments. The arrays may contain less than about 500, about 1000, about 1500, about 2000, about 2500, or about 3000 immobilized oligonucleotides in total. As used herein, a DNA microarray is an array of nucleic acids e.g. oligonucleotides or oligonucleotides placed on a chip or other surfaces used to hybridize to amplified or cloned oligonucleotides from a sample. Because the position of each particular group of oligonucleotides in the array is known, the identities of a sample oligonucleotides can be determined based on their binding to a particular position in the microarray.

Probe: In molecular biology, a hybridization probe is a fragment of DNA or RNA of variable length. It can be used in DNA or RNA samples to detect the presence of nucleotide sequences (the target) that are complementary to the nucleic acid sequence in the probe. The probe thereby hybridizes to single-stranded nucleic acid (DNA or RNA) whose base sequence allows probe-target base pairing due to complementarity between the probe and target.

Amplify: The term “amplify” is used in the broad sense to mean creating an amplification product can be made enzymatically with DNA or RNA polymerases. “Amplification,” as used herein, generally refers to the process of producing multiple copies of a desired sequence, particularly those of a sample. “Multiple copies” mean at least 2 copies. A “copy” does not necessarily mean perfect sequence complementarity or identity to the template sequence. It is possible to further use any sequencing method known in the art to identify the sequences of marker genes.

Protein: A protein typically comprises one or more peptides or polypeptides. A protein is typically folded into a 3-dimensional form, which may be required for the protein to exert its biological function. The sequence of a protein or peptide is typically understood to be the order, i.e. the succession of its amino acids.

Peptide: A peptide or polypeptide is typically a polymer of amino acid monomers, linked by peptide bonds. It typically contains less than 50 monomer units. Nevertheless, the term peptide is not a disclaimer for molecules having more than 50 monomer units. Long peptides are also called polypeptides, typically having between 50 and 600 monomeric units.

Fragment or part of a protein: Fragments or parts of a protein in the context of the present invention are typically understood to be peptides corresponding to a continuous part of the amino acid sequence of a protein, preferably having a length of about 6 to about 20 or even more amino acids, e.g. parts as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule, i.e. the fragments are typically not recognized in their native form. Fragments or parts of the proteins as defined herein may also comprise epitopes or functional sites of those proteins. Preferably, fragments or parts of a proteins in the context of the invention are antigens, particularly immunogens, e.g. antigen determinants (also called ‘epitopes’), or do have antigenic characteristics, eliciting an adaptive immune response. Therefore, fragments of proteins or peptides may comprise at least one epitope of those proteins or peptides. Furthermore, also domains of a protein, like the extracellular domain, the intracellular domain or the transmembrane domain, and shortened or truncated versions of a protein may be understood to comprise a fragment of a protein.

RNA, mRNA: RNA is the usual abbreviation for ribonucleic acid. It is a nucleic acid molecule, i.e. a polymer consisting of nucleotide monomers. These nucleotides are usually adenosine-monophosphate, uridine-monophosphate, guanosine-monophosphate and cytidine-monophosphate monomers, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA-sequence. Usually RNA may be obtainable by transcription of a DNA-sequence, e.g., inside a cell. In eukaryotic cells, transcription is typically performed inside the nucleus or the mitochondria. In vivo, transcription of DNA usually results in the so-called premature RNA, which has to be processed into so-called messenger-RNA, usually abbreviated as mRNA. Processing of the premature RNA, e.g. in eukaryotic organisms, comprises a variety of different posttranscriptional-modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) sequence. Aside from messenger RNA, several non-coding types of RNA exist which may be involved in regulation of transcription and/or translation, and immunostimulation. The term “RNA” further encompass other coding RNA molecules, such as viral RNA, retroviral RNA and replicon RNA, small interfering RNA (siRNA), antisense RNA, CRISPR RNA, ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), and Piwi-interacting RNA (piRNA).

DNA: DNA is the usual abbreviation for deoxyribonucleic acid. It is a nucleic acid molecule, i.e. a polymer consisting of nucleotide monomers. These nucleotides are usually deoxy-adenosine-monophosphate, deoxy-thymidine-monophosphate, deoxy-guanosine-monophosphate and deoxy-cytidine-monophosphate monomers which are—by themselves—composed of a sugar moiety (deoxyribose), a base moiety and a phosphate moiety, and polymerized by a characteristic backbone structure. The backbone structure is, typically, formed by phosphodiester bonds between the sugar moiety of the nucleotide, i.e. deoxyribose, of a first and a phosphate moiety of a second, adjacent monomer. The specific order of the monomers, i.e. the order of the bases linked to the sugar/phosphate-backbone, is called the DNA-sequence. DNA may be single-stranded or double-stranded. In the double stranded form, the nucleotides of the first strand typically hybridize with the nucleotides of the second strand, e.g. by A/T-base-pairing and G/C-base-pairing.

Sequence of a nucleic acid molecule/nucleic acid sequence: The sequence of a nucleic acid molecule is typically understood to be the particular and individual order, i.e. the succession of its nucleotides.

Sequence of amino acid molecules/amino acid sequence: The sequence of a protein or peptide is typically understood to be the order, i.e. the succession of its amino acids.

Vaccine: A vaccine is a biological preparation that provides active acquired immunity to a particular disease. A vaccine typically contains an agent comprising an antigen which induces an adaptive immune response.

Immunotherapeutic agents: The term immunotherapeutic agent refers to agents capable of modulating the components of the immune system. Examples of immunotherapeutic agents are without limitation dendritic cells, T cells, antibodies, checkpoint modulators or cytokines, chemokines, interleukins, immunostimulatory agents and adjuvants. Immunotherapeutic agents include checkpoint modulators, therapeutic antibodies, immune cell therapy, T cell receptors (including CAR T cell therapy), immune system modulators such as cytokines and chemokines and vaccines.

Antigen: In the context of the present invention “antigen” refers typically to a substance, which may be recognized by the immune system, preferably by the adaptive immune system, and is capable of triggering an antigen-specific immune response, e.g. by formation of antibodies and/or antigen-specific T cells as part of an adaptive immune response. Typically, an antigen may be or may comprise a peptide or protein which may be presented by the MHC to T-cells and comprises at least one epitope. In particular, the antigen may be a pathogenic antigen or a tumor antigen.

Tumor antigens: In a further embodiment, the mRNA according to the present invention may encode a protein or a peptide, which comprises a peptide or protein comprising a tumor antigen, a fragment, variant or derivative of said tumor antigen, preferably, wherein the tumor antigen is a melanocyte-specific antigen, a cancer-testis antigen or a tumor-specific antigen, preferably a CT-X antigen, a non-X CT-antigen, a binding partner for a CT-X antigen or a binding partner for a non-X CT-antigen or a tumor-specific antigen, more preferably a CT-X antigen, a binding partner for a non-X CT-antigen or a tumor-specific antigen or a fragment, variant or derivative of said tumor antigen; and wherein each of the nucleic acid sequences encodes a different peptide or protein; and wherein at least one of the nucleic acid sequences encodes for 1A01_HLA-A/m; 1A02; 5T4; ACRBP; AFP; AKAP4; alpha-actinin-_4/m; alpha-methylacyl-coenzyme_A_racemase; ANDR; ART-4; ARTC1/m; AURKB; B2MG; B3GN5; B4GN1; B7H4; BAGE-1; BASI; BCL-2; bcr/abl; beta-catenin/m; BING-4; BIRC7; BRCA1/m; BY55; calreticulin; CAMEL; CASP-8/m; CASPA; cathepsin_B; cathepsin_L; CD1A; CD1B; CD1C; CD1D; CD1E; CD20; CD22; CD276; CD33; CD3E; CD3Z; CD44_Isoform_1; CD44_Isoform_6; CD4; CD52; CD55; CD56; CD80; CD86; CD8A; CDC27/m; CDE30; CDK4/m; CDKN2A/m; CEA; CEAM6; CH3L2; CLCA2; CML28; CML66; COA-1/m; coactosin-like_protein; collagen_XXIII; COX-2; CP1B1; CSAG2; CT45A1; CT55; CT-_9/BRD6; CTAG2_Isoform_LAGE-1A; CTAG2_Isoform_LAGE-1B; CTCFL; Cten; cyclin_B1; cyclin_D1; cyp-B; DAM-10; DEP1A; E7; EF1A2; EFTUD2/m; EGFR; EGLN3; ELF2/m; EMMPRIN; EpCam; EphA2; EphA3; ErbB3; ERBB4; ERG; ETV6; EWS; EZH2; FABP7; FCGR3A_Version_1; FCGR3A_Version_2; FGF5; FGFR2; fibronectin; FOS; FOXP3; FUT1; G250; GAGE-1; GAGE-2; GAGE-3; GAGE-4; GAGE-5; GAGE-6; GAGE7b; GAGE-8_(GAGE-2D); GASR; GnT-V; GPC3; GPNMB/m; GRM3; HAGE; hepsin; Her2/neu; HLA-A2/m; homeobox_NKX3.1; HOM-TES-85; HPG1; HS71A; HS71B; HST-2; hTERT; iCE; IF2B3; IL10; IL-13Ra2; IL2-RA; IL2-RB; IL2-RG; IL-5; IMP3; ITA5; ITB1; ITB6; kallikrein-2; kallikrein-4; KI20A; KIAA0205; KIF2C; KK-LC-1; LDLR; LGMN; LIRB2; LY6K; MAGA5; MAGA8; MAGAB; MAGE-A10; MAGE-A12; MAGE-A1; MAGE-A2; MAGE-A3; MAGE-A4; MAGE-A6; MAGE-A9; MAGE-B10; MAGE-B16; MAGE-B17; MAGE-B1; MAGE-B2; MAGE-B3; MAGE-B4; MAGE-B5; MAGE-B6; MAGE-C1; MAGE-C2; MAGE-C3; MAGE-D1; MAGE-D2; MAGE-D4; MAGE-_E1; MAGE-E1_(MAGE1); MAGE-E2; MAGE-F1; MAGE-H1; MAGEL2; mammaglobin_A; MART-1/melan-A; MART-2; MC1_R; M-CSF; mesothelin; MITF; MMP1_1; MMP7; MUC-1; MUM-1/m; MUM-2/m; MYCN; MYO1A; MYO1B; MYO1C; MYO1D; MYO1E; MYO1F; MYO1G; MYO1H; NA17; NA88-A; Neo-PAP; NFYC/m; NGEP; NPM; NRCAM; NSE; NUF2; NY-ESO-1; OA1; OGT; OS-9; osteocalcin; osteopontin; p53; PAGE-4; PAI-1; PAI-2; PAP; PATE; PAX3; PAX5; PD1L1; PDCD1; PDEF; PECA1; PGCB; PGFRB; Pim-1_-Kinase; Pin-1; PLAC1; PMEL; PML; POTEF; POTE; PRAME; PRDX5/m; PRM2; prostein; proteinase-3; PSA; PSB9; PSCA; PSGR; PSM; PTPRC; RAB8A; RAGE-1; RARA; RASH; RASK; RASN; RGS5; RHAMM/CD168; RHOC; RSSA; RU1; RU2; RUNX1; S-100; SAGE; SART-_1; SART-2; SART-3; SEPR; SERPINB5; SIA7F; SIA8A; SIAT9; SIRT2/m; SOX10; SP17; SPNXA; SPXN3; SSX-1; SSX-2; SSX3; SSX-4; ST1A1; STAG2; STAMP-1; STEAP-1; Survivin-2B; survivin; SYCP1; SYT-SSX-1; SYT-SSX-2; TARP; TCRg; TF2AA; TGFB1; TGFR2; TGM-4; TIE2; TKTL1; TPI/m; TRGV11; TRGV9; TRPC1; TRP-p8; TSG10; TSPY1; TVC_(TRGV3); TX101; tyrosinase; TYRP1; TYRP2; UPA; VEGFR1; WT1; and XAGE1, and a immunoglobulin idiotype of a lymphoid blood cell or a T cell receptor idiotype of a lymphoid blood cell, or a fragment, variant or derivative of said tumor antigen; preferably survivin or a homologue thereof, an antigen from the MAGE-family or a binding partner thereof or a fragment, variant or derivative of said tumor antigen.

Particularly preferred in this context are the tumor antigens NY-ESO-1, 5T4, MAGE-C1, MAGE-C2, Survivin, Muc-1, PSA, PSMA, PSCA, STEAP and PAP.

Most preferred is the combination of the antigens NY-ESO-1, 5T4, MAGE-C1, MAGE-C2, and Survivin.

Therapeutic proteins: Therapeutic proteins as defined herein are peptides or proteins which are beneficial for the treatment of any inherited or acquired disease or which improves the condition of an individual. Particularly, therapeutic proteins plays a big role in the creation of therapeutic agents that could modify and repair genetic errors, destroy cancer cells or pathogen infected cells, treat immune system disorders, treat metabolic or endocrine disorders, among other functions. Furthermore adjuvant proteins, therapeutic antibodies are encompassed by therapeutic proteins and also hormone replacement therapy which is e.g. used in the therapy of women in the menopause. In newer approaches somatic cells of a patient are used to reprogram them into pluripotent stem cells which replace the disputed stem cell therapy. Also these proteins used for reprogramming of somatic cells or used for differentiating of stem cells are defined herein as therapeutic proteins. Furthermore therapeutic proteins may be used for other purposes e.g. wound healing, tissue regeneration, angiogenesis, etc.

Therefore therapeutic proteins can be used for various purposes including treatment of various diseases like e.g. infectious diseases, neoplasms (e.g. cancer or tumor diseases), diseases of the blood and blood-forming organs, endocrine, nutritional and metabolic diseases, diseases of the nervous system, diseases of the circulatory system, diseases of the respiratory system, diseases of the digestive system, diseases of the skin and subcutaneous tissue, diseases of the musculoskeletal system and connective tissue, and diseases of the genitourinary system, independently if they are inherited or acquired.

These and other proteins are understood to be therapeutic, as they are meant to treat the subject by replacing its defective endogenous production of a functional protein in sufficient amounts. Accordingly, such therapeutic proteins are typically mammalian, in particular human proteins.

Furthermore, adjuvant or immunostimulating proteins are also encompassed in the term therapeutic proteins. Adjuvant or immunostimulating proteins may be used in this context to induce, alter or improve an immune response in an individual to treat a particular disease or to ameliorate the condition of the individual.

In this context, adjuvant proteins may be selected from mammalian, in particular human adjuvant proteins, which typically comprise any human protein or peptide, which is capable of eliciting an innate immune response (in a mammal), e.g. as a reaction of the binding of an exogenous TLR ligand to a TLR. More preferably, human adjuvant proteins are selected from the group consisting of proteins, which are components and ligands of the signalling networks of the pattern recognition receptors including TLR, NLR and RLH, including TLR1, TLR2, TLR3, TLR4, TLR5, TLR6, TLR7, TLR8, TLR9, TLR10, TLR11; NOD1, NOD2, NOD3, NOD4, NOD5, NALP1, NALP2, NALP3, NALP4, NALP5, NALP6, NALP6, NALP7, NALP7, NALP8, NALP9, NALP10, NALP11, NALP12, NALP13, NALP14,1 IPAF, NAIP, CIITA, RIG-I, MDA5 and LGP2, the signal transducers of TLR signaling including adaptor proteins including e.g. Trif and Cardif; components of the Small-GTPases signalling (RhoA, Ras, Rac1, Cdc42, Rab etc.), components of the PIP signalling (PI3K, Src-Kinases, etc.), components of the MyD88-dependent signalling (MyD88, IRAK1, IRAK2, IRAK4, TIRAP, TRAF6 etc.), components of the MyD88-independent signalling (TICAM1, TICAM2, TRAF6, TBK1, IRF3, TAK1, IRAK1 etc.); the activated kinases including e.g. Akt, MEKK1, MKK1, MKK3, MKK4, MKK6, MKK7, ERK1, ERK2, GSK3, PKC kinases, PKD kinases, GSK3 kinases, JNK, p38MAPK, TAK1, IKK, and TAK1; the activated transcription factors including e.g. NF-kB, c-Fos, c-Jun, c-Myc, CREB, AP-1, Elk-1, ATF2, IRF-3, IRF-7.

Mammalian, in particular human adjuvant proteins may furthermore be selected from the group consisting of heat shock proteins, such as HSP10, HSP60, HSP65, HSP70, HSP75 and HSP90, gp96, Fibrinogen, TypIII repeat extra domain A of fibronectin; or components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1INH, C4 bp, MCP, DAF, H, I, P and CD59, or induced target genes including e.g. Beta-Defensin, cell surface proteins; or human adjuvant proteins including trif, flt-3 ligand, Gp96 or fibronectin, etc., or any species homolog of any of the above human adjuvant proteins.

Mammalian, in particular human adjuvant proteins may furthermore comprise cytokines which induce or enhance an innate immune response, including IL-1 alpha, IL1 beta, IL-2, IL-6, IL-7, IL-8, IL-9, IL-12, IL-13, IL-15, IL-16, IL-17, IL-18, IL-21, IL-23, TNFalpha, IFNalpha, IFNbeta, IFNgamma, GM-CSF, G-CSF, M-CSF; chemokines including IL-8, IP-10, MCP-1, MIP-1alpha, RANTES, Eotaxin, CCL21; cytokines which are released from macrophages, including IL-1, IL-6, IL-8, IL-12 and TNF-alpha; as well as IL-1R1 and IL-1 alpha.

Therapeutic proteins for the treatment of blood disorders, diseases of the circulatory system, diseases of the respiratory system, cancer or tumor diseases, infectious diseases or immunedeficiencies or adjuvant proteins are typically proteins of mammalian origin, preferably of human origin, depending on which animal shall be treated. A human subject, for example, is preferably treated by a therapeutic protein of human origin.

Pathogenic adjuvant proteins, typically comprise a pathogenic adjuvant protein, which is capable of eliciting an innate immune response (in a mammal), more preferably selected from pathogenic adjuvant proteins derived from bacteria, protozoa, viruses, or fungi, etc., e.g., bacterial (adjuvant) proteins, protozoan (adjuvant) proteins (e.g. profilin-like protein of Toxoplasma gondii), viral (adjuvant) proteins, or fungal (adjuvant) proteins, etc.

Particularly, bacterial (adjuvant) proteins may be selected from the group consisting of bacterial heat shock proteins or chaperons, including Hsp60, Hsp70, Hsp90, Hsp100; OmpA (Outer membrane protein) from gram-negative bacteria; bacterial porins, including OmpF; bacterial toxins, including pertussis toxin (PT) from Bordetella pertussis, pertussis adenylate cyclase toxin CyaA and CyaC from Bordetella pertussis, PT-9K/129G mutant from pertussis toxin, pertussis adenylate cyclase toxin CyaA and CyaC from Bordetella pertussis, tetanus toxin, cholera toxin (CT), cholera toxin B-subunit, CTK63 mutant from cholera toxin, CTE112K mutant from CT, Escherichia coli heat-labile enterotoxin (LT), B subunit from heat-labile enterotoxin (LTB) Escherichia coli heat-labile enterotoxin mutants with reduced toxicity, including LTK63, LTR72; phenol-soluble modulin; neutrophil-activating protein (HP-NAP) from Helicobacter pylori; Surfactant protein D; Outer surface protein A lipoprotein from Borrelia burgdorferi, Ag38 (38 kDa antigen) from Mycobacterium tuberculosis; proteins from bacterial fimbriae; Enterotoxin CT of Vibrio cholerae, Pilin from pili from gram negative bacteria, and Surfactant protein A; etc., or any species homolog of any of the above bacterial (adjuvant) proteins.

Bacterial (adjuvant) proteins may also comprise bacterial flagellins. In the context of the present invention, bacterial flagellins may be selected from flagellins from organisms including, without being limited thereto, Agrobacterium, Aquifex, Azospirillum, Bacillus, Bartonella, Bordetella, Borrelia, Burkholderia, Campylobacter, Caulobacte, Clostridium, Escherichia, Helicobacter, Lachnospiraceae, Legionella, Listeria, Proteus, Pseudomonas, Rhizobium, Rhodobacter, Roseburia, Salmonella, Serpulina, Serratia, Shigella, Treponema, Vibrio, Wolinella, Yersinia, more preferably from flagellins from the species including, without being limited thereto, Agrobacterium tumefaciens, Aquifex pyrophilus, Azospirillum brasilense, Bacillus subtilis, Bacillus thuringiensis, Bartonella bacilliformis, Bordetella bronchiseptica, Borrelia burgdorferi, Burkholderia cepacia, Campylobacter jejuni, Caulobacter crescentus, Clostridium botulinum strain Bennett clone 1, Escherichia coli, Helicobacter pylori, Lachnospiraceae bacterium, Legionella pneumophila, Listeria monocytogenes, Proteus mirabilis, Pseudomonas aeroguinosa, Pseudomonas syringae, Rhizobium meliloti, Rhodobacter sphaeroides, Roseburia cecicola, Roseburis hominis, Salmonella typhimurium, Salmonella bongori, Salmonella typhi, Salmonella enteritidis, Serpulina hyodysenteriae, Serratia marcescens, Shigella boydii, Treponema phagedenis, Vibrio alginolyticus, Vibrio cholerae, Vibrio parahaemolyticus, Wolinella succinogenes and Yersinia enterocolitica.

Protozoan (adjuvant) proteins are a further example of pathogenic adjuvant proteins. Protozoan (adjuvant) proteins may be selected in this context from any protozoan protein showing adjuvant properties, more preferably, from the group consisting of, without being limited thereto, Tc52 from Trypanosoma cruzi, PFTG from Trypanosoma gondii, Protozoan heat shock proteins, LeIF from Leishmania spp., profiling-like protein from Toxoplasma gondii, etc.

Viral (adjuvant) proteins are another example of pathogenic adjuvant proteins. In this context, viral (adjuvant) proteins may be selected from any viral protein showing adjuvant properties, more preferably, from the group consisting of, without being limited thereto, Respiratory Syncytial Virus fusion glycoprotein (F-protein), envelope protein from MMT virus, mouse leukemia virus protein, Hemagglutinin protein of wild-type measles virus, etc.

Fungal (adjuvant) proteins are even a further example of pathogenic adjuvant proteins. In the context of the present invention, fungal (adjuvant) proteins may be selected from any fungal protein showing adjuvant properties, more preferably, from the group consisting of, fungal immunomodulatory protein (FIP; LZ-8), etc.

Finally, adjuvant proteins may furthermore be selected from the group consisting of, Keyhole limpet hemocyanin (KLH), OspA, etc.

As mentioned above, also therapeutic antibodies are defined herein as therapeutic proteins. These therapeutic antibodies are preferably selected from antibodies, which are used inter alia for the treatment of cancer or tumor diseases, e.g. 131I-tositumomab (Follicular lymphoma, B cell lymphomas, leukemias), 3F8 (Neuroblastoma), 8H9, Abagovomab (Ovarian cancer), Adecatumumab (Prostate and breast cancer), Afutuzumab (Lymphoma), Alacizumab pegol, Alemtuzumab (B-cell chronic lymphocytic leukaemia, T-cell-Lymphoma), Amatuximab, AME-133v (Follicular lymphoma, cancer), AMG 102 (Advanced Renal Cell Carcinoma), Anatumomab mafenatox (Non-small cell lung carcinoma), Apolizumab (Solid Tumors, Leukemia, Non-Hodgkin-Lymphoma, Lymphoma), Bavituximab (Cancer, viral infections), Bectumomab (Non-Hodgkin's lymphoma), Belimumab (Non-Hodgkin lymphoma), Bevacizumab (Colon Cancer, Breast Cancer, Brain and Central Nervous System Tumors, Lung Cancer, Hepatocellular Carcinoma, Kidney Cancer, Breast Cancer, Pancreatic Cancer, Bladder Cancer, Sarcoma, Melanoma, Esophageal Cancer; Stomach Cancer, Metastatic Renal Cell Carcinoma; Kidney Cancer, Glioblastoma, Liver Cancer, Proliferative Diabetic Retinopathy, Macular Degeneration), Bivatuzumab mertansine (Squamous cell carcinoma), Blinatumomab, Brentuximab vedotin (Hematologic cancers), Cantuzumab (Colon Cancer, Gastric Cancer, Pancreatic Cancer, NSCLC), Cantuzumab mertansine (Colorectal cancer), Cantuzumab ravtansine (Cancers), Capromab pendetide (Prostate cancer), Carlumab, Catumaxomab (Ovarian Cancer, Fallopian Tube Neoplasms, Peritoneal Neoplasms), Cetuximab (Metastatic colorectal cancer and head and neck cancer), Citatuzumab bogatox (Ovarian cancer and other solid tumors), Cixutumumab (Solid tumors), Clivatuzumab tetraxetan (Pancreatic cancer), CNTO 328 (B-Cell Non-Hodgkin's Lymphoma, Multiple Myeloma, Castleman's Disease, ovarian cancer), CNTO 95 (Melanoma), Conatumumab, Dacetuzumab (Hematologic cancers), Dalotuzumab, Denosumab (Myeloma, Giant Cell Tumor of Bone, Breast Cancer, Prostate Cancer, Osteoporosis), Detumomab (Lymphoma), Drozitumab, Ecromeximab (Malignant melanoma), Edrecolomab (Colorectal carcinoma), Elotuzumab (Multiple myeloma), Elsilimomab, Enavatuzumab, Ensituximab, Epratuzumab (Autoimmune diseases, Systemic Lupus Erythematosus, Non-Hodgkin-Lymphoma, Leukemia), Ertumaxomab (Breast cancer), Ertumaxomab (Breast Cancer), Etaracizumab (Melanoma, prostate cancer, ovarian cancer), Farletuzumab (Ovarian cancer), FBTA05 (Chronic lymphocytic leukaemia), Ficlatuzumab (Cancer), Figitumumab (Adrenocortical carcinoma, non-small cell lung carcinoma), Flanvotumab (Melanoma), Galiximab (B-cell lymphoma), Galiximab (Non-Hodgkin-Lymphoma), Ganitumab, GC1008 (Advanced Renal Cell Carcinoma; Malignant Melanoma, Pulmonary Fibrosis), Gemtuzumab (Leukemia), Gemtuzumab ozogamicin (Acute myelogenous leukemia), Girentuximab (Clear cell renal cell carcinoma), Glembatumumab vedotin (Melanoma, breast cancer), GS6624 (Idiopathic pulmonary fibrosis and solid tumors), HuC242-DM4 (Colon Cancer, Gastric Cancer, Pancreatic Cancer), HuHMFG1 (Breast Cancer), HuN901-DM1 (Myeloma), Ibritumomab (Relapsed or refractory low-grade, follicular, or transformed B-cell non-Hodgkin's lymphoma (NHL)), Icrucumab, ID09C3 (Non-Hodgkin-Lymphoma), Indatuximab ravtansine, Inotuzumab ozogamicin, Intetumumab (Solid tumors (Prostate cancer, melanoma)), Ipilimumab (Sarcoma, Melanoma, Lung cancer, Ovarian Cancer leucemia, Lymphoma, Brain and Central Nervous System Tumors, Testicular Cancer, Prostate Cancer, Pancreatic Cancer, Breast Cancer), Iratumumab (Hodgkin's lymphoma), Labetuzumab (Colorectal cancer), Lexatumumab, Lintuzumab, Lorvotuzumab mertansine, Lucatumumab (Multiple myeloma, non-Hodgkin's lymphoma, Hodgkin's lymphoma), Lumiliximab (Chronic lymphocytic leukemia), Mapatumumab (Colon Cancer, Myeloma), Matuzumab (Lung Cancer, Cervical Cancer, Esophageal Cancer), MDX-060 (Hodgkin-Lymphoma, Lymphoma), MEDI 522 (Solid Tumors, Leukemia, Lymphoma, Small Intestine Cancer, Melanoma), Mitumomab (Small cell lung carcinoma), Mogamulizumab, MORab-003 (Ovarian Cancer, Fallopian Tube Cancer, Peritoneal Cancer), MORab-009 (Pancreatic Cancer, Mesothelioma, Ovarian Cancer, Non-Small Cell Lung Cancer, Fallopian Tube Cancer, Peritoneal Cavity Cancer), Moxetumomab pasudotox, MT103 (Non-Hodgkin-Lymphoma), Nacolomab tafenatox (Colorectal cancer), Naptumomab estafenatox (Non-small cell lung carcinoma, renal cell carcinoma), Narnatumab, Necitumumab (Non-small cell lung carcinoma), Nimotuzumab (Squamous cell carcinoma, head and neck cancer, nasopharyngeal cancer, glioma), Nimotuzumab (Squamous cell carcinomas, Glioma, Solid Tumors, Lung Cancer), Olaratumab, Onartuzumab (Cancer), Oportuzumab monatox, Oregovomab (Ovarian cancer), Oregovomab (Ovarian Cancer, Fallopian Tube Cancer, Peritoneal Cavity Cancer), PAM4 (Pancreatic Cancer), Panitumumab (Colon Cancer, Lung Cancer, Breast Cancer; Bladder Cancer; Ovarian Cancer), Patritumab, Pemtumomab, Pertuzumab (Breast Cancer, Ovarian Cancer, Lung Cancer, Prostate Cancer), Pritumumab (Brain cancer), Racotumomab, Radretumab, Ramucirumab (Solid tumors), Rilotumumab (Solid tumors), Rituximab (Urticaria, Rheumatoid Arthritis, Ulcerative Colitis, Chronic Focal Encephalitis, Non-Hodgkin-Lymphoma, Lymphoma, Chronic Lymphocytic Leukemia), Robatumumab, Samalizumab, SGN-30 (Hodgkin-Lymphoma, Lymphoma), SGN-40 (Non-Hodgkin-Lymphoma, Myeloma, Leukemia, Chronic Lymphocytic Leukemia), Sibrotuzumab, Siltuximab, Tabalumab (B-cell cancers), Tacatuzumab tetraxetan, Taplitumomab paptox, Tenatumomab, Teprotumumab (Hematologic tumors), TGN1412 (Chronic lymphocytic leukemia, rheumatoid arthritis), Ticilimumab (=tremelimumab), Tigatuzumab, TNX-650 (Hodgkin's lymphoma), Tositumomab (Follicular lymphoma, B cell lymphomas, Leukemias, Myeloma), Trastuzumab (Breast Cancer, Endometrial Cancer, Solid Tumors), TRBS07 (Melanoma), Tremelimumab, TRU-016 (Chronic lymphocytic leukemia), TRU-016 (Non-Hodgkin lymphoma), Tucotuzumab celmoleukin, Ublituximab, Urelumab, Veltuzumab (Non-Hodgkin's lymphoma), Veltuzumab (IMMU-106) (Non-Hodgkin's lymphoma), Volociximab (Renal Cell Carcinoma, Pancreatic Cancer, Melanoma), Votumumab (Colorectal tumors), WX-G250 (Renal Cell Carcinoma), Zalutumumab (Head and Neck Cancer, Squamous Cell Cancer), and Zanolimumab (T-Cell-Lymphoma);

Epitope: Epitopes (also called ‘antigen determinant’) can be distinguished in T cell epitopes and B cell epitopes. T cell epitopes or parts of the proteins in the context of the present invention may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule, i.e. the fragments are typically not recognized in their native form. B cell epitopes are typically fragments located on the outer surface of (native) protein or peptide antigens as defined herein, preferably having 5 to 15 amino acids, more preferably having 5 to 12 amino acids, even more preferably having 6 to 9 amino acids, which may be recognized by antibodies, i.e. in their native form.

Such epitopes of proteins or peptides may furthermore be selected from any of the herein mentioned variants of such proteins or peptides. In this context, antigenic determinants can be conformational or discontinuous epitopes, which are composed of segments of the proteins or peptides as defined herein that are discontinuous in the amino acid sequence of the proteins or peptides as defined herein, but are brought together in the three-dimensional structure or continuous or linear epitopes, which are composed of a single polypeptide chain.

Northern Blot: The northern blot is a technique used in molecular biology research to study gene expression by detection of RNA (or isolated mRNA) in a sample. Northern blotting involves the use of electrophoresis to separate RNA samples by size, and detection with a hybridization probe complementary to part of or the entire target sequence. The term ‘northern blot’ actually refers specifically to the capillary transfer of RNA from the electrophoresis gel to the blotting membrane. A general blotting procedure starts with extraction of total RNA from a sample or from cells. Eukaryotic mRNA can then be isolated through the use of oligo (dT) cellulose chromatography to isolate only those RNAs with a poly(A) tail. RNA samples are then separated by gel electrophoresis. Since the gels are fragile and the probes are unable to enter the matrix, the RNA samples, now separated by size, are transferred to a nylon membrane through a capillary or vacuum blotting system. A nylon membrane with a positive charge is the most effective for use in northern blotting since the negatively charged nucleic acids have a high affinity for them. The transfer buffer used for the blotting usually contains formamide because it lowers the annealing temperature of the probe-RNA interaction, thus eliminating the need for high temperatures, which could cause RNA degradation. Once the RNA has been transferred to the membrane, it is immobilized through covalent linkage to the membrane by UV light or heat. After a probe has been labeled, it is hybridized to the RNA on the membrane. The membrane is washed to ensure that the probe has bound specifically and to prevent background signals from arising. The hybrid signals are then detected by X-ray film and can be quantified by densitometry. The RNA samples are most commonly separated on agarose gels containing formaldehyde as a denaturing agent for the RNA to limit secondary structure. The gels can be stained with ethidium bromide (EtBr) and viewed under UV light to observe the quality and quantity of RNA before blotting. Polyacrylamide gel electrophoeresis with urea can also be used in RNA separation but it is most commonly used for fragmented RNA or microRNAs. Probes for northern blotting are composed of nucleic acids with a complementary sequence to all or part of the RNA of interest, they can be DNA, RNA, or oligonucleotides with a minimum of 25 complementary bases to the target sequence. RNA probes (riboprobes) that are transcribed in vitro are able to withstand more rigorous washing steps preventing some of the background noise. Commonly cDNA is created with labelled primers for the RNA sequence of interest to act as the probe in the northern blot. The probes must be labelled either with radioactive isotopes (32P) or with chemiluminescence in which alkaline phosphatase or horseradish peroxidase (HRP) break down chemiluminescent substrates producing a detectable emission of light. The chemiluminescent labelling can occur in two ways: either the probe is attached to the enzyme, or the probe is labelled with a ligand (e.g. biotin) for which the ligand (e.g., avidin or streptavidin) is attached to the enzyme (e.g. HRP). X-ray film can detect both the radioactive and chemiluminescent signals.

RNA Sequencing: RNA-seq (RNA sequencing), also called whole transcriptome shotgun sequencing, uses next-generation sequencing (NGS) to reveal the presence and quantity of RNA in a biological sample at a given moment in time. The high demand for low-cost sequencing has driven the development of high-throughput sequencing (or next-generation sequencing) technologies that parallelize the sequencing process, producing thousands or millions of sequences concurrently. In order to sequence RNA, the usual method is first to reverse transcribe the sample to generate cDNA fragments.

Reverse Transcriptase: A Reverse transcriptase (RT) is an enzyme used to generate complementary DNA (cDNA) from an RNA template, a process termed reverse transcription.

It is mainly associated with retroviruses. Retroviral RT has three sequential biochemical activities: RNA-dependent DNA polymerase activity, ribonuclease H, and DNA-dependent DNA polymerase activity.

Reverse Transcription: Reverse transcription is the process of generating a complementary DNA form an RNA template by a reverse transcriptase.

RT-PCR (Reverse transcription polymerase chain reaction): In RT-PCR, the RNA template is first converted into a complementary DNA (cDNA) using a reverse transcriptase. The cDNA is then used as a template for exponential amplification using PCR.

Quantitative Polymerase chain reaction (qPCR) or real-time polymerase chain reaction: A real-time polymerase chain reaction is a laboratory technique of molecular biology based on the polymerase chain reaction (PCR), which is used to amplify and simultaneously detect or quantify a targeted DNA molecule. The procedure follows the general principle of polymerase chain reaction (PCR); its key feature is that the amplified DNA is detected as the reaction progresses in “real time”. Two common methods for the detection of products in quantitative PCR are: (1) non-specific fluorescent dyes that intercalate with any double-stranded DNA, and (2) sequence-specific DNA probes consisting of oligonucleotides that are labelled with a fluorescent reporter, which permits detection only after hybridization of the probe with its complementary sequence to quantify nucleic acids. Quantitative PCR is carried out in a thermal cycler with the capacity to illuminate each sample with a beam of light of a specified wavelength and detect the fluorescence emitted by the excited fluorophore. The thermal cycler is also able to rapidly heat and chill samples, thereby taking advantage of the physicochemical properties of the nucleic acids and DNA polymerase. The PCR process generally consists of a series of temperature changes that are repeated 25-40 times. These cycles normally consist of three stages: the first, at around 95° C., allows the separation of the nucleic acid's double chain; the second, at a temperature of around 50-60° C., allows the binding of the primers with the DNA template; the third, at between 68-72° C., facilitates the polymerization carried out by the DNA polymerase. Due to the small size of the fragments the last step is usually omitted in this type of PCR as the enzyme is able to increase their number during the change between the alignment stage and the denaturing stage. In addition, some thermal cyclers add another short temperature phase lasting only a few seconds to each cycle, with a temperature of, for example, 80° C., in order to reduce the noise caused by the presence of primer dimers when a non-specific dye is used. The temperatures and the timings used for each cycle depend on a wide variety of parameters, such as: the enzyme used to synthesize the DNA, the concentration of divalent ions and deoxyribonucleotides (dNTPs) in the reaction and the bonding temperature of the primers. The type of quantitative PCR technique used depends on the DNA sequence in the samples, the technique can either use non-specific fluorochromes or hybridization probes.

### DETAILED DESCRIPTION OF THE INVENTION

The present invention relates to a method of diagnosing, monitoring a subject or determining the prognosis of a subject comprising the steps of (a) determining the expression level of at least one marker gene selected from the group consisting of the genes set out in table 1 in a sample of the subject to obtain a gene expression profile; (b) diagnosing, monitoring a subject or determining the prognosis of the subject based on the gene expression profile obtained in step (a).

In particular, the invention relates to a method of determining the prognosis of a patient comprising the steps of (a) determining the expression level of at least one marker gene selected from the group consisting of the marker genes set out in table 1 in a sample of the patient to obtain a gene expression profile; (b) determining the prognosis of the patient based on the gene expression profile obtained in step (a).

The patient may be a patient suffering from cancer or tumor. In preferred embodiments, the patient is a lung cancer patient. Most preferably the patient is a non-small cell lung cancer (NSCLC) patient.

Therefore, the invention in particular relates to a method of determining the prognosis of a cancer or tumor patient comprising the steps of (a) determining the expression level of at least one marker gene selected from the group consisting of the marker genes set out in table 1 in a sample of the cancer patient to obtain a gene expression profile; (b) determining the prognosis of the cancer patient based on the gene expression profile obtained in step (a).

In preferred embodiments, the invention relates to a method of determining the prognosis of a NSCLC patient comprising the steps of (a) determining the expression level of at least one marker gene selected from the group consisting of the marker genes set out in table 1 in a sample of the NSCLC cancer patient to obtain a gene expression profile; (b) determining the prognosis of the NSCLC cancer patient based on the gene expression profile obtained in step (a).

The expression level may be determined for at least one marker gene, at least 2 marker genes, at least 3 marker genes, at least 4 marker genes, at least 5 marker genes, at least 6 marker genes, at least 7 marker genes, at least 8 marker genes, at least 9 marker genes, at least 10 marker genes, at least 11 marker genes, at least 12 marker genes, at least 13 marker genes, at least 14 marker genes, at least 15 marker genes, at least 16 marker genes, at least 17 marker genes, at least 18 marker genes, at least 19 marker genes, at least 20 marker genes, at least 25 marker genes, at least 30 marker genes, at least 35 marker genes, at least 40 marker genes, at least 45 marker genes, at least 50 marker genes, at least 55 marker genes, at least 60 marker genes, at least 70 marker genes, at least 80 marker genes, at least 90 marker genes at least 100 marker genes.

The expression level may be determined for at least one marker gene, preferably at least 15 marker genes, more preferably at least 20 marker genes, even more preferably at least 30 marker genes, still more preferably at least 50 marker genes, most preferably at least 100 marker genes.

The expression level may be determined for example for one marker gene, 2 marker genes, 3 marker genes, 4 marker genes, 5 marker genes, 6 marker genes, 7 marker genes, 8 marker genes, 9 marker genes, 10 marker genes, 11 marker genes, 12 marker genes, 13 marker genes, 14 marker genes, 15 marker genes, 16 marker genes, 17 marker genes, 18 marker genes, 19 marker genes, 20 marker genes, 25 marker genes, 30 marker genes, 35 marker genes, 40 marker genes, 45 marker genes, 50 marker genes, 55 marker genes, 60 marker genes, 70 marker genes, 80 marker genes, 90 marker genes or 100 marker genes.

In specific embodiments, the at least one marker gene is selected from the group consisting of genes set out in table 2. In preferred embodiments, the at least one marker gene is selected from the group consisting of marker genes set out in table 3. In more preferred embodiments, the at least one marker gene is selected from group consisting of marker genes set out in table 4. In even more preferred embodiments, the at least one marker gene is selected from group consisting of marker genes set out in table 5.

In some embodiments at least 10 marker genes, such as 15, 25 or 25 marker genes, are selected from table 1. Preferably at least 30 marker genes, such as 35, 40 or 45 marker genes, more preferably at least 50 marker genes, such as 55, 60, 65, 70, 75, 80, 85, 90 or 95 marker genes, most preferably at least 100 marker genes are selected from table 1.

In some embodiments at least 10 marker genes, such as 15, 25 or 25 marker genes, are selected from table 2. Preferably at least 30 marker genes, such as 35, 40 or 45 marker genes, more preferably at least 50 marker genes, such as 55, 60, 65, 70, 75, 80, 85, 90 or 95 marker genes, most preferably at least 100 marker genes are selected from table 2.

In some embodiments at least 10 genes, such as 15, 25 or 25 marker genes, are selected from table 3. Preferably at least 30 marker genes, such as 35, 40 or 45 marker genes, more preferably at least 50 marker genes are selected from table 3.

In some embodiments at least 10 genes, such as 15 marker genes, are selected from table 4. Preferably at least 20 marker genes, such as 25 marker genes, more preferably at least 30 marker genes are selected from table 4.

In some embodiments at least 10 marker genes, such as 15 marker genes are selected from table 5.

The expression level of the at least one marker gene may be determined by any means known in the art. In a preferred embodiment of the present invention the determination of the expression level of the marker gene is accomplished by the measurement of nucleic acid. Thus, the expression level(s) may be determined by a method involving the detection of an mRNA encoded by the gene. Such methods include e.g. Nothem Blot analysis, (quantitative or semi-quantitative) RT-PCR, microarray analysis, and RNA sequencing (“next generation sequencing”).

For example, the measurement of the nucleic acid level of marker gene(s) expression may be assessed by purification of nucleic acid molecules (e.g. RNA) obtained from the sample, followed by hybridization with specific nucleic acid based probes selective for determining the expression level of the marker genes as defined herein. Comparison of expression levels may be accomplished visually or by means of an appropriate device. Methods for the detection of mRNA or expression products are known to the person skilled in the art.

Alternatively, the nucleic acid level of marker gene(s) expression may be detected in a microarray approach. Typically, sample nucleic acids derived from patients to be tested are processed and labeled, preferably with a fluorescent label. Subsequently, such nucleic acid molecules may be used in a hybridization approach with immobilized capture probes corresponding to the marker genes of the present invention. Suitable means for carrying out microarray analyses are known to the person skilled in the art. In a standard setup a microarray comprises immobilized high-density probes to detect a number of genes. The probes on the array are complementary to one or more parts of the mRNA sequence of the marker genes.

A microarray-based detection method typically comprises the following steps: (1) Isolating mRNA from a sample and optionally converting the mRNA to cDNA by reverse transcription, and subsequently labeling this RNA or cDNA. Methods for isolating RNA, converting it into cDNA by reverse transcription and for labeling nucleic acids are described in manuals for microarray technology. (2) Hybridizing the nucleic acids from step 1 with probes for the marker genes. The nucleic acids from a sample can be labeled with a dye, such as the fluorescent dyes Cy3 (red) or Cy5 (blue). Generally, a control sample is labeled with a different dye. (3) Detecting the hybridization of the nucleic acids from the sample with the probes and determining at least qualitatively, and more particularly quantitatively, the amounts of mRNA in the sample for marker genes investigated. The difference in the expression level between sample and control can be estimated based on a difference in the signal intensity. These can be measured and analyzed by appropriate software.

There is no limitation on the number of probes corresponding to the marker genes used, which are spotted on a microarray. Also, a marker gene can be represented by two or more probes, the probes hybridizing to different parts of a gene. Probes are designed for each selected marker gene. Such a probe is typically an oligonucleotide comprising 5-50 nucleotide residues. Longer DNAs can be synthesized by PCR or chemically. Methods for synthesizing such oligonucleotides and applying them on a substrate are well known in the field of microarrays. Genes other than the marker genes may be also spotted on the DNA array. For example, a probe for a gene whose expression level is not significantly altered may be spotted on the DNA array to normalize assay results or to compare assay results of multiple arrays or different assays. Such a gene is also termed herein as “reference gene”.

Alternatively, the nucleic acid level of marker gene(s) expression may be detected in a quantitative RT-PCR approach, preferably in a real-time PCR approach following the reverse transcription of the transcripts of interest. Typically, as first step, a transcript is reverse transcribed into a cDNA molecule according to any suitable method known to the person skilled in the art. A quantitative or real-time PCR approach may subsequently be carried out based on a first DNA strand obtained as described above.

Preferably, Taqman or Molecular Beacon probes as principal FRET-based probes of this type may be used for quantitative PCR detection. In both cases, the probes, serve as internal probes which are used in conjunction with a pair of opposing primers that flank the target region of interest, preferably a set of marker gene(s) specific oligonucleotides as defined herein above. Upon amplification of a target segment, the probe may selectively bind to the products at an identifying sequence in between the primer sites, thereby causing increases in FRET signaling relative to increases in target frequency.

Preferably, a Taqman probe to be used for a quantitative PCR approach according to the present invention may comprises a specific oligonucleotide as defined above of about 22 to 30 bases that is labeled on both ends with a FRET pair. Typically, the 5′ end will have a shorter wavelength fluorophore such as fluorescein (e.g. FAM) and the 3′ end is commonly labeled with a longer wavelength fluorescent quencher (e.g. TAMRA) or a non-fluorescent quencher compound (e.g. Black Hole Quencher). It is preferred that the probes to be used for quantitative PCR, in particular probes as defined herein above, have no guanine (G) at the 5′ end adjacent to the reporter dye in order to avoid quenching of the reporter fluorescence after the probe is degraded.

A Molecular Beacon probe to be used for a quantitative PCR approach according to the present invention preferably uses FRET interactions to detect and quantify a PCR product, with each probe having a 5′ fluorescent-labeled end and a 3′ quencher-labeled end. This hairpin or stem-loop configuration of the probe structure comprises preferably a stem with two short self-binding ends and a loop with a long internal target-specific region of about 20 to 30 bases.

Alternative detection mechanisms which may also be employed in the context of the present invention are directed to a probe fabricated with only a loop structure and without a short complementary stem region. An alternative FRET-based approach for quantitative PCR which may also be used in the context of the present invention is based on the use of two hybridization probes that bind to adjacent sites on the target wherein the first probe has a fluorescent donor label at the 3′ end and the second probe has a fluorescent acceptor label at its 5′ end.

Also detection techniques using molecular barcodes, for example color coded molecular barcodes (such as nCounter from Nanostring technologies) may be used. Typically, in such methods a reporter probe carrying the signal and a capture probe are used. After hybridization, the excess probes are removed and the complexes containing the target sequence hybridized to the signal probe and the capture probe are aligned, immobilized in a cartridge and the color codes of the signal probe are counted.

Alternatively, the nucleic acid level of marker gene(s) expression may be detected by RNA sequencing. RNA sequencing, also called whole transcriptome shotgun sequencing (WTSS), uses next-generation sequencing (NGS) to reveal the presence and quantity of RNA in a biological sample at a given moment in time. Different methods used for next-generation sequencing are known in the art.

In specific embodiments the expression level is measured before administration and/or after administration of a least one dose of a therapeutic agent.

In preferred embodiments the gene expression profile of step (a) is obtained by determining the difference of the expression level of the at least one marker gene measured before administration of the therapeutic agent and after the administration of at least one dose of the therapeutic agent.

Therefore, some embodiments of the invention relate to a method of determining the prognosis of a patient comprising the steps of (a) determining the difference of the expression level of the at least one marker gene before administration of the therapeutic agent and after the administration of at least one dose of the therapeutic agent in sample of the patient to obtain a gene expression profile; (b) determining the prognosis of the patient based on the gene expression profile obtained in step (a).

In particular, some embodiments of the present invention relate to a method of determining the prognosis of a cancer patient, preferably a lung cancer patient, and most preferably a NSCLC patient, comprising the steps of (a) determining the difference of the expression level of the at least one marker gene before administration of the therapeutic agent and after the administration of at least one dose of the therapeutic agent in sample of the cancer patient to obtain a gene expression profile; (b) determining the prognosis of the cancer patient based on the gene expression profile obtained in step (a).

In this context therapeutic agents include all agents and therapies used for therapy or prevention of a disease, particularly of tumor or cancer diseases. Particularly preferred are surgery, radiation therapy, chemotherapy, chemoradiation, and/or treatment with kinase inhibitors, inhibitory and/or stimulatory checkpoint molecules (checkpoint modulators) or antibodies. Most preferably therapeutic agents are selected from vaccines and/or immunostimulatory compositions and/or immunotherapeutic agents, preferably as defined herein.

In one embodiment it is particularly preferred that the vaccine or immunostimulatory composition comprises at least one antigen, preferably a tumor antigen as defined herein.

A specific embodiment of the invention relates to a method of determining the prognosis of a cancer patient, preferably a lung cancer patient and more preferably a NSCLC patient comprising the steps of (a) determining the difference of the expression level of the at least one marker gene before administration of a vaccine or immunostimulatory composition comprising at least one tumor antigen of the group consisting of MAGE-C1, MAGE-C2, NY-ESO-1, Survivin and 5T4 and after the administration of at least one dose of said vaccine or immunotherapeutic agent in a sample of the cancer patient to obtain a gene expression profile; (b) determining the prognosis of the cancer patient based on the gene expression profile obtained in step (a).

A specific embodiment of the invention relates to a method of determining the prognosis of a cancer patient, preferably a lung cancer patient and more preferably a NSCLC patient comprising the steps of (a) determining the difference of the expression level of the at least one marker gene before administration of a vaccine or immunostimulatory composition comprising the tumor antigens MAGE-C1, MAGE-C2, NY-ESO-1, Survivin and 5T4 and after the administration of at least one dose of said vaccine or immunostimulatory composition in a sample of the cancer patient to obtain a gene expression profile; (b) determining the prognosis of the cancer patient based on the gene expression profile obtained in step (a).

Another specific embodiment of the invention relates to a method of determining the prognosis of a cancer patient, preferably a lung cancer patient, more preferably a NSCLC patient comprising the steps of (a) determining the difference of the expression level of the at least one marker gene before administration of a vaccine or immunostimulatory composition comprising the antigens MAGE-C1, MAGE-C2, NY-ESO-1, Survivin, 5T4, and Muc-1 and after the administration of at least one dose of said vaccine or immunotherapeutic agent in a sample of the NSCLC patient to obtain a gene expression profile; (b) determining the prognosis of the cancer patient based on the gene expression profile obtained in step (a).

The sample of the individual or patient can be any sample suitable for the analysis of the expression of the marker genes as disclosed herein. The sample of the individual or patient may be without limitation whole blood or fractions thereof, such as peripheral blood mono-nuclear cells (PBMCs). Preferably, the sample of the individual or patient comprises PBMCs. Alternatively, the sample may be any tissue of the patient, preferably tumor tissue.

In step (b) the prognosis of a cancer patient may be obtained based on the gene expression profile obtained in step (a). For example, the increase or decrease of the expression of the at least one marker gene may relate to a certain course or outcome of a diagnosed or detected disease. For example, the increase or decrease of the expression of the at least one marker gene may relate to a chance of survival or recovery from the disease, to an expected survival time of a subject. For example, the increase or decrease of the expression of the at least one marker gene as described herein may indicate the expected survival time of a subject. In particular, if the prognosis in step (b) is determined based on an expression profile of several genes, for example 30, 50 or 100 or more genes, algorithms can be used to determine the prognosis of a cancer patient. Preferably, clustering algorithms, more preferably hierarchical clustering algorithms can be used in step (b).

Typically, the therapeutic agent may be an immunostimulatory composition and/or a vaccine and/or an immunotherapeutic agent. The therapeutic agent may be a therapeutic protein as defined above or may comprise additionally a therapeutic protein. Preferably the therapeutic agent comprises the antigens MAGE-C1, MAGE-C2, NY-ESO-1, Survivin and 5T4 or fragments thereof. The antigen(s) may be present as peptides or proteins and/or are encoded by at least one nucleotide sequence. Preferably the antigens are encoded by at least one mRNA molecule. In a specific embodiment the therapeutic agent is at least one mRNA molecule encoding the antigens MAGE-C1, MAGE-C2, NY-ESO-1, Survivin and 5T4 or fragments thereof.

In the context of this invention “MAGE-C1” is the melanoma antigen family C, 1 and the preferred sequence of the RNA, preferably of the mRNA, encoding “MAGE-C1”—if being used in the immunostimulatory composition—is shown in SEQ ID NO: 1, more preferably in SEQ ID NO: 2, and even more preferably in SEQ ID NO: 3.

In the context of this invention “MAGE-C2” is the melanoma antigen family C2 and the preferred sequence of the RNA, preferably of the mRNA, encoding “MAGE-C2”—if being used in the immunostimulatory composition—is shown in SEQ ID NO: 4, and even more preferably SEQ ID NO: 5.

In the context of this invention “NY-ESO-1” is cancer/testis antigen 1B and the preferred sequence of the RNA, preferably of the mRNA, encoding “NY-ESO-1”—if being used in the immunostimulatory composition—is shown in SEQ ID NO: 6, and in SEQ ID NO: 7.

In the context of this invention “Survivin” is baculoviral IAP repeat-containing 5 (survivin) and the preferred sequence of the RNA, preferably of the mRNA, encoding “survivin”—if being used in the immunostimulatory composition—is shown in SEQ ID NO: 8, and even more preferably in SEQ ID NO: 9.

In the context of this invention “5T4” is trophoblast glycoprotein and the preferred sequence of the RNA, preferably of the mRNA, encoding “5T4”—if being used in the vaccine—is shown in SEQ ID NO: 10, and even more preferably in SEQ ID NO: 11.

The immunostimulatory composition may comprise antigens, antigenic proteins or antigenic peptides or nucleic acids such as DNA or RNA encoding antigens, antigenic proteins or antigenic peptides capable to effectively stimulate the (adaptive) immune system to allow treatment of cancer, preferably lung cancer, especially of non-small cell lung cancer (NSCLC). Preferably the immunostimulatory composition comprises at least one RNA encoding at least one antigen, antigenic protein or antigenic peptide.

Preferably, the immunostimulatory composition or vaccine comprises at least one RNA encoding at least one antigen selected from the group consisting of MAGE-C1, MAGE-C2, NY-ESO-1, Survivin and 5T4, wherein the at least one RNA is complexed to a complexing agent, preferably protamine.

The vaccine may contain the active immunostimulatory composition. The vaccine may additionally contain a pharmaceutically acceptable carrier and/or further auxiliary substances and additives and/or adjuvants. According to a particularly preferred embodiment, the antigens are selected from the group consisting of MAGE-C1, MAGE-C2, NY-ESO-1, Survivin and 5T4.

According to another embodiment, the antigens are selected from the group consisting of
STEAP (Six Transmembrane Epithelial Antigen of the Prostate);
PSA (Prostate-Specific Antigen),
PSMA (Prostate-Specific Membrane Antigen),
PSCA (Prostate Stem Cell Antigen);
PAP (Prostatic Acid Phosphatase), and
MUC1 (Mucin 1).

The antigens are described in detail in WO2015024664, which is incorporated herein by reference. Also the multi-antigenic CV9104 is an mRNA-based immunotherapeutic which encodes for the six antigens Muc-1, PSA, PSCA, PSMA, STEAP-1 and PAP is described in detail in WO2015024664.

In a preferred embodiment, the vaccine comprises a safe and effective amount of antigen encoding RNA of the immunostimulatory composition as defined above. As used herein, “safe and effective amount” means an amount of the RNA of the immunostimulatory composition in the vaccine as defined above, that is sufficient to significantly induce a positive modification of the disease, preferably cancer, more preferably lung cancer, even more preferably of a non-small-cell lung cancer (NSCLC) related condition to be treated, more preferably of conditions related to the three main sub-types of non-small-cell lung cancer (NSCLC) including, without being restricted thereto, squamous cell lung carcinoma, adenocarcinoma and large cell lung carcinoma. At the same time, however, a “safe and effective amount” is small enough to avoid serious side-effects, that is to say to permit a sensible relationship between advantage and risk. The determination of these limits typically lies within the scope of sensible medical judgment. In relation to the vaccine, the expression “safe and effective amount” preferably means an amount of the RNA that is suitable for stimulating the adaptive immune system in such a manner that no excessive or damaging immune reactions are achieved but, preferably, also no such immune reactions below a measurable level. Such a “safe and effective amount” of the at least one RNA of the immunostimulatory composition in the vaccine as defined above may furthermore be selected in dependence of the type of RNA, e.g. monocistronic, bi- or even multicistronic RNA, since a bi- or even multicistronic RNA may lead to a significantly higher expression of the encoded antigen(s) than use of an equal amount of a monocistronic RNA. A “safe and effective amount” of the at least one RNA of the immunostimulatory composition as defined above, which is contained in the vaccine, will furthermore vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor.

The vaccine typically contains a pharmaceutically acceptable carrier. The expression “pharmaceutically acceptable carrier” as used herein preferably includes the liquid or non-liquid basis of the vaccine. If the vaccine is provided in liquid form, the carrier will typically be pyrogen-free water; isotonic saline or buffered (aqueous) solutions, e.g. phosphate-, citrate-buffered solutions, etc.

The vaccine can additionally contain one or more auxiliary substances in order to further increase the immunogenicity. A synergistic action of the at least one RNA of the active (immunostimulatory) composition as defined above and of an auxiliary substance, which may be optionally also contained in the vaccine as described above, is preferably achieved thereby. Depending on the various types of auxiliary substances, various mechanisms can come into consideration in this respect. For example, compounds that permit the maturation of dendritic cells (DCs), for example lipopolysaccharides, TNF-alpha or CD40 ligand, form a first class of suitable auxiliary substances. In general, it is possible to use as auxiliary substance any agent that influences the immune system in the manner of a “danger signal” (LPS, GP96, etc.) or cytokines, such as GM-CSF, which allow an immune response produced by the immune-stimulating adjuvant according to the invention to be enhanced and/or influenced in a targeted manner. Particularly preferred auxiliary substances are cytokines, such as monokines, lymphokines, interleukins or chemokines, that—additional to induction of the adaptive immune response by the encoded at least two antigens—promote the innate immune response, such as IL-1, IL-2, IL-3, IL-4, IL-5, IL-6, IL-7, IL-8, IL-9, IL-10, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, INF-alpha, IFN-beta, INF-gamma, GM-CSF, G-CSF, M-CSF, LT-beta or TNF-alpha, growth factors, such as hGH.

Further additives which may be included in the vaccine are emulsifiers, such as, for example, Tween®; wetting agents, such as, for example, sodium lauryl sulfate; colouring agents; taste-imparting agents, pharmaceutical carriers; tablet-forming agents; stabilizers; antioxidants; preservatives. The vaccine can also additionally contain any further compound, which is known to be immune-stimulating due to its binding affinity (as ligands) to human Toll-like receptors or due to its binding affinity (as ligands) to murine Toll-like receptors. Another class of compounds, which may be added to the vaccine, may be CpG nucleic acids, in particular CpG-RNA or CpG-DNA. A CpG-RNA or CpG-DNA can be a single-stranded CpG-DNA (ss CpG-DNA), a double-stranded CpG-DNA (dsDNA), a single-stranded CpG-RNA (ss CpG-RNA) or a double-stranded CpG-RNA (ds CpG-RNA).

Moreover, the invention relates to a method of determining whether a cancer patient responds to a therapeutic agent comprising the steps of
(a) determining the difference of the expression level measured before administration of the therapeutic agent and after administration of at least one dose of the therapeutic agent of at least one marker gene selected from the group consisting of the marker genes set out in table 1 in a sample of the cancer patient to obtain a gene expression profile;
(b) determining whether the cancer patient responds to a therapeutic agent based on the gene expression profile obtained in step (a);

Another aspect of the invention refers to a kit, diagnostic composition or device for the analysis of the expression of at least one marker gene as described herein. In one embodiment, the invention refers to a kit, diagnostic composition or device for the analysis of the expression of at least one marker gene of table 1 comprising at least one primer and/or probe selective for determining the expression level of at least one marker gene of table 1. Some embodiments relate to a kit, diagnostic composition or device comprising at least 10 primers and/or probes, preferably at least 30 primers and/or probes, more preferably at least 50 primers and/or probes, most preferably at least 100 primers and/or probes selective for determining the expression level of at least 10, 30, 50 or 100 marker genes of table 1. A further embodiment refers to a kit, diagnostic composition or device for the analysis of the expression of at least one marker gene of table 2 comprising at least one primer and/or probe selective for determining the expression level of at least one marker gene of table 2. Some embodiments relate to a kit, diagnostic composition or device comprising at least 10 primers and/or probes, preferably at least 30 primers and/or probes, more preferably at least 50 primers and/or probes, most preferably at least 100 primers and/or probes selective for determining the expression level of at least 10, 30, 50 or 100 marker genes of table 2. In a further embodiment, the invention refers to a kit, diagnostic composition or device for the analysis of the expression of at least one marker gene of table 3 comprising at least one primer and/or probe selective for determining the expression level of at least one marker gene of table 3. Further embodiments relate to a kit, diagnostic composition or device comprising at least 10 primers and/or probes, preferably at least 30 primers and/or probes, more preferably at least 40 primers and/or probes, most preferably at least 50 primers and/or probes selective for determining the expression level of at least 10, 30, 40 or 50 marker genes of table 3. In another embodiment, the invention refers to a kit, diagnostic composition or device for the analysis of the expression of at least one marker gene of table 4 comprising at least one primer and/or probe selective for determining the expression level of at least one marker gene of table 4. Further embodiments relate to a kit, diagnostic composition or device comprising at least 10 primers and/or probes, preferably at least 20 primers and/or probes, more preferably at least 25 primers and/or probes, most preferably at least 30 primers and/or probes selective for determining the expression level of at least 10, 20, 25 or 30 marker genes of table 4. In another embodiment, the invention refers to a kit, diagnostic composition or device for the analysis of the expression of at least one marker gene, preferably at least 5 marker genes, more preferably at least 10 marker genes of table 5 comprising at least one primer and/or probe, preferably at least 5 primers and/or probes, more preferably at least 10 primers and/or probes selective for determining the expression level of at least one marker gene of table 5.

A further embodiment refers to a kit, diagnostic composition or device for the analysis of the expression of at least one marker gene of table 2B comprising at least one primer and/or probe selective for determining the expression level of at least one marker gene of table 2B. Some embodiments relate to a kit, diagnostic composition or device comprising at least 10 primers and/or probes, preferably at least 30 primers and/or probes, more preferably at least 50 primers and/or probes, most preferably at least 90 primers and/or probes selective for determining the expression level of at least 10, 30, 50 or 90 marker genes of table 2B. In a further embodiment, the invention refers to a kit, diagnostic composition or device for the analysis of the expression of at least one marker gene of table 3B comprising at least one primer and/or probe selective for determining the expression level of at least one marker gene of table 3B. Further embodiments relate to a kit, diagnostic composition or device comprising at least 10 primers and/or probes, preferably at least 30 primers and/or probes, more preferably at least 40 primers and/or probes, most preferably at least 40 primers and/or probes selective for determining the expression level of at least 10, 30 or 40 marker genes of table 3B. In another embodiment, the invention refers to a kit, diagnostic composition or device for the analysis of the expression of at least one marker gene of table 4B comprising at least one primer and/or probe selective for determining the expression level of at least one marker gene of table 4B. Further embodiments relate to a kit, diagnostic composition or device comprising at least 10 primers and/or probes, preferably at least 20 primers and/or probes, more preferably at least 25 primers and/or probes selective for determining the expression level of at least 10, 20 or 25 marker genes of table 4B. In another embodiment, the invention refers to a kit, diagnostic composition or device for the analysis of the expression at least one marker gene, preferably at least 5 marker genes, more preferably at least 8 marker genes of table 5 comprising at least one primer and/or probe, preferably at least 5 primers and/or probes, more preferably at least 8 primers and/or probes selective for determining the expression level of at least of at least 5 or 8 marker genes of table 5B.

The kit as described herein may further comprise an enzyme for primer elongation, nucleotides and/or labeling agents.

Another aspect of the invention refers to a microarray for the analysis of the expression of at least one marker gene as described herein. In one embodiment, the invention refers to a microarray for the analysis of the expression of at least one marker gene of table 1 comprising at least one probe selective for determining the expression level of at least one marker gene of table 1. Some embodiments relate to a microarray comprising at least 10 probes, preferably at least 30 probes, more preferably at least 50 probes, most preferably at least 100 probes selective for determining the expression level of at least 10, 30, 50 or 100 marker genes of table 1. A further embodiment refers to a microarray for the analysis of the expression of at least one marker gene of table 2 comprising at least one probe selective for determining the expression level of at least one marker gene of table 2. Some embodiments relate to a microarray comprising at least 10 probes, preferably at least 30 probes, more preferably at least 50 probes, most preferably at least 100 probes selective for determining the expression level of at least 10, 30, 50 or 100 marker genes of table 2. In a further embodiment, the invention refers to a microarray for the analysis of the expression of at least one marker gene of table 3 comprising at least one probe selective for determining the expression level of at least one marker gene of table 3. Further embodiments relate to a microarray comprising at least 10 probes, preferably at least 30 probes, more preferably at least 40 probes, most preferably at least 50 probes selective for determining the expression level of at least 10, 30, 40 or 50 marker genes of table 3. In another embodiment, the invention refers to a microarray for the analysis of the expression of at least one marker gene of table 4 comprising at least one probe selective for determining the expression level of at least one marker gene of table 4. Further embodiments relate to a microarray comprising at least 10 probes, preferably at least 20 probes, more preferably at least 25 probes, most preferably at least 30 probes selective for determining the expression level of at least 10, 20, 25 or 30 marker genes of table 4. In another embodiment, the invention refers to a microarray for the analysis of the expression of at least one marker gene, preferably at least 5 marker genes, more preferably at least 10 marker genes of table 5 comprising at least one probe, preferably at least 5 probes, more preferably at least 10 probes selective for determining the expression level of at least one marker gene of table 5.

A further embodiment refers to a microarray for the analysis of the expression of at least one marker gene of table 2B comprising at least one probe selective for determining the expression level of at least one marker gene of table 2B. Some embodiments relate to a microarray comprising at least 10 probes, preferably at least 30 probes, more preferably at least 50 probes, most preferably at least 90 probes selective for determining the expression level of at least 10, 30, 50 or 90 marker genes of table 2B. In a further embodiment, the invention refers to a microarray for the analysis of the expression of at least one marker gene of table 3B comprising at least one probe selective for determining the expression level of at least one marker gene of table 3B. Further embodiments relate to a microarray comprising at least 10 probes, preferably at least 30 probes, more preferably at least 40 probes selective for determining the expression level of at least 10, 30 or 40 marker genes of table 3B. In another embodiment, the invention refers to a microarray for the analysis of the expression of at least one marker gene of table 4B comprising at least one probe selective for determining the expression level of at least one marker gene of table 4B. Further embodiments relate to a microarray comprising at least 10 probes, preferably at least 20 probes, more preferably at least 25 probes selective for determining the expression level of at least 10, 20 or 25 marker genes of table 4B. In another embodiment, the invention refers to a microarray for the analysis of the expression of at least one marker gene, preferably at least 5 marker genes, more preferably at least 8 marker genes of table 5B comprising at least one probe, preferably at least 5 probes, more preferably at least 8 probes selective for determining the expression level of at least one marker gene of table 5B.

Another aspect of the invention refers to the use of a microarray as described herein for determining the prognosis of a cancer patient, preferably a lung cancer patient and more preferably a NSCLC patient. A further aspect of the invention refers to the use of a kit as described herein for determining the prognosis of a cancer patient, preferably a lung cancer patient and more preferably a NSCLC patient.

Another aspect refers to a non-transitory computer readable storage medium with an executable program stored thereon, wherein the program is for diagnosing, monitoring a subject, preferably a lung cancer patient and more preferably a NSCLC patient or determining the prognosis of a subject, preferably a lung cancer patient and more preferably a NSCLC patient and wherein the program instructs a microprocessor to perform one or more of the steps of any of the methods described herein.

TABLE 1
marker genes for cancer, in particular lung cancer

| Description | Gene Symbol |
|---|---|
| uncharacterized LOC572558 (LOC572558), non-coding RNA [NR_015423] | LOC572558 |
| Usher syndrome 1G (autosomal recessive) (USH1G), mRNA [NM_173477] | USH1G |
| bone morphogenetic protein 2 (BMP2), mRNA [NM_001200] | BMP2 |
| kinesin family member 18A (KIF18A), mRNA [NM_031217] | KIF18A |
| anillin, actin binding protein (ANLN), mRNA [NM_018685] | ANLN |
| glycoprotein V (platelet) (GPS), mRNA [NM_004488] | GPS |
| spindle and kinetochore associated complex subunit 3 (SKA3), transcript variant | SKA3 |
| 1, mRNA [NM_145061] | |
| TTK protein kinase (TTK), transcript variant 1, mRNA [NM_003318] | TTK |
| olfactomedin 4 (OLFM4), mRNA [NM_006418] | OLFM4 |
| discs, large (Drosophila) homolog-associated protein 5 (DLGAP5), transcript | DLGAP5 |
| variant 1, mRNA [NM_014750] | |
| minichromosome maintenance complex component 10 (MCM10), transcript | MCM10 |
| variant 1, mRNA [NM_182751] | |
| secretory leukocyte peptidase inhibitor (SLPI), mRNA [NM_003064] | SLPI |
| Fas ligand (TNF superfamily, member 6) (FASLG), mRNA [NM_000639] | FASLG |
| solute carrier family 7 (amino acid transporter light chain, L system), member 8 | SLC7A8 |
| (SLC7A8), transcript variant 2, mRNA [NM_182728] | |
| exonuclease 1 (EXO1), transcript variant 3, mRNA [NM_003686] | EXO1 |
| fibroblast growth factor binding protein 2 (FGFBP2), mRNA [NM_031950] | FGFBP2 |
| chemokine (C-X-C motif) ligand 1 (melanoma growth stimulating activity, alpha) | CXCL1 |
| (CXCL1), mRNA [NM_001511] | |
| maternal embryonic leucine zipper kinase (MELK), mRNA [NM_014791] | MELK |
| ribonucleotide reductase M2 (RRM2), transcript variant 2, mRNA [NM_001034] | RRM2 |
| KIAA0101 (KIAA0101), transcript variant 1, mRNA [NM_014736] | KIAA0101 |
| interferon, alpha-inducible protein 27 (IFI27), transcript variant 2, mRNA | IFI27 |
| [NM_005532] | |
| RAD51 homolog (S. cerevisiae) (RAD51), transcript variant 1, mRNA | RAD51 |
| [NM_002875] | |
| thymidylate synthetase (TYMS), mRNA [NM_001071] | TYMS |
| E2F transcription factor 8 (E2F8), mRNA [NM_024680] | E2F8 |
| platelet-derived growth factor receptor, beta polypeptide (PDGFRB), mRNA | PDGFRB |
| [NM_002609] | |
| chitinase 3-like 1 (cartilage glycoprotein-39) (CHI3L1), mRNA [NM_001276] | CHI3L1 |
| interferon, gamma (IFNG), mRNA [NM_000619] | IFNG |
| chromatin licensing and DNA replication factor 1 (CDT1), mRNA [NM_030928] | CDT1 |
| chemokine (C-C motif) ligand 20 (CCL20), transcript variant 1, mRNA | CCL20 |
| [NM_004591] | |
| denticleless homolog (Drosophila) (DTL), mRNA [NM_016448] | DTL |
| uncharacterized LOC643650 (LOC643650), non-coding RNA [NR_033957] | LOC643650 |
| cyclin A2 (CCNA2), mRNA [NM_001237] | CCNA2 |
| regulator of G-protein signaling 9 (RGS9), transcript variant 3, mRNA | RGS9 |
| [NM_001165933] | |
| centrosomal protein 55 kDa (CEP55), transcript variant 1, mRNA [NM_018131] | CEP55 |
| polymerase (DNA directed), epsilon 2 (p59 subunit) (POLE2), transcript variant | POLE2 |
| 1, mRNA [NM_002692] | |
| asp (abnormal spindle) homolog, microcephaly associated (Drosophila) (ASPM), | ASPM |
| transcript variant 1, mRNA [NM_018136] | |
| transforming growth factor, beta receptor III (TGFBR3), transcript variant 1, | TGFBR3 |
| mRNA [NM_003243] | |
| retinitis pigmentosa GTPase regulator interacting protein 1 (RPGRIP1), mRNA | RPGRIP1 |
| [NM_020366] | |
| killer cell immunoglobulin-like receptor, two domains, long cytoplasmic tail, 2 | KIR2DL2 |
| (KIR2DL2), mRNA [NM_014219] | |
| Holliday junction recognition protein (HJURP), mRNA [NM_018410] | HJURP |
| signal transducing adaptor family member 1 (STAP1), mRNA [NM_012108] | STAP1 |
| topoisomerase (DNA) II alpha 170 kDa (TOP2A), mRNA [NM_001067] | TOP2A |
| TLR2_HUMAN (O60603) Toll-like receptor 2 precursor (Toll/interleukin 1 | XLOC_I2_010897 |
| receptor-like protein 4) (CD282 antigen), partial (33%) [THC2499145] | |
| protein phosphatase 2, regulatory subunit B, beta (PPP2R2B), transcript variant | PPP2R2B |
| 1, mRNA [NM_004576] | |
| SPC25, NDC80 kinetochore complex component, homolog (S. cerevisiae) | SPC25 |
| (SPC25), mRNA [NM_020675] | |
| KIAA1671 (KIAA1671), mRNA [NM_001145206] | KIAA1671 |
| budding uninhibited by benzimidazoles 1 homolog (yeast) (BUB1), mRNA | BUB1 |
| [NM_004336] | |
| G protein-coupled receptor 56 (GPR56), transcript variant 3, mRNA | GPR56 |
| [NM_201525] | |
| granzyme M (lymphocyte met-ase 1) (GZMM), mRNA [NM_005317] | GZMM |
| sterile alpha motif domain containing 3 (SAMD3), transcript variant 1, mRNA | SAMD3 |
| [NM_001017373] | |
| cathepsin W (CTSW), mRNA [NM_001335] | CTSW |
| prostaglandin F2 receptor negative regulator (PTGFRN), mRNA [NM_020440] | PTGFRN |
| BX111111 Soares_total_fetus_Nb2HF8_9w cDNA clone IMAGp998C062578, | XLOC_006752 |
| mRNA sequence [BX111111] | |
| granulysin (GNLY), transcript variant NKG5, mRNA [NM_006433] | GNLY |
| long intergenic non-protein coding RNA 239 (LINC00239), non-coding RNA | LINC00239 |
| [NR_026774] | |
| cell division cycle associated 2 (CDCA2), mRNA [NM_152562] | CDCA2 |
| granzyme H (cathepsin G-like 2, protein h-CCPX) (GZMH), mRNA [NM_033423] | GZMH |
| budding uninhibited by benzimidazoles 1 homolog beta (yeast) (BUB1B), mRNA | BUB1B |
| [NM_001211] | |
| ubiquitin-conjugating enzyme E2C (UBE2C), transcript variant 6, mRNA | UBE2C |
| [NM_181803] | |
| killer cell immunoglobulin-like receptor, two domains, short cytoplasmic tail, 4 | KIR2DS4 |
| (KIR2DS4), mRNA [NM_012314] | |
| SLAM family member 6 (SLAMF6), transcript variant 2, mRNA [NM_052931] | SLAMF6 |
| killer cell immunoglobulin-like receptor, two domains, short cytoplasmic tail, 2 | KIR2DS2 |
| (KIR2DS2), mRNA [NM_012312] | |
| BROAD Institute lincRNA (XLOC_004032), lincRNA [TCONS_00007807] | XLOC_004032 |
| perforin 1 (pore forming protein) (PRF1), transcript variant 1, mRNA | PRF1 |
| [NM_005041] | |
| GINS complex subunit 2 (Psf2 homolog) (GINS2), mRNA [NM_016095] | GINS2 |
| cell division cycle 45 homolog (S. cerevisiae) (CDC45), transcript variant 2, | CDC45 |
| mRNA [NM_003504] | |
| T-box 21 (TBX21), mRNA [NM_013351] | TBX21 |
| granzyme A (granzyme 1, cytotoxic T-lymphocyte-associated serine esterase 3) | GZMA |
| (GZMA), mRNA [NM_006144] | |
| natural killer cell group 7 sequence (NKG7), mRNA [NM_005601] | NKG7 |
| serum/glucocorticoid regulated kinase 1 (SGK1), transcript variant 1, mRNA | SGK1 |
| [NM_005627] | |
| poliovirus receptor related immunoglobulin domain containing (PVRIG), mRNA | PVRIG |
| [NM_024070] | |
| NIMA (never in mitosis gene a)-related kinase 2 (NEK2), transcript variant 1, | NEK2 |
| mRNA [NM_002497] | |
| TCR gamma alternate reading frame protein (TARP), nuclear gene encoding | TARP |
| mitochondrial protein, transcript variant 1, mRNA [NM_001003799] | |
| baculoviral IAP repeat containing 5 (BIRC5), transcript variant 3, mRNA | BIRC5 |
| [NM_001012271] | |
| cystatin F (leukocystatin) (CST7), mRNA [NM_003650] | CST7 |
| interleukin 2 receptor, beta (IL2RB), mRNA [NM_000878] | IL2RB |
| meiotic nuclear divisions 1 homolog (S. cerevisiae) (MND1), mRNA | MND1 |
| [NM_032117] | |
| sphingosine-1-phosphate receptor 5 (S1PR5), transcript variant 1, mRNA | S1PR5 |
| [NM_030760] | |
| serine/threonine kinase 32B (STK32B), mRNA [NM_018401] | STK32B |
| killer cell lectin-like receptor subfamily F, member 1 (KLRF1), mRNA | KLRF1 |
| [NM_016523] | |
| ectonucleotide pyrophosphatase/phosphodiesterase 5 (putative) (ENPP5), | ENPP5 |
| mRNA [NM_021572] | |
| interleukin 18 receptor accessory protein (IL18RAP), mRNA [NM_003853] | IL18RAP |
| GRB2-related adaptor protein 2 (GRAP2), mRNA [NM_004810] | GRAP2 |
| SH2 domain containing 2A (SH2D2A), transcript variant 5, mRNA | SH2D2A |
| [NM_001161444] | |
| complement component (3b/4b) receptor 1 (Knops blood group) (CR1), | CR1 |
| transcript variant S, mRNA [NM_000651] | |
| IQ motif containing D [Source: HGNC Symbol;Acc: 25168] [ENST00000392574] | IQCD |
| chemokine (C-C motif) ligand 5 (CCL5), mRNA [NM_002985] | CCL5 |
| killer cell lectin-like receptor subfamily D, member 1 (KLRD1), transcript variant | KLRD1 |
| 1, mRNA [NM_002262] | |
| chemokine (C-X-C motif) receptor 3 (CXCR3), transcript variant 1, mRNA | CXCR3 |
| [NM_001504] | |
| CD247 molecule [Source: HGNC Symbol;Acc: 1677] [ENST00000483825] | CD247 |
| zinc finger protein 697 (ZNF697), mRNA [NM_001080470] | ZNF697 |
| HOP homeobox (HOPX), transcript variant 2, mRNA [NM_139211] | HOPX |
| interleukin 1, beta (IL1B), mRNA [NM_000576] | IL1B |
| killer cell lectin-like receptor subfamily C, member 1 (KLRC1), transcript variant | KLRC1 |
| 1, mRNA [NM_002259] | |
| zinc finger protein 503 (ZNF503), mRNA [NM_032772] | ZNF503 |
| BROAD Institute lincRNA (XLOC_004924), lincRNA [TCONS_00010404] | XLOC_004924 |
| zeta-chain (TCR) associated protein kinase 70 kDa (ZAP70), transcript variant 1, | ZAP70 |
| mRNA [NM_001079] | |
| centromere protein F, 350/400 kDa (mitosin) (CENPF), mRNA [NM_016343] | CENPF |
| protein tyrosine phosphatase, receptor type, C-associated protein (PTPRCAP), | PTPRCAP |
| mRNA [NM_005608] | |
| killer cell lectin-like receptor subfamily C, member 3 (KLRC3), transcript variant | KLRC3 |
| 2, mRNA [NM_007333] | |
| Opa interacting protein 5 (OIP5), mRNA [NM_007280] | OIP5 |
| pyrin and HIN domain family, member 1 [Source: HGNC Symbol;Acc: 28894] | PYHIN1 |
| [ENST00000368135] | |
| PREDICTED: killer cell immunoglobulin-like receptor, three domains, long | KIR3DL1 |
| cytoplasmic tail, 1 (KIR3DL1), mRNA [XM_003403406] | |
| free fatty acid receptor 2 (FFAR2), mRNA [NM_005306] | FFAR2 |
| dedicator of cytokinesis 4 (DOCK4), mRNA [NM_014705] | DOCK4 |
| killer cell immunoglobulin-like receptor, two domains, long cytoplasmic tail, 5A | KIR2DL5A |
| (KIR2DL5A), mRNA [NM_020535] | |
| signal transducer and activator of transcription 4 (STAT4), transcript variant 1, | STAT4 |
| mRNA [NM_003151] | |
| cyclin B1 (CCNB1), mRNA [NM_031966] | CCNB1 |
| NLR family, CARD domain containing 3 (NLRC3), mRNA [NM_178844] | NLRC3 |
| CD226 molecule (CD226), mRNA [NM_006566] | CD226 |
| lin-7 homolog A (C. elegans) (LIN7A), mRNA [NM_004664] | LIN7A |
| coiled-coil domain containing 102A (CCDC102A), mRNA [NM_033212] | CCDC102A |
| SH3-binding domain kinase 1 (SBK1), mRNA [NM_001024401] | SBK1 |
| CD1d molecule (CD1D), mRNA [NM_001766] | CD1D |
| minichromosome maintenance complex component 2 (MCM2), mRNA | MCM2 |
| [NM_004526] | |
| cell division cycle associated 5 | CDCA5 |
| cysteine-rich secretory protein LCCL domain containing 2 (CRISPLD2), mRNA | CRISPLD2 |
| [NM_031476] | |
| cDNA FLJ90806 fis, clone Y79AA1000750. [AK075287] | SPC24 |
| TXK tyrosine kinase (TXK), mRNA [NM_003328] | TXK |
| v-myb myeloblastosis viral oncogene homolog (avian)-like 1 (MYBL1), transcript | MYBL1 |
| variant 2, mRNA [NM_001144755] | |
| uncharacterized protein FLJ23867 (FLJ23867), non-coding RNA [NR_026900] | FLJ23867 |
| cathelicidin antimicrobial peptide (CAMP), mRNA [NM_004345] | CAMP |
| delta-like 1 (Drosophila) (DLL1), mRNA [NM_005618] | DLL1 |
| stathmin 1 (STMN1), transcript variant 4, mRNA [NM_001145454] | STMN1 |
| prostaglandin D2 receptor (DP) (PTGDR), mRNA [NM_000953] | PTGDR |
| CD7 molecule (CD7), mRNA [NM_006137] | CD7 |
| BROAD Institute lincRNA (XLOC_011068), lincRNA [TCONS_00022791] | XLOC_011068 |
| triggering receptor expressed on myeloid cells 1 (TREM1), transcript variant 1, | TREM1 |
| mRNA [NM_018643] | |
| calmin (calponin-like, transmembrane) (CLMN), mRNA [NM_024734] | CLMN |
| T cell immunoreceptor with Ig and ITIM domains (TIGIT), mRNA [NM_173799] | TIGIT |
| chloride intracellular channel 3 (CLIC3), mRNA [NM_004669] | CLIC3 |
| interleukin 32 (IL32), transcript variant 1, mRNA [NM_001012631] | IL32 |
| adenomatosis polyposis coli down-regulated 1 (APCDD1), mRNA [NM_153000] | APCDD1 |
| zinc finger homeobox 3 (ZFHX3), transcript variant A, mRNA [NM_006885] | ZFHX3 |
| ST3 beta-galactoside alpha-2,3-sialyltransferase 6 (ST3GAL6), mRNA | ST3GAL6 |
| [NM_006100] | |
| chemokine (C-X-C motif) receptor 6 (CXCR6), mRNA [NM_006564] | CXCR6 |
| ecotropic viral integration site 5 (EVI5), mRNA [NM_005665] | EVI5 |
| cyclin-dependent kinase 1 (CDK1), transcript variant 1, mRNA [NM_001786] | CDK1 |
| Q2H1E0_CHAGB (Q2H1E0) Predicted protein, partial (5%) [THC2723627] | XLOC_002344 |
| growth arrest-specific 2 like 3 (GAS2L3), mRNA [NM_174942] | GAS2L3 |
| BRCA1 interacting protein C-terminal helicase 1 (BRIP1), mRNA [NM_032043] | BRIP1 |
| chromosome 15 open reading frame 38 (C15orf38), mRNA [NM_182616] | C15orf38 |
| ST6 (alpha-N-acetyl-neuraminyl-2,3-beta-galactosyl-1,3)-N- | ST6GALNAC2 |
| acetylgalactosaminide alpha-2,6-sialyltransferase 2 (ST6GALNAC2), mRNA | |
| [NM_006456] | |
| cDNA FLJ42228 fis, clone THYMU2041252. [AK124222] | LOC100131043 |
| NLR family, pyrin domain containing 3 (NLRP3), transcript variant 3, mRNA | NLRP3 |
| [NM_001079821] | |
| transmembrane protein 144 (TMEM144), mRNA [NM_018342] | TMEM144 |
| killer cell lectin-like receptor subfamily K, member 1 (KLRK1), mRNA | KLRK1 |
| [NM_007360] | |
| ELOVL fatty acid elongase 4 (ELOVL4), mRNA [NM_022726] | ELOVL4 |
| chemokine (C motif) ligand 1 (XCL1), mRNA [NM_002995] | XCL1 |
| Nedd4 family interacting protein 2 (NDFIP2), transcript variant 1, mRNA | NDFIP2 |
| [NM_019080] | |
| aryl hydrocarbon receptor (AHR), mRNA [NM_001621] | AHR |
| BROAD Institute lincRNA (XLOC_001266), lincRNA [TCONS_00001887] | XLOC_001266 |
| cDNA clone IMAGE: 5312439. [BC066361] | SETD7 |
| granzyme B (granzyme 2, cytotoxic T-lymphocyte-associated serine esterase 1) | GZMB |
| (GZMB), mRNA [NM_004131] | |
| coagulation factor II (thrombin) receptor-like 1 (F2RL1), mRNA [NM_005242] | F2RL1 |
| Src-like-adaptor 2 (SLA2), transcript variant 1, mRNA [NM_032214] | SLA2 |
| protein tyrosine phosphatase, non-receptor type 4 (megakaryocyte) (PTPN4), | PTPN4 |
| mRNA [NM_002830] | |
| chromatin assembly factor 1, subunit B (p60) (CHAF1B), mRNA [NM_005441] | CHAF1B |
| CD3d molecule, delta (CD3-TCR complex) (CD3D), transcript variant 1, mRNA | CD3D |
| [NM_000732] | |
| protein kinase C, eta (PRKCH), mRNA [NM_006255] | PRKCH |
| kinesin family member 20A (KIF20A), mRNA [NM_005733] | KIF20A |
| kinesin family member C1 (KIFC1), mRNA [NM_002263] | KIFC1 |
| haptoglobin (HP), transcript variant 1, mRNA [NM_005143] | HP |
| cell division cycle associated 7 (CDCA7), transcript variant 1, mRNA | CDCA7 |
| [NM_031942] | |
| integral membrane protein 2A (ITM2A), transcript variant 1, mRNA | ITM2A |
| [NM_004867] | |
| BROAD Institute lincRNA (XLOC_011350), lincRNA [TCONS_00023505] | XLOC__011350 |
| interleukin 8 (IL8), mRNA [NM_000584] | IL8 |
| uncharacterized LOC79015 (LOC79015), non-coding RNA [NR_034104] | LOC79015 |
| ubiquitin-conjugating enzyme E2T (putative) (UBE2T), mRNA [NM_014176] | UBE2T |
| pleckstrin homology domain containing, family F (with FYVE domain) member 1 | PLEKHF1 |
| (PLEKHF1), mRNA [NM_024310] | |
| killer cell lectin-like receptor subfamily B, member 1 (KLRB1), mRNA | KLRB1 |
| [NM_002258] | |
| phospholipase B1 [Source: HGNC Symbol;Acc: 30041] [ENST00000329020] | PLB1 |
| CD3g molecule, gamma (CD3-TCR complex) (CD3G), mRNA [NM_000073] | CD3G |
| NDC80 homolog, kinetochore complex component (S. cerevisiae) (NDC80), | NDC80 |
| mRNA [NM_006101] | |
| ELOVL fatty acid elongase 6 [Source: HGNC Symbol;Acc: 15829] | ELOVL6 |
| [ENST00000394607] | |
| cyclin-dependent kinase inhibitor 3 (CDKN3), transcript variant 1, mRNA | CDKN3 |
| [NM_005192] | |
| centromere protein A (CENPA), transcript variant 1, mRNA [NM_001809] | CENPA |
| ring finger protein 217 (RNF217), mRNA [NM_152553] | RNF217 |
| orosomucoid 1 (ORM1), mRNA [NM_000607] | ORM1 |
| ATPase, H+ transporting, lysosomal V0 subunit a1 (ATP6V0A1), transcript | ATP6V0A1 |
| variant 3, mRNA [NM_005177] | |
| asialoglycoprotein receptor 2 (ASGR2), transcript variant H2′, mRNA | ASGR2 |
| [NM_080912] | |
| ZW10 interactor (ZWINT), transcript variant 2, mRNA [NM_032997] | ZWINT |
| low density lipoprotein receptor-related protein 1 (LRP1), mRNA [NM_002332] | LRP1 |
| CD6 molecule (CD6), mRNA [NM_006725] | CD6 |
| G protein-coupled receptor 114 [Source: HGNC Symbol;Acc: 19010] | GPR114 |
| [ENST00000349457] | |
| aquaporin 9 (AQP9), mRNA [NM_020980] | AQP9 |
| CD3e molecule, epsilon (CD3-TCR complex) (CD3E), mRNA [NM_000733] | CD3E |
| RAS guanyl releasing protein 1 (calcium and DAG-regulated) (RASGRP1), | RASGRP1 |
| transcript variant 1, mRNA [NM_005739] | |
| CD2 molecule [Source: HGNC Symbol;Acc: 1639] [ENST00000369477] | CD2 |
| minichromosome maintenance complex component 4 (MCM4), transcript | MCM4 |
| variant 1, mRNA [NM_005914] | |
| family with sequence similarity 20, member C (FAM20C), mRNA [NM_020223] | FAM20C |
| interleukin 13 receptor, alpha 1 (IL13RA1), mRNA [NM_001560] | IL13RA1 |
| cyclin B2 (CCNB2), mRNA [NM_004701] | CCNB2 |
| GOSR1_HUMAN (O95249) Golgi SNAP receptor complex member 1 (28 kDa | XLOC_I2_015034 |
| Golgi SNARE protein) (28 kDa cis-Golgi SNARE p28) (GOS-28), partial (51%) | |
| [THC2694920] | |
| protein kinase C, theta (PRKCQ), transcript variant 1, mRNA [NM_006257] | PRKCQ |
| C-type lectin domain family 4, member E (CLEC4E), mRNA [NM_014358] | CLEC4E |
| acid phosphatase, prostate (ACPP), transcript variant 1, mRNA [NM_001099] | ACPP |
| WD repeat and FYVE domain containing 3 (WDFY3), mRNA [NM_014991] | WDFY3 |
| SH2 domain containing 1A (SH2D1A), transcript variant 2, mRNA | SH2D1A |
| [NM_001114937] | |
| ubiquitin-like with PHD and ring finger domains 1 (UHRF1), transcript variant 2, | UHRF1 |
| mRNA [NM_013282] | |
| SMAD family member 1 (SMAD1), transcript variant 1, mRNA [NM_005900] | SMAD1 |
| family with sequence similarity 198, member B (FAM198B), transcript variant 2, | FAM198B |
| mRNA [NM_016613] | |
| glycoprotein, alpha-galactosyltransferase 1 pseudogene (GGTA1P), transcript | GGTA1P |
| variant 1, non-coding RNA [NR_003191] | |
| tandem C2 domains, nuclear (TC2N), transcript variant 1, mRNA [NM_152332] | TC2N |
| BROAD Institute lincRNA (XLOC_014211), lincRNA [TCONS_00029412] | XLOC__014211 |
| defective in sister chromatid cohesion 1 homolog (S. cerevisiae) (DSCC1), mRNA | DSCC1 |
| [NM_024094] | |
| v-ets erythroblastosis virus E26 oncogene homolog 1 (avian) (ETS1), transcript | ETS1 |
| variant 2, mRNA [NM_005238] | |
| RNA binding motif protein 47 (RBM47), transcript variant 2, mRNA | RBM47 |
| [NM_019027] | |
| solute carrier family 22 (organic cation/ergothioneine transporter), member 4 | SLC22A4 |
| (SLC22A4), mRNA [NM_003059] | |
| ubiquitin associated and SH3 domain containing A (UBASH3A), transcript | UBASH3A |
| variant 1, mRNA [NM_018961] | |
| centromere protein M (CENPM), transcript variant 2, mRNA [NM_001002876] | CENPM |
| toll-like receptor 2 (TLR2), mRNA [NM_003264] | TLR2 |
| DENN/MADD domain containing 2D (DENND2D), mRNA [NM_024901] | DENND2D |
| killer cell immunoglobulin-like receptor, two domains, short cytoplasmic tail, 3 | KIR2DS3 |
| (KIR2DS3), mRNA [NM_012313] | |
| CD163 molecule (CD163), transcript variant 1, mRNA [NM_004244] | CD163 |
| zinc finger protein 516 (ZNF516), mRNA [NM_014643] | ZNF516 |
| minichromosome maintenance complex component 6 (MCM6), mRNA | MCM6 |
| [NM_005915] | |
| MANSC domain containing 1 (MANSC1), mRNA [NM_018050] | MANSC1 |
| SLAM family member 7 (SLAMF7), mRNA [NM_021181] | SLAMF7 |
| linker for activation of T cells (LAT), transcript variant 1, mRNA [NM_014387] | LAT |
| lymphocyte-activation gene 3 (LAG3), mRNA [NM_002286] | LAG3 |
| homeobox B4 (HOXB4), mRNA [NM_024015] | HOXB4 |
| CD96 molecule (CD96), transcript variant 1, mRNA [NM_198196] | CD96 |
| cytochrome P450, family 1, subfamily B, polypeptide 1 (CYP1B1), mRNA | CYP1B1 |
| [NM_000104] | |
| wntless homolog (Drosophila) (WLS), transcript variant 2, mRNA | WLS |
| [NM_001002292] | |
| interleukin 1 receptor, type I (IL1R1), mRNA [NM_000877] | IL1R1 |
| C-type lectin domain family 4, member D (CLEC4D), mRNA [NM_080387] | CLEC4D |
| sparc/osteonectin, cwcv and kazal-like domains proteoglycan (testican) 2 | SPOCK2 |
| (SPOCK2), transcript variant 2, mRNA [NM_014767] | |
| membrane bound O-acyltransferase domain containing 2 (MBOAT2), mRNA | MBOAT2 |
| [NM_138799] | |
| sortilin 1 (SORT1), transcript variant 1, mRNA [NM_002959] | SORT1 |
| kinesin family member 15 (KIF15), mRNA [NM_020242] | KIF15 |
| colony stimulating factor 3 receptor (granulocyte) (CSF3R), transcript variant 3, | CSF3R |
| mRNA [NM_156039] | |
| IL2-inducible T-cell kinase (ITK), mRNA [NM_005546] | ITK |
| histamine N-methyltransferase (HNMT), transcript variant 1, mRNA | HNMT |
| [NM_006895] | |
| tyrosine kinase with immunoglobulin-like and EGF-like domains 1 (TIE1), mRNA | TIE1 |
| [NM_005424] | |
| protein kinase, cAMP-dependent, catalytic, beta (PRKACB), transcript variant 3, | PRKACB |
| mRNA [NM_207578] | |
| CBP80/20-dependent translation initiation factor (CTIF), transcript variant 1, | CTIF |
| mRNA [NM_014772] | |
| CKLF-like MARVEL transmembrane domain containing 2 (CMTM2), transcript | CMTM2 |
| variant 1, mRNA [NM_144673] | |
| TBC1 domain family, member 9 (with GRAM domain) (TBC1D9), mRNA | TBC1D9 |
| [NM_015130] | |
| serine peptidase inhibitor, Kunitz type 1 (SPINT1), transcript variant 1, mRNA | SPINT1 |
| [NM_181642] | |
| limb bud and heart development homolog (mouse) (LBH), mRNA [NM_030915] | LBH |
| kinesin family member 2C (KIF2C), mRNA [NM_006845] | KIF2C |
| RUN and SH3 domain containing 2 (RUSC2), mRNA [NM_014806] | RUSC2 |
| granzyme K (granzyme 3; tryptase II) (GZMK), mRNA [NM_002104] | GZMK |
| CD320 molecule (CD320), transcript variant 1, mRNA [NM_016579] | CD320 |
| neural cell adhesion molecule 1 (NCAM1), transcript variant 4, mRNA | NCAM1 |
| [NM_001242608] | |
| transmembrane and tetratricopeptide repeat containing 2 (TMTC2), mRNA | TMTC2 |
| [NM_152588] | |
| asialoglycoprotein receptor 1 (ASGR1), transcript variant 1, mRNA | ASGR1 |
| [NM_001671] | |
| FXYD domain containing ion transport regulator 6 (FXYD6), transcript variant 1, | FXYD6 |
| mRNA [NM_022003] | |
| cytoskeleton associated protein 2-like (CKAP2L), mRNA [NM_152515] | CKAP2L |
| oxoeicosanoid (OXE) receptor 1 (OXER1), mRNA [NM_148962] | OXER1 |
| phosphorylase, glycogen, liver (PYGL), transcript variant 1, mRNA [NM_002863] | PYGL |
| sphingosine-1-phosphate receptor 3 (S1PR3), mRNA [NM_005226] | S1PR3 |
| Kruppel-like factor 12 [Source: HGNC Symbol;Acc: 6346] [ENST00000472022] | KLF12 |
| annexin A6 (ANXA6), transcript variant 1, mRNA [NM_001155] | ANXA6 |
| B-cell CLL/lymphoma 6 (BCL6), transcript variant 2, mRNA [NM_001130845] | BCL6 |
| Kruppel-like factor 5 (intestinal) (KLF5), mRNA [NM_001730] | KLF5 |
| enolase 2 (gamma, neuronal) (ENO2), mRNA [NM_001975] | ENO2 |
| CD27 molecule (CD27), mRNA [NM_001242] | CD27 |
| retinol binding protein 7, cellular (RBP7), mRNA [NM_052960] | RBP7 |
| cDNA FLJ42271 fis, clone TKIDN2015788. [AK124265] | LOC100131490 |
| protein tyrosine phosphatase, non-receptor type 7 (PTPN7), transcript variant | PTPN7 |
| 2, mRNA [NM_080588] | |
| hydroxy-delta-5-steroid dehydrogenase, 3 beta- and steroid delta-isomerase 7 | HSD3B7 |
| (HSD3B7), transcript variant 2, mRNA [NM_001142777] | |
| ectonucleoside triphosphate diphosphohydrolase 1 [Source: HGNC | ENTPD1 |
| Symbol;Acc: 3363] [ENST00000371206] | |
| centromere protein K (CENPK), mRNA [NM_022145] | CENPK |
| histidine ammonia-lyase (HAL), mRNA [NM_002108] | HAL |
| G protein-coupled receptor 125 (GPR125), mRNA [NM_145290] | GPR125 |
| uncharacterized LOC100289495 (LOC100289495), non-coding RNA | LOC100289495 |
| [NR_040022] | |
| ral guanine nucleotide dissociation stimulator-like 4 (RGL4), mRNA | RGL4 |
| [NM_153615] | |
| CD8b molecule (CD8B), transcript variant 5, mRNA [NM_004931] | CD8B |
| deleted in lymphocytic leukemia 1 (non-protein coding) (DLEU1), non-coding | DLEU1 |
| RNA [NR_002605] | |
| interleukin 18 receptor 1 (IL18R1), mRNA [NM_003855] | IL18R1 |
| dachshund homolog 1 (Drosophila) (DACH1), transcript variant 1, mRNA | DACH1 |
| [NM_080759] | |
| CD8a molecule (CD8A), transcript variant 3, mRNA [NM_001145873] | CD8A |
| E2F transcription factor 7 (E2F7), mRNA [NM_203394] | E2F7 |
| tumor necrosis factor receptor superfamily, member 10c, decoy without an | TNFRSF10C |
| intracellular domain (TNFRSF10C), mRNA [NM_003841] | |
| TPX2, microtubule-associated, homolog (Xenopus laevis) (TPX2), mRNA | TPX2 |
| [NM_012112] | |
| toll-like receptor 5 (TLR5), mRNA [NM_003268] | TLR5 |
| CD5 molecule (CD5), mRNA [NM_014207] | CD5 |
| uncharacterized LOC387895 (LOC387895), non-coding RNA [NR_033878] | LOC387895 |
| stabilin 1 (STABI), mRNA [NM_015136] | STAB1 |
| arrestin domain containing 4 (ARRDC4), mRNA [NM_183376] | ARRDC4 |
| PREDICTED: hypothetical LOC100506119, transcript variant 1 (LOC100506119), | LOC100506119 |
| miscRNA [XR_108951] | |
| pituitary tumor-transforming 1 (PTTG1), mRNA [NM_004219] | PTTG1 |
| solute carrier family 11 (proton-coupled divalent metal ion transporters), | SLC11A1 |
| member 1 (SLC11A1), mRNA [NM_000578] | |
| peptidyl arginine deiminase, type IV (PADI4), mRNA [NM_012387] | PADI4 |
| V-set and transmembrane domain containing 1 (VSTM1), mRNA [NM_198481] | VSTM1 |
| 5-oxoprolinase (ATP-hydrolysing) (OPLAH), mRNA [NM_017570] | OPLAH |
| peptidyl arginine deiminase, type II (PADI2), mRNA [NM_007365] | PADI2 |
| solute carrier family 38, member 1 [Source: HGNC Symbol;Acc: 13447] | SLC38A1 |
| [ENST00000550173] | |
| carbohydrate (chondroitin 4) sulfotransferase 12 (CHST12), transcript variant 3, | CHST12 |
| mRNA [NM_018641] | |
| actin binding LIM protein 1 (ABLIM1), transcript variant 3, mRNA | ABLIM1 |
| [NM_001003408] | |
| cation channel, sperm associated 1 (CATSPER1), mRNA [NM_053054] | CATSPER1 |
| cat eye syndrome chromosome region, candidate 6 (CECR6), transcript variant | CECR6 |
| 1, mRNA [NM_031890] | |
| toll-like receptor 6 (TLR6), mRNA [NM_006068] | TLR6 |
| sialic acid binding Ig-like lectin 9 (SIGLEC9), transcript variant 2, mRNA | SIGLEC9 |
| [NM_014441] | |
| homeobox B2 (HOXB2), mRNA [NM_002145] | HOXB2 |
| plexin domain containing 2 (PLXDC2), mRNA [NM_032812] | PLXDC2 |
| cAMP responsive element binding protein 5 (CREB5), transcript variant 1, | CREB5 |
| mRNA [NM_182898] | |
| uncharacterized LOC100240735 (LOC100240735), non-coding RNA | LOC100240735 |
| [NR_026658] | |
| 3-hydroxyanthranilate 3,4-dioxygenase [Source: HGNC Symbol;Acc: 4796] | HAAO |
| [ENST00000402268] | |
| solute carrier family 8 (sodium/calcium exchanger), member 1 (SLC8A1), | SLC8A1 |
| transcript variant A, mRNA [NM_021097] | |
| signaling threshold regulating transmembrane adaptor 1 (SIT1), mRNA | SIT1 |
| [NM_014450] | |
| NUF2, NDC80 kinetochore complex component, homolog (S. cerevisiae) | NUF2 |
| (NUF2), transcript variant 1, mRNA [NM_145697] | |
| B-cell CLL/lymphoma 11B (zinc finger protein) (BCL11B), transcript variant 1, | BCL11B |
| mRNA [NM_138576] | |
| activated leukocyte cell adhesion molecule (ALCAM), transcript variant 1, | ALCAM |
| mRNA [NM_001627] | |
| BAH domain and coiled-coil containing 1 (BAHCC1), mRNA [NM_001080519] | BAHCC1 |
| lipase, hormone-sensitive (LIPE), mRNA [NM_005357] | LIPE |
| B and T lymphocyte associated (BTLA), transcript variant 1, mRNA | BTLA |
| [NM_181780] | |
| family with sequence similarity 169, member A (FAM169A), mRNA | FAM169A |
| [NM_015566] | |
| RAD54-like (S. cerevisiae) (RAD54L), transcript variant 1, mRNA [NM_003579] | RAD54L |
| proline/serine-rich coiled-coil 1 (PSRC1), transcript variant 1, mRNA | PSRC1 |
| [NM_032636] | |
| poliovirus receptor (PVR), transcript variant 1, mRNA [NM_006505] | PVR |
| inducible T-cell co-stimulator (ICOS), mRNA [NM_012092] | ICOS |
| RAD51 associated protein 1 (RAD51AP1), transcript variant 2, mRNA | RAD51AP1 |
| [NM_006479] | |
| ras homolog gene family, member H (RHOH), mRNA [NM_004310] | RHOH |
| cytidine deaminase (CDA), mRNA [NM_001785] | CDA |
| WD repeat domain 67 (WDR67), transcript variant 2, mRNA [NM_001145088] | WDR67 |
| lymphoid enhancer-binding factor 1 (LEF1), transcript variant 1, mRNA | LEF1 |
| [NM_016269] | |
| CD40 ligand (CD40LG), mRNA [NM_000074] | CD40LG |
| CD93 molecule (CD93), mRNA [NM_012072] | CD93 |
| solute carrier family 16, member 5 (monocarboxylic acid transporter 6) | SLC16A5 |
| (SLC16A5), mRNA [NM_004695] | |
| family with sequence similarity 7, member A1 (non-protein coding) (FAM7A1), | FAM7A1 |
| non-coding RNA [NR_026858] | |
| H2.0-like homeobox (HLX), mRNA [NM_021958] | HLX |
| frizzled family receptor 1 (FZD1), mRNA [NM_003505] | FZD1 |
| transcription factor 7 (T-cell specific, HMG-box) (TCF7), transcript variant 1, | TCF7 |
| mRNA [NM_003202] | |
| C-type lectin domain family 7, member A (CLEC7A), transcript variant 6, mRNA | CLEC7A |
| [NM_197954] | |
| RAS guanyl releasing protein 4 (RASGRP4), transcript variant a, mRNA | RASGRP4 |
| [NM_170604] | |
| S100 calcium binding protein Z (S100Z), mRNA [NM_130772] | S100Z |
| solute carrier family 36 (proton/amino acid symporter), member 1 (SLC36A1), | SLC36A1 |
| mRNA [NM_078483] | |
| interleukin-1 receptor-associated kinase 3 (IRAK3), transcript variant 1, mRNA | IRAK3 |
| [NM_007199] | |
| RALBP1 associated Eps domain containing 2 (REPS2), transcript variant 1, | REPS2 |
| mRNA [NM_004726] | |
| cell division cycle associated 8 (CDCA8), mRNA [NM_018101] | CDCA8 |
| calcium channel, voltage-dependent, alpha 2/delta subunit 3 (CACNA2D3), | CACNA2D3 |
| mRNA [NM_018398] | |
| ATPase, class II, type 9A (ATP9A), mRNA [NM_006045] | ATP9A |
| toll-like receptor 4 (TLR4), transcript variant 1, mRNA [NM_138554] | TLR4 |
| complement component 5a receptor 1 (C5AR1), mRNA [NM_001736] | C5AR1 |
| junction plakoglobin (JUP), transcript variant 1, mRNA [NM_002230] | JUP |
| ribonuclease, RNase A family, k6 (RNASE6), mRNA [NM_005615] | RNASE6 |
| steroid 5 alpha-reductase 3 (SRD5A3), mRNA [NM_024592] | SRD5A3 |
| vanin 3 (VNN3), transcript variant 1, non-coding RNA [NR_028291] | VNN3 |
| neutrophil cytosolic factor 4, 40 kDa (NCF4), transcript variant 1, mRNA | NCF4 |
| [NM_000631] | |
| C3 and PZP-like, alpha-2-macroglobulin domain containing 8 (CPAMD8), mRNA | CPAMD8 |
| [NM_015692] | |
| inositol(myo)-1(or 4)-monophosphatase 2 (IMPA2), mRNA [NM_014214] | IMPA2 |
| dual specificity phosphatase 6 (DUSP6), transcript variant 1, mRNA | DUSP6 |
| [NM_001946] | |
| solute carrier family 1 (glial high affinity glutamate transporter), member 3 | SLC1A3 |
| (SLC1A3), transcript variant 1, mRNA [NM_004172] | |
| STEAP family member 4 (STEAP4), transcript variant 2, mRNA [NM_001205315] | STEAP4 |
| signal-regulatory protein alpha (SIRPA), transcript variant 1, mRNA | SIRPA |
| [NM_001040022] | |
| prokineticin 2 (PROK2), transcript variant 2, mRNA [NM_021935] | PROK2 |
| homer homolog 3 (Drosophila) (HOMER3), transcript variant 2, mRNA | HOMER3 |
| [NM_004838] | |
| proliferating cell nuclear antigen (PCNA), transcript variant 1, mRNA | PCNA |
| [NM_002592] | |
| ALU5_HUMAN (P39192) Alu subfamily Sc sequence contamination warning | LOC100506190 |
| entry, partial (9%) [THC2542147] | |
| leucine rich repeat neuronal 3 (LRRN3), transcript variant 3, mRNA | LRRN3 |
| [NM_018334] | |
| pellino homolog 3 (Drosophila) (PELI3), transcript variant 1, mRNA | PELI3 |
| [NM_145065] | |
| glycosyltransferase 1 domain containing 1 (GLT1D1), mRNA [NM_144669] | GLT1D1 |
| T cell receptor associated transmembrane adaptor 1 (TRAT1), mRNA | TRAT1 |
| [NM_016388] | |
| interferon gamma receptor 2 (interferon gamma transducer 1) (IFNGR2), mRNA | IFNGR2 |
| [NM_005534] | |
| versican (VCAN), transcript variant 1, mRNA [NM_004385] | VCAN |
| fructose-1,6-bisphosphatase 1 (FBP1), transcript variant 1, mRNA [NM_000507] | FBP1 |
| activating transcription factor 3 (ATF3), transcript variant 4, mRNA | ATF3 |
| [NM_001040619] | |
| thymocyte selection associated (THEMIS), transcript variant 1, mRNA | THEMIS |
| [NM_001164685] | |
| aldehyde dehydrogenase 18 family, member A1 (ALDH18A1), nuclear gene | ALDH18A1 |
| encoding mitochondrial protein, transcript variant 1, mRNA [NM_002860] | |
| platelet-activating factor receptor (PTAFR), transcript variant 2, mRNA | PTAFR |
| [NM_001164722] | |
| kinesin family member 14 (KIF14), mRNA [NM_014875] | KIF14 |
| cyclin E2 (CCNE2), mRNA [NM_057749] | CCNE2 |
| potassium voltage-gated channel, Isk-related family, member 3 (KCNE3), mRNA | KCNE3 |
| [NM_005472] | |
| fidgetin-like 1 (FIGNL1), transcript variant 1, mRNA [NM_001042762] | FIGNL1 |
| myeloid cell nuclear differentiation antigen (MNDA), mRNA [NM_002432] | MNDA |
| PREDICTED: hypothetical protein LOC100652853 (LOC100652853), mRNA | HP07349 |
| [XM_003403534] | |
| carboxypeptidase, vitellogenic-like (CPVL), transcript variant 2, mRNA | CPVL |
| [NM_019029] | |
| claudin 23 (CLDN23), mRNA [NM_194284] | CLDN23 |
| cancer susceptibility candidate 5 (CASC5), transcript variant 1, mRNA | CASC5 |
| [NM_170589] | |
| inositol 1,4,5-trisphosphate receptor, type 3 (ITPR3), mRNA [NM_002224] | ITPR3 |
| ral guanine nucleotide dissociation stimulator-like 1 (RGL1), mRNA | RGL1 |
| [NM_015149] | |
| transmembrane 6 superfamily member 1 (TM6SF1), transcript variant 1, mRNA | TM6SF1 |
| [NM_023003] | |
| solute carrier family 22, member 15 (SLC22A15), mRNA [NM_018420] | SLC22A15 |
| BMP2 inducible kinase (BMP2K), transcript variant 2, mRNA [NM_017593] | BMP2K |
| RAR-related orphan receptor A (RORA), transcript variant 2, mRNA | RORA |
| [NM_134260] | |
| ectonucleotide pyrophosphatase/phosphodiesterase 4 (putative) (ENPP4), | ENPP4 |
| mRNA [NM_014936] | |
| minichromosome maintenance complex component 3 (MCM3), mRNA | MCM3 |
| [NM_002388] | |
| complement component 3a receptor 1 (C3AR1), mRNA [NM_004054] | C3AR1 |
| B-cell CLL/lymphoma 2 (BCL2), nuclear gene encoding mitochondrial protein, | BCL2 |
| transcript variant alpha, mRNA [NM_000633] | |
| leucine-rich alpha-2-glycoprotein 1 (LRG1), mRNA [NM_052972] | LRG1 |
| NACC family member 2, BEN and BTB (POZ) domain containing (NACC2), mRNA | NACC2 |
| [NM_144653] | |
| growth arrest-specific 2 like 1 (GAS2L1), transcript variant 3, mRNA | GAS2L1 |
| [NM_152237] | |
| RAS p21 protein activator 3 (RASA3), mRNA [NM_007368] | RASA3 |
| C-type lectin domain family 12, member A (CLEC12A), transcript variant 1, | CLEC12A |
| mRNA [NM_138337] | |
| centromere protein O (CENPO), transcript variant 1, mRNA [NM_024322] | CENPO |
| phospholipase B domain containing 1 (PLBD1), mRNA [NM_024829] | PLBD1 |
| interferon gamma receptor 1 (IFNGR1), mRNA [NM_000416] | IFNGR1 |
| aurora kinase A (AURKA), transcript variant 1, mRNA [NM_198433] | AURKA |
| fms-related tyrosine kinase 3 ligand (FLT3LG), transcript variant 3, mRNA | FLT3LG |
| [NM_001459] | |
| cytoplasmic FMR1 interacting protein 1 (CYFIP1), transcript variant 1, mRNA | CYFIP1 |
| [NM_014608] | |
| kallikrein-related peptidase 3 (KLK3), transcript variant 3, mRNA | KLK3 |
| [NM_001030047] | |
| diacylglycerol O-acyltransferase 2 (DGAT2), mRNA [NM_032564] | DGAT2 |
| KIAA1598 (KIAA1598), transcript variant 1, mRNA [NM_001127211] | KIAA1598 |
| lymphocyte-specific protein tyrosine kinase (LCK), transcript variant 2, mRNA | LCK |
| [NM_005356] | |
| thyroid hormone receptor interactor 13 (TRIP13), transcript variant 1, mRNA | TRIP13 |
| [NM_004237] | |
| disrupted in renal carcinoma 2 (DIRC2), mRNA [NM_032839] | DIRC2 |
| RAB13, member RAS oncogene family (RAB13), mRNA [NM_002870] | RAB13 |
| leucine rich repeat containing 4 (LRRC4), mRNA [NM_022143] | LRRC4 |
| PREDICTED: hypothetical protein LOC100130458 (LOC100130458), mRNA | LOC100130458 |
| [XM_001716901] | |
| mal, T-cell differentiation protein (MAL), transcript variant a, mRNA | MAL |
| [NM_002371] | |
| tensin 3 (TNS3), mRNA [NM_022748] | TNS3 |
| lysophosphatidylcholine acyltransferase 2 (LPCAT2), mRNA [NM_017839] | LPCAT2 |
| DENN/MADD domain containing 1A (DENND1A), transcript variant 2, mRNA | DENND1A |
| [NM_024820] | |
| protein phosphatase 1, regulatory subunit 32 (PPP1R32), nuclear gene | PPP1R32 |
| encoding mitochondrial protein, transcript variant 1, mRNA [NM_145017] | |
| cDNA FLJ45829 fis, clone NT2RP8006452. [AK127729] | LRRK2 |
| membrane-associated ring finger (C3HC4) 1 (MARCH1), transcript variant 2, | MARCH1 |
| mRNA [NM_017923] | |
| cold shock domain protein A (CSDA), transcript variant 1, mRNA [NM_003651] | CSDA |
| GATA binding protein 3 (GATA3), transcript variant 1, mRNA [NM_001002295] | GATA3 |
| microsomal glutathione S-transferase 1 (MGST1), transcript variant 1c, mRNA | MGST1 |
| [NM_145791] | |
| calcium/calmodulin-dependent protein kinase IV (CAMK4), mRNA | CAMK4 |
| [NM_001744] | |
| minichromosome maintenance complex component 8 (MCM8), transcript | MCM8 |
| variant 2, mRNA [NM_182802] | |
| BROAD Institute lincRNA (XLOC_014161), lincRNA [TCONS_00029333] | XLOC__014161 |
| coatomer protein complex, subunit gamma 2 (COPG2), mRNA [NM_012133] | COPG2 |
| tumor protein p53 inducible protein 3 (TP53I3), transcript variant 1, mRNA | TP53I3 |
| [NM_004881] | |
| egf-like module containing, mucin-like, hormone receptor-like 2 (EMR2), | EMR2 |
| transcript variant 1, mRNA [NM_013447] | |
| vanin 1 (VNN1), mRNA [NM_004666] | VNN1 |
| kinesin family member 11 (KIF11), mRNA [NM_004523] | KIF11 |
| mutS homolog 2, colon cancer, nonpolyposis type 1 (E. coli) (MSH2), mRNA | MSH2 |
| [NM_000251] | |
| WEE1 homolog (S. pombe) (WEE1), transcript variant 1, mRNA [NM_003390] | WEE1 |
| purinergic receptor P2Y, G-protein coupled, 13 (P2RY13), mRNA [NM_176894] | P2RY13 |
| helicase (DNA) B (HELB), mRNA [NM_033647] | HELB |
| tetratricopeptide repeat and ankyrin repeat containing 1 (TRANK1), mRNA | TRANK1 |
| [NM_014831] | |
| chemokine (C-C motif) receptor 1 (CCR1), mRNA [NM_001295] | CCR1 |
| PREDICTED: hypothetical LOC100505921 (LOC100505921), miscRNA | LOC100505921 |
| [XR_108739] | |
| lipoma HMGIC fusion partner-like 2 (LHFPL2), mRNA [NM_005779] | LHFPL2 |
| solute carrier family 24 (sodium/potassium/calcium exchanger), member 4 | SLC24A4 |
| (SLC24A4), transcript variant 2, mRNA [NM_153647] | |
| lactotransferrin (LTF), transcript variant 1, mRNA [NM_002343] | LTF |
| Fc receptor-like 5 (FCRL5), transcript variant 2, mRNA [NM_001195388] | FCRL5 |
| protocadherin 9 (PCDH9), transcript variant 1, mRNA [NM_203487] | PCDH9 |
| retinoblastoma-like 1 (p107) (RBL1), transcript variant 1, mRNA [NM_002895] | RBL1 |
| MAD2 mitotic arrest deficient-like 1 (yeast) (MAD2L1), mRNA [NM_002358] | MAD2L1 |
| SHC SH2-domain binding protein 1 (SHCBP1), mRNA [NM_024745] | SHCBP1 |
| folate receptor 3 (gamma) (FOLR3), mRNA [NM_000804] | FOLR3 |
| aldehyde dehydrogenase 2 family (mitochondrial) (ALDH2), nuclear gene | ALDH2 |
| encoding mitochondrial protein, transcript variant 1, mRNA [NM_000690] | |
| transforming growth factor, beta-induced, 68 kDa (TGFBI), mRNA [NM_000358] | TGFBI |
| lymphocyte antigen 96 (LY96), transcript variant 1, mRNA [NM_015364] | LY96 |
| galactosidase, beta 1-like (GLB1L), mRNA [NM_024506] | GLB1L |
| NEL-like 2 (chicken) (NELL2), transcript variant 2, mRNA [NM_006159] | NELL2 |
| serine carboxypeptidase 1 (SCPEP1), mRNA [NM_021626] | SCPEP1 |
| v-myb myeloblastosis viral oncogene homolog (avian)-like 2 (MYBL2), mRNA | MYBL2 |
| [NM_002466] | |
| dysferlin, limb girdle muscular dystrophy 2B (autosomal recessive) (DYSF), | DYSF |
| transcript variant 8, mRNA [NM_003494] | |
| NLR family, apoptosis inhibitory protein (NAIP), transcript variant 1, mRNA | NAIP |
| [NM_004536] | |
| CD36 molecule (thrombospondin receptor) (CD36), transcript variant 2, mRNA | CD36 |
| [NM_001001547] | |
| lymphocyte antigen 86 (LY86), mRNA [NM_004271] | LY86 |
| PML-RARA regulated adaptor molecule 1 (PRAM1), mRNA [NM_032152] | PRAM1 |
| interleukin 7 receptor (IL7R), mRNA [NM_002185] | IL7R |
| PDZ and LIM domain 7 (enigma) (PDLIM7), transcript variant 1, mRNA | PDLIM7 |
| [NM_005451] | |
| membrane-spanning 4-domains, subfamily A, member 4 (MS4A4A), transcript | MS4A4A |
| variant 2, mRNA [NM_024021] | |
| Fc fragment of IgG, low affinity IIa, receptor (CD32) (FCGR2A), transcript variant | FCGR2A |
| 2, mRNA [NM_021642] | |
| dedicator of cytokinesis 5 (DOCK5), mRNA [NM_024940] | DOCK5 |
| RAB34, member RAS oncogene family (RAB34), transcript variant 3, mRNA | RAB34 |
| [NM_001142625] | |
| cytochrome b reductase 1 (CYBRD1), transcript variant 1, mRNA [NM_024843] | CYBRD1 |
| ribonuclease, RNase A family, 3 (RNASE3), mRNA [NM_002935] | RNASE3 |
| heparanase (HPSE), transcript variant 1, mRNA [NM_006665] | HPSE |
| elastase, neutrophil expressed (]LANE), mRNA [NM_001972] | ]LANE |
| ring finger protein 24 (RNF24), transcript variant 1, mRNA [NM_007219] | RNF24 |
| myeloid-associated differentiation marker (MYADM), transcript variant 1, | MYADM |
| mRNA [NM_001020818] | |
| SWI/SNF related, matrix associated, actin dependent regulator of chromatin, | SMARCD3 |
| subfamily d, member 3 (SMARCD3), transcript variant 2, mRNA [NM_003078] | |
| membrane-spanning 4-domains, subfamily A, member 6A (MS4A6A), transcript | MS4A6A |
| variant 1, mRNA [NM_152852] | |
| leukocyte immunoglobulin-like receptor, subfamily B (with TM and ITIM | LILRB3 |
| domains), member 3 (LILRB3), transcript variant 2, mRNA [NM_006864] | |
| lipid phosphate phosphatase-related protein type 2 (LPPR2), transcript variant | LPPR2 |
| 1, mRNA [NM_022737] | |
| flap structure-specific endonuclease 1 (FEN1), mRNA [NM_004111] | FEN1 |
| toll-like receptor 8 (TLR8), mRNA [NM_138636] | TLR8 |
| G protein-coupled receptor 171 (GPR171), mRNA [NM_013308] | GPR171 |
| platelet/endothelial cell adhesion molecule (PECAM1), mRNA [NM_000442] | PECAM1 |
| renin binding protein (RENBP), mRNA [NM_002910] | RENBP |
| interleukin 18 (interferon-gamma-inducing factor) (IL18), transcript variant 1, | IL18 |
| mRNA [NM_001562] | |
| complement factor D (adipsin) (CFD), mRNA [NM_001928] | CFD |
| ArfGAP with dual PH domains 2 (ADAP2), mRNA [NM_018404] | ADAP2 |
| proline rich Gla (G-carboxyglutamic acid) 4 (transmembrane) (PRRG4), mRNA | PRRG4 |
| [NM_024081] | |
| LIM domain only 2 (rhombotin-like 1) (LMO2), transcript variant 1, mRNA | LMO2 |
| [NM_005574] | |
| replication protein A2, 32 kDa (RPA2), mRNA [NM_002946] | RPA2 |
| IMP1 inner mitochondrial membrane peptidase-like (S. cerevisiae) (IMMP1L), | IMMP1L |
| nuclear gene encoding mitochondrial protein, mRNA [NM_144981] | |
| RAB31, member RAS oncogene family (RAB31), mRNA [NM_006868] | RAB31 |
| multiple C2 domains, transmembrane 1 (MCTP1), transcript variant L, mRNA | MCTP1 |
| [NM_024717] | |
| biliverdin reductase B (flavin reductase (NADPH)) (BLVRB), mRNA [NM_000713] | BLVRB |
| signaling lymphocytic activation molecule family member 1 (SLAMF1), mRNA | SLAMF1 |
| [NM_003037] | |
| serglycin (SRGN), transcript variant 1, mRNA [NM_002727] | SRGN |
| potassium channel tetramerisation domain containing 12 (KCTD12), mRNA | KCTD12 |
| [NM_138444] | |
| TIMP metallopeptidase inhibitor 2 (TIMP2), mRNA [NM_003255] | TIMP2 |
| eomesodermin (EOMES), mRNA [NM_005442] | EOMES |
| solute carrier family 7 (amino acid transporter light chain, y + L system), member | SLC7A7 |
| 7 (SLC7A7), transcript variant 3, mRNA [NM_001126106] | |
| leukocyte immunoglobulin-like receptor, subfamily A (without TM domain), | LILRA3 |
| member 3 (LILRA3), transcript variant 1, mRNA [NM_006865] | |
| sialic acid binding Ig-like lectin 15 (SIGLEC15), mRNA [NM_213602] | SIGLEC15 |
| DNA-damage regulated autophagy modulator 1 (DRAM1), mRNA [NM_018370] | DRAM1 |
| cell division cycle associated 3 (CDCA3), mRNA [NM_031299] | CDCA3 |
| sulfatase 2 (SULF2), transcript variant 1, mRNA [NM_018837] | SULF2 |
| lectin, galactoside-binding, soluble, 3 (LGALS3), transcript variant 1, mRNA | LGALS3 |
| [NM_002306] | |
| NLR family, pyrin domain containing 12 (NLRP12), transcript variant 1, mRNA | NLRP12 |
| [NM_033297] | |
| CD33 molecule (CD33), transcript variant 1, mRNA [NM_001772] | CD33 |
| inositol 1,4,5-trisphosphate receptor interacting protein-like 2 (ITPRIPL2), | ITPRIPL2 |
| transcript variant 1, mRNA [NM_001034841] | |
| cell division cycle 20 homolog (S. cerevisiae) (CDC20), mRNA [NM_001255] | CDC20 |
| transmembrane protein 127 (TMEM127), transcript variant 1, mRNA | TMEM127 |
| [NM_017849] | |
| GTPase, IMAP family member 7 (GIMAP7), mRNA [NM_153236] | GIMAP7 |
| calcineurin-like phosphoesterase domain containing 1 (CPPED1), transcript | CPPED1 |
| variant 1, mRNA [NM_018340] | |
| Fanconi anemia, complementation group I (FANCI), transcript variant 2, mRNA | FANCI |
| [NM_018193] | |
| akirin 2 (AKIRIN2), mRNA [NM_018064] | AKIRIN2 |
| S100 calcium binding protein A12 (S100A12), mRNA [NM_005621] | S100A12 |
| serine threonine kinase 39 (STK39), mRNA [NM_013233] | STK39 |
| C-type lectin domain family 10, member A (CLEC10A), transcript variant 1, | CLEC10A |
| mRNA [NM_182906] | |
| CD68 molecule (CD68), transcript variant 1, mRNA [NM_001251] | CD68 |
| E2F transcription factor 5, p130-binding (E2F5), transcript variant 1, mRNA | E2F5 |
| [NM_001951] | |
| carbohydrate (N-acetylgalactosamine 4-sulfate 6-O) sulfotransferase 15 | CHST15 |
| (CHST15), transcript variant 2, mRNA [NM_014863] | |
| zinc finger protein 668 (ZNF668), transcript variant 1, mRNA [NM_001172668] | ZNF668 |
| C-type lectin domain family 4, member A (CLEC4A), transcript variant 1, mRNA | CLEC4A |
| [NM_016184] | |
| osteoclast associated, immunoglobulin-like receptor (OSCAR), transcript variant | OSCAR |
| 1, mRNA [NM_206818] | |
| CD28 molecule (CD28), transcript variant 1, mRNA [NM_006139] | CD28 |
| cystatin A (stefin A) (CSTA), mRNA [NM_005213] | CSTA |
| transmembrane protein 64 (TMEM64), transcript variant 1, mRNA | TMEM64 |
| [NM_001008495] | |
| glycosyltransferase 25 domain containing 1 (GLT25D1), mRNA [NM_024656] | GLT25D1 |
| lectin, galactoside-binding, soluble, 2 (LGALS2), mRNA [NM_006498] | LGALS2 |
| 6-phosphofructo-2-kinasegructose-2,6-biphosphatase 4 (PFKFB4), mRNA | PFKFB4 |
| [NM_004567] | |
| adhesion molecule, interacts with CXADR antigen 1 (AMICA1), transcript variant | AMICA1 |
| 2, mRNA [NM_153206] | |
| syntaxin 10 (STX10), mRNA [NM_003765] | STX10 |
| spleen tyrosine kinase (SYK), transcript variant 1, mRNA [NM_003177] | SYK |
| ArfGAP with GTPase domain, ankyrin repeat and PH domain 2 (AGAP2), | AGAP2 |
| transcript variant 1, mRNA [NM_001122772] | |
| acyl-CoA synthetase long-chain family member 1 (ACSL1), mRNA [NM_001995] | ACSL1 |
| replication factor C (activator 1) 4, 37 kDa (RFC4), transcript variant 1, mRNA | RFC4 |
| [NM_002916] | |
| immunoglobulin superfamily, member 6 (IGSF6), mRNA [NM_005849] | IGSF6 |
| colony stimulating factor 2 receptor, alpha, low-affinity (granulocyte- | CSF2RA |
| macrophage) (CSF2RA), transcript variant 6, mRNA [NM_172249] | |
| death-associated protein kinase 1 (DAPK1), mRNA [NM_004938] | DAPK1 |
| complement component (3d/Epstein Barr virus) receptor 2 (CR2), transcript | CR2 |
| variant 1, mRNA [NM_001006658] | |
| caspase recruitment domain family, member 9 (CARD9), transcript variant 2, | CARD9 |
| mRNA [NM_052814] | |
| zinc finger protein 790 (ZNF790), transcript variant 4, mRNA [NM_001242802] | ZNF790 |
| neutrophil cytosolic factor 2 (NCF2), transcript variant 1, mRNA [NM_000433] | NCF2 |
| Morf4 family associated protein 1-like 1 (MRFAP1L1), mRNA [NM_203462] | MRFAP1L1 |
| aurora kinase B (AURKB), mRNA [NM_004217] | AURKB |
| alanyl (membrane) aminopeptidase (ANPEP), mRNA [NM_001150] | ANPEP |
| lymphotoxin beta receptor (TNFR superfamily, member 3) (LTBR), mRNA | LTBR |
| [NM_002342] | |
| solute carrier family 15, member 3 (SLC15A3), transcript variant 1, mRNA | SLC15A3 |
| [NM_016582] | |
| guanine nucleotide binding protein (G protein), beta polypeptide 4 (GNB4), | GNB4 |
| mRNA [NM_021629] | |
| protein kinase, cAMP-dependent, regulatory, type I, beta (PRKAR1B), transcript | PRKAR1B |
| variant 1, mRNA [NM_001164761] | |
| multiple EGF-like-domains 9 (MEGF9), mRNA [NM_001080497] | MEGF9 |
| ATP-binding cassette, sub-family D (ALD), member 1 (ABCD1), mRNA | ABCD1 |
| [NM_000033] | |
| S100 calcium binding protein All (S100A11), mRNA [NM_005620] | S100A11 |
| platelet derived growth factor C (PDGFC), transcript variant 1, mRNA | PDGFC |
| [NM_016205] | |
| ATPase family, AAA domain containing 1 (ATAD1), mRNA [NM_032810] | ATAD1 |
| sialic acid binding Ig-like lectin 1, sialoadhesin (SIGLEC1), mRNA [NM_023068] | SIGLEC1 |
| tumor necrosis factor (ligand) superfamily, member 13 (TNFSF13), transcript | TNFSF13 |
| variant gamma, mRNA [NM_172088] | |
| CD302 molecule (CD302), transcript variant 1, mRNA [NM_014880] | CD302 |
| Rho guanine nucleotide exchange factor (GEF) 10-like (ARHGEF10L), transcript | ARHGEF10L |
| variant 1, mRNA [NM_018125] | |
| hematopoietically expressed homeobox (HHEX), mRNA [NM_002729] | HHEX |
| keratin 23 (histone deacetylase inducible) (KRT23), mRNA [NM_015515] | KRT23 |
| establishment of cohesion 1 homolog 2 (S. cerevisiae) (ESCO2), mRNA | ESCO2 |
| [NM_001017420] | |
| retinoic acid receptor responder (tazarotene induced) 3 (RARRES3), mRNA | RARRES3 |
| [NM_004585] | |
| neutral cholesterol ester hydrolase 1 (NCEH1), transcript variant 2, mRNA | NCEH1 |
| [NM_020792] | |
| anoctamin 10 (ANO10), transcript variant 2, mRNA [NM_001204831] | ANO10 |
| RAB32, member RAS oncogene family (RAB32), mRNA [NM_006834] | RAB32 |
| Fc fragment of IgG, receptor, transporter, alpha (FCGRT), transcript variant 2, | FCGRT |
| mRNA [NM_004107] | |
| chromosome 3 open reading frame 26 (C3orf26), transcript variant 1, mRNA | C3orf26 |
| [NM_032359] | |
| ArfGAP with RhoGAP domain, ankyrin repeat and PH domain 3 (ARAP3), mRNA | ARAP3 |
| [NM_022481] | |
| thromboxane A synthase 1 (platelet) (TBXAS1), transcript variant 5, mRNA | TBXAS1 |
| [NM_001166254] | |
| formyl peptide receptor 2 (FPR2), transcript variant 1, mRNA [NM_001462] | FPR2 |
| angiotensin II receptor-associated protein (AGTRAP), transcript variant 4, | AGTRAP |
| mRNA [NM_001040196] | |
| FYVE, RhoGEF and PH domain containing 4 (FGD4), mRNA [NM_139241] | FGD4 |
| chemokine (C-C motif) receptor 7 (CCR7), mRNA [NM_001838] | CCR7 |
| colony stimulating factor 1 receptor (CSF1R), mRNA [NM_005211] | CSF1R |
| tocopherol (alpha) transfer protein-like (TTPAL), transcript variant 1, mRNA | TTPAL |
| [NM_024331] | |
| toll-like receptor 7 (TLR7), mRNA [NM_016562] | TLR7 |
| leukocyte immunoglobulin-like receptor, subfamily A (with TM domain), | LILRA5 |
| member 5 (LILRA5), transcript variant 3, mRNA [NM_181879] | |
| potassium inwardly-rectifying channel, subfamily J, member 15 (KCNJ15), | KCNJ15 |
| transcript variant 1, mRNA [NM_170736] | |
| HD domain containing 2 (HDDC2), mRNA [NM_016063] | HDDC2 |
| centromere protein E, 312 kDa (CENPE), mRNA [NM_001813] | CENPE |
| DEP domain containing 1B (DEPDC1B), transcript variant 1, mRNA | DEPDC1B |
| [NM_018369] | |
| coagulation factor XIII, A1 polypeptide (F13A1), mRNA [NM_000129] | F13A1 |
| transketolase (TKT), transcript variant 1, mRNA [NM_001064] | TKT |
| sialic acid acetylesterase (SIAE), transcript variant 1, mRNA [NM_170601] | SIAE |
| phospholipase A2, group VII (platelet-activating factor acetylhydrolase, plasma) | PLA2G7 |
| (PLA2G7), transcript variant 1, mRNA [NM_005084] | |
| hydrogen voltage-gated channel 1 (HVCN1), transcript variant 1, mRNA | HVCN1 |
| [NM_001040107] | |
| vimentin (VIM), mRNA [NM_003380] | VIM |
| SET and MYND domain containing 3 (SMYD3), transcript variant 2, mRNA | SMYD3 |
| [NM_022743] | |
| leukocyte immunoglobulin-like receptor, subfamily B (with TM and ITIM | LILRB1 |
| domains), member 1 (LILRB1), transcript variant 1, mRNA [NM_006669] | |
| solute carrier family 46, member 2 (SLC46A2), mRNA [NM_033051] | SLC46A2 |
| cell division cycle 25 homolog A (S. pombe) (CDC25A), transcript variant 1, | CDC25A |
| mRNA [NM_001789] | |
| Morf4 family associated protein 1-like 1 pseudogene (LOC93622), non-coding | LOC93622 |
| RNA [NR_015433] | |
| transcription factor EC (TFEC), transcript variant 1, mRNA [NM_012252] | TFEC |
| bactericidal/permeability-increasing protein (BPI), mRNA [NM_001725] | BPI |
| macrophage receptor with collagenous structure (MARCO), mRNA | MARCO |
| [NM_006770] | |
| phosphotyrosine interaction domain containing 1 (PID1), transcript variant 1, | PID1 |
| mRNA [NM_017933] | |
| IKAROS family zinc finger 1 (Ikaros) (IKZF1), transcript variant 1, mRNA | IKZF1 |
| [NM_006060] | |
| G patch domain containing 4 [Source: HGNC Symbol;Acc: 25982] | GPATCH4 |
| [ENST00000334588] | |
| butyrophilin, subfamily 3, member A2 (BTN3A2), transcript variant 3, mRNA | BTN3A2 |
| [NM_001197247] | |
| lymphocyte antigen 9 (LY9), transcript variant 2, mRNA [NM_001033667] | LY9 |
| tetratricopeptide repeat domain 7A (TTC7A), mRNA [NM_020458] | TTC7A |
| serpin peptidase inhibitor, clade A (alpha-1 antiproteinase, antitrypsin), | SERPINA1 |
| member 1 [Source: HGNC Symbol;Acc: 8941] [ENST00000402629] | |
| AKT interacting protein (AKTIP), transcript variant 1, mRNA [NM_001012398] | AKTIP |
| sperm associated antigen 5 (SPAG5), mRNA [NM_006461] | SPAG5 |
| interleukin 1 receptor antagonist (IL1RN), transcript variant 4, mRNA | IL1RN |
| [NM_173843] | |
| ATG7 autophagy related 7 homolog (S. cerevisiae) (ATG7), transcript variant 1, | ATG7 |
| mRNA [NM_006395] | |
| prosaposin (PSAP), transcript variant 2, mRNA [NM_001042465] | PSAP |
| phosphogluconate dehydrogenase (PGD), mRNA [NM_002631] | PGD |
| complement factor properdin (CFP), transcript variant 1, mRNA [NM_002621] | CFP |
| hexokinase 3 (white cell) (HK3), nuclear gene encoding mitochondrial protein, | HK3 |
| mRNA [NM_002115] | |
| sialic acid binding Ig-like lectin 7 (SIGLEC7), transcript variant 1, mRNA | SIGLEC7 |
| [NM_014385] | |
| granulin (GRN), mRNA [NM_002087] | GRN |
| quiescin Q6 sulfhydryl oxidase 1 (QSOX1), transcript variant 2, mRNA | QSOX1 |
| [NM_001004128] | |
| ubiquitin-conjugating enzyme E2D 1 (UBE2D1), transcript variant 1, mRNA | UBE2D1 |
| [NM_003338] | |
| heme binding protein 2 (HEBP2), mRNA [NM_014320] | HEBP2 |
| BCL2-related protein A1 (BCL2A1), transcript variant 1, mRNA [NM_004049] | BCL2A1 |
| branched chain ketoacid dehydrogenase kinase (BCKDK), nuclear gene | BCKDK |
| encoding mitochondrial protein, transcript variant 1, mRNA [NM_005881] | |
| membrane bound O-acyltransferase domain containing 7 (MBOAT7), transcript | MBOAT7 |
| variant 4, mRNA [NM_001146082] | |
| arachidonate 5-lipoxygenase (ALOX5), mRNA [NM_000698] | ALOX5 |
| NLR family, CARD domain containing 4 (NLRC4), transcript variant 1, mRNA | NLRC4 |
| [NM_021209] | |
| phospholipase D family, member 3 (PLD3), transcript variant 2, mRNA | PLD3 |
| [NM_012268] | |
| HEN1 methyltransferase homolog 1 (Arabidopsis) (HENMT1), transcript variant | HENMT1 |
| 1, mRNA [NM_144584] | |
| chromosome 20 open reading frame 194 (C20orf194), mRNA [NM_001009984] | C20orf194 |
| nucleotide-binding oligomerization domain containing 2 (NOD2), mRNA | NOD2 |
| [NM_022162] | |
| glutaminyl-peptide cyclotransferase (QPCT), mRNA [NM_012413] | QPCT |
| minichromosome maintenance complex component 7 (MCM7), transcript | MCM7 |
| variant 2, mRNA [NM_182776] | |
| protein tyrosine phosphatase, receptor type, K (PTPRK), transcript variant 2, | PTPRK |
| mRNA [NM_002844] | |
| ribonuclease, RNase A family, 2 (liver, eosinophil-derived neurotoxin) | RNASE2 |
| (RNASE2), mRNA [NM_002934] | |
| Niemann-Pick disease, type C2 (NPC2), mRNA [NM_006432] | NPC2 |
| CD9 molecule (CD9), mRNA [NM_001769] | CD9 |
| brain protein I3 (BRI3), transcript variant 1, mRNA [NM_015379] | BRI3 |
| transmembrane protein 55A (TMEM55A), mRNA [NM_018710] | TMEM55A |
| macrophage expressed 1 (MPEG1), mRNA [NM_001039396] | MPEG1 |
| two pore segment channel 1 (TPCN1), transcript variant 1, mRNA | TPCN1 |
| [NM_001143819] | |
| glucosidase, alpha; acid (GAA), transcript variant 1, mRNA [NM_000152] | GAA |
| dicer 1, ribonuclease type III (DICER1), transcript variant 3, mRNA | DICER1 |
| [NM_001195573] | |
| tweety homolog 3 (Drosophila) (TTYH3), mRNA [NM_025250] | TTYH3 |
| ring finger protein 130 (RNF130), mRNA [NM_018434] | RNF130 |
| sterile alpha motif domain containing 13 (SAMD13), transcript variant 1, mRNA | SAMD13 |
| [NM_001010971] | |
| solute carrier organic anion transporter family, member 4C1 (SLCO4C1), mRNA | SLCO4C1 |
| [NM_180991] | |
| CD14 molecule (CD14), transcript variant 3, mRNA [NM_001174104] | CD14 |
| tubulin tyrosine ligase-like family, member 4 (TTLL4), mRNA [NM_014640] | TTLL4 |
| frizzled family receptor 5 (FZD5), mRNA [NM_003468] | FZD5 |
| mitogen-activated protein kinase kinase kinase 3 (MAP3K3), transcript variant | MAP3K3 |
| 1, mRNA [NM_203351] | |
| cathepsin B (CTSB), transcript variant 2, mRNA [NM_147780] | CTSB |
| structural maintenance of chromosomes 2 (SMC2), transcript variant 1, mRNA | SMC2 |
| [NM_001042550] | |
| transmembrane protein 51 (TMEM51), transcript variant 1, mRNA | TMEM51 |
| [NM_001136216] | |
| interleukin 21 receptor (IL21R), transcript variant 2, mRNA [NM_181078] | IL21R |
| vanin 2 (VNN2), transcript variant 1, mRNA [NM_004665] | VNN2 |
| bone marrow stromal cell antigen 1 (BST1), mRNA [NM_004334] | BST1 |
| non-SMC condensin II complex, subunit G2 (NCAPG2), mRNA [NM_017760] | NCAPG2 |
| glutathione peroxidase 1 (GPX1), transcript variant 2, mRNA [NM_201397] | GPX1 |
| potassium inwardly-rectifying channel, subfamily J, member 2 (KCNJ2), mRNA | KCNJ2 |
| [NM_000891] | |
| feline sarcoma oncogene (FES), transcript variant 1, mRNA [NM_002005] | FES |
| dipeptidyl-peptidase 4 (DPP4), mRNA [NM_001935] | DPP4 |
| butyrophilin, subfamily 3, member A1 (BTN3A1), transcript variant 1, mRNA | BTN3A1 |
| [NM_007048] | |
| azurocidin 1 (AZU1), mRNA [NM_001700] | AZU1 |
| geminin, DNA replication inhibitor (GMNN), transcript variant 1, mRNA | GMNN |
| [NM_015895] | |
| gelsolin (GSN), transcript variant 4, mRNA [NM_001127663] | GSN |
| cytoplasmic polyadenylation element binding protein 4 (CPEB4), mRNA | CPEB4 |
| [NM_030627] | |
| interleukin 12 receptor, beta 2 (IL12RB2), mRNA [NM_001559] | IL12RB2 |
| allograft inflammatory factor 1 (AIF1), transcript variant 2, mRNA [NM_004847] | AIF1 |
| apoptosis-inducing, TAF9-like domain 1 (APITD1), transcript variant A, mRNA | APITD1 |
| [NM_199294] | |
| Bruton agammaglobulinemia tyrosine kinase (BTK), mRNA [NM_000061] | BTK |
| nurim (nuclear envelope membrane protein) (NRM), mRNA [NM_007243] | NRM |
| membrane-spanning 4-domains, subfamily A, member 1 (M54A1), transcript | MS4A1 |
| variant 1, mRNA [NM_152866] | |
| major facilitator superfamily domain containing 1 (MFSD1), transcript variant 1, | MFSD1 |
| mRNA [NM_022736] | |
| REX4, RNA exonuclease 4 homolog (S. cerevisiae) (REXO4), mRNA | REXO4 |
| [NM_020385] | |
| sialic acid binding Ig-like lectin 5 (SIGLEC5), mRNA [NM_003830] | SIGLEC5 |
| glutamate-rich WD repeat containing 1 (GRWD1), mRNA [NM_031485] | GRWD1 |
| transmembrane protein 176A (TMEM176A), mRNA [NM_018487] | TMEM176A |
| chemokine (C-C motif) receptor-like 2 (CCRL2), transcript variant 1, mRNA | CCRL2 |
| [NM_003965] | |
| early B-cell factor 1 (EBF1), mRNA [NM_024007] | EBF1 |
| bromodomain and WD repeat domain containing 3 (BRWD3), mRNA | BRWD3 |
| [NM_153252] | |
| Zwilch, kinetochore associated, homolog (Drosophila) (ZWILCH), transcript | ZWILCH |
| variant 1, mRNA [NM_017975] | |
| cyclin-dependent kinase 2 (CDK2), transcript variant 1, mRNA [NM_001798] | CDK2 |
| kelch-like 18 (Drosophila) (KLHL18), mRNA [NM_025010] | KLHL18 |
| zinc finger protein 367 (ZNF367), mRNA [NM_153695] | ZNF367 |
| nuclear factor of kappa light polypeptide gene enhancer in B-cells inhibitor, | NFKBIA |
| alpha (NFKBIA), mRNA [NM_020529] | |
| dihydropyrimidine dehydrogenase (DPYD), transcript variant 2, mRNA | DPYD |
| [NM_001160301] | |
| zinc finger protein 36, C3H type, homolog (mouse) (ZFP36), mRNA | ZFP36 |
| [NM_003407] | |
| coagulation factor V (proaccelerin, labile factor) (F5), mRNA [NM_000130] | F5 |
| SMEK homolog 2, suppressor of mek1 (Dictyostelium) (SMEK2), transcript | SMEK2 |
| variant 2, mRNA [NM_020463] | |
| GINS complex subunit 4 (Sld5 homolog) (GINS4), mRNA [NM_032336] | GINS4 |
| CD22 molecule (CD22), transcript variant 1, mRNA [NM_001771] | CD22 |
| mRNA for very long-chain acyl-CoA synthetase homologue 3 (VLCS-3 gene). | SLC27A3 |
| [AJ577572] | |
| cell division cycle 7 homolog (S. cerevisiae) (CDC7), transcript variant 1, mRNA | CDC7 |
| [NM_003503] | |
| grancalcin, EF-hand calcium binding protein (GCA), mRNA [NM_012198] | GCA |
| signal-regulatory protein beta 1 (SIRPB1), transcript variant 3, mRNA | SIRPB1 |
| [NM_001135844] | |
| CDC42 binding protein kinase beta (DMPK-like) (CDC42BPB), mRNA | CDC42BPB |
| [NM_006035] | |
| spinster homolog 1 (Drosophila) (SPNS1), transcript variant 1, mRNA | SPNS1 |
| [NM_032038] | |
| tetraspanin 15 (TSPAN15), mRNA [NM_012339] | TSPAN15 |
| cystatin C (CST3), mRNA [NM_000099] | CST3 |
| plexin B2 (PLXNB2), mRNA [NM_012401] | PLXNB2 |
| Ras association (RalGDS/AF-6) domain family member 4 (RASSF4), mRNA | RASSF4 |
| [NM_032023] | |
| PREDICTED: peptidyl-prolyl cis-trans isomerase A-like (LOC100288602), mRNA | LOC100288602 |
| [XM_003403504] | |
| protein kinase (cAMP-dependent, catalytic) inhibitor alpha (PKIA), transcript | PKIA |
| variant 1, mRNA [NM_006823] | |
| chromosome 9 open reading frame 7 (C9orf7), transcript variant 3, mRNA | C9orf7 |
| [NM_001242369] | |
| toll-like receptor 1 (TLR1), mRNA [NM_003263] | TLR1 |
| centrosomal protein 76 kDa (CEP76), mRNA [NM_024899] | CEP76 |
| protein kinase C, delta (PRKCD), transcript variant 1, mRNA [NM_006254] | PRKCD |
| CD1a molecule (CD1A), mRNA [NM_001763] | CD1A |
| plectin (PLEC), transcript variant 6, mRNA [NM_201380] | PLEC |
| leukocyte immunoglobulin-like receptor, subfamily A (with TM domain), | LILRA6 |
| member 6 (LILRA6), mRNA [NM_024318] | |
| RCC1 domain containing 1 (RCCD1), transcript variant 1, mRNA [NM_033544] | RCCD1 |
| dihydrofolate reductase (DHFR), mRNA [NM_000791] | DHFR |
| solute carrier family 31 (copper transporters), member 2 (SLC31A2), mRNA | SLC31A2 |
| [NM_001860] | |
| protein arginine methyltransferase 1 (PRMT1), transcript variant 1, mRNA | PRMT1 |
| [NM_001536] | |
| NEAT activating protein with ITAM motif 1 (NFAM1), mRNA [NM_145912] | NFAM1 |
| adducin 3 (gamma) (ADD3), transcript variant 1, mRNA [NM_016824] | ADD3 |
| acyloxyacyl hydrolase (neutrophil) (AOAH), transcript variant 1, mRNA | AOAH |
| [NM_001637] | |
| TYRO protein tyrosine kinase binding protein (TYROBP), transcript variant 1, | TYROBP |
| mRNA [NM_003332] | |
| leukocyte immunoglobulin-like receptor, subfamily A (with TM domain), | LILRA2 |
| member 2 (LILRA2), transcript variant 2, mRNA [NM_006866] | |
| Gardner-Rasheed feline sarcoma viral (v-fgr) oncogene homolog (FGR), | FGR |
| transcript variant 2, mRNA [NM_001042747] | |
| neutrophil cytosolic factor 1 (NCF1), mRNA [NM_000265] | NCF1 |
| Rac GTPase activating protein 1 (RACGAP1), transcript variant 1, mRNA | RACGAP1 |
| [NM_013277] | |
| shugoshin-like 2 (S. pombe) (SGOL2), transcript variant 1, mRNA [NM_152524] | SGOL2 |
| G protein-coupled receptor 84 (GPR84), mRNA [NM_020370] | GPR84 |
| Fc receptor-like 1 (FCRL1), transcript variant 2, mRNA [NM_001159397] | FCRL1 |
| ninjurin 2 (NINJ2), mRNA [NM_016533] | NINJ2 |
| membrane protein, palmitoylated 1, 55 kDa (MPP1), transcript variant 3, mRNA | MPP1 |
| [NM_001166461] | |
| formyl peptide receptor 1 (FPR1), transcript variant 2, mRNA [NM_002029] | FPR1 |
| phosphatidylinositol transfer protein, beta (PITPNB), mRNA [NM_012399] | PITPNB |
| RAD21 homolog (S. pombe) (RAD21), mRNA [NM_006265] | RAD21 |
| glutamate receptor, ionotropic, N-methyl D-aspartate-associated protein 1 | GRINA |
| (glutamate binding) (GRINA), transcript variant 1, mRNA [NM_000837] | |
| histone deacetylase 8 (HDAC8), transcript variant 1, mRNA [NM_018486] | HDAC8 |
| guanine nucleotide binding protein (G protein), gamma 10 (GNG10), transcript | GNG10 |
| variant 1, mRNA [NM_001017998] | |
| CD19 molecule (CD19), transcript variant 2, mRNA [NM_001770] | CD19 |
| elastin microfibril interfacer 2 (EMILIN2), mRNA [NM_032048] | EMILIN2 |
| chemokine (C-C motif) receptor 2 (CCR2), transcript variant A, mRNA | CCR2 |
| [NM_001123041] | |
| mitochondrial translation optimization 1 homolog (S. cerevisiae) (MTO1), | MTO1 |
| nuclear gene encoding mitochondrial protein, transcript variant 2, mRNA | |
| [NM_012123] | |
| leukocyte immunoglobulin-like receptor, subfamily B (with TM and ITIM | LILRB2 |
| domains), member 2 (LILRB2), transcript variant 1, mRNA [NM_005874] | |
| docking protein 3 (DOK3), transcript variant 2, mRNA [NM_001144875] | DOK3 |
| actinin, alpha 1 (ACTN1), transcript variant 2, mRNA [NM_001102] | ACTN1 |
| antigen identified by monoclonal antibody Ki-67 (MKI67), transcript variant 1, | MKI67 |
| mRNA [NM_002417] | |
| Ran GTPase activating protein 1 (RANGAP1), mRNA [NM_002883] | RANGAP1 |
| neural proliferation, differentiation and control, 1 (NPDC1), mRNA | NPDC1 |
| [NM_015392] | |
| spleen focus forming virus (SFFV) proviral integration oncogene soil (SPI1), | SPI1 |
| transcript variant 1, mRNA [NM_001080547] | |
| neural precursor cell expressed, developmentally down-regulated 1 (NEDD1), | NEDD1 |
| transcript variant 2, mRNA [NM_152905] | |
| primase, DNA, polypeptide 2 (58 kDa) (PRIM2), mRNA [NM_000947] | PRIM2 |
| hemopoietic cell kinase (HCK), transcript variant 1, mRNA [NM_002110] | HCK |
| primase, DNA, polypeptide 1 (49 kDa) (PRIM1), mRNA [NM_000946] | PRIM1 |
| ficolin (collagen/fibrinogen domain containing) 1 (FCN1), mRNA [NM_002003] | FCN1 |
| timeless homolog (Drosophila) (TIMELESS), mRNA [NM_003920] | TIMELESS |
| dermatan sulfate epimerase (DSE), transcript variant 1, mRNA [NM_013352] | DSE |
| SUM01 activating enzyme subunit 1 (SAE1), transcript variant 1, mRNA | SAE1 |
| [NM_005500] | |
| S100 calcium binding protein A8 (5100A8), mRNA [NM_002964] | S100A8 |
| tubulin, gamma 1 (TUBG1), mRNA [NM_001070] | TUBG1 |
| ATP synthase mitochondrial F1 complex assembly factor 2 (ATPAF2), nuclear | ATPAF2 |
| gene encoding mitochondrial protein, mRNA [NM_145691] | |
| zinc finger protein 131 (ZNF131), mRNA [NM_003432] | ZNF131 |
| replication protein A3, 14 kDa (RPA3), mRNA [NM_002947] | RPA3 |
| cDNA FLJ42230 fis, clone THYMU3000036. [AK124224] | SIRPG |
| CDC28 protein kinase regulatory subunit 1B (CKS1B), transcript variant 1, mRNA | CKS1B |
| [NM_001826] | |
| guanine nucleotide binding protein (G protein), q polypeptide (GNAQ), mRNA | GNAQ |
| [NM_002072] | |
| KIAA0125 (KIAA0125), non-coding RNA [NR_026800] | KIAA0125 |
| S100 calcium binding protein A9 (S100A9), mRNA [NM_002965] | S100A9 |
| SP140 nuclear body protein (SP140), transcript variant 1, mRNA [NM_007237] | SP140 |
| cyclin-dependent kinase inhibitor 2A (melanoma, p16, inhibits CDK4) (CDKN2A), | CDKN2A |
| transcript variant 3, mRNA [NM_058197] | |
| major histocompatibility complex, class II, DQ beta 1 (HLA-DQB1), transcript | HLA-DQB1 |
| variant 2, mRNA [NM_001243961] | |
| B lymphoid tyrosine kinase (BLK), mRNA [NM_001715] | BLK |
| protein kinase, membrane associated tyrosine/threonine 1 (PKMYT1), | PKMYT1 |
| transcript variant 2, mRNA [NM_182687] | |
| trafficking protein particle complex 5 (TRAPPC5), transcript variant 1, mRNA | TRAPPC5 |
| [NM_174894] | |
| linker for activation of T cells family, member 2 (LAT2), transcript variant 1, | LAT2 |
| mRNA [NM_032464] | |
| transmembrane protein 176B (TMEM176B), transcript variant 1, mRNA | TMEM176B |
| [NM_014020] | |
| zyxin (ZYX), transcript variant 1, mRNA [NM_003461] | ZYX |
| interferon, gamma-inducible protein 30 (IFI30), mRNA [NM_006332] | IFI30 |
| cathepsin H (CTSH), mRNA [NM_004390] | CTSH |
| tumor necrosis factor (ligand) superfamily, member 13b (TNFSF13B), transcript | TNFSF13B |
| variant 1, mRNA [NM_006573] | |
| retinol dehydrogenase 14 (all-trans/9-cis/11-cis) (RDH14), mRNA [NM_020905] | RDH14 |
| guanylate binding protein 3 (GBP3), mRNA [NM_018284] | GBP3 |
| centrosomal protein 72 kDa (CEP72), mRNA [NM_018140] | CEP72 |
| extracellular matrix protein 1 (ECM1), transcript variant 1, mRNA [NM_004425] | ECM1 |
| uridine monophosphate synthetase (UMPS), transcript variant 1, mRNA | UMPS |
| [NM_000373] | |
| purinergic receptor P2X, ligand-gated ion channel, 5 (P2RX5), transcript variant | P2RX5 |
| 2, mRNA [NM_175080] | |
| catenin, beta interacting protein 1 (CTNNBIP1), transcript variant 1, mRNA | CTNNBIP1 |
| [NM_020248] | |
| structural maintenance of chromosomes 4 (SMC4), transcript variant 1, mRNA | SMC4 |
| [NM_005496] | |
| H2A histone family, member J (H2AFJ), transcript variant 1, mRNA | H2AFJ |
| [NM_177925] | |
| integrin, alpha M (complement component 3 receptor 3 subunit) (ITGAM), | ITGAM |
| transcript variant 2, mRNA [NM_000632] | |
| ras homolog gene family, member U (RHOU), transcript variant 1, mRNA | RHOU |
| [NM_021205] | |
| leucine rich repeat containing 8 family, member A (LRRC8A), transcript variant | LRRC8A |
| 2, mRNA [NM_019594] | |
| family with sequence similarity 105, member A (FAM105A), mRNA | FAM105A |
| [NM_019018] | |
| family with sequence similarity 185, member A (FAM185A), transcript variant 1, | FAM185A |
| mRNA [NM_001145268] | |
| replication factor C (activator 1) 5, 36.5 kDa (RFC5), transcript variant 2, mRNA | RFC5 |
| [NM_181578] | |
| tumor necrosis factor (ligand) superfamily, member 14 (TNFSF14), transcript | TNFSF14 |
| variant 1, mRNA [NM_003807] | |
| chemokine (C-X3-C motif) receptor 1 (CX3CR1), transcript variant 4, mRNA | CX3CR1 |
| [NM_001337] | |
| PYD and CARD domain containing (PYCARD), transcript variant 1, mRNA | PYCARD |
| [NM_013258] | |
| transmembrane protein 154 (TMEM154), mRNA [NM_152680] | TMEM154 |
| shugoshin-like 1 (S. pombe) (SGOL1), transcript variant A2, mRNA | SGOL1 |
| [NM_001012410] | |
| poly (ADP-ribose) polymerase family, member 9 (PARP9), transcript variant 1, | PARP9 |
| mRNA [NM_031458] | |
| cathepsin D (CTSD), mRNA [NM_001909] | CTSD |
| dual specificity phosphatase 22 (DUSP22), mRNA [NM_020185] | DUSP22 |
| protein tyrosine phosphatase, non-receptor type 22 (lymphoid) (PTPN22), | PTPN22 |
| transcript variant 2, mRNA [NM_012411] | |
| 1-acylglycerol-3-phosphate O-acyltransferase 9 (AGPAT9), mRNA [NM_032717] | AGPAT9 |
| C-type lectin domain family 2, member D (CLEC2D), transcript variant 2, mRNA | CLEC2D |
| [NM_001004419] | |
| cyclin-dependent kinase 14 (CDK14), mRNA [NM_012395] | CDK14 |
| kinetochore associated 1 (KNTC1), mRNA [NM_014708] | KNTC1 |
| phosphorylase kinase, beta (PHKB), transcript variant 2, mRNA | PHKB |
| [NM_001031835] | |
| extra spindle pole bodies homolog 1 (S. cerevisiae) (ESPL1), mRNA | ESPL1 |
| [NM_012291] | |
| heparan-alpha-glucosaminide N-acetyltransferase (HGSNAT), mRNA | HGSNAT |
| [NM_152419] | |
| ERI1 exoribonuclease family member 2 (ERI2), transcript variant 2, mRNA | ERI2 |
| [NM_080663] | |
| endoglin (ENG), transcript variant 2, mRNA [NM_000118] | ENG |
| polymerase (DNA directed), alpha 1, catalytic subunit (POLA1), mRNA | POLA1 |
| [NM_016937] | |
| adaptor-related protein complex 1, sigma 2 subunit (AP1S2), mRNA | AP1S2 |
| [NM_003916] | |
| ADAM metallopeptidase domain 12 (ADAM12), transcript variant 2, mRNA | ADAM12 |
| [NM_021641] | |
| Fc fragment of IgG, high affinity 1b, receptor (CD64) (FCGR1B), transcript variant | FCGR1B |
| 3, mRNA [NM_001244910] | |
| TIMELESS interacting protein (TIPIN), mRNA [NM_017858] | TIPIN |
| nucleolar and spindle associated protein 1 (NUSAP1), transcript variant 1, | NUSAP1 |
| mRNA [NM_016359] | |
| TAF7 RNA polymerase II, TATA box binding protein (TBP)-associated factor, | TAF7 |
| 55 kDa (TAF7), mRNA [NM_005642] | |
| GTPase, IMAP family member 6 (GIMAP6), transcript variant 1, mRNA | GIMAP6 |
| [NM_024711] | |
| DnaJ (Hsp40) homolog, subfamily A, member 3 (DNAJA3), nuclear gene | DNAJA3 |
| encoding mitochondrial protein, transcript variant 1, mRNA [NM_005147] | |
| p21 protein (Cdc42/Rac)-activated kinase 1 (PAK1), transcript variant 1, mRNA | PAK1 |
| [NM_001128620] | |
| alcohol dehydrogenase 5 (class III), chi polypeptide (ADH5), mRNA | ADH5 |
| [NM_000671] | |
| CD79a molecule, immunoglobulin-associated alpha (CD79A), transcript variant | CD79A |
| 1, mRNA [NM_001783] | |
| Fc receptor-like 2 (FCRL2), mRNA [NM_030764] | FCRL2 |
| uridine phosphorylase 1 [Source: HGNC Symbol;Acc: 12576] [ENST00000457596] | UPP1 |
| sialic acid binding Ig-like lectin 16 (gene/pseudogene) (SIGLEC16), non-coding | SIGLEC16 |
| RNA [NR_002825] | |
| PREDICTED: peptidyl-prolyl cis-trans isomerase A-like (LOC729313), miscRNA | LOC729313 |
| [XR_132614] | |
| T-cell leukemia/lymphoma 1A (TCL1A), transcript variant 1, mRNA | TCL1A |
| [NM_021966] | |
| retinoblastoma-like 2 (p130) (RBL2), mRNA [NM_005611] | RBL2 |
| RAN, member RAS oncogene family (RAN), mRNA [NM_006325] | RAN |
| adrenergic, beta-2-, receptor, surface (ADRB2), mRNA [NM_000024] | ADRB2 |
| solute carrier family 16, member 3 (monocarboxylic acid transporter 4) | SLC16A3 |
| (SLC16A3), transcript variant 2, mRNA [NM_001042422] | |
| myosin IF (MYO1F), mRNA [NM_012335] | MYO1F |
| myeloid differentiation primary response gene (88) (MYD88), transcript variant | MYD88 |
| 2, mRNA [NM_002468] | |
| solute carrier family 38, member 6 (SLC38A6), transcript variant 2, mRNA | SLC38A6 |
| [NM_153811] | |
| polymerase (DNA directed), alpha 2 (70 kD subunit) (POLA2), mRNA | POLA2 |
| [NM_002689] | |
| cytochrome b-245, beta polypeptide (CYBB), mRNA [NM_000397] | CYBB |
| WD repeat domain 92 (WDR92), mRNA [NM_138458] | WDR92 |
| Spi-B transcription factor (Spi-1/PU.1 related) (SPIB), transcript variant 1, mRNA | SPIB |
| [NM_003121] | |
| nucleoporin 88 kDa (NUP88), mRNA [NM_002532] | NUP88 |
| serpin peptidase inhibitor, clade G (C1 inhibitor), member 1 (SERPING1), | SERPING1 |
| transcript variant 1, mRNA [NM_000062] | |
| serine peptidase inhibitor, Kunitz type, 2 (5PINT2), transcript variant a, mRNA | SPINT2 |
| [NM_021102] | |
| mutS homolog 6 (E. coli) (MSH6), mRNA [NM_000179] | MSH6 |
| HESX homeobox 1 (HESX1), mRNA [NM_003865] | HESX1 |
| non-SMC condensin 11 complex, subunit D3 (NCAPD3), mRNA [NM_015261] | NCAPD3 |
| natural cytotoxicity triggering receptor 1 (NCR1), transcript variant 1, mRNA | NCR1 |
| [NM_004829] | |
| ATPase, class I, type 8B, member 4 (ATP8B4), mRNA [NM_024837] | ATP8B4 |
| sideroflexin 3 (SFXN3), mRNA [NM_030971] | SFXN3 |
| vaccinia related kinase 1 (VRK1), mRNA [NM_003384] | VRK1 |
| G protein-coupled receptor 82 (GPR82), mRNA [NM_080817] | GPR82 |
| nuclear factor (erythroid-derived 2), 45 kDa (NFE2), transcript variant 1, mRNA | NFE2 |
| [NM_006163] | |
| pseudouridylate synthase 7 homolog (S. cerevisiae) (PUS7), mRNA | PUS7 |
| [NM_019042] | |
| E2F transcription factor 2 (E2F2), mRNA [NM_004091] | E2F2 |
| transcription termination factor, RNA polymerase II (TTF2), mRNA | TTF2 |
| [NM_003594] | |
| SCL/TAL1 interrupting locus (STIL), transcript variant 1, mRNA [NM_001048166] | STIL |
| sterile alpha motif domain containing 9-like (SAMD9L), mRNA [NM_152703] | SAMD9L |
| NADH dehydrogenase (ubiquinone) 1 alpha subcomplex, 8, 19 kDa (NDUFA8), | NDUFA8 |
| nuclear gene encoding mitochondrial protein, mRNA [NM_014222] | |
| processing of precursor 5, ribonuclease P/MRP subunit (S. cerevisiae) (POP5), | POP5 |
| transcript variant 1, mRNA [NM_015918] | |
| centrosomal protein 152 kDa (CEP152), transcript variant 2, mRNA | CEP152 |
| [NM_014985] | |
| anaphase promoting complex subunit 10 (ANAPC10), mRNA [NM_014885] | ANAPC10 |
| CD200 molecule (CD200), transcript variant 2, mRNA [NM_001004196] | CD200 |
| leucine rich repeat and fibronectin type III domain containing 4 (LRFN4), mRNA | LRFN4 |
| [NM_024036] | |
| cyclin-dependent kinase 4 (CDK4), mRNA [NM_000075] | CDK4 |
| structural maintenance of chromosomes 3 (SMC3), mRNA [NM_005445] | SMC3 |
| retinoic acid receptor, alpha (RARA), transcript variant 2, mRNA | RARA |
| [NM_001024809] | |
| chemokine (C-C motif) receptor 5 (CCR5), transcript variant A, mRNA | CCR5 |
| [NM_000579] | |
| superoxide dismutase 2, mitochondrial (SOD2), nuclear gene encoding | SOD2 |
| mitochondrial protein, transcript variant 2, mRNA [NM_001024465] | |
| interferon-induced protein with tetratricopeptide repeats 1 (IFIT1), transcript | IFIT1 |
| variant 2, mRNA [NM_001548] | |
| cytoskeleton associated protein 5 (CKAP5), transcript variant 1, mRNA | CKAP5 |
| [NM_001008938] | |
| myxovirus (influenza virus) resistance 2 (mouse) (MX2), mRNA [NM_002463] | MX2 |
| aryl hydrocarbon receptor nuclear translocator-like 2 (ARNTL2), transcript | ARNTL2 |
| variant 1, mRNA [NM_020183] | |
| cyclin F (CCNF), mRNA [NM_001761] | CCNF |
| cytotoxic and regulatory T cell molecule (CRTAM), mRNA [NM_019604] | CRTAM |
| SWI/SNF related, matrix associated, actin dependent regulator of chromatin, | SMARCAL1 |
| subfamily a-like 1 (SMARCAL1), transcript variant 1, mRNA [NM_014140] | |
| prostaglandin I2 (prostacyclin) receptor (IP) (PTGIR), mRNA [NM_000960] | PTGIR |
| MHC class I polypeptide-related sequence B (MICB), mRNA [NM_005931] | MICB |
| caspase 7, apoptosis-related cysteine peptidase (CASP7), transcript variant | CASP7 |
| delta, mRNA [NM_033338] | |
| ataxia telangiectasia and Rad3 related (ATR), mRNA [NM_001184] | ATR |
| tumor necrosis factor, alpha-induced protein 6 (TNFAIP6), mRNA [NM_007115] | TNFAIP6 |
| chemokine (C-X-C motif) receptor 5 (CXCR5), transcript variant 2, mRNA | CXCR5 |
| [NM_032966] | |
| eukaryotic translation initiation factor 2B, subunit 3 gamma, 58 kDa (EIF2B3), | EIF2B3 |
| transcript variant 1, mRNA [NM_020365] | |
| topoisomerase (DNA) II binding protein 1 (TOPBP1), mRNA [NM_007027] | TOPBP1 |
| jumonji domain containing 4 (JMJD4), transcript variant 1, mRNA [NM_023007] | JMJD4 |
| polo-like kinase 1 (PLK1), mRNA [NM_005030] | PLK1 |
| colony stimulating factor 2 receptor, beta, low-affinity (granulocyte- | CSF2RB |
| macrophage) (CSF2RB), mRNA [NM_000395] | |
| AF4/FMR2 family, member 3 [Source: HGNC Symbol;Acc: 6473] | AFF3 |
| [ENST00000483600] | |
| 3-hydroxyisobutyryl-CoA hydrolase (HIBCH), nuclear gene encoding | HIBCH |
| mitochondrial protein, transcript variant 2, mRNA [NM_198047] | |
| S-phase kinase-associated protein 2 (p45) (SKP2), transcript variant 2, mRNA | SKP2 |
| [NM_032637] | |
| myosin VIIB (MYO7B), mRNA [NM_001080527] | MYO7B |
| CCAAT/enhancer binding protein (C/EBP), epsilon (CEBPE), mRNA | CEBPE |
| [NM_001805] | |
| Ral GEF with PH domain and SH3 binding motif 2 (RALGPS2), mRNA | RALGPS2 |
| [NM_152663] | |
| minichromosome maintenance complex component 5 (MCM5), mRNA | MCM5 |
| [NM_006739] | |
| interferon induced with helicase C domain 1 (IFIH1), mRNA [NM_022168] | IFIH1 |
| presenilin 2 (Alzheimer disease 4) (PSEN2), transcript variant 1, mRNA | PSEN2 |
| [NM_000447] | |
| single-stranded DNA binding protein 1 (SSBP1), nuclear gene encoding | SSBP1 |
| mitochondrial protein, mRNA [NM_003143] | |
| CHK2 checkpoint homolog (S. pombe) (CHEK2), transcript variant 2, mRNA | CHEK2 |
| [NM_145862] | |
| thymopoietin (TMPO), transcript variant 2, mRNA [NM_001032283] | TMPO |
| cell division cycle 23 homolog (S. cerevisiae) (CDC23), mRNA [NM_004661] | CDC23 |
| trichoplein, keratin filament binding (TCHP), transcript variant 1, mRNA | TCHP |
| [NM_032300] | |
| pre-B lymphocyte 3 (VPREB3), mRNA [NM_013378] | VPREB3 |
| CD72 molecule [Source: HGNC Symbol;Acc: 1696] [ENST00000378431] | CD72 |
| N-acetylneuraminate pyruvate lyase (dihydrodipicolinate synthase) (NPL), | NPL |
| transcript variant 3, mRNA [NM_001200056] | |
| deoxythymidylate kinase (thymidylate kinase) (DTYMK), transcript variant 1, | DTYMK |
| mRNA [NM_012145] | |
| TRAF3 interacting protein 3 (TRAF3IP3), mRNA [NM_025228] | TRAF3IP3 |
| coronin, actin binding protein, 1A (CORO1A), transcript variant 2, mRNA | CORO1A |
| [NM_007074] | |
| G protein-coupled receptor 137B (GPR137B), mRNA [NM_003272] | GPR137B |
| polymerase (DNA-directed), delta 3, accessory subunit (POLD3), mRNA | POLD3 |
| [NM_006591] | |
| Fc receptor-like A (FCRLA), transcript variant 2, mRNA [NM_032738] | FCRLA |
| excision repair cross-complementing rodent repair deficiency, | ERCC6L |
| complementation group 6-like (ERCC6L), mRNA [NM_017669] | |
| kinesin family member 23 (KIF23), transcript variant 1, mRNA [NM_138555] | KIF23 |
| major histocompatibility complex, class II, DO beta (HLA-DOB), mRNA | HLA-DOB |
| [NM_002120] | |
| asparagine-linked glycosylation 8, alpha-1,3-glucosyltransferase homolog (S. | ALG8 |
| cerevisiae) (ALG8), transcript variant 2, mRNA [NM_001007027] | |
| proteasome (prosome, macropain) subunit, alpha type, 3 (PSMA3), transcript | PSMA3 |
| variant 1, mRNA [NM_002788] | |
| centromere protein N (CENPN), transcript variant 3, mRNA [NM_018455] | CENPN |
| acyl-CoA dehydrogenase, C-4 to C-12 straight chain (ACADM), nuclear gene | ACADM |
| encoding mitochondrial protein, transcript variant 1, mRNA [NM_000016] | |
| radical S-adenosyl methionine domain containing 2 (RSAD2), mRNA | RSAD2 |
| [NM_080657] | |
| tetraspanin 13 (TSPAN13), mRNA [NM_014399] | TSPAN13 |
| breast cancer 1, early onset (BRCA1), transcript variant 2, mRNA [NM_007300] | BRCA1 |
| ATP-binding cassette, sub-family C (CFTR/MRP), member 3 (ABCC3), transcript | ABCC3 |
| variant 1, mRNA [NM_003786] | |
| major histocompatibility complex, class II, DO alpha (HLA-DOA), mRNA | HLA-DOA |
| [NM_002119] | |
| V-set and immunoglobulin domain containing 10 (VSIG10), mRNA | VSIG10 |
| [NM_019086] | |
| WAS protein homolog associated with actin, golgi membranes and | WHAMM |
| microtubules (WHAMM), mRNA [NM_001080435] | |
| carcinoembryonic antigen-related cell adhesion molecule 1 (biliary | CEACAM1 |
| glycoprotein) (CEACAM1), transcript variant 5, mRNA [NM_001184816] | |
| transcription factor Dp-1 (TFDP1), transcript variant 1, mRNA [NM_007111] | TFDP1 |
| kinesin family member 20B (KIF20B), mRNA [NM_016195] | KIF20B |
| family with sequence similarity 129, member C (FAM129C), transcript variant 2, | FAM129C |
| mRNA [NM_001098524] | |
| MACRO domain containing 2 (MACROD2), transcript variant 1, mRNA | MACROD2 |
| [NM_080676] | |
| peptidylprolyl isomerase (cyclophilin)-like 4 (PPIL4), mRNA [NM_139126] | PPIL4 |
| CD24 molecule (CD24), mRNA [NM_013230] | CD24 |
| family with sequence similarity 129, member A (FAM129A), transcript variant 2, | FAM129A |
| mRNA [NM_052966] | |
| zinc finger protein 467 (ZNF467), mRNA [NM_207336] | ZNF467 |
| ADAM metallopeptidase domain 28 (ADAM28), transcript variant 3, mRNA | ADAM28 |
| [NM_021777] | |
| SAM domain and HD domain 1 (SAMHD1), mRNA [NM_015474] | SAMHD1 |
| epithelial cell transforming sequence 2 oncogene (ECT2), mRNA [NM_018098] | ECT2 |
| kynurenine 3-monooxygenase (kynurenine 3-hydroxylase) (KMO), mRNA | KMO |
| [NM_003679] | |
| ASF1 anti-silencing function 1 homolog B (S. cerevisiae) (ASF1B), mRNA | ASF1B |
| [NM_018154] | |
| proline-serine-threonine phosphatase interacting protein 1 (PSTPIP1), mRNA | PSTPIP1 |
| [NM_003978] | |
| RMI1, RecQ mediated genome instability 1, homolog (S. cerevisiae) (RMI1), | RMI1 |
| mRNA [NM_024945] | |

TABLE 2
marker genes (full name and accession number as set out in table 1)

| | | | | |
|---|---|---|---|---|
| LOC572558 | KIAA0101 | HJURP | SLAMF6 | ENPP5 |
| USH1G | IFI27 | STAP1 | KIR2DS2 | IL18RAP |
| BMP2 | RAD51 | TOP2A | XLOC_ | GRAP2 |
| | | | 004032 | |
| KIF18A | TYMS | XLOC_l2_ | PRF1 | SH2D2A |
| | | 010897 | | |
| ANLN | E2F8 | PPP2R2B | GINS2 | CR1 |
| GP5 | PDGFRB | SPC25 | CDC45 | IQCD |
| SKA3 | CHI3L1 | KIAA1671 | TBX21 | CCL5 |
| TTK | IFNG | BUB1 | GZMA | KLRD1 |
| OLFM4 | CDT1 | GPR56 | NKG7 | CXCR3 |
| DLGAP5 | CCL20 | GZMM | SGK1 | CD247 |
| MCM10 | DTL | SAMD3 | PVRIG | ZNF697 |
| SLPI | LOC643650 | CTSW | NEK2 | HOPX |
| FASLG | CCNA2 | PTGFRN | TARP | IL1B |
| SLC7A8 | RGS9 | XLOC_ | BIRC5 | KLRC1 |
| | | 006752 | | |
| EXO1 | CEP55 | GNLY | CST7 | ZNF503 |
| FGFBP2 | POLE2 | LINC00239 | IL2RB | XLOC_ |
| | | | | 004924 |
| CXCL1 | ASPM | CDCA2 | MND1 | ZAP70 |
| MELK | TGFBR3 | GZMH | S1PR5 | CENPF |
| RRM2 | RPGRIP1 | BUB1B | STK32B | PTPRCAP |
| LOC572558 | KIR2DL2 | UBE2C | KLRF1 | KLRC3 |

TABLE 2B
marker genes (full name and accession number as set out in table 1)

| | | | | |
|---|---|---|---|---|
| LOC572558 | TYMS | KIAA1671 | NKG7 | CXCR3 |
| USH1G | E2F8 | BUB1 | SGK1 | CD247 |
| BMP2 | PDGFRB | GPR56 | PVRIG | ZNF697 |
| KIF18A | CHI3L1 | GZMM | NEK2 | HOPX |
| ANLN | IFNG | SAMD3 | TARP | IL1B |
| GP5 | CCL20 | CTSW | BIRC5 | KLRC1 |
| SKA3 | DTL | PTGFRN | CST7 | ZNF503 |
| TTK | LOC643650 | XLOC_ | IL2RB | XLOC_ |
| | | 006752 | | 004924 |
| OLFM4 | RGS9 | GNLY | MND1 | ZAP70 |
| DLGAP5 | CEP55 | LINC00239 | S1PR5 | CENPF |
| SLPI | POLE2 | GZMH | STK32B | PTPRCAP |
| FASLG | ASPM | BUB1B | KLRF1 | KLRC3 |
| SLC7A8 | TGFBR3 | SLAMF6 | ENPP5 | |
| EXO1 | RPGRIP1 | KIR2DS2 | IL18RAP | |
| FGFBP2 | KIR2DL2 | XLOC_ | GRAP2 | |
| | | 004032 | | |
| CXCL1 | HJURP | PRF1 | SH2D2A | |
| LOC572558 | STAP1 | GINS2 | CR1 | |
| KIAA0101 | XLOC_l2_ | CDC45 | IQCD | |
| | 010897 | | | |
| IFI27 | PPP2R2B | TBX21 | CCL5 | |
| RAD51 | SPC25 | GZMA | KLRD1 | |

TABLE 3
marker genes (full name and accession number as set out in table 1)

| | | | | |
|---|---|---|---|---|
| LOC572558 | MCM10 | IFI27 | LOC643650 | STAP1 |
| USH1G | SLPI | RAD51 | CCNA2 | TOP2A |
| BMP2 | FASLG | TYMS | RGS9 | XLOC_l2_ |
| | | | | 010897 |
| KIF18A | SLC7A8 | E2F8 | CEP55 | PPP2R2B |
| ANLN | EXO1 | PDGFRB | POLE2 | SPC25 |
| GP5 | FGFBP2 | CHI3L1 | ASPM | KIAA1671 |
| SKA3 | CXCL1 | IFNG | TGFBR3 | BUB1 |
| TTK | MELK | CDT1 | RPGRIP1 | GPR56 |
| OLFM4 | RRM2 | CCL20 | KIR2DL2 | GZMM |
| DLGAP5 | KIAA0101 | DTL | HJURP | SAMD3 |

TABLE 3B
marker genes (full name and accession number as set out in table 1)

| | | | | |
|---|---|---|---|---|
| LOC572558 | SLPI | E2F8 | ASPM | BUB1 |
| USH1G | FASLG | PDGFRB | TGFBR3 | GPR56 |
| BMP2 | SLC7A8 | CHI3L1 | RPGRIP1 | GZMM |
| KIF18A | EXO1 | IFNG | KIR2DL2 | SAMD3 |
| ANLN | FGFBP2 | CCL20 | HJURP | |
| GP5 | CXCL1 | DTL | STAP1 | |
| SKA3 | KIAA0101 | LOC643650 | XLOC_l2_010897 | |
| TTK | IFI27 | RGS9 | PPP2R2B | |
| OLFM4 | RAD51 | CEP55 | SPC25 | |
| DLGAP5 | TYMS | POLE2 | KIAA1671 | |

TABLE 4
marker genes (full name and accession number as set out in table 1)

| | | | | | |
|---|---|---|---|---|---|
| LOC572558 | GP5 | MCM10 | FGFBP2 | IFI27 | CHI3L1 |
| USH1G | SKA3 | SLPI | CXCL1 | RAD51 | IFNG |
| BMP2 | TTK | FASLG | MELK | TYMS | CDT1 |
| KIF18A | OLFM4 | SLC7A8 | RRM2 | E2F8 | CCL20 |
| ANLN | DLGAP5 | EXO1 | KIAA0101 | PDGFRB | DTL |

TABLE 4B
marker genes (full name and accession number as set out in table 1)

| | | | | | |
|---|---|---|---|---|---|
| LOC572558 | GP5 | SLPI | CXCL1 | E2F8 | DTL |
| USH1G | SKA3 | FASLG | KIAA0101 | PDGFRB | |
| BMP2 | TTK | SLC7A8 | IFI27 | CHI3L1 | |
| KIF18A | OLFM4 | EXO1 | RAD51 | IFNG | |
| ANLN | DLGAP5 | FGFBP2 | TYMS | CCL20 | |

TABLE 5
marker genes (full name and accession number as set out in table 1)

| | | | | |
|---|---|---|---|---|
| LOC572558 | KIF18A | SKA3 | DLGAP5 | FASLG |
| USH1G | ANLN | TTK | MCM10 | SLC7A8 |
| BMP2 | GP5 | OLFM4 | SLPI | EXO1 |

TABLE 5B
marker genes (full name and accession number as set out in table 1)

| | | | | |
|---|---|---|---|---|
| LOC572558 | KIF18A | SKA3 | DLGAP5 | SLC7A8 |
| USH1G | ANLN | TTK | SLPI | EXO1 |
| BMP2 | GP5 | OLFM4 | FASLG | |

TABLE 6
marker genes for cancer, in particular prostate cancer

| Gene | |
|---|---|
| Symbol | Gene Title |
| COX7B | cytochrome c oxidase subunit VIIb, mRNA |
| | [NM_001866] |
| LY96 | lymphocyte antigen 96, mRNA [NM_015364] |
| FIGNL1 | fidgetin-like 1, mRNA [NM_001042762] |
| FOSL1 | FOS-like antigen 1, mRNA [NM_005438] |
| S100A8 | S100 calcium binding protein A8, mRNA |
| | [NM_002964] |
| GFRA3 | GDNF family receptor alpha 3, mRNA [NM_001496] |
| REEP1 | receptor accessory protein 1, mRNA [NM_022912] |
| SUV39H2 | suppressor of variegation 3-9 homolog 2 (Drosophila), |
| | mRNA [NM_024670] |
| ZFYVE16 | zinc finger, FYVE domain containing 16, mRNA |
| | [NM_001105251] |
| NDUFA4 | NADH dehydrogenase (ubiquinone) 1 alpha |
| | subcomplex, 4, 9 kDa, mRNA [NM_002489] |
| FOXN1 | forkhead box N1, mRNA [NM_003593] |
| KIR2DL4 | killer cell immunoglobulin-like receptor, two domains, |
| | long cytoplasmic tail, 4, mRNA [NM_002255] |
| GOLT1B | golgi transport 1B, mRNA [NM_016072] |
| NLRC3 | NLR family, CARD domain containing 3, mRNA |
| | [NM_178844, AK090476] |
| ACTR6 | ARP6 actin-related protein 6 homolog (yeast), mRNA |
| | [NM_022496] |
| PLEKHF2 | pleckstrin homology domain containing, family F |
| | (with FYVE domain) member 2, mRNA [NM_024613] |
| TFEC | transcription factor EC, mRNA [NM_012252] |
| LGALS2 | lectin, galactoside-binding, soluble, 2, mRNA |
| | [AK130682, NM_006498] |
| MSH6 | mutS homolog 6, mRNA [NM_000179] |
| TARP | TCR gamma alternate reading frame protein, mRNA |
| | [NM_001003799] |
| MND1 | meiotic nuclear divisions 1 homolog (S. cerevisiae), |
| | mRNA [NM_032117] |
| S100A12 | S100 calcium binding protein A12, mRNA |
| | [NM_005621] |
| AGL | amylo-alpha-1, 6-glucosidase, 4-alpha- |
| | glucanotransferase, mRNA [NM_000028] |
| NUPL2 | nucleoporin like 2, mRNA [BC039333, NM_007342] |
| PKMYT1 | protein kinase, membrane associated tyrosine/threonine |
| | 1, mRNA [NM_182687] |
| PTAFR | platelet-activating factor receptor, mRNA |
| | [NM_000952, NM_001164722] |
| VSTM1 | V-set and transmembrane domain containing 1, mRNA |
| | [NM_198481] |
| SAMHD1 | SAM domain and HD domain 1, mRNA [NM_015474] |
| ASPA | aspartoacylase, mRNA [NM_000049] |
| PPP1R12A | protein phosphatase 1, regulatory subunit 12A, mRNA |
| | [NM_002480] |
| UQCRQ | ubiquinol-cytochrome c reductase, complex III subunit |
| | VII, 9.5 kDa, mRNA [NM_014402] |
| OSMR | oncostatin M receptor, mRNA [NM_003999, |
| | NM_001168355] |
| SP3 | Sp3 transcription factor, mRNA [NM_003111] |
| JPH2 | junctophilin 2, mRNA [NM_175913, NM_020433] |
| APCDD1 | adenomatosis polyposis coli down-regulated 1, mRNA |
| | [NM_153000] |
| ABCG2 | ATP-binding cassette, sub-family G (WHITE), |
| | member 2, mRNA [NM_004827] |
| TLR7 | toll-like receptor 7, mRNA [NM_016562] |
| ATP5C1 | ATP synthase, H+ transporting, mitochondrial F1 |
| | complex, gamma polypeptide 1, mRNA |
| | [NM_005174] |
| GINS1 | GINS complex subunit 1 (Psf1 homolog), mRNA |
| | [NM_021067] |
| CKS2 | CDC28 protein kinase regulatory subunit 2, mRNA |
| | [NM_001827] |
| NOC3L | nucleolar complex associated 3 homolog |
| | (S. cerevisiae), mRNA [NM_022451] |
| DEK | DEK oncogene, mRNA [NM_003472] |
| HNMT | histamine N-methyltransferase, mRNA [NM_006895, |
| | NM_001024074, NM_001024075] |
| MS4A4A | membrane-spanning 4-domains, subfamily A, member |
| | 4A, mRNA [NM_024021] |
| TMEM55A | transmembrane protein 55A, mRNA [NM_018710] |
| TMX1 | thioredoxin-related transmembrane protein 1, mRNA |
| | [NM_030755] |
| USP1 | ubiquitin specific peptidase 1, mRNA [NM_003368] |
| CCDC80 | coiled-coil domain containing 80, mRNA |
| | [NM_199511] |
| PCNP | PEST proteolytic signal containing nuclear protein, |
| | mRNA [NM_020357] |
| SHCBP1 | SHC SH2-domain binding protein 1, mRNA |
| | [NM_024745] |
| PANK3 | pantothenate kinase 3, mRNA [BX648891, |
| | NM_024594] |
| PAQR3 | progestin and adipoQ receptor family member III, |
| | mRNA [NM_001040202, BC031256] |
| RNF138 | ring finger protein 138, E3 ubiquitin protein ligase, |
| | mRNA [NM_016271] |
| CD302 | CD302 molecule, mRNA [NM_014880] |
| NUP35 | nucleoporin 35 kDa, mRNA [NM_138285, |
| | NM_001287585] |
| IMPA1 | inositol(myo)-1(or 4)-monophosphatase 1, mRNA |
| | [NM_001144879, NM_005536] |
| MS4A7 | membrane-spanning 4-domains, subfamily A, member |
| | 7, mRNA [NM_021201] |
| SGMS1 | sphingomyelin synthase 1, mRNA [NM_147156] |
| GALNT7 | UDP-N-acetyl-alpha-D-galactosamine:polypeptide N- |
| | acetylgalactosaminyltransferase 7 (GalNAc-T7), |
| | mRNA [NM_017423] |
| CA14 | carbonic anhydrase XIV, mRNA [NM_012113] |
| RHOU | ras homolog family member U, mRNA [NM_021205] |
| PHTF2 | putative homeodomain transcription factor 2, mRNA |
| | [NM_001127360, NM_020432] |
| S100A9 | S100 calcium binding protein A9, mRNA |
| | [NM_002965] |
| FAM198B | family with sequence similarity 198, member B, |
| | mRNA [NM_016613] |
| ST3GAL6 | ST3 beta-galactoside alpha-2,3-sialyltransferase 6, |
| | mRNA [NM_006100] |
| CLK4 | CDC-like kinase 4, mRNA [NM_020666] |
| CLEC12A | C-type lectin domain family 12, member A, mRNA |
| | [NM_138337] |
| GCA | grancalcin, EF-hand calcium binding protein, mRNA |
| | [NM_012198] |
| VNN2 | vanin 2, mRNA [NM_004665] |
| C1GALT1C1 | C1GALT1-specific chaperone 1, mRNA |
| | [NM_152692] |
| DUSP6 | dual specificity phosphatase 6, mRNA [NM_001946] |
| TNFAIP8 | tumor necrosis factor, alpha-induced protein 8, mRNA |
| | [NM_001286813, NM_014350] |
| TLR4 | toll-like receptor 4, mRNA [NM_138554] |
| FBLN1 | fibulin 1, mRNA [NM_001996, NM_006486] |
| LRRK2 | leucine-rich repeat kinase 2, mRNA [NM_198578, |
| | AK127729] |
| SLC46A2 | solute carrier family 46, member 2, mRNA |
| | [NM_033051] |
| KIAA1468 | KIAA1468, mRNA [NM_020854] |
| AIF1 | allograft inflammatory factor 1, mRNA [NM_004847] |
| HK3 | hexokinase 3 (white cell), mRNA [NM_002115] |
| SFRP4 | secreted frizzled-related protein 4, mRNA |
| | [NM_003014] |
| ARFIP1 | ADP-ribosylation factor interacting protein 1, mRNA |
| | [NM_001025595] |
| COX6C | cytochrome c oxidase subunit VIc, mRNA |
| | [NM_004374] |
| DMD | dystrophin, mRNA [NM_004021, BC036103, |
| | NM_004010] |
| KYNU | kynureninase, mRNA [NM_001032998] |
| PIK3C2A | phosphatidylinositol-4-phosphate 3-kinase, catalytic |
| | subunit type 2 alpha, mRNA [NM_002645] |
| NDUFB1 | NADH dehydrogenase (ubiquinone) 1 beta |
| | subcomplex, 1, 7 kDa, mRNA [NM_004545] |
| VNN1 | vanin 1, mRNA [NM_004666] |
| MBNL1 | muscleblind-like splicing regulator 1, mRNA |
| | [NM_021038] |
| ABCA1 | ATP-binding cassette, sub-family A (ABC1), member |
| | 1, mRNA [NM_005502] |
| PID1 | phosphotyrosine interaction domain containing 1, |
| | mRNA [NM_017933] |
| SRP9 | signal recognition particle 9 kDa, mRNA |
| | [NM_001130440, NM_003133] |
| MAD2L1 | MAD2 mitotic arrest deficient-like 1 (yeast), mRNA |
| | [NM_002358] |
| GLIPR1 | GLI pathogenesis-related 1, mRNA [NM_006851] |
| NUP107 | nucleoporin 107 kDa, mRNA [NM_020401] |
| GZMK | granzyme K (granzyme 3; tryptase II), mRNA |
| | [NM_002104] |
| STAG2 | stromal antigen 2, mRNA [NM_001042749, |
| | NM_001042751] |
| NDUFB3 | NADH dehydrogenase (ubiquinone) 1 beta |
| | subcomplex, 3, 12 kDa, mRNA [NM_001257102] |
| CYP1B1 | cytochrome P450, family 1, subfamily B, polypeptide |
| | 1, mRNA [NM_000104] |
| MYBL2 | v-myb avian myeloblastosis viral oncogene homolog- |
| | like 2, mRNA [NM_002466] |
| KLF1 | Kruppel-like factor 1 (erythroid), mRNA |
| | [NM_006563] |
| IFI30 | interferon, gamma-inducible protein 30, mRNA |
| | [NM_006332] |
| ICOS | inducible T-cell co-stimulator, mRNA [NM_012092] |
| TNFSF11 | tumor necrosis factor (ligand) superfamily, member 11, |
| | mRNA [NM_003701, NM_033012] |
| NDUFB2 | NADH dehydrogenase (ubiquinone) 1 beta |
| | subcomplex, 2, 8 kDa, mRNA [NM_004546] |
| TGM2 | transglutaminase 2, mRNA [NM_004613, |
| | NM_198951] |
| CUL5 | cullin 5, mRNA [NM_003478] |
| CD28 | CD28 molecule, mRNA [NM_006139] |
| SGK1 | serum/glucocorticoid regulated kinase 1, mRNA |
| | [NM_005627] |
| PLA2G7 | phospholipase A2, group VII (platelet-activating |
| | factor acetylhydrolase, plasma), mRNA [NM_005084] |
| RMI1 | RecQ mediated genome instability 1, mRNA |
| | [NM_024945] |
| GPSM2 | G-protein signaling modulator 2, mRNA |
| | [NM_013296] |
| ZDHHC17 | zinc finger, DHHC-type containing 17, mRNA |
| | [NM_015336] |
| CCR2 | chemokine (C-C motif) receptor 2, mRNA |
| | [NM_001123041, NM_001123396] |
| FCN1 | ficolin (collagen/fibrinogen domain containing) 1, |
| | mRNA [NM_002003] |
| ZWINT | ZW10 interacting kinetochore protein, mRNA |
| | [NM_032997] |
| AMICA1 | adhesion molecule, interacts with CXADR antigen 1, |
| | mRNA [AK057590, NM_153206] |
| CLEC7A | C-type lectin domain family 7, member A, mRNA |
| | [NM_197947, NM_197954] |
| ERBB2IP | ebb2 interacting protein, mRNA [NM_018695] |
| MSH2 | mutS homolog 2, mRNA [NM_000251] |
| RIN2 | Ras and Rab interactor 2, mRNA [NM_018993] |
| MORC3 | MORC family CW-type zinc finger 3, mRNA |
| | [NM_015358] |
| MFSD1 | major facilitator superfamily domain containing 1, |
| | mRNA [NM_022736] |
| KCNJ2 | potassium inwardly-rectifying channel, subfamily J, |
| | member 2, mRNA [NM_000891] |
| LMNB1 | lamin B1, mRNA [NM_005573] |
| CCT2 | chaperonin containing TCP1, subunit 2 (beta), mRNA |
| | [NM_006431] |
| IFNG | interferon, gamma, mRNA [NM_000619] |
| EEF1G | eukaryotic translation elongation factor 1 gamma, |
| | mRNA [NM_001404] |
| CSTA | cystatin A (stefin A), mRNA [NM_005213] |
| MYBL1 | v-myb avian myeloblastosis viral oncogene homolog- |
| | like 1, mRNA [NM_001294282, NM_001080416] |
| MNDA | myeloid cell nuclear differentiation antigen, mRNA |
| | [NM_002432] |
| PAQR9 | progestin and adipoQ receptor family member IX, |
| | mRNA [NM_198504] |
| ZBTB11 | zinc finger and BTB domain containing 11, mRNA |
| | [NM_014415] |
| RBP7 | retinol binding protein 7, cellular, mRNA |
| | [NM_052960] |
| ZNF700 | zinc finger protein 700, mRNA [NM_144566] |
| BST1 | bone marrow stromal cell antigen 1, mRNA |
| | [NM_004334] |
| ATP5O | ATP synthase, H+ transporting, mitochondrial F1 |
| | complex, O subunit, mRNA [NM_001697] |
| ATAD2 | ATPase family, AAA domain containing 2, mRNA |
| | [NM_014109] |
| DLGAP5 | discs, large (Drosophila) homolog-associated protein |
| | 5, mRNA [NM_014750] |
| NOD2 | nucleotide-binding oligomerization domain containing |
| | 2, mRNA [NM_022162] |
| LILRA3 | leukocyte immunoglobulin-like receptor, subfamily A |
| | (without TM domain), member 3, mRNA |
| | [NM_006865] |
| CLEC4A | C-type lectin domain family 4, member A, mRNA |
| | [NM_016184] |
| TNFSF13B | tumor necrosis factor (ligand) superfamily, member |
| | 13b, mRNA [NM_006573] |
| CSF2RA | colony stimulating factor 2 receptor, alpha, low-affinity |
| | (granulocyte-macrophage), mRNA [NM_172249] |
| BRI3 | brain protein I3, mRNA [NM_001159491, |
| | NM_015379] |
| RNF6 | ring finger protein (C3H2C3 type) 6, mRNA |
| | [NM_005977] |
| PTTG3P | pituitary tumor-transforming 3, pseudogene, mRNA |
| | [NR_002734] |
| HSPD1 | heat shock 60 kDa protein 1 (chaperonin), mRNA |
| | [NM_002156] |
| GTF2H1 | general transcription factor IIH, polypeptide 1, 62 kDa, |
| | mRNA [NM_005316] |
| LMO2 | LIM domain only 2 (rhombotin-like 1), mRNA |
| | [NM_005574] |
| KIAA0101 | KIAA0101, mRNA [NM_014736] |
| RFC5 | replication factor C (activator 1) 5, 36.5 kDa, mRNA |
| | [NM_181578] |
| RAD50 | RAD50 homolog (S. cerevisiae), mRNA |
| | [NM_005732] |
| FEN1 | flap structure-specific endonuclease 1, mRNA |
| | [NM_004111] |
| NUP54 | nucleoporin 54 kDa, mRNA [NM_017426] |
| NDUFS4 | NADH dehydrogenase (ubiquinone) Fe—S protein 4, |
| | 18 kDa (NADH-coenzyme Q reductase), mRNA |
| | [NM_002495] |
| PTPN22 | protein tyrosine phosphatase, non-receptor type 22 |
| | (lymphoid), mRNA [NM_012411, NM_015967] |
| CDKN3 | cyclin-dependent kinase inhibitor 3, mRNA |
| | [NM_005192] |
| MAGEA12 | melanoma antigen family A, 12, mRNA |
| | [NM_005367] |
| VPS4B | vacuolar protein sorting 4 homolog B (S. cerevisiae), |
| | mRNA [NM_004869] |
| EIF3E | eukaryotic translation initiation factor 3, subunit E, |
| | mRNA [NM_001568] |
| HLA-DPB1 | major histocompatibility complex, class II, DP beta 1, |
| | mRNA [XM_006715078, NM_002121] |
| NCAPG | non-SMC condensin I complex, subunit G, mRNA |
| | [NM_022346] |
| CREBZF | CREB/ATF bZIP transcription factor, mRNA |
| | [NM_001039618] |
| ITSN2 | intersectin 2, mRNA [NM_147152] |
| QPCT | glutaminyl-peptide cyclotransferase, mRNA |
| | [NM_012413] |
| BZW1 | basic leucine zipper and W2 domains 1, mRNA |
| | [NM_014670] |
| NDC80 | NDC80 kinetochore complex component, mRNA |
| | [NM_006101] |
| ZFAND6 | zinc finger, AN1-type domain 6, mRNA [NM_019006] |
| KIR2DS2 | killer cell immunoglobulin-like receptor, two domains, |
| | short cytoplasmic tail, 2, mRNA [NM_012312] |
| CCNC | cyclin C, mRNA [BC026272, NM_005190] |
| EIF3H | eukaryotic translation initiation factor 3, subunit H, |
| | mRNA [NM_003756] |
| FPR1 | formyl peptide receptor 1, mRNA [NM_002029] |
| SLC40A1 | solute carrier family 40 (iron-regulated transporter), |
| | member 1, mRNA [NM_014585] |
| NUP160 | nucleoporin 160 kDa, mRNA [NM_015231] |
| ENPP5 | ectonucleotide pyrophosphatase/phosphodiesterase 5 |
| | (putative), mRNA [NM_021572] |
| MARCH1 | membrane-associated ring finger (C3HC4) 1, E3 |
| | ubiquitin protein ligase, mRNA [NM_017923] |
| DPYD | dihydropyrimidine dehydrogenase, mRNA |
| | [NM_001160301, NM_000110] |
| TGFBI | transforming growth factor, beta-induced, 68 kDa, |
| | mRNA [NM_000358] |
| FBXO5 | F-box protein 5, mRNA [NM_001142522] |
| ATP5L | ATP synthase, H+ transporting, mitochondrial Fo |
| | complex, subunit G, mRNA [NM_006476] |
| G3BP2 | GTPase activating protein (SH3 domain) binding |
| | protein 2, mRNA [NM_203505] |
| MARCKS | myristoylated alanine-rich protein kinase C substrate, |
| | mRNA [D10522] |
| SEC63 | SEC63 homolog (S. cerevisiae), mRNA [NM_007214] |
| COX6A1 | cytochrome c oxidase subunit VIa polypeptide 1, |
| | mRNA [NM_004373] |
| PDGFA | platelet-derived growth factor alpha polypeptide, |
| | mRNA [NM_002607, NM_033023] |
| CDT1 | chromatin licensing and DNA replication factor 1, |
| | mRNA [NM_030928] |
| KLRB1 | killer cell lectin-like receptor subfamily B, member 1, |
| | mRNA [NM_002258] |
| SMC2 | structural maintenance of chromosomes 2, mRNA |
| | [NM_001042550] |
| HLA-DMB | major histocompatibility complex, class II, DM beta, |
| | mRNA [NM_002118] |
| TYROBP | TYRO protein tyrosine kinase binding protein, mRNA |
| | [NM_003332] |
| GLT8D2 | glycosyltransferase 8 domain containing 2, mRNA |
| | [NM_031302] |
| GMNN | geminin, DNA replication inhibitor, mRNA |
| | [NM_015895] |
| FGR | feline Gardner-Rasheed sarcoma viral oncogene |
| | homolog, mRNA [NM_001042747] |
| COX5B | cytochrome c oxidase subunit Vb, mRNA |
| | [NM_001862] |
| KIF18A | kinesin family member 18A, mRNA [NM_031217] |
| KIF20B | kinesin family member 20B, mRNA [NM_016195, |
| | NM_001284259] |
| HOXD10 | homeobox D10, mRNA [NM_002148] |
| RAD21 | RAD21 homolog (S. pombe), mRNA [NM_006265] |
| MATR3 | matrin 3, mRNA [NM_199189] |
| APOBEC3B | apolipoprotein B mRNA editing enzyme, catalytic |
| | polypeptide-like 3B, mRNA [NM_004900] |
| HMOX1 | heme oxygenase (decycling) 1, mRNA [NM_002133] |
| SLC31A2 | solute carrier family 31 (copper transporter), member |
| | 2, mRNA [NM_001860] |
| VNN3 | vanin 3, mRNA [NM_001291703] |
| TSPAN5 | tetraspanin 5, mRNA [NM_005723] |
| GATA1 | GATA binding protein 1 (globin transcription factor 1), |
| | mRNA [NM_002049] |
| MARCO | macrophage receptor with collagenous structure, |
| | mRNA [NM_006770] |
| SIRPB2 | signal-regulatory protein beta 2, mRNA |
| | [NM_001122962] |
| LST1 | leukocyte specific transcript 1, mRNA [NM_007161] |
| CEACAM7 | carcinoembryonic antigen-related cell adhesion |
| | molecule 7, mRNA [NM_006890] |
| KIF20A | kinesin family member 20A, mRNA [NM_005733] |
| EED | embryonic ectoderm development, mRNA |
| | [NM_152991] |
| RAB31 | RAB31, member RAS oncogene family, mRNA |
| | [NM_006868] |
| NDUFB6 | NADH dehydrogenase (ubiquinone) 1 beta |
| | subcomplex, 6, 17 kDa, mRNA [NM_002493, |
| | NM_182739] |
| HSPE1 | heat shock 10 kDa protein 1, mRNA [NM_002157] |
| TMEM154 | transmembrane protein 154, mRNA [NM_152680] |
| TLR1 | toll-like receptor 1, mRNA [NM_003263] |
| CD14 | CD14 molecule, mRNA [NM_001174104] |
| SPC25 | SPC25, NDC80 kinetochore complex component, |
| | mRNA [NM_020675] |
| ASGR1 | asialoglycoprotein receptor 1, mRNA [AK308677, |
| | NM_001671] |
| STK26 | serine/threonine protein kinase MST4, mRNA |
| | [NM_016542] |
| EVI5 | ecotropic viral integration site 5, mRNA [NM_005665] |
| SLC35B3 | solute carrier family 35 (adenosine 3′-phospho |
| | 5′-phosphosulfate transporter), member B3, mRNA |
| | [NM_015948] |
| FBXW2 | F-box and WD repeat domain containing 2, mRNA |
| | [NM_012164] |
| TLR6 | toll-like receptor 6, mRNA [NM_006068] |
| EMILIN2 | elastin microfibril interfacer 2, mRNA [NM_032048] |
| CCNB1 | cyclin B1, mRNA [NM_031966] |
| HLA-DRB1 | major histocompatibility complex, class II, DR beta 1, |
| | mRNA [NM_002124] |
| GAS1 | growth arrest-specific 1, mRNA [NM_002048] |
| RNASE2 | ribonuclease, RNase A family, 2 (liver, eosinophil- |
| | derived neurotoxin), mRNA [NM_002934] |
| CTSH | cathepsin H, mRNA [NM_004390] |
| PILRA | paired immunoglobin-like type 2 receptor alpha, |
| | mRNA [NM_178273] |
| S1PR3 | sphingosine-1-phosphate receptor 3, mRNA |
| | [NM_005226] |
| TNF | tumor necrosis factor, mRNA [NM_000594] |
| IL1RN | interleukin 1 receptor antagonist, mRNA |
| | [NM_173843] |
| CEP55 | centrosomal protein 55 kDa, mRNA [NM_018131] |
| ECT2 | epithelial cell transforming sequence 2 oncogene, |
| | mRNA [NM_018098] |
| AQP9 | aquaporin 9, mRNA [NM_020980] |
| NDUFAB1 | NADH dehydrogenase (ubiquinone) 1, alpha/beta |
| | subcomplex, 1, 8 kDa, mRNA [NM_005003] |
| SLA | Src-like-adaptor, mRNA [NM_001045556] |
| FES | feline sarcoma oncogene, mRNA [NM_002005] |
| HSPA6 | heat shock 70 kDa protein 6 (HSP70B′), mRNA |
| | [NM_002155] |
| RNF130 | ring finger protein 130, mRNA [NM_018434, |
| | NM_001280801] |
| CEP120 | centrosomal protein 120 kDa, mRNA [CR627324, |
| | NM_153223] |
| PNPLA8 | patatin-like phospholipase domain containing 8, |
| | mRNA [NM_015723] |
| HLA-DRB4 | major histocompatibility complex, class II, DR beta 4, |
| | mRNA [NM_021983] |
| ZBED5 | zinc finger, BED-type containing 5, mRNA |
| | [NM_021211] |
| RANBP1 | RAN binding protein 1, mRNA [NM_002882] |
| MORF4L2 | mortality factor 4 like 2, mRNA [NM_012286] |
| PADI2 | peptidyl arginine deiminase, type II, mRNA |
| | [NM_007365] |
| TPX2 | TPX2, microtubule-associated, mRNA [NM_012112] |
| NEK2 | NIMA-related kinase 2, mRNA [NM_002497] |
| EPB42 | erythrocyte membrane protein band 4.2, mRNA |
| | [NM_000119] |
| ARMC1 | armadillo repeat containing 1, mRNA [NM_018120] |
| SMARCA5 | SWI/SNF related, matrix associated, actin dependent |
| | regulator of chromatin, subfamily a, member 5, |
| | mRNA [NM_003601] |
| CSF1R | colony stimulating factor 1 receptor, mRNA |
| | [NM_005211] |
| ATP5J2 | ATP synthase, H+ transporting, mitochondrial Fo |
| | complex, subunit F2, mRNA [NM_004889, |
| | NM_001003714] |
| RPA3 | replication protein A3, 14 kDa, mRNA [NM_002947] |
| SMARCD3 | SWI/SNF related, matrix associated, actin dependent |
| | regulator of chromatin, subfamily d, member 3, mRNA |
| | [NM_003078] |
| EIF3K | eukaryotic translation initiation factor 3, subunit K, |
| | mRNA [NM_013234] |
| GPR56 | G protein-coupled receptor 56, mRNA [NM_201525] |
| ATP5I | ATP synthase, H+ transporting, mitochondrial Fo |
| | complex, subunit E, mRNA [NM_007100] |
| NIF3L1 | NIF3 NGG1 interacting factor 3-like 1 (S. cerevisiae), |
| | mRNA [NM_021824, NM_001142356] |
| CD53 | CD53 molecule, mRNA [NM_001040033] |
| MGST1 | microsomal glutathione S-transferase 1, mRNA |
| | [NM_145791] |
| COX7A2L | cytochrome c oxidase subunit VIIa polypeptide 2 like, |
| | mRNA [NM_004718] |
| FCGR1B | Fc fragment of IgG, high affinity Ib, receptor (CD64), |
| | mRNA [NM_001244910, NM_001017986] |
| KCNE3 | potassium voltage-gated channel, Isk-related family, |
| | member 3, mRNA [NM_005472] |
| XPO1 | exportin 1 (CRM1 homolog, yeast), mRNA |
| | [NM_003400] |
| PDE6D | phosphodiesterase 6D, cGMP-specific, rod, delta, |
| | mRNA [NM_002601] |
| MS4A6A | membrane-spanning 4-domains, subfamily A, member |
| | 6A, mRNA [NM_152852, NM_022349] |
| FBL | fibrillarin, mRNA [NM_001436] |
| SIRPB1 | signal-regulatory protein beta 1, mRNA |
| | [NM_001135844, XM_006710250, NM_006065] |
| RAD51 | RAD51 recombinase, mRNA [NM_002875] |
| PLBD1 | phospholipase B domain containing 1, mRNA |
| | [NM_024829] |
| LAT2 | linker for activation of T cells family, member 2, |
| | mRNA [NM_032464, XM_005250562] |
| LILRB3 | leukocyte immunoglobulin-like receptor, subfamily B |
| | (with TM and ITIM domains), member 3, mRNA |
| | [NM_006864] |
| LILRA5 | leukocyte immunoglobulin-like receptor, subfamily A |
| | (with TM domain), member 5, mRNA [NM_181879] |
| PRIM1 | primase, DNA, polypeptide 1 (49 kDa), mRNA |
| | [NM_000946] |
| CFD | complement factor D (adipsin), mRNA [NM_001928] |
| NCF2 | neutrophil cytosolic factor 2, mRNA [NM_000433] |
| NDUFA2 | NADH dehydrogenase (ubiquinone) 1 alpha |
| | subcomplex, 2, 8 kDa, mRNA [NM_002488] |
| SMPDL3A | sphingomyelin phosphodiesterase, acid-like 3A, |
| | mRNA [NM_006714] |
| AGTRAP | angiotensin II receptor-associated protein, mRNA |
| | [NM_001040196] |
| GIMAP7 | GTPase, IMAP family member 7, mRNA |
| | [NM_153236] |
| IRAK3 | interleukin-1 receptor-associated kinase 3, mRNA |
| | [NM_007199] |
| ASGR2 | asialoglycoprotein receptor 2, mRNA [NM_080912] |
| UQCRC1 | ubiquinol-cytochrome c reductase core protein I, |
| | mRNA [NM_003365] |
| CD36 | CD36 molecule (thrombospondin receptor), mRNA |
| | [NM_001001547] |
| CD33 | CD33 molecule, mRNA [NM_001772, |
| | NM_001177608] |
| PSAP | prosaposin, mRNA [NM_001042465] |
| RAD51C | RAD51 paralog C, mRNA [NM_002876] |
| LCP1 | lymphocyte cytosolic protein 1 (L-plastin), mRNA |
| | [NM_002298] |
| BCL6 | B-cell CLL/lymphoma 6, mRNA [NM_001130845] |
| LILRA6 | leukocyte immunoglobulin-like receptor, subfamily B |
| | (with TM and ITIM domains), member 3, mRNA |
| | [NR_104098, NM_024318] |
| CCT8 | chaperonin containing TCP1, subunit 8 (theta), mRNA |
| | [NM_006585] |
| ACSL1 | acyl-CoA synthetase long-chain family member 1, |
| | mRNA [NM_001995] |
| HCLS1 | hematopoietic cell-specific Lyn substrate 1, mRNA |
| | [NM_005335] |
| ITGA4 | integrin, alpha 4 (antigen CD49D, alpha 4 subunit of |
| | VLA-4 receptor), mRNA [NM_000885] |
| SULF2 | sulfatase 2, mRNA [NM_018837] |
| CRTAM | cytotoxic and regulatory T cell molecule, mRNA |
| | [NM_019604] |
| CCT4 | chaperonin containing TCP1, subunit 4 (delta), mRNA |
| | [NM_006430] |
| CHRDL1 | chordin-like 1, mRNA [NM_145234, NM_001143981] |
| GZMA | granzyme A (granzyme 1, cytotoxic T-lymphocyte- |
| | associated serine esterase 3), mRNA [NM_006144] |
| PGD | phosphogluconate dehydrogenase, mRNA |
| | [NM_002631] |
| ARID4B | AT rich interactive domain 4B (RBP1-like), mRNA |
| | [NM_016374, AB030181] |
| RFC4 | replication factor C (activator 1) 4, 37 kDa, mRNA |
| | [NM_002916] |
| TNFRSF10B | tumor necrosis factor receptor superfamily, member |
| | 10b, mRNA [NM_003842] |
| POLD3 | polymerase (DNA-directed), delta 3, accessory subunit, |
| | mRNA [NM_006591] |
| UQCRFS1 | ubiquinol-cytochrome c reductase, Rieske iron-sulfur |
| | polypeptide 1, mRNA [NM_006003] |
| ROCK1 | Rho-associated, coiled-coil containing protein kinase 1, |
| | mRNA [NM_005406] |
| NDUFS3 | NADH dehydrogenase (ubiquinone) Fe—S protein 3, |
| | 30 kDa (NADH-coenzyme Q reductase), mRNA |
| | [NM_004551] |
| CCT3 | chaperonin containing TCP1, subunit 3 (gamma), |
| | mRNA [NM_005998] |
| HLA-DMA | major histocompatibility complex, class II, DM alpha, |
| | mRNA [NM_006120] |
| GP9 | glycoprotein IX (platelet), mRNA [NM_000174] |
| MYD88 | myeloid differentiation primary response 88, mRNA |
| | [NM_002468] |
| SH2D1A | SH2 domain containing 1A, mRNA [NM_001114937, |
| | NM_002351] |
| NCF1 | neutrophil cytosolic factor 1, mRNA [NM_000265] |
| CD27 | CD27 molecule, mRNA [NM_001242] |
| SPRY4 | sprouty homolog 4 (Drosophila), mRNA |
| | [NM_001293289, NM_030964] |
| NLGN1 | neuroligin 1, mRNA [NM_014932] |
| HPSE | heparanase, mRNA [NM_006665] |
| MED23 | mediator complex subunit 23, mRNA |
| | [NM_001270522, AF104255, NM_015979, |
| | NM_004830] |
| UBE2T | ubiquitin-conjugating enzyme E2T (putative), mRNA |
| | [NM_014176] |
| LILRA2 | leukocyte immunoglobulin-like receptor, subfamily A |
| | (with TM domain), member 2, mRNA [NM_006866] |
| LEF1 | lymphoid enhancer-binding factor 1, mRNA |
| | [NM_016269] |
| KNTC1 | kinetochore associated 1, mRNA [NM_014708] |
| ACPP | acid phosphatase, prostate, mRNA [NM_001099] |
| APOBEC3A | apolipoprotein B mRNA editing enzyme, catalytic |
| | polypeptide-like 3A, mRNA [NM_145699] |
| GOLPH3L | golgi phosphoprotein 3-like, mRNA [NM_018178] |
| UQCRH | ubiquinol-cytochrome c reductase hinge protein, |
| | mRNA [NM_006004, NM_001297565] |
| SLC7A7 | solute carrier family 7 (amino acid transporter light |
| | chain, y + L system), member 7, mRNA |
| | [NM_001126106] |
| FGFBP2 | fibroblast growth factor binding protein 2, mRNA |
| | [NM_031950] |
| TMPO | thymopoietin, mRNA [NM_003276, NM_001032283] |
| CEP76 | centrosomal protein 76 kDa, mRNA [NM_024899] |
| CPVL | carboxypeptidase, vitellogenic-like, mRNA |
| | [NM_019029] |
| IL10RA | interleukin 10 receptor, alpha, mRNA [NM_001558] |
| TYMS | thymidylate synthetase, mRNA [NM_001071] |
| TSN | translin, mRNA [NM_004622] |
| CDC25A | cell division cycle 25A, mRNA [NM_001789] |
| CKS1B | CDC28 protein kinase regulatory subunit 1B, mRNA |
| | [NM_001826] |
| GPR171 | G protein-coupled receptor 171, mRNA [NM_013308] |
| POLA2 | polymerase (DNA directed), alpha 2, accessory |
| | subunit, mRNA [AK307831] |
| CLK1 | CDC-like kinase 1, mRNA [NM_004071, |
| | NM_001162407] |
| RAN | RAN, member RAS oncogene family, mRNA |
| | [NM_006325] |
| F5 | coagulation factor V (proaccelerin, labile factor), |
| | mRNA [NM_000130] |
| CD2 | CD2 molecule, mRNA [AK310676, NM_001767] |
| NDUFA8 | NADH dehydrogenase (ubiquinone) 1 alpha |
| | subcomplex, 8, 19 kDa, mRNA [NM_014222] |
| RBM47 | RNA binding motif protein 47, mRNA [NM_019027] |
| CST3 | cystatin C, mRNA [NM_001288614, NM_000099] |
| NDUFA11 | NADH dehydrogenase (ubiquinone) 1 alpha |
| | subcomplex, 11, 14.7 kDa, mRNA [NM_175614] |
| SKA3 | spindle and kinetochore associated complex subunit 3, |
| | mRNA [NM_145061] |
| EIF3D | eukaryotic translation initiation factor 3, subunit D, |
| | mRNA [NM_003753] |
| FHL2 | four and a half LIM domains 2, mRNA [CR936682, |
| | NM_001039492, NM_201555] |
| MAFB | v-maf avian musculoaponeurotic fibrosarcoma |
| | oncogene homolog B, mRNA [NM_005461] |
| TLK1 | tousled-like kinase 1, mRNA [NM_012290] |
| CHAC2 | ChaC, cation transport regulator homolog 2 (E. coli), |
| | mRNA [NM_001008708] |
| RAB21 | RAB21, member RAS oncogene family, mRNA |
| | [NM_014999] |
| COX6B1 | cytochrome c oxidase subunit VIb polypeptide 1 |
| | (ubiquitous), mRNA [NM_001863] |
| GPR18 | G protein-coupled receptor 18, mRNA [NM_005292] |
| SLAMF1 | signaling lymphocytic activation molecule family |
| | member 1, mRNA [NM_003037] |
| NUP88 | nucleoporin 88 kDa, mRNA [NM_002532] |
| UQCRC2 | ubiquinol-cytochrome c reductase core protein II, |
| | mRNA [NM_003366] |
| BRCA1 | breast cancer 1, early onset, mRNA [NM_007300] |
| CYFIP1 | cytoplasmic FMR1 interacting protein 1, mRNA |
| | [NM_014608] |
| TMOD1 | tropomodulin 1, mRNA [NM_003275] |
| TIPIN | TIMELESS interacting protein, mRNA [NM_017858] |
| COX8A | cytochrome c oxidase subunit VIIIA (ubiquitous), |
| | mRNA [NM_004074] |
| LILRB2 | leukocyte immunoglobulin-like receptor, subfamily B |
| | (with TM and ITIM domains), member 2, mRNA |
| | [NM_005874] |
| RFC2 | replication factor C (activator 1) 2, 40 kDa, mRNA |
| | [NM_181471] |
| IL13RA1 | interleukin 13 receptor, alpha 1, mRNA [NM_001560] |
| CD300LF | CD300 molecule-like family member f, mRNA |
| | [NM_139018] |
| NCF4 | neutrophil cytosolic factor 4, 40 kDa, mRNA |
| | [NM_000631] |
| RRM1 | ribonucleotide reductase M1, mRNA [NM_001033] |
| ELAVL3 | ELAV like neuron-specific RNA binding protein 3, |
| | mRNA [NM_001420] |
| LTBR | lymphotoxin beta receptor (TNFR superfamily, |
| | member 3), mRNA [NM_002342] |
| ALOX5 | arachidonate 5-lipoxygenase, mRNA [NM_000698, |
| | AB208946] |
| PLAUR | plasminogen activator, urokinase receptor, mRNA |
| | [NM_001005377] |
| CRISPLD2 | cysteine-rich secretory protein LCCL domain |
| | containing 2, mRNA [NM_031476] |
| PYGL | phosphorylase, glycogen, liver, mRNA [NM_002863] |
| MDFIC | MyoD family inhibitor domain containing, mRNA |
| | [NM_199072, NM_001166346] |
| CFP | complement factor properdin, mRNA [NM_002621] |
| KIF2C | kinesin family member 2C, mRNA [NM_006845] |
| HCK | hemopoietic cell kinase, mRNA [NM_002110] |
| THEMIS | thymocyte selection associated, mRNA |
| | [NM_001164685] |
| KIAA1598 | KIAA1598, mRNA [NM_018330, NM_001127211, |
| | NM_001258299] |
| IL1R2 | interleukin 1 receptor, type II, mRNA [NM_004633] |
| FAM83D | family with sequence similarity 83, member D, mRNA |
| | [NM_030919] |
| CXCR2 | chemokine (C-X-C motif) receptor 2, mRNA |
| | [NM_001557] |
| RTN1 | reticulon 1, mRNA [NM_021136] |
| TIMP2 | TIMP metallopeptidase inhibitor 2, mRNA |
| | [NM_003255] |
| GATA3 | GATA binding protein 3, mRNA [NM_001002295] |
| UBA3 | ubiquitin-like modifier activating enzyme 3, mRNA |
| | [NM_003968] |
| PRRX1 | paired related homeobox 1, mRNA [NM_006902] |
| NLRP3 | NLR family, pyrin domain containing 3, mRNA |
| | [NM_001243133, NM_001079821] |
| UHRF1 | ubiquitin-like with PHD and ring finger domains 1, |
| | mRNA [NM_013282, NM_001290050] |
| OIP5 | Opa interacting protein 5, mRNA [NM_007280] |
| PAK1 | p21 protein (Cdc42/Rac)-activated kinase 1, mRNA |
| | [NM_001128620] |
| LY86 | lymphocyte antigen 86, mRNA [NM_004271] |
| KLRD1 | killer cell lectin-like receptor subfamily D, member 1, |
| | mRNA [NM_002262] |
| DTL | denticleless E3 ubiquitin protein ligase homolog |
| | (Drosophila), mRNA [NM_016448] |
| SIRPA | signal-regulatory protein alpha, mRNA |
| | [NM_001040022] |
| TRAT1 | T cell receptor associated transmembrane adaptor 1, |
| | mRNA [NM_016388] |
| RASSF4 | Ras association (RalGDS/AF-6) domain family |
| | member 4, mRNA [NM_032023] |
| PECAM1 | platelet/endothelial cell adhesion molecule 1, mRNA |
| | [NM_000442] |
| RFC3 | replication factor C (activator 1) 3, 38 kDa, mRNA |
| | [NM_002915, NM_181558] |
| ACTL6A | actin-like 6A, mRNA [AK223212] |
| CHEK1 | checkpoint kinase 1, mRNA [NM_001114121] |
| BUB1 | BUB1 mitotic checkpoint serine/threonine kinase, |
| | mRNA [NM_004336] |
| PYCARD | PYD and CARD domain containing, mRNA |
| | [NM_013258] |
| EIF3F | eukaryotic translation initiation factor 3, subunit F, |
| | mRNA [NM_003754] |
| ARHGEF10L | Rho guanine nucleotide exchange factor (GEF) 10-like, |
| | mRNA [NM_018125] |
| ITM2A | integral membrane protein 2A, mRNA [NM_004867] |
| LILRB1 | leukocyte immunoglobulin-like receptor, subfamily B |
| | (with TM and ITIM domains), member 1, mRNA |
| | [NM_006669] |
| NUP37 | nucleoporin 37 kDa, mRNA [NM_024057] |
| GIMAP6 | GTPase, IMAP family member 6, mRNA |
| | [NM_024711] |
| EVI2B | ecotropic viral integration site 2B, mRNA |
| | [NM_006495] |
| C1S | complement component 1, s subcomponent, mRNA |
| | [NM_201442, NM_001734] |
| RASGRP1 | RAS guanyl releasing protein 1 (calcium and DAG- |
| | regulated), mRNA [NM_005739] |
| PRKAR2B | protein kinase, cAMP-dependent, regulatory, type II, |
| | beta, mRNA [NM_002736] |
| AGPAT9 | 1-acylglycerol-3-phosphate O-acyltransferase 9, mRNA |
| | [NM_032717] |
| FPR2 | formyl peptide receptor 2, mRNA [NM_001462] |
| AURKB | aurora kinase B, mRNA [NM_004217] |
| TNFAIP2 | tumor necrosis factor, alpha-induced protein 2, mRNA |
| | [NM_006291] |
| NUP62 | nucleoporin 62 kDa, mRNA [NM_153719, |
| | NM_016553] |
| EIF3G | eukaryotic translation initiation factor 3, subunit G, |
| | mRNA [NM_003755] |
| GOPC | golgi-associated PDZ and coiled-coil motif containing, |
| | mRNA [NM_020399] |
| CCT7 | chaperonin containing TCP1, subunit 7 (eta), mRNA |
| | [NM_006429] |
| IMPA2 | inositol(myo)-1(or 4)-monophosphatase 2, mRNA |
| | [NM_014214] |
| UPF3B | UPF3 regulator of nonsense transcripts homolog B |
| | (yeast), mRNA [NM_080632] |
| SACM1L | SAC1 suppressor of actin mutations 1-like (yeast), |
| | mRNA [NM_014016] |
| CSF3R | colony stimulating factor 3 receptor (granulocyte), |
| | mRNA [NM_156039] |
| PVRIG | poliovirus receptor related immunoglobulin domain |
| | containing, mRNA [NM_024070] |
| RAD54L | RAD54-like (S. cerevisiae), mRNA [NM_003579] |
| ATP9A | ATPase, class II, type 9A, mRNA [NM_006045] |
| NDUFB4 | NADH dehydrogenase (ubiquinone) 1 beta |
| | subcomplex, 4, 15 kDa, mRNA [NM_004547] |
| HOMER3 | homer homolog 3 (Drosophila), mRNA |
| | [NM_001145722, NM_001145724, NM_004838] |
| TNS3 | tensin 3, mRNA [NM_022748] |
| NLRP12 | NLR family, pyrin domain containing 12, mRNA |
| | [NM_001277126] |
| ADRB2 | adrenoceptor beta 2, surface, mRNA [NM_000024] |
| NUP43 | nucleoporin 43 kDa, mRNA [NM_198887] |
| CCT6A | chaperonin containing TCP1, subunit 6A (zeta 1), |
| | mRNA [NM_001762] |
| TLR2 | toll-like receptor 2, mRNA [NM_003264] |
| FAM72A | family with sequence similarity 72, member A, mRNA |
| | [NM_001123168] |
| SERPINA1 | serpin peptidase inhibitor, clade A (alpha-1 |
| | antiproteinase, antitrypsin), member 1, mRNA |
| | [NM_001002236, BX248257] |
| SLC24A4 | solute carrier family 24 (sodium/potassium/calcium |
| | exchanger), member 4, mRNA [NM_153647, |
| | NM_153646] |
| GLT1D1 | glycosyltransferase 1 domain containing 1, mRNA |
| | [NM_144669] |
| HSD17B4 | hydroxysteroid (17-beta) dehydrogenase 4, mRNA |
| | [NM_000414] |
| UXT | ubiquitously-expressed, prefoldin-like chaperone, |
| | mRNA [NM_004182] |
| CEBPA | CCAAT/enhancer binding protein (C/EBP), alpha, |
| | mRNA [NM_004364] |
| FCGR2C | Fc fragment of IgG, low affinity IIa, receptor (CD32), |
| | mRNA [NM_201563] |
| PTPRC | protein tyrosine phosphatase, receptor type, C, mRNA |
| | [NR_052021, NM_002838] |
| KLRK1 | killer cell lectin-like receptor subfamily K, member 1, |
| | mRNA [NM_007360] |
| CX3CR1 | chemokine (C-X3-C motif) receptor 1, mRNA |
| | [NM_001337] |
| LYL1 | lymphoblastic leukemia derived sequence 1, mRNA |
| | [NM_005583] |
| C7 | complement component 7, mRNA [NM_000587] |
| BCL2 | B-cell CLL/lymphoma 2, mRNA [NM_000633, |
| | NM_000657] |
| MBOAT7 | membrane bound O-acyltransferase domain containing |
| | 7, mRNA [NM_024298, NM_001146082] |
| KIR2DL5A | killer cell immunoglobulin-like receptor, two domains, |
| | long cytoplasmic tail, 5A, mRNA [NM_020535] |
| MELK | maternal embryonic leucine zipper kinase, mRNA |
| | [NM_014791] |
| CTNNA1 | catenin (cadherin-associated protein), alpha 1, 102 kDa, |
| | mRNA [NM_001903] |
| PLXNB2 | plexin B2, mRNA [NM_012401] |
| SELL | selectin L, mRNA [NM_000655] |
| CRYBB3 | crystallin, beta B3, mRNA [NM_004076] |
| KLRF1 | killer cell lectin-like receptor subfamily F, member 1, |
| | mRNA [NM_001291823, NM_016523] |
| ANLN | anillin, actin binding protein, mRNA [NM_018685] |
| NDUFC2 | NADH dehydrogenase (ubiquinone) 1, subcomplex |
| | unknown, 2, 14.5 kDa, mRNA [NM_004549, |
| | NM_001204054] |
| PLK4 | polo-like kinase 4, mRNA [NM_014264] |
| DOK2 | docking protein 2, 56 kDa, mRNA [NM_003974] |
| DOK3 | docking protein 3, mRNA [NM_024872, |
| | NM_001144875] |
| TBCK | TBC1 domain containing kinase, mRNA [NM_033115] |
| PRKCH | protein kinase C, eta, mRNA [NM_006255] |
| CYC1 | cytochrome c-1, mRNA [NM_001916] |
| ABLIM1 | actin binding LIM protein 1, mRNA [NM_001003408] |
| EMR2 | egf-like module containing, mucin-like, hormone |
| | receptor-like 2, mRNA [NM_013447] |
| P2RY13 | purinergic receptor P2Y, G-protein coupled, 13, mRNA |
| | [NM_176894] |
| KIR3DL1 | killer cell immunoglobulin-like receptor, three |
| | domains, long cytoplasmic tail, 1, mRNA |
| | [NM_013289] |
| CD163 | CD163 molecule, mRNA [NM_004244] |
| CDCA7 | cell division cycle associated 7, mRNA [NM_031942] |
| PDGFRA | platelet-derived growth factor receptor, alpha |
| | polypeptide, mRNA [NM_006206] |
| CCL4L2 | chemokine (C-C motif) ligand 4, mRNA |
| | [NM_001291470] |
| HHEX | hematopoietically expressed homeobox, mRNA |
| | [NM_002729] |
| KLRC3 | killer cell lectin-like receptor subfamily C, member 3, |
| | mRNA [NM_007333] |
| NDUFS7 | NADH dehydrogenase (ubiquinone) Fe—S protein 7, |
| | 20 kDa (NADH-coenzyme Q reductase), mRNA |
| | [NM_024407] |
| GINS4 | GINS complex subunit 4 (Sld5 homolog), mRNA |
| | [NM_032336] |
| PRAM1 | PML-RARA regulated adaptor molecule 1, mRNA |
| | [NM_032152] |
| IFNGR2 | interferon gamma receptor 2 (interferon gamma |
| | transducer 1), mRNA [NM_005534] |
| KIAA0513 | KIAA0513, mRNA [NM_014732] |
| SPI1 | spleen focus forming virus (SFFV) proviral integration |
| | oncogene, mRNA [NM_001080547] |
| POC1A | POC centriolar protein A, mRNA [NM_015426] |
| DONSON | downstream neighbor of SON, mRNA [NM_017613] |
| MEGF9 | multiple EGF-like-domains 9, mRNA |
| | [NM_001080497] |
| CDC25C | cell division cycle 25 C, mRNA [NM_001790] |
| PTGER2 | prostaglandin E receptor 2 (subtype EP2), 53 kDa, |
| | mRNA [NM_000956] |
| CDCA8 | cell division cycle associated 8, mRNA [NM_018101] |
| UQCR10 | ubiquinol-cytochrome c reductase, complex III subunit |
| | X, mRNA [NM_013387, NM_001003684] |
| COPB2 | coatomer protein complex, subunit beta 2 (beta prime), |
| | mRNA [NM_004766] |
| ALDH2 | aldehyde dehydrogenase 2 family (mitochondrial), |
| | mRNA [NM_000690] |
| RNASE6 | ribonuclease, RNase A family, k6, mRNA |
| | [NM_005615] |
| STAT4 | signal transducer and activator of transcription 4, |
| | mRNA [NM_003151] |
| SLC15A3 | solute carrier family 15 (oligopeptide transporter), |
| | member 3, mRNA [NM_016582] |
| SLC35A5 | solute carrier family 35, member A5, mRNA |
| | [NM_017945] |
| PLK1 | polo-like kinase 1, mRNA [AB084459, AK310544] |
| SMC1A | structural maintenance of chromosomes 1A, mRNA |
| | [NM_006306, NM_001281463] |
| CDK1 | cyclin-dependent kinase 1, mRNA [NM_001786] |
| FBP1 | fructose-1,6-bisphosphatase 1, mRNA [NM_000507] |
| CLIC3 | chloride intracellular channel 3, mRNA [NM_004669] |
| ETV1 | ets variant 1, mRNA [NM_004956] |
| MYO1F | myosin IF, mRNA [NM_012335] |
| S100A11 | S100 calcium binding protein A11, mRNA |
| | [NM_005620] |
| PIKFYVE | phosphoinositide kinase, FYVE finger containing, |
| | mRNA [NM_001178000, NM_015040] |
| MYOF | myoferlin, mRNA [NM_133337, NM_013451] |
| AOAH | acyloxyacyl hydrolase (neutrophil), mRNA |
| | [NM_001637] |
| CD3D | CD3d molecule, delta (CD3-TCR complex), mRNA |
| | [NM_000732] |
| TMTC2 | transmembrane and tetratricopeptide repeat containing |
| | 2, mRNA [NM_152588] |
| SUCLG1 | succinate-CoA ligase, alpha subunit, mRNA |
| | [NM_003849] |
| PTGDR | prostaglandin D2 receptor (DP), mRNA [NM_000953] |
| TREM1 | triggering receptor expressed on myeloid cells 1, |
| | mRNA [NM_018643, NM_001242589] |
| FCGRT | Fc fragment of IgG, receptor, transporter, alpha, mRNA |
| | [NM_004107] |
| NUP133 | nucleoporin 133 kDa, mRNA [BX387507, |
| | NM_018230] |
| ACBD3 | acyl-CoA binding domain containing 3, mRNA |
| | [NM_022735] |
| CD8B | CD8b molecule, mRNA [NM_172102, NM_004931] |
| HELLS | helicase, lymphoid-specific, mRNA [NM_018063, |
| | NM_001289072] |
| NFIA | nuclear factor I/A, mRNA [NM_005595, |
| | NM_001134673] |
| PTPN11 | protein tyrosine phosphatase, non-receptor type 11, |
| | mRNA [NM_002834] |
| PRF1 | perforin 1 (pore forming protein), mRNA |
| | [NM_005041] |
| NCAM1 | neural cell adhesion molecule 1, mRNA |
| | [NM_001076682, NM_001242608, NM_001242607] |
| CYBB | cytochrome b-245, beta polypeptide, mRNA |
| | [NM_000397] |
| SYK | spleen tyrosine kinase, mRNA [NM_003177] |
| TBXAS1 | thromboxane A synthase 1 (platelet), mRNA |
| | [NM_001166254, NM_030984] |
| ZAP70 | zeta-chain (TCR) associated protein kinase 70 kDa, |
| | mRNA [NM_001079] |
| SEMA4A | sema domain, immunoglobulin domain (Ig), |
| | transmembrane domain (TM) and short cytoplasmic |
| | domain, (semaphorin) 4A, mRNA [NM_022367] |
| LYN | v-yes-1 Yamaguchi sarcoma viral related oncogene |
| | homolog, mRNA [NM_002350] |
| TRIM10 | tripartite motif containing 10, mRNA [NM_006778, |
| | NM_052828] |
| HJURP | Holliday junction recognition protein, mRNA |
| | [NM_018410] |
| PRKCD | protein kinase C, delta, mRNA [NM_006254] |
| KIF11 | kinesin family member 11, mRNA [NM_004523] |
| RHOH | ras homolog family member H, mRNA [NM_004310] |
| GRN | granulin, mRNA [NM_002087] |
| TTC1 | tetratricopeptide repeat domain 1, mRNA |
| | [NM_003314] |
| ITGAM | integrin, alpha M (complement component 3 receptor 3 |
| | subunit), mRNA [NM_000632] |
| RXRA | retinoid X receptor, alpha, mRNA [NM_002957] |
| NUP85 | nucleoporin 85 kDa, mRNA [NM_024844] |
| XCL1 | chemokine (C motif) ligand 1, mRNA [NM_002995] |
| BTK | Bruton agammaglobulinemia tyrosine kinase, mRNA |
| | [NM_001287344, NM_000061] |
| KIR2DS4 | killer cell immunoglobulin-like receptor, two domains, |
| | short cytoplasmic tail, 4, mRNA [NM_012314] |
| GINS2 | GINS complex subunit 2 (Psf2 homolog), mRNA |
| | [NM_016095] |
| CARD11 | caspase recruitment domain family, member 11, mRNA |
| | [NM_032415, AK097139] |
| NUP205 | nucleoporin 205 kDa, mRNA [NM_015135] |
| COX5A | cytochrome c oxidase subunit Va, mRNA |
| | [NM_004255] |
| IL12RB2 | interleukin 12 receptor, beta 2, mRNA [NM_001559] |
| ITGB2 | integrin, beta 2 (complement component 3 receptor 3 |
| | and 4 subunit), mRNA [L78790, NM_000211] |
| FXYD6 | FXYD domain containing ion transport regulator 6, |
| | mRNA [NM_022003] |
| NKG7 | natural killer cell group 7 sequence, mRNA |
| | [NM_005601] |
| CLEC12B | C-type lectin domain family 12, member B, mRNA |
| | [NM_205852, NM_001129998] |
| TBX21 | T-box 21, mRNA [NM_013351] |
| TMEM106C | transmembrane protein 106C, mRNA [NM_024056] |
| EOMES | eomesodermin, mRNA [NM_005442] |
| CPPED1 | calcineurin-like phosphoesterase domain containing 1, |
| | mRNA [NM_018340] |
| HEPACAM2 | HEPACAM family member 2, mRNA |
| | [NM_001039372] |
| BMI1 | BMI1 polycomb ring finger oncogene, mRNA |
| | [NM_005180] |
| CST7 | cystatin F (leukocystatin), mRNA [NM_003650] |
| KIR2DL2 | killer cell immunoglobulin-like receptor, two domains, |
| | long cytoplasmic tail, 2, mRNA [NM_014219] |
| PROS1 | protein S (alpha), mRNA [NM_000313] |
| SLC28A1 | solute carrier family 28 (concentrative nucleoside |
| | transporter), member 1, mRNA [NM_004213, |
| | NM_201651] |
| STEAP4 | STEAP family member 4, mRNA [NM_001205315] |
| RORA | RAR-related orphan receptor A, mRNA [NM_134260] |
| MATK | megakaryocyte-associated tyrosine kinase, mRNA |
| | [NM_139355] |
| TCF7 | transcription factor 7 (T-cell specific, HMG-box), |
| | mRNA [NM_003202] |
| MCTP1 | multiple C2 domains, transmembrane 1, mRNA |
| | [NM_024717] |
| CCNB2 | cyclin B2, mRNA [NM_004701] |
| CCL5 | chemokine (C-C motif) ligand 5, mRNA [NM_002985] |
| TRIP13 | thyroid hormone receptor interactor 13, mRNA |
| | [NM_004237] |
| TNFSF12 | tumor necrosis factor (ligand) superfamily, member 12, |
| | mRNA [NM_003809] |
| PDGFRB | platelet derived growth factor receptor beta, mRNA |
| | [NM_002609] |
| NDUFB11 | NADH dehydrogenase (ubiquinone) 1 beta |
| | subcomplex, 11, 17.3 kDa, mRNA [NM_019056] |
| ELP4 | elongator acetyltransferase complex subunit 4, mRNA |
| | [NM_019040] |
| KLRC1 | killer cell lectin-like receptor subfamily C, member 1, |
| | mRNA [NM_007328, NM_002259] |
| PMS1 | PMS1 postmeiotic segregation increased 1 |
| | (S. cerevisiae), mRNA [NM_000534, NM_001128144] |
| SCPEP1 | serine carboxypeptidase 1, mRNA [NM_021626] |
| SP140 | SP140 nuclear body protein, mRNA [NM_001005176, |
| | NM_007237] |
| POLA1 | polymerase (DNA directed), alpha 1, catalytic subunit, |
| | mRNA [NM_016937] |
| FASLG | Fas ligand (TNF superfamily, member 6), mRNA |
| | [NM_000639] |
| SLC35A3 | solute carrier family 35 (UDP-N-acetylglucosamine |
| | (UDP-GlcNAc) transporter), member A3, mRNA |
| | [NM_012243] |
| SORT1 | sortilin 1, mRNA [NM_002959] |
| ABCA8 | ATP-binding cassette, sub-family A (ABC1), member |
| | 8, mRNA [NM_007168] |
| DTYMK | deoxythymidylate kinase (thymidylate kinase), mRNA |
| | [NM_012145] |
| NOP56 | NOP56 ribonucleoprotein, mRNA [NM_006392] |
| SMARCAL1 | SWI/SNF related, matrix associated, actin dependent |
| | regulator of chromatin, subfamily a-like 1, mRNA |
| | [NM_014140] |
| PLEK | pleckstrin, mRNA [NM_002664] |
| SEH1L | SEH1-like (S. cerevisiae), mRNA [NM_031216, |
| | NM_001013437] |
| ITK | IL2-inducible T-cell kinase, mRNA [NM_005546] |
| NFAM1 | NEAT activating protein with ITAM motif 1, mRNA |
| | [NM_145912] |
| ITPR3 | inositol 1,4,5-trisphosphate receptor, type 3, mRNA |
| | [NM_002224] |
| TNFRSF18 | tumor necrosis factor receptor superfamily, member 18, |
| | mRNA [NM_148901] |
| FCAR | Fc fragment of IgA, receptor for, mRNA [NM_002000, |
| | NM_133271] |
| E2E7 | E2F transcription factor 7, mRNA [NM_203394] |
| KCNQ1 | potassium voltage-gated channel, KQT-like subfamily, |
| | member 1, mRNA [NM_000218] |
| CENPW | centromere protein W, mRNA [NM_001012507] |
| SIGLEC7 | sialic acid binding Ig-like lectin 7, mRNA |
| | [NM_014385] |
| PCNA | proliferating cell nuclear antigen, mRNA [NM_002592] |
| ANO10 | anoctamin 10, mRNA [NM_001204831] |
| PTPRE | protein tyrosine phosphatase, receptor type, E, mRNA |
| | [NM_006504] |
| ABCC13 | ATP-binding cassette, sub-family C (CFTR/MRP), |
| | member 13, pseudogene, mRNA [NR_003088, |
| | NR_003087] |
| TSNAX | translin-associated factor X, mRNA [NM_005999] |
| SSBP1 | single-stranded DNA binding protein 1, mitochondrial, |
| | mRNA [NM_003143] |
| TGFBR3 | transforming growth factor, beta receptor III, mRNA |
| | [NM_003243] |
| CENPE | centromere protein E, 312 kDa, mRNA [NM_001813] |
| NDUFS8 | NADH dehydrogenase (ubiquinone) Fe—S protein 8, 23 |
| | kDa (NADH-coenzyme Q reductase), mRNA |
| | [NM_002496] |
| CARD9 | caspase recruitment domain family, member 9, mRNA |
| | [NM_052813, NM_052814] |
| CHAF1B | chromatin assembly factor 1, subunit B (p60), mRNA |
| | [NM_005441] |
| IL6R | interleukin 6 receptor, mRNA [NM_000565, |
| | NM_001206866] |
| CD8A | CD8a molecule, mRNA [NM_001768, |
| | NM_001145873] |
| CDCA5 | cell division cycle associated 5, mRNA [NM_080668] |
| CEP72 | centrosomal protein 72 kDa, mRNA [NM_018140] |
| KIAA1109 | KIAA1109, mRNA [NM_015312] |
| H2AFX | H2A histone family, member X, mRNA [NM_002105] |
| HOPX | HOP homeobox, mRNA [NM_001145460, |
| | NM_139211] |
| ARL4C | ADP-ribosylation factor-like 4C, mRNA |
| | [NM_001282431] |
| TLR8 | toll-like receptor 8, mRNA [NM_016610] |
| SLC16A3 | solute carrier family 16 (monocarboxylate transporter), |
| | member 3, mRNA [NM_001042422] |
| CD96 | CD96 molecule, mRNA [NM_198196] |
| FMR1 | fragile X mental retardation 1, mRNA [NM_002024] |
| CAMK4 | calcium/calmodulin-dependent protein kinase IV, |
| | mRNA [NM_001744] |
| CENPF | centromere protein F, 350/400 kDa, mRNA |
| | [NM_016343] |
| ANP32E | acidic (leucine-rich) nuclear phosphoprotein 32 family, |
| | member E, mRNA [NM_030920] |
| LCK | lymphocyte-specific protein tyrosine kinase, mRNA |
| | [NM_005356] |
| KCTD12 | potassium channel tetramerization domain containing |
| | 12, mRNA [NM_138444] |
| CASC5 | cancer susceptibility candidate 5, mRNA [NM_170589] |
| SGOL2 | shugoshin-like 2 (S. pombe), mRNA [NM_152524] |
| TNFRSF1B | tumor necrosis factor receptor superfamily, member 1B, |
| | mRNA [NM_001066] |
| GZMM | granzyme M (lymphocyte met-ase 1), mRNA |
| | [NM_005317] |
| IL7R | interleukin 7 receptor, mRNA [NM_002185] |
| MCM6 | minichromosome maintenance complex component 6, |
| | mRNA [NM_005915] |
| CD1D | CD1d molecule, mRNA [NM_001766] |
| ASPM | asp (abnormal spindle) homolog, microcephaly |
| | associated (Drosophila), mRNA [NM_018136] |
| PAX2 | paired box 2, mRNA [NM_003990, NM_003988] |
| ZMYM2 | zinc finger, MYM-type 2, mRNA [NM_003453, |
| | AL136621] |
| IL2RB | interleukin 2 receptor, beta, mRNA [NM_000878] |
| PTTG1 | pituitary tumor-transforming 1, mRNA [NM_004219] |
| FANCI | Fanconi anemia, complementation group I, mRNA |
| | [NM_018193] |
| PCSK6 | proprotein convertase subtilisin/kexin type 6, mRNA |
| | [NM_138322, NM_002570] |
| GZMH | granzyme H (cathepsin G-like 2, protein h-CCPX), |
| | mRNA [NM_033423] |
| SLC11A1 | solute carrier family 11 (proton-coupled divalent metal |
| | ion transporter), member 1, mRNA [NM_000578, |
| | AK294707] |
| ETS1 | v-ets avian erythroblastosis virus E26 oncogene |
| | homolog 1, mRNA [NM_005238] |
| CKAP5 | cytoskeleton associated protein 5, mRNA |
| | [NM_001008938] |
| WDFY3 | WD repeat and FYVE domain containing 3, mRNA |
| | [NM_014991, AK092019] |
| IL18R1 | interleukin 18 receptor 1, mRNA [NM_003855] |
| HAVCR2 | hepatitis A virus cellular receptor 2, mRNA |
| | [NM_032782] |
| SH3BGRL2 | SH3 domain binding glutamic acid-rich protein like 2, |
| | mRNA [NM_031469] |
| CENPN | centromere protein N, mRNA [NM_001100625, |
| | NM_018455, NM_001100624] |
| ATF2 | activating transcription factor 2, mRNA [NM_001880] |
| EXOC1 | exocyst complex component 1, mRNA |
| | [NM_001024924] |
| KIR2DS3 | killer cell immunoglobulin-like receptor, two domains, |
| | short cytoplasmic tail, 3, mRNA [NM_012313] |
| TUBG1 | tubulin, gamma 1, mRNA [NM_001070] |
| NUF2 | NUF2, NDC80 kinetochore complex component, |
| | mRNA [NM_145697] |
| SMC4 | structural maintenance of chromosomes 4, mRNA |
| | [NM_005496] |
| NUSAP1 | nucleolar and spindle associated protein 1, mRNA |
| | [NM_016359] |
| CHST15 | carbohydrate (N-acetylgalactosamine 4-sulfate 6-O) |
| | sulfotransferase 15, mRNA [NM_001270765, |
| | NM_015892] |
| PTPN4 | protein tyrosine phosphatase, non-receptor type 4 |
| | (megakaryocyte), mRNA [NM_002830] |
| TACC3 | transforming, acidic coiled-coil containing protein 3, |
| | mRNA [NM_006342] |
| FGD4 | FYVE, RhoGEF and PH domain containing 4, mRNA |
| | [NM_139241, AL713762] |
| PLXND1 | plexin D1, mRNA [NM_015103] |
| FCGR2A | Fc fragment of IgG, low affinity IIa, receptor (CD32), |
| | mRNA [NM_021642] |
| CCR7 | chemokine (C-C motif) receptor 7, mRNA |
| | [NM_001838] |
| FANCD2 | Fanconi anemia, complementation group D2, mRNA |
| | [NM_001018115, NM_033084] |
| S1PR5 | sphingosine-1-phosphate receptor 5, mRNA |
| | [NM_030760] |
| HMBS | hydroxymethylbilane synthase, mRNA [NM_000190] |
| UBE2S | ubiquitin-conjugating enzyme E2S, mRNA |
| | [NM_014501] |
| CD247 | CD247 molecule, mRNA [NM_198053, AK128376] |
| FOXM1 | forkhead box M1, mRNA [NM_202002] |
| NUP155 | nucleoporin 155 kDa, mRNA [NM_153485] |
| CD7 | CD7 molecule, mRNA [NM_006137] |
| RAB32 | RAB32, member RAS oncogene family, mRNA |
| | [NM_006834] |
| PLEKHF1 | pleckstrin homology domain containing, family F (with |
| | FYVE domain) member 1, mRNA [NM_024310] |
| EXO1 | exonuclease 1, mRNA [NM_003686] |
| NNMT | nicotinamide N-methyltransferase, mRNA |
| | [NM_006169] |
| CTSW | cathepsin W, mRNA [NM_001335] |
| TNFSF13 | tumor necrosis factor (ligand) superfamily, member 13, |
| | mRNA [NM_172088] |
| SKA1 | spindle and kinetochore associated complex subunit 1, |
| | mRNA [NM_001039535] |
| TXK | TXK tyrosine kinase, mRNA [NM_003328] |
| CDCA2 | cell division cycle associated 2, mRNA [NM_152562] |
| DHFR | dihydrofolate reductase, mRNA [NM_000791] |
| MCM4 | minichromosome maintenance complex component 4, |
| | mRNA [NM_005914] |
| NPL | N-acetylneuraminate pyruvate lyase |
| | (dihydrodipicolinate synthase), mRNA |
| | [BC034966, NM_001200056, NM_001200050] |
| PRC1 | protein regulator of cytokinesis 1, mRNA |
| | [NM_003981] |
| ATIC | 5-aminoimidazole-4-carboxamide ribonucleotide |
| | formyltransferase/IMP cyclohydrolase, mRNA |
| | [NM_004044] |
| AFF2 | AF4/FMR2 family, member 2, mRNA [X95463, |
| | NM_002025] |
| SLA2 | Src-like-adaptor 2, mRNA [NM_032214] |
| SH2D2A | SH2 domain containing 2A, mRNA [NM_003975, |
| | NM_001161444] |
| NCAPD3 | non-SMC condensin II complex, subunit D3, mRNA |
| | [NM_015261] |
| GPR114 | G protein-coupled receptor 114, mRNA |
| | [XM_006721169] |
| DPP4 | dipeptidyl-peptidase 4, mRNA [NM_001935] |
| LAT | linker for activation of T cells, mRNA [NM_014387] |
| CLINT1 | clathrin interactor 1, mRNA [NM_014666] |
| SPOCK2 | sparc/osteonectin, cwcv and kazal-like domains |
| | proteoglycan (testican) 2, mRNA [NM_014767, |
| | NM_001134434] |
| TIGIT | T cell immunoreceptor with Ig and ITIM domains, |
| | mRNA [NM_173799] |
| CXCR3 | chemokine (C-X-C motif) receptor 3, mRNA |
| | [NM_001504, NM_001142797] |
| CYBRD1 | cytochrome b reductase 1, mRNA [NM_024843] |
| TMEM71 | transmembrane protein 71, mRNA [NM_144649] |
| MCM5 | minichromosome maintenance complex component 5, |
| | mRNA [NM_006739] |
| TCHP | trichoplein, keratin filament binding, mRNA |
| | [NM_032300] |
| SIRT1 | sirtuin 1, mRNA [NM_012238] |
| ECM2 | extracellular matrix protein 2, female organ and |
| | adipocyte specific, mRNA [NM_001393, |
| | NM_001197296, BC036806] |
| NACC2 | NACC family member 2, BEN and BTB (POZ) domain |
| | containing, mRNA [NM_144653] |
| CD5 | CD5 molecule, mRNA [NM_014207] |
| PBX1 | pre-B-cell leukemia homeobox 1, mRNA |
| | [NM_002585, AK093508] |
| MPEG1 | macrophage expressed 1, mRNA [NM_001039396] |
| STMN1 | stathmin 1, mRNA [NM_203401, NM_001145454] |
| SLAMF7 | SLAM family member 7, mRNA [NM_021181] |
| CLEC4E | C-type lectin domain family 4, member E, mRNA |
| | [NM_014358] |
| ITGAX | integrin, alpha X (complement component 3 receptor 4 |
| | subunit), mRNA [AK309642, NM_000887] |
| UBASH3A | ubiquitin associated and SH3 domain containing A, |
| | mRNA [NM_018961, NM_001243467] |
| TTC7B | tetratricopeptide repeat domain 7B, mRNA |
| | [NM_001010854] |
| SPAG5 | sperm associated antigen 5, mRNA [NM_006461] |
| IL21R | interleukin 21 receptor, mRNA [NM_181078] |
| GNLY | granulysin, mRNA [NM_006433, AK310057] |
| PRKCQ | protein kinase C, theta, mRNA [NM_006257] |
| KIF23 | kinesin family member 23, mRNA [NM_138555] |
| NDUFA3 | NADH dehydrogenase (ubiquinone) 1 alpha |
| | subcomplex, 3, 9 kDa, mRNA [NM_004542] |
| F13A1 | coagulation factor XIII, A1 polypeptide, mRNA |
| | [NM_000129] |
| SAMD3 | sterile alpha motif domain containing 3, mRNA |
| | [NM_001258275, NM_001017373] |
| IKZF1 | IKAROS family zinc finger 1 (Ikaros), mRNA |
| | [AF116605, NM_006060] |
| RARRES3 | retinoic acid receptor responder (tazarotene induced) 3, |
| | mRNA [NM_004585] |
| GZMB | granzyme B (granzyme 2, cytotoxic T-lymphocyte- |
| | associated serine esterase 1), mRNA [NM_004131] |
| EZH2 | enhancer of zeste homolog 2 (Drosophila), mRNA |
| | [NM_004456] |

TABLE 7
marker genes for cancer, in particular prostate cancer

| | | | | |
|---|---|---|---|---|
| COX7B | MND1 | NOC3L | RHOU | ARFIP1 |
| LY96 | S100A12 | DEK | PHTF2 | COX6C |
| FIGNL1 | AGL | HNMT | S100A9 | DMD |
| FOSL1 | NUPL2 | MS4A4A | FAM198B | KYNU |
| S100A8 | PKMYT1 | TMEM55A | ST3GAL6 | PIK3C2A |
| GFRA3 | PTAFR | TMX1 | CLK4 | NDUFB1 |
| REEP1 | VSTM1 | USP1 | CLEC12A | VNN1 |
| SUV39H2 | SAMHD1 | CCDC80 | GCA | MBNL1 |
| ZFYVE16 | ASPA | PCNP | VNN2 | ABCA1 |
| NDUFA4 | PPP1R12A | SHCBP1 | C1GALT1C1 | PID1 |
| FOXN1 | UQCRQ | PANK3 | DUSP6 | SRP9 |
| KIR2DL4 | OSMR | PAQR3 | TNFAIP8 | MAD2L1 |
| GOLT1B | SP3 | RNF138 | TLR4 | GLIPR1 |
| NLRC3 | JPH2 | CD302 | FBLN1 | NUP107 |
| ACTR6 | APCDD1 | NUP35 | LRRK2 | GZMK |
| PLEKHF2 | ABCG2 | IMPA1 | SLC46A2 | STAG2 |
| TFEC | TLR7 | MS4A7 | KIAA1468 | NDUFB3 |
| LGALS2 | ATP5C1 | SGMS1 | AIF1 | CYP1B1 |
| MSH6 | GINS1 | GALNT7 | HK3 | MYBL2 |
| TARP | CKS2 | CA14 | SFRP4 | KLF1 |

TABLE 8
marker genes for cancer, in particular prostate cancer

| | | | | |
|---|---|---|---|---|
| COX7B | FOXN1 | MND1 | UQCRQ | NOC3L |
| LY96 | KIR2DL4 | S100A12 | OSMR | DEK |
| FIGNL1 | GOLT1B | AGL | SP3 | HNMT |
| FOSL1 | NLRC3 | NUPL2 | JPH2 | MS4A4A |
| S100A8 | ACTR6 | PKMYT1 | APCDD1 | TMEM55A |
| GFRA3 | PLEKHF2 | PTAFR | ABCG2 | TMX1 |
| REEP1 | TFEC | VSTM1 | TLR7 | USP1 |
| SUV39H2 | LGALS2 | SAMHD1 | ATP5C1 | CCDC80 |
| ZFYVE16 | MSH6 | ASPA | GINS1 | PCNP |
| NDUFA4 | TARP | PPP1R12A | CKS2 | SHCBP1 |

TABLE 9
marker genes for cancer, in particular prostate cancer

| | | | | | |
|---|---|---|---|---|---|
| COX7B | GFRA3 | FOXN1 | PLEKHF2 | MND1 | PTAFR |
| LY96 | REEP1 | KIR2DL4 | TFEC | S100A12 | VSTM1 |
| FIGNL1 | SUV39H2 | GOLT1B | LGALS2 | AGL | SAMHD1 |
| FOSL1 | ZFYVE16 | NLRC3 | MSH6 | NUPL2 | ASPA |
| S100A8 | NDUFA4 | ACTR6 | TARP | PKMYT1 | PPP1R12A |

TABLE 10
marker genes for cancer, in particular prostate cancer

| | | | | |
|---|---|---|---|---|
| COX7B | FIGNL1 | S100A8 | REEP1 | ZFYVE16 |
| LY96 | FOSL1 | GFRA3 | SUV39H2 | NDUFA4 |

TABLE 11
marker genes that occur in both the PCA and NSCLC cluster

| | | | | | |
|---|---|---|---|---|---|
| LY96 | LMO2 | IRAK3 | SIRPA | FCGRT | GZMM |
| FIGNL1 | KIAA0101 | ASGR2 | TRAT1 | CD8B | IL7R |
| S100A8 | RFC5 | CD36 | RASSF4 | PRF1 | MCM6 |
| NLRC3 | FEN1 | CD33 | PECAM1 | NCAM1 | CD1D |
| TFEC | PTPN22 | PSAP | BUB1 | CYBB | ASPM |
| LGALS2 | CDKN3 | BCL6 | PYCARD | SYK | IL2RB |
| MSH6 | QPCT | LILRA6 | ARHGEF10L | TBXAS1 | PTTG1 |
| TARP | NDC80 | ACSL1 | ITM2A | ZAP70 | FANCI |
| MND1 | KIR2DS2 | SULF2 | LILRB1 | HJURP | GZMH |
| S100A12 | FPR1 | CRTAM | GIMAP6 | PRKCD | SLC11A1 |
| PKMYT1 | ENPP5 | GZMA | RASGRP1 | KIF11 | ETS1 |
| PTAFR | DPYD | PGD | AGPAT9 | RHOH | CKAP5 |
| VSTM1 | TGFBI | RFC4 | FPR2 | GRN | WDFY3 |
| SAMHD1 | CDT1 | POLD3 | AURKB | ITGAM | IL18R1 |
| APCDD1 | KLRB1 | MYD88 | IMPA2 | XCL1 | CENPN |
| TLR7 | SMC2 | SH2D1A | CSF3R | BTK | KIR2DS3 |
| HNMT | TYROBP | NCF1 | PVRIG | KIR2DS4 | TUBG1 |
| MS4A4A | GMNN | CD27 | RAD54L | GINS2 | NUF2 |
| TMEM55A | FGR | HPSE | ATP9A | IL12RB2 | SMC4 |
| SHCBP1 | KIF18A | UBE2T | HOMER3 | FXYD6 | NUSAP1 |
| CD302 | KIF20B | LILRA2 | TNS3 | NKG7 | CHST15 |
| RHOU | RAD21 | LEF1 | NLRP12 | TBX21 | PTPN4 |
| S100A9 | SLC31A2 | KNTC1 | ADRB2 | EOMES | FGD4 |
| FAM198B | VNN3 | ACPP | TLR2 | CPPED1 | FCGR2A |
| ST3GAL6 | MARCO | SLC7A7 | SERPINA1 | CST7 | CCR7 |
| CLEC12A | KIF20A | FGFBP2 | SLC24A4 | KIR2DL2 | S1PR5 |
| GCA | RAB31 | TMPO | GLT1D1 | STEAP4 | CD247 |
| VNN2 | TMEM154 | CEP76 | KLRK1 | RORA | CD7 |
| DUSP6 | TLR1 | CPVL | CX3CR1 | TCF7 | RAB32 |
| TLR4 | CD14 | TYMS | BCL2 | MCTP1 | PLEKHF1 |
| LRRK2 | SPC25 | CDC25A | MBOAT7 | CCNB2 | EXO1 |
| SLC46A2 | ASGR1 | CKS1B | KIR2DL5A | CCL5 | CTSW |
| AIF1 | EVI5 | GPR171 | MELK | TRIP13 | TNFSF13 |
| HK3 | TLR6 | POLA2 | PLXNB2 | PDGFRB | TXK |
| VNN1 | EMILIN2 | RAN | KLRF1 | KLRC1 | CDCA2 |
| PID1 | CCNB1 | F5 | ANLN | SCPEP1 | DHFR |
| MAD2L1 | RNASE2 | CD2 | DOK3 | SP140 | MCM4 |
| GZMK | CTSH | NDUFA8 | PRKCH | POLA1 | NPL |
| CYP1B1 | S1PR3 | RBM47 | ABLIM1 | FASLG | SLA2 |
| MYBL2 | IL1RN | CST3 | EMR2 | SORT1 | SH2D2A |
| IFI30 | CEP55 | SKA3 | P2RY13 | DTYMK | NCAPD3 |
| ICOS | ECT2 | SLAMF1 | KIR3DL1 | SMARCAL1 | GPR114 |
| CD28 | AQP9 | NUP88 | CD163 | ITK | DPP4 |
| SGK1 | FES | BRCA1 | CDCA7 | NFAM1 | LAT |
| PLA2G7 | RNF130 | CYFIP1 | HHEX | ITPR3 | SPOCK2 |
| RMI1 | PADI2 | TIPIN | KLRC3 | E2F7 | TIGIT |
| CCR2 | TPX2 | LILRB2 | GINS4 | SIGLEC7 | CXCR3 |
| FCN1 | NEK2 | IL13RA1 | PRAM1 | PCNA | CYBRD1 |
| ZWINT | CSF1R | NCF4 | IFNGR2 | ANO10 | MCM5 |
| AMICA1 | RPA3 | LTBR | SPI1 | SSBP1 | TCHP |
| CLEC7A | SMARCD3 | ALOX5 | MEGF9 | TGFBR3 | NACC2 |
| MSH2 | GPR56 | CRISPLD2 | CDCA8 | CENPE | CD5 |
| MFSD1 | MGST1 | PYGL | ALDH2 | CARD9 | MPEG1 |
| KCNJ2 | FCGR1B | CFP | RNASE6 | CHAF1B | STMN1 |
| IFNG | KCNE3 | KIF2C | STAT4 | CD8A | SLAMF7 |
| CSTA | MS4A6A | HCK | SLC15A3 | CDCA5 | CLEC4E |
| MYBL1 | SIRPB1 | THEMIS | PLK1 | CEP72 | UBASH3A |
| MNDA | RAD51 | KIAA1598 | CDK1 | HOPX | SPAG5 |
| RBP7 | PLBD1 | TIMP2 | FBP1 | TLR8 | IL21R |
| BST1 | LAT2 | GATA3 | CLIC3 | SLC16A3 | GNLY |
| DLGAP5 | LILRB3 | NLRP3 | MYO1F | CD96 | PRKCQ |
| NOD2 | LILRA5 | UHRF1 | S100A11 | CAMK4 | KIF23 |
| LILRA3 | PRIM1 | OIP5 | AOAH | CENPF | F13A1 |
| CLEC4A | CFD | PAK1 | CD3D | LCK | SAMD3 |
| TNFSF13B | NCF2 | LY86 | TMTC2 | KCTD12 | IKZF1 |
| CSF2RA | AGTRAP | KLRD1 | PTGDR | CASC5 | RARRES3 |
| BRI3 | GIMAP7 | DTL | TREM1 | SGOL2 | GZMB |

TABLE 12
selected marker genes that occur in both the PCA and NSCLC cluster

| | | | | |
|---|---|---|---|---|
| LY96 | CD302 | IFI30 | DLGAP5 | CDT1 |
| FIGNL1 | RHOU | ICOS | NOD2 | KLRB1 |
| S100A8 | S100A9 | CD28 | LILRA3 | SMC2 |
| NLRC3 | FAM198B | SGK1 | CLEC4A | TYROBP |
| TFEC | ST3GAL6 | PLA2G7 | TNFSF13B | GMNN |
| LGALS2 | CLEC12A | RMI1 | CSF2RA | FGR |
| MSH6 | GCA | CCR2 | BRI3 | KIF18A |
| TARP | VNN2 | FCN1 | LMO2 | KIF20B |
| MND1 | DUSP6 | ZWINT | KIAA0101 | RAD21 |
| S100A12 | TLR4 | AMICA1 | RFC5 | SLC31A2 |
| PKMYT1 | LRRK2 | CLEC7A | FEN1 | VNN3 |
| PTAFR | SLC46A2 | MSH2 | PTPN22 | MARCO |
| VSTM1 | AIF1 | MFSD1 | CDKN3 | KIF20A |
| SAMHD1 | HK3 | KCNJ2 | QPCT | RAB31 |
| APCDD1 | VNN1 | IFNG | NDC80 | TMEM154 |
| TLR7 | PID1 | CSTA | KIR2DS2 | TLR1 |
| HNMT | MAD2L1 | MYBL1 | FPR1 | CD14 |
| MS4A4A | GZMK | MNDA | ENPP5 | SPC25 |
| TMEM55A | CYP1B1 | RBP7 | DPYD | ASGR1 |
| SHCBP1 | MYBL2 | BST1 | TGFBI | EVI5 |

TABLE 13
selected marker genes that occur in both the PCA and NSCLC cluster

| | | | | |
|---|---|---|---|---|
| LY96 | PKMYT1 | CD302 | LRRK2 | IFI30 |
| FIGNL1 | PTAFR | RHOU | SLC46A2 | ICOS |
| S100A8 | VSTM1 | S100A9 | AIF1 | CD28 |
| NLRC3 | SAMHD1 | FAM198B | HK3 | SGK1 |
| TFEC | APCDD1 | ST3GAL6 | VNN1 | PLA2G7 |
| LGALS2 | TLR7 | CLEC12A | PID1 | RMI1 |
| MSH6 | HNMT | GCA | MAD2L1 | CCR2 |
| TARP | MS4A4A | VNN2 | GZMK | FCN1 |
| MND1 | TMEM55A | DUSP6 | CYP1B1 | ZWINT |
| S100A12 | SHCBP1 | TLR4 | MYBL2 | AMICA1 |

TABLE 14
selected marker genes that occur in both the PCA and NSCLC cluster

| | | | | | |
|---|---|---|---|---|---|
| LY96 | LGALS2 | PKMYT1 | TLR7 | CD302 | CLEC12A |
| FIGNL1 | MSH6 | PTAFR | HNMT | RHOU | GCA |
| S100A8 | TARP | VSTM1 | MS4A4A | S100A9 | VNN2 |
| NLRC3 | MND1 | SAMHD1 | TMEM55A | FAM198B | DUSP6 |
| TFEC | S100A12 | APCDD1 | SHCBP1 | ST3GAL6 | TLR4 |

TABLE 15
selected marker genes that occur in both the PCA and NSCLC cluster

| | | | | |
|---|---|---|---|---|
| LY96 | S100A8 | TFEC | MSH6 | MND1 |
| FIGNL1 | NLRC3 | LGALS2 | TARP | S100A12 |

### EXAMPLES

### Study 1

The Examples shown in the following are merely illustrative and shall describe the present invention in a further way. These Examples shall not be construed to limit the present invention thereto.

CV9201 as described in WO2009046974 and Sebastian et al. BMC Cancer 2014, 14:748) is an mRNA-based cancer immunotherapeutic agent/vaccine comprising following cancer antigens: MAGE-C1, MAGE-C2, NY-ESO-1, Survivin and 5T4. Repeated vaccinations of NSCLC patients with 320 μg RNA for each antigen (1.600 μg RNA in total) were carried out at weeks 1, 2, 3, 5, and 7. Peripheral blood samples were taken at weeks 0 (baseline), 5 and 9 (2 weeks post 3rd treatment and 2 weeks post 5th treatment, respectively).

Peripheral blood mono-nuclear cells (PBMCs) were isolated by Ficoll density centrifugation. Briefly, around 20 ml of blood were pipetted layered onto a LeucoSep tube (greiner bio-one) and centrifuged for 20 minutes without brake. PBMCs from the interphase were washed three times in PBS and suspended in cryo SFM (PromoCell) and cryopreserved in liquid nitrogen. Cells were thawed for immune analyses and 1 million left-over PBMCs were suspended in PBS and frozen.

RNA was isolated from re-thawed PBMCs using RNEasy (Qiagen) according to the manufacturer's instructions. RNA quality control was performed on a 2100 Bioanalyzer (Agilent Technologies) and a RIN threshold of 6.5 was used as cutoff. Cyanine-3-labelling of cRNA and hybridization using the Agilent SurePrint G3 Human Gene Expression 8×60K v2 Microarray platform was performed by IMGM according to manufacturer's manuals (Agilent Technologies). Raw data was processed using following software tools: Feature Extraction 10.7.3.1 and GeneSpring GX 12.6.1. Quantile normalization was performed and log 2-transformed and filtered for probes detectable in at least one sample.

Altogether 22 stage IV non-small cell lung cancer patients were evaluable in the transcriptional profiling study and gene expression data was obtained for 48,605 probes. To reduce the data set, we made use of blood transcriptional modules (BTMs) developed by the Pulendran group [1]. These altogether 346 BTMs represent sets of genes, which are transcriptionally co-regulated. Most of these BTMs reflect an immunological process, such as signaling pathways or represent sets of genes specifically enriched in immune cells, such as T or B cells. We first calculated the BTM activity scores, which represent the mean expression of all genes contained in a module. Probes with the highest average expression across all samples were chosen for genes targeted by multiple probes.

To monitor changes between baseline and post vaccine time points, BTM activity score differences between week 0 (pre-treatment time point) and week 5 (2 weeks post 3rd treatment) were calculated (Table 6). More specifically, gene expression differences between week 5 and week 0 were calculated for each patient in cluster 1. Subsequently, the mean of the week 5 to 0 differences was determined for all patients in cluster 1 (Week5_0 GE difference_mean_pat_clust1). Analogously, gene expression differences between week 5 and week 0 were calculated for each patient in cluster 2. Subsequently, the mean of the week 5 to 0 differences was determined for all patients in cluster 2 (Week5_0 GE difference_mean_pat_clust2). Both values (“Week5_0 GE difference_mean_pat_clust1” and “Week5_0 GE difference_mean_pat_clust2”) thus signify the mean change in gene expression comparing post treatment week 5 with the baseline value week 0 within a given group of patients. The difference between “Week5_0 GE difference_mean_pat_clust1” and “Week5_0 GE difference_mean_pat_clust2” was calculated (difference).

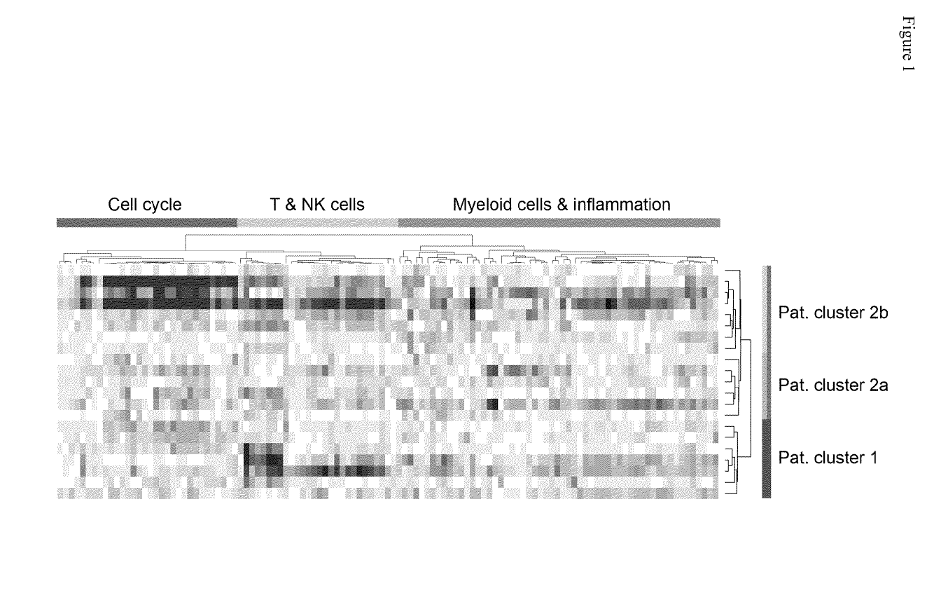

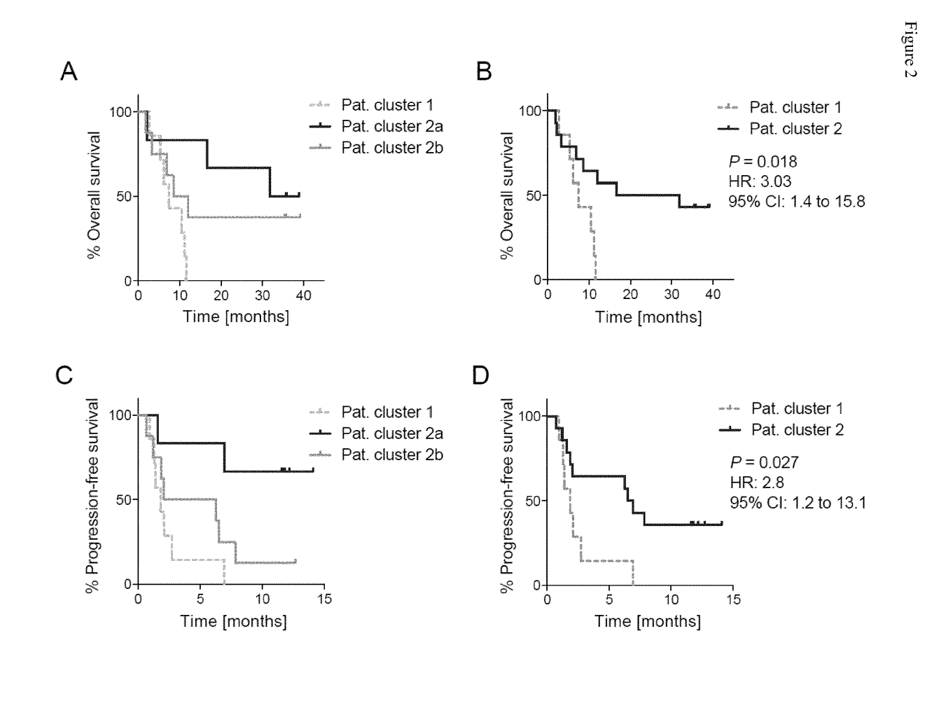

Unsupervised hierarchical clustering of module activity scores identified three distinct clusters of BTM week 5 to week 0 changes: a T/NK cell cluster, a cell cycle cluster and a myeloid/immune activation cluster (FIG. 1). Furthermore, unsupervised hierarchical clustering of samples identified three clusters of patients (patient cluster 1, 2a and 2b). Patients belonging to clusters 2a and 2b exhibited prolonged survival compared to patients from cluster 1 (FIG. 2).

We next sought to determine the single genes either driving or being associated with the clustering of the BTMs. To this end, gene expression differences between week 5 and corresponding week 0 samples were calculated for the entire transcriptome. As described above, probes with highest average expression were chosen if genes were targeted by multiple probes.

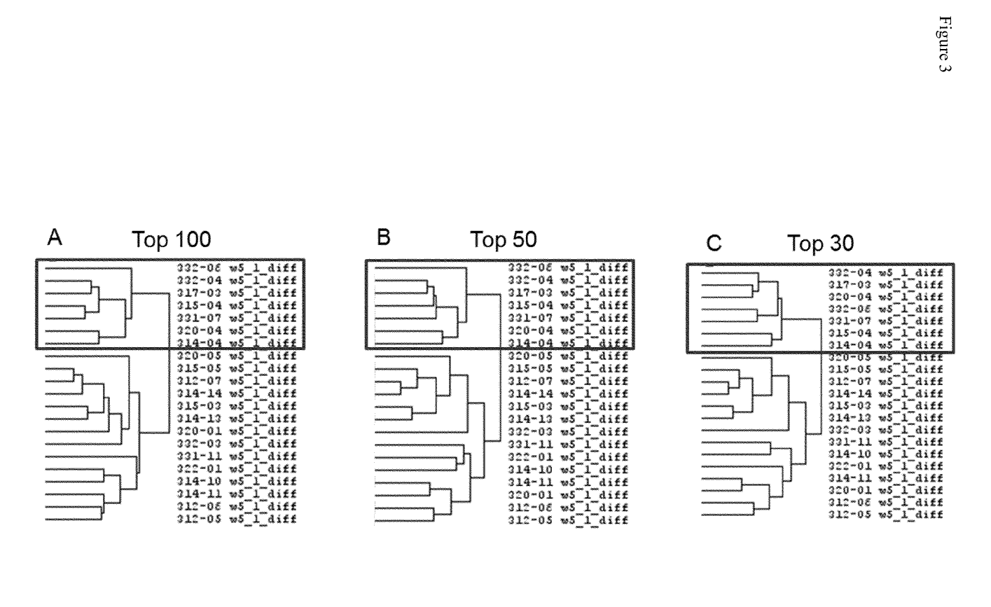

To identify genes differentially expressed in patient cluster 1 compared to cluster 2, following approaches were utilized: patients were grouped according to aforementioned clusters Genes were ranked according to p values comparing patient clusters 1 and 2 (t test) or clusters 1 versus 2a and 2b (ANOVA). Thus, altogether four comparisons were performed. The first top 100 hits were picked from each comparison. In addition, gene set enrichment analysis was performed to identify the top 15 BTMs enriched at week 5 in the patient clusters 1, 2a and 2b. Leading edge genes for these top 15 BTMs were identified and all duplicate genes were removed. Thus, a final list was generated comprising 914 single genes (as shown in Table 1). By unsupervised hierarchical clustering of these single gene expression changes (914 genes) (week 5 to week 0 differences) the patients could be segregated into the two groups of short term survivors and moderate/longterm survivors. Unsupervised hierarchical clustering of BTM activity scores was performed using Euclidean distance and average linkage clustering. The 915 genes were ranked based on their ability to differentiate between patients derived from cluster 1 and patients derived from cluster 2. Top 100 (Table 2; FIG. 3 A), top 50 (Table 3, FIG. 3 B), or top 30 (Table 4; FIG. 3 C) genes were selected, and gene expression differences between week 5 and 0 were calculated followed by average-linkage hierarchical clustering. Clustering trees of patients are shown and short-term survivors are highlighted.

TABLE 16
Study 1 - gene expression differences between week 5 and
week 0 of marker genes (full name and accession number as set
out in Table 1)

| | Week5_0 GE | Week5_0 GE | |
|---|---|---|---|
| | difference_ | difference_ | |
| | mean_pat_ | mean_pat_ | |
| Gene Symbol | clust1 | clust2 | Difference |
| LOC572558 | 0.836493129 | −0.56501209 | 1.40150522 |
| USH1G | 0.5566099 | −0.82634535 | 1.38295525 |
| BMP2 | 0.598134986 | −0.73352584 | 1.33166082 |
| KIF18A | −0.4757864 | 0.79021046 | 1.26599686 |
| ANLN | −0.52647626 | 0.72441581 | 1.25089207 |
| GP5 | −0.68564021 | 0.49620231 | 1.18184253 |
| SKA3 | −0.3279785 | 0.82917226 | 1.15715076 |
| TTK | −0.22210423 | 0.89190909 | 1.11401331 |
| OLFM4 | 0.5396805 | −0.55518684 | 1.09486734 |
| DLGAP5 | −0.43158276 | 0.63624947 | 1.06783223 |
| MCM10 | −0.27769329 | 0.78038119 | 1.05807447 |
| SLPI | 0.750556771 | −0.29516672 | 1.04572349 |
| FASLG | −0.40907543 | 0.63064534 | 1.03972076 |
| SLC7A8 | 0.507136586 | −0.52383601 | 1.03097259 |
| EXO1 | −0.4247823 | 0.59886942 | 1.02365172 |
| FGFBP2 | −0.52265307 | 0.47477268 | 0.99742575 |
| CXCL1 | 0.356745571 | −0.64063769 | 0.99738326 |
| MELK | −0.45311947 | 0.54402785 | 0.99714732 |
| RRM2 | −0.32322949 | 0.65163608 | 0.97486556 |
| KIAA0101 | −0.47635057 | 0.48525963 | 0.9616102 |
| IF127 | 0.176026157 | 1.11120696 | 0.9351808 |
| RAD51 | −0.3247114 | 0.60031006 | 0.92502146 |
| TYMS | −0.437593 | 0.48260443 | 0.92019743 |
| E2F8 | 0.040218886 | 0.95494481 | 0.91472592 |
| PDGFRB | −0.39981491 | 0.51231926 | 0.91213417 |
| CHI3L1 | 0.588718757 | −0.31388162 | 0.90260038 |
| IFNG | −0.42609171 | 0.47086009 | 0.8969518 |
| CDT1 | −0.39202781 | 0.50043445 | 0.89246226 |
| CCL20 | 0.556402414 | −0.32817589 | 0.88457831 |
| DTL | −0.32041199 | 0.56387945 | 0.88429144 |
| LOC643650 | 0.665895186 | −0.2064908 | 0.87238599 |
| CCNA2 | −0.35663496 | 0.51559274 | 0.8722277 |
| RGS9 | −0.39346829 | 0.47757099 | 0.87103927 |
| CEP55 | −0.24708361 | 0.61351535 | 0.86059896 |
| POLE2 | −0.5204712 | 0.33988834 | 0.86035954 |
| ASPM | −0.32645329 | 0.53236356 | 0.85881685 |
| TGFBR3 | −0.52647194 | 0.3242523 | 0.85072424 |
| RPGRIP1 | 0.447854257 | −0.40147147 | 0.84932573 |
| KIR2DL2 | −0.5014236 | 0.33998994 | 0.84141354 |
| HJURP | −0.34120807 | 0.49581671 | 0.83702478 |
| STAP1 | −0.39942023 | 0.43645486 | 0.83587509 |
| TOP2A | −0.4450913 | 0.38674186 | 0.83183316 |
| XLOC_l2_010897 | 0.547097857 | −0.28466364 | 0.8317615 |
| PPP2R2B | −0.37462457 | 0.45606007 | 0.83068464 |
| SPC25 | −0.38262983 | 0.44521026 | 0.82784009 |
| KIAA1671 | −0.35329513 | 0.47447439 | 0.82776952 |
| BUB1 | −0.25203216 | 0.5736033 | 0.82563546 |
| GPR56 | −0.39415557 | 0.42937814 | 0.82353371 |
| GZMM | −0.52771514 | 0.29230996 | 0.82002511 |
| SAMD3 | −0.372721 | 0.44468854 | 0.81740954 |
| CTSW | −0.487019 | 0.32969107 | 0.81671007 |
| PTGFRN | 0.551742114 | −0.25487401 | 0.80661612 |
| XLOC_006752 | 0.348433857 | −0.45524939 | 0.80368325 |
| GNLY | −0.44419471 | 0.35938757 | 0.80358229 |
| LINC00239 | −0.41751591 | 0.38543004 | 0.80294595 |
| CDCA2 | −0.38017363 | 0.42248241 | 0.80265604 |
| GZMH | −0.36358736 | 0.43763271 | 0.80122007 |
| BUB1B | −0.3748953 | 0.42602996 | 0.80092526 |
| UBE2C | −0.38013587 | 0.41798133 | 0.7981172 |
| KIR2DS4 | −0.46300439 | 0.33428446 | 0.79728885 |
| SLAMF6 | −0.3265845 | 0.46976723 | 0.79635173 |
| KIR2DS2 | −0.512763 | 0.28105043 | 0.79381343 |
| XLOC_004032 | 0.388846443 | −0.40493636 | 0.7937828 |
| PRF1 | −0.36652071 | 0.42110561 | 0.78762632 |
| GINS2 | −0.31950007 | 0.46183131 | 0.78133139 |
| CDC45 | −0.36169197 | 0.41810126 | 0.77979323 |
| TBX21 | −0.38747943 | 0.3888075 | 0.77628693 |
| GZMA | −0.32334621 | 0.44860282 | 0.77194904 |
| NKG7 | −0.40032814 | 0.36869686 | 0.769025 |
| SGK1 | 0.316197857 | −0.45204007 | 0.76823793 |
| PVRIG | −0.33004371 | 0.43754479 | 0.7675885 |
| NEK2 | −0.4797225 | 0.28285472 | 0.76257722 |
| TARP | −0.32536107 | 0.43529539 | 0.76065646 |
| BIRC5 | −0.36301359 | 0.39512951 | 0.7581431 |
| CST7 | −0.40688907 | 0.35034489 | 0.75723396 |
| IL2RB | −0.43837371 | 0.31879714 | 0.75717086 |
| MND1 | −0.239129 | 0.51650061 | 0.75562961 |
| S1PR5 | −0.4823231 | 0.27190594 | 0.75422904 |
| STK32B | 0.403095857 | −0.34853537 | 0.75163123 |
| KLRF1 | −0.42285957 | 0.32693082 | 0.74979039 |
| ENPP5 | −0.19809159 | 0.55005255 | 0.74814414 |
| IL18RAP | −0.43409771 | 0.31250082 | 0.74659854 |
| GRAP2 | −0.39740214 | 0.34823431 | 0.74563645 |
| SH2D2A | −0.37704021 | 0.36344946 | 0.74048968 |
| CR1 | 0.513004429 | −0.22538764 | 0.73839207 |
| IQCD | 0.443972843 | −0.29392262 | 0.73789546 |
| CCL5 | −0.37457179 | 0.36271357 | 0.73728536 |
| KLRD1 | −0.39966243 | 0.33700039 | 0.73666282 |
| CXCR3 | −0.33505586 | 0.40025993 | 0.73531579 |
| CD247 | −0.42550657 | 0.30918132 | 0.73468789 |
| ZNF697 | 0.343343186 | −0.38604165 | 0.72938484 |
| HOPX | −0.40795114 | 0.31981293 | 0.72776407 |
| IL1B | 0.430601643 | −0.29520746 | 0.72580911 |
| KLRC1 | −0.40147829 | 0.32408429 | 0.72556257 |
| ZNF503 | 0.260294643 | −0.46513275 | 0.72542739 |
| XLOC_004924 | 0.442685629 | −0.28242409 | 0.72510971 |
| ZAP70 | −0.37333843 | 0.35043057 | 0.723769 |
| CENPF | −0.26337054 | 0.45853571 | 0.72190625 |
| PTPRCAP | −0.32277993 | 0.39478421 | 0.71756414 |
| KLRC3 | −0.44044429 | 0.27508348 | 0.71552776 |
| OIP5 | −0.21582867 | 0.49771236 | 0.71354104 |
| PYHIN1 | −0.40307107 | 0.31045202 | 0.71352309 |
| KIR3DL1 | −0.45761089 | 0.25522369 | 0.71283457 |
| FFAR2 | 0.403203914 | −0.3020409 | 0.70524481 |
| DOCK4 | 0.5071245 | −0.19795293 | 0.70507743 |
| KIR2DL5A | −0.36701536 | 0.33725142 | 0.70426678 |
| STAT4 | −0.36686936 | 0.33649532 | 0.70336468 |
| CCNB1 | −0.22058387 | 0.48166149 | 0.70224536 |
| NLRC3 | −0.32309807 | 0.37479546 | 0.69789354 |
| CD226 | −0.3092581 | 0.38837247 | 0.69763057 |
| LIN7A | 0.384615571 | −0.31028161 | 0.69489718 |
| CCDC102A | −0.25091279 | 0.44355494 | 0.69446773 |
| SBK1 | −0.32368543 | 0.36739386 | 0.69107929 |
| CD1D | 0.332267257 | −0.35830728 | 0.69057454 |
| MCM2 | −0.26376014 | 0.42386507 | 0.68762521 |
| CDCA5 | −0.32649949 | 0.35916156 | 0.68566105 |
| CRISPLD2 | 0.325558914 | −0.35866663 | 0.68422554 |
| SPC24 | −0.20801669 | 0.47344263 | 0.68145931 |
| TXK | −0.39899106 | 0.28206181 | 0.68105287 |
| MYBL1 | −0.32378307 | 0.35550657 | 0.67928964 |
| FLJ23867 | 0.2892186 | −0.38841893 | 0.67763753 |
| CAMP | 0.331554029 | −0.34489608 | 0.67645011 |
| DLL1 | −0.40856636 | 0.26751013 | 0.67607649 |
| STMN1 | −0.32491957 | 0.34691546 | 0.67183504 |
| PTGDR | −0.24607819 | 0.42568543 | 0.67176361 |
| CD7 | −0.44135829 | 0.228816 | 0.67017429 |
| XLOC_011068 | 0.358870857 | −0.31087044 | 0.6697413 |
| TREM1 | 0.319241571 | −0.34985461 | 0.66909618 |
| CLMN | 0.417658286 | −0.24783318 | 0.66549146 |
| TIGIT | −0.22869957 | 0.43677026 | 0.66546983 |
| CLIC3 | −0.46189586 | 0.20322614 | 0.665122 |
| IL32 | −0.28410986 | 0.37717586 | 0.66128571 |
| APCDD1 | 0.5716404 | −0.08941537 | 0.66105577 |
| ZFHX3 | 0.258988 | −0.39967111 | 0.65865911 |
| ST3GAL6 | 0.454685 | −0.20187069 | 0.65655569 |
| CXCR6 | −0.1687038 | 0.48423314 | 0.65293694 |
| EVI5 | 0.304583 | −0.34727826 | 0.65186126 |
| CDK1 | −0.29766186 | 0.35394821 | 0.65161007 |
| XLOC_002344 | 0.278257714 | −0.37277171 | 0.65102942 |
| GAS2L3 | 0.2892733 | −0.36166317 | 0.65093647 |
| BRIP1 | −0.06473094 | 0.58398673 | 0.64871767 |
| C15orf38 | 0.352214186 | −0.29639379 | 0.64860798 |
| ST6GALNAC2 | 0.362440414 | −0.28541456 | 0.64785498 |
| LOC100131043 | 0.497533014 | −0.14947804 | 0.64701106 |
| NLRP3 | 0.198566786 | −0.44595868 | 0.64452546 |
| TMEM144 | 0.445366286 | −0.19525502 | 0.64062131 |
| KLRK1 | −0.29429236 | 0.34556071 | 0.63985307 |
| ELOVL4 | 0.575955571 | −0.06378644 | 0.63974201 |
| XCL1 | −0.46850007 | 0.17072114 | 0.63922121 |
| NDFIP2 | −0.13406246 | 0.50343696 | 0.63749941 |
| AHR | 0.425404357 | −0.20952718 | 0.63493154 |
| XLOC_001266 | 0.341852057 | −0.29172474 | 0.6335768 |
| SETD7 | 0.278564214 | −0.35465161 | 0.63321582 |
| GZMB | −0.38500943 | 0.24324889 | 0.62825832 |
| F2RL1 | 0.430577814 | −0.19752631 | 0.62810412 |
| SLA2 | −0.37827891 | 0.24943868 | 0.62771759 |
| PTPN4 | −0.33267557 | 0.29473275 | 0.62740832 |
| CHAF1B | −0.28010579 | 0.34651381 | 0.6266196 |
| CD3D | −0.23348186 | 0.39212054 | 0.62560239 |
| PRKCH | −0.35848186 | 0.26582529 | 0.62430714 |
| KIF20A | −0.21280003 | 0.41019729 | 0.62299732 |
| KIFC1 | −0.10751596 | 0.51517141 | 0.62268736 |
| HP | 0.596373071 | −0.02630196 | 0.62267504 |
| CDCA7 | −0.21515 | 0.40727161 | 0.62242161 |
| ITM2A | −0.26605336 | 0.35634646 | 0.62239982 |
| XLOC_011350 | 0.295312529 | −0.32692951 | 0.62224204 |
| IL8 | 0.216571714 | −0.40485764 | 0.62142936 |
| LOC79015 | −0.27151643 | 0.34920865 | 0.62072508 |
| UBE2T | −0.11525863 | 0.50473483 | 0.61999346 |
| PLEKHF1 | −0.27916886 | 0.33819318 | 0.61736204 |
| KLRB1 | −0.38298343 | 0.23405564 | 0.61703907 |
| PLB1 | 0.396175286 | −0.22081686 | 0.61699214 |
| CD3G | −0.28115043 | 0.33538586 | 0.61653629 |
| NDC80 | −0.26965786 | 0.34558996 | 0.61524782 |
| ELOVL6 | −0.2569174 | 0.3583198 | 0.6152372 |
| CDKN3 | −0.23962386 | 0.37401346 | 0.61363731 |
| CENPA | −0.28117887 | 0.3316836 | 0.61286247 |
| RNF217 | 0.367217271 | −0.24210219 | 0.60931946 |
| ORM1 | 0.327330243 | −0.28103712 | 0.60836736 |
| ATP6V0A1 | 0.410282 | −0.19580129 | 0.60608329 |
| ASGR2 | 0.394543714 | −0.20919582 | 0.60373954 |
| ZWINT | −0.22816706 | 0.37430662 | 0.60247368 |
| LRP1 | 0.342941071 | −0.25913879 | 0.60207986 |
| CD6 | −0.23678907 | 0.36355446 | 0.60034354 |
| GPR114 | −0.29362574 | 0.30667602 | 0.60030176 |
| AQP9 | 0.451633286 | −0.14842921 | 0.6000625 |
| CD3E | −0.351057 | 0.24763504 | 0.59869204 |
| RASGRP1 | −0.29033786 | 0.30770064 | 0.5980385 |
| CD2 | −0.21646543 | 0.38027496 | 0.59674039 |
| MCM4 | −0.23200734 | 0.36402665 | 0.59603399 |
| FAM20C | 0.354564429 | −0.24098214 | 0.59554657 |
| IL13RA1 | 0.222207429 | −0.37244486 | 0.59465229 |
| CCNB2 | −0.19466363 | 0.39873935 | 0.59340298 |
| XLOC_l2_015034 | 0.194223386 | −0.39885572 | 0.59307911 |
| PRKCQ | −0.30476429 | 0.28771886 | 0.59248314 |
| CLEC4E | 0.2837066 | −0.30689578 | 0.59060238 |
| ACPP | 0.338642571 | −0.25159625 | 0.59023882 |
| WDFY3 | 0.227156 | −0.36272518 | 0.58988118 |
| SH2D1A | −0.21825836 | 0.37141254 | 0.58967089 |
| UHRF1 | −0.20136976 | 0.38829452 | 0.58966428 |
| SMAD1 | 0.287765043 | −0.29998998 | 0.58775502 |
| FAM198B | 0.399518214 | −0.18798039 | 0.58749861 |
| GGTA1P | 0.167619286 | −0.41747589 | 0.58509518 |
| TC2N | −0.26454679 | 0.32010684 | 0.58465362 |
| XLOC_014211 | 0.011044229 | −0.57262589 | 0.58367012 |
| DSCC1 | −0.2080774 | 0.37472837 | 0.58280577 |
| ETS1 | −0.30955529 | 0.270924 | 0.58047929 |
| RBM47 | 0.349116429 | −0.23082243 | 0.57993886 |
| SLC22A4 | 0.244807786 | −0.33498348 | 0.57979126 |
| UBASH3A | −0.22020343 | 0.35947714 | 0.57968057 |
| CENPM | −0.12387793 | 0.4553175 | 0.57919543 |
| TLR2 | 0.444897714 | −0.13343436 | 0.57833207 |
| DENND2D | −0.17553779 | 0.40131875 | 0.57685654 |
| KIR2DS3 | −0.44095089 | 0.13590208 | 0.57685296 |
| CD163 | 0.284595286 | −0.2914575 | 0.57605279 |
| ZNF516 | 0.341899714 | −0.23192736 | 0.57382707 |
| MCM6 | −0.2673995 | 0.30639182 | 0.57379132 |
| MANSC1 | 0.358897314 | −0.21444576 | 0.57334307 |
| SLAMF7 | −0.23002979 | 0.34091768 | 0.57094746 |
| LAT | −0.25102107 | 0.318139 | 0.56916007 |
| LAG3 | −0.15927029 | 0.40855932 | 0.56782961 |
| HOXB4 | −0.18688931 | 0.38039065 | 0.56727996 |
| CD96 | −0.29548743 | 0.27057925 | 0.56606668 |
| CYP1B1 | 0.327229714 | −0.23867029 | 0.5659 |
| WLS | 0.307582429 | −0.25765561 | 0.56523804 |
| IL1R1 | 0.240469186 | −0.32374119 | 0.56421038 |
| CLEC4D | 0.360743286 | −0.20266225 | 0.56340554 |
| SPOCK2 | −0.31221307 | 0.25077596 | 0.56298904 |
| MBOAT2 | 0.183023886 | −0.37933214 | 0.56235603 |
| SORT1 | 0.327330143 | −0.23349196 | 0.56082211 |
| KIF15 | −0.2177303 | 0.34251854 | 0.56024884 |
| CSF3R | 0.314176714 | −0.24596696 | 0.56014368 |
| ITK | −0.294615 | 0.26540107 | 0.56001607 |
| HNMT | 0.232559143 | −0.32677368 | 0.55933282 |
| TIE1 | −0.32596631 | 0.23241353 | 0.55837984 |
| PRKACB | −0.25575786 | 0.30210143 | 0.55785929 |
| CTIF | 0.221119357 | −0.33316384 | 0.55428319 |
| CMTM2 | 0.304997757 | −0.24824621 | 0.55324397 |
| TBC1D9 | 0.249669857 | −0.30340593 | 0.55307579 |
| SPINT1 | 0.333112 | −0.21939014 | 0.55250214 |
| LBH | −0.24474714 | 0.30730196 | 0.55204911 |
| KIF2C | −0.18447456 | 0.36618417 | 0.55065873 |
| RUSC2 | 0.293138643 | −0.25688511 | 0.55002375 |
| GZMK | −0.29421721 | 0.25558446 | 0.54980168 |
| CD320 | −0.3055135 | 0.24368532 | 0.54919882 |
| NCAM1 | −0.36557571 | 0.18317471 | 0.54875043 |
| TMTC2 | 0.383854214 | −0.16442957 | 0.54828379 |
| ASGR1 | 0.3071775 | −0.24050568 | 0.54768318 |
| FXYD6 | 0.2127035 | −0.33376146 | 0.54646496 |
| CKAP2L | −0.45269184 | 0.09253379 | 0.54522564 |
| OXER1 | 0.277124214 | −0.26681871 | 0.54394293 |
| PYGL | 0.291343071 | −0.25168736 | 0.54303043 |
| S1PR3 | 0.391696929 | −0.15085943 | 0.54255636 |
| KLF12 | −0.28678086 | 0.25544246 | 0.54222332 |
| ANXA6 | −0.29465314 | 0.24746879 | 0.54212193 |
| BCL6 | 0.2903715 | −0.25103257 | 0.54140407 |
| KLF5 | 0.304956671 | −0.23619437 | 0.54115104 |
| ENO2 | −0.2168789 | 0.3236394 | 0.5405183 |
| CD27 | −0.19185293 | 0.347574 | 0.53942693 |
| RBP7 | 0.233317143 | −0.3036785 | 0.53699564 |
| LOC100131490 | 0.307047286 | −0.22785415 | 0.53490144 |
| PTPN7 | −0.14836743 | 0.38641711 | 0.53478454 |
| HSD3B7 | 0.517880143 | −0.01636599 | 0.53424613 |
| ENTPD1 | 0.217274 | −0.31624082 | 0.53351482 |
| CENPK | −0.21435279 | 0.31886286 | 0.53321564 |
| HAL | 0.242564014 | −0.29026531 | 0.53282932 |
| GPR125 | −0.35057997 | 0.18160325 | 0.53218322 |
| LOC100289495 | 0.292156871 | −0.23932099 | 0.53147786 |
| RGL4 | −0.25457907 | 0.27637839 | 0.53095746 |
| CD8B | −0.18820371 | 0.34228961 | 0.53049332 |
| DLEU1 | −0.2542565 | 0.27575675 | 0.53001325 |
| IL18R1 | −0.29159811 | 0.23804614 | 0.52964426 |
| DACH1 | 0.337819657 | −0.19158154 | 0.52940119 |
| CD8A | −0.20150093 | 0.32647193 | 0.52797286 |
| E2F7 | −0.17232293 | 0.35509695 | 0.52741988 |
| TNFRSF10C | 0.297923571 | −0.2294785 | 0.52740207 |
| TPX2 | −0.13689606 | 0.39044902 | 0.52734508 |
| TLR5 | 0.291976214 | −0.23228036 | 0.52425657 |
| CD5 | −0.24713221 | 0.27680911 | 0.52394132 |
| LOC387895 | −0.21919861 | 0.30467782 | 0.52387644 |
| STAB1 | 0.346968429 | −0.17626314 | 0.52323157 |
| ARRDC4 | 0.2649595 | −0.25809061 | 0.52305011 |
| LOC100506119 | 0.294396 | −0.22709296 | 0.52148896 |
| PTTG1 | −0.16892521 | 0.35232025 | 0.52124546 |
| SLC11A1 | 0.253459714 | −0.26535271 | 0.51881243 |
| PADI4 | 0.1552918 | −0.36114964 | 0.51644144 |
| VSTM1 | 0.224156143 | −0.29217904 | 0.51633518 |
| OPLAH | 0.3919475 | −0.12385536 | 0.51580286 |
| PADI2 | 0.241451286 | −0.27375796 | 0.51520925 |
| SLC38A1 | −0.23439729 | 0.28044493 | 0.51484221 |
| CHST12 | −0.21952229 | 0.29463357 | 0.51415586 |
| ABLIM1 | −0.19230614 | 0.3216015 | 0.51390764 |
| CATSPER1 | 0.230605429 | −0.28283689 | 0.51344232 |
| CECR6 | 0.309074 | −0.20393032 | 0.51300432 |
| TLR6 | 0.329424271 | −0.18336559 | 0.51278986 |
| SIGLEC9 | 0.370637257 | −0.14214075 | 0.51277801 |
| HOXB2 | −0.20811757 | 0.30407686 | 0.51219443 |
| PLXDC2 | 0.263196429 | −0.24899125 | 0.51218768 |
| CREB5 | 0.371664286 | −0.14022161 | 0.51188589 |
| LOC100240735 | −0.11060654 | 0.40042911 | 0.51103565 |
| HAAO | 0.259200929 | −0.25162175 | 0.51082268 |
| SLC8A1 | 0.314681643 | −0.19613093 | 0.51081257 |
| SIT1 | −0.10533347 | 0.40479136 | 0.51012484 |
| NUF2 | −0.11992784 | 0.38920616 | 0.509134 |
| BCL11B | −0.20831614 | 0.30066271 | 0.50897886 |
| ALCAM | 0.356605357 | −0.15212025 | 0.50872561 |
| BAHCC1 | 0.191048329 | −0.31752701 | 0.50857534 |
| LIPE | −0.15525814 | 0.35290129 | 0.50815943 |
| BTLA | 0.063817314 | 0.57193829 | 0.50812097 |
| FAM169A | −0.25192357 | 0.25607334 | 0.50799691 |
| RAD54L | −0.18824769 | 0.31971504 | 0.50796273 |
| PSRC1 | 0.332147143 | −0.17503814 | 0.50718529 |
| PVR | 0.315551843 | −0.19133553 | 0.50688737 |
| ICOS | −0.17266071 | 0.33378096 | 0.50644168 |
| RAD51AP1 | −0.15825344 | 0.34733869 | 0.50559213 |
| RHOH | −0.26363393 | 0.24160936 | 0.50524329 |
| CDA | 0.2362715 | −0.26884225 | 0.50511375 |
| WDR67 | −0.19719257 | 0.30739107 | 0.50458364 |
| LEF1 | −0.23067757 | 0.27287407 | 0.50355164 |
| CD40LG | −0.11914729 | 0.38176804 | 0.50091532 |
| CD93 | 0.293264357 | −0.20619332 | 0.49945768 |
| SLC16A5 | 0.237339 | −0.26199429 | 0.49933329 |
| FAM7A1 | 0.207298014 | −0.29015196 | 0.49744997 |
| HLX | 0.416788886 | −0.07845021 | 0.4952391 |
| FZD1 | 0.202783143 | −0.29188636 | 0.4946695 |
| TCF7 | −0.2205655 | 0.27400575 | 0.49457125 |
| CLEC7A | 0.174408714 | −0.32003786 | 0.49444657 |
| RASGRP4 | 0.225120857 | −0.26896 | 0.49408086 |
| S100Z | 0.278363429 | −0.21444639 | 0.49280982 |
| SLC36A1 | 0.362534357 | −0.12964436 | 0.49217871 |
| IRAK3 | 0.177265714 | −0.31149632 | 0.48876204 |
| REPS2 | 0.167204686 | −0.32112792 | 0.48833261 |
| CDCA8 | −0.22826156 | 0.2586116 | 0.48687316 |
| CACNA2D3 | 0.147184729 | −0.33929497 | 0.4864797 |
| ATP9A | −0.19487714 | 0.29154147 | 0.48641861 |
| TLR4 | 0.283682 | −0.2026975 | 0.4863795 |
| C5AR1 | 0.088096071 | −0.39689704 | 0.48499311 |
| JUP | 0.156214714 | −0.32745473 | 0.48366944 |
| RNASE6 | 0.242342286 | −0.24121675 | 0.48355904 |
| SRD5A3 | 0.1854806 | −0.29805416 | 0.48353476 |
| VNN3 | 0.089851229 | −0.39324561 | 0.48309684 |
| NCF4 | 0.287130143 | −0.19360982 | 0.48073996 |
| CPAMD8 | 0.213523286 | −0.26710254 | 0.48062582 |
| IMPA2 | 0.300314357 | −0.17852368 | 0.47883804 |
| DUSP6 | 0.405228286 | −0.07084136 | 0.47606964 |
| SLC1A3 | 0.399558143 | −0.07517946 | 0.47473761 |
| STEAP4 | 0.455998671 | −0.01826882 | 0.47426749 |
| SIRPA | 0.300369143 | −0.17279968 | 0.47316882 |
| PROK2 | 0.391483571 | −0.081551 | 0.47303457 |
| HOMER3 | 0.268185429 | −0.20481154 | 0.47299696 |
| PCNA | −0.20712936 | 0.26508754 | 0.47221689 |
| LOC100506190 | 0.264811214 | −0.20677068 | 0.47158189 |
| LRRN3 | −0.14819123 | 0.32129771 | 0.46948894 |
| PELI3 | 0.322259214 | −0.14640793 | 0.46866714 |
| GLT1D1 | 0.290086143 | −0.17845936 | 0.4685455 |
| TRAT1 | −0.13320714 | 0.33377432 | 0.46698146 |
| IFNGR2 | 0.221502643 | −0.24534257 | 0.46684521 |
| VCAN | 0.32093 | −0.14525382 | 0.46618382 |
| FBP1 | 0.245210214 | −0.22077964 | 0.46598986 |
| ATF3 | 0.227069857 | −0.23813082 | 0.46520068 |
| THEMIS | −0.16147286 | 0.303302 | 0.46477486 |
| ALDH18A1 | −0.23830643 | 0.22640319 | 0.46470961 |
| PTAFR | 0.367958286 | −0.09622964 | 0.46418793 |
| KIF14 | −0.12679421 | 0.33703227 | 0.46382649 |
| CCNE2 | −0.23386037 | 0.22907573 | 0.4629361 |
| KCNE3 | 0.286638071 | −0.17603007 | 0.46266814 |
| FIGNL1 | −0.39112519 | 0.07060053 | 0.46172571 |
| MNDA | 0.304120571 | −0.15425471 | 0.45837529 |
| HP07349 | 0.224252871 | −0.23307984 | 0.45733271 |
| CPVL | 0.190771357 | −0.265568 | 0.45633936 |
| CLDN23 | 0.192351557 | −0.26357798 | 0.45592954 |
| CASC5 | −0.0853667 | 0.3705584 | 0.4559251 |
| ITPR3 | −0.14814271 | 0.30532818 | 0.45347089 |
| RGL1 | 0.292098071 | −0.16081871 | 0.45291678 |
| TM6SF1 | 0.172928171 | −0.27996275 | 0.45289092 |
| SLC22A15 | 0.256408929 | −0.19479271 | 0.45120164 |
| BMP2K | 0.340658 | −0.11052889 | 0.45118689 |
| RORA | −0.31763243 | 0.13335904 | 0.45099146 |
| ENPP4 | −0.22791429 | 0.22254214 | 0.45045643 |
| MCM3 | −0.1810385 | 0.26783636 | 0.44887486 |
| C3AR1 | 0.236919 | −0.21150971 | 0.44842871 |
| BCL2 | −0.19330557 | 0.25353029 | 0.44683586 |
| LRG1 | 0.218137786 | −0.22740541 | 0.44554319 |
| NACC2 | 0.184041143 | −0.26108996 | 0.44513111 |
| GAS2L1 | 0.211091286 | −0.23393936 | 0.44503064 |
| RASA3 | −0.22732943 | 0.21659846 | 0.44392789 |
| CLEC12A | 0.203698143 | −0.23979146 | 0.44348961 |
| CENPO | −0.15994436 | 0.28329822 | 0.44324258 |
| PLBD1 | 0.308567143 | −0.13360864 | 0.44217579 |
| IFNGR1 | 0.206392429 | −0.23572343 | 0.44211586 |
| AURKA | −0.06510166 | 0.376225 | 0.44132666 |
| FLT3LG | −0.24143607 | 0.19988446 | 0.44132054 |
| CYFIP1 | 0.278670429 | −0.16178471 | 0.44045514 |
| KLK3 | 0.317315214 | −0.12297611 | 0.44029133 |
| DGAT2 | 0.383776214 | −0.05560687 | 0.43938309 |
| KIAA1598 | 0.196053071 | −0.24282182 | 0.43887489 |
| LCK | −0.19461843 | 0.24340629 | 0.43802471 |
| TRIP13 | −0.11449776 | 0.32300382 | 0.43750158 |
| DIRC2 | 0.209791143 | −0.22762654 | 0.43741768 |
| RAB13 | 0.253744629 | −0.18336525 | 0.43710988 |
| LRRC4 | 0.381947829 | −0.05513704 | 0.43708487 |
| LOC100130458 | 0.116891071 | 0.55288541 | 0.43599434 |
| MAL | −0.16839007 | 0.26727318 | 0.43566325 |
| TNS3 | 0.278597929 | −0.1568785 | 0.43547643 |
| LPCAT2 | 0.168471571 | −0.26599285 | 0.43446442 |
| DENND1A | 0.187873214 | −0.24632518 | 0.43419839 |
| PPP1R32 | 0.246556914 | −0.18738554 | 0.43394245 |
| LRRK2 | 0.179330429 | −0.25345061 | 0.43278104 |
| MARCH1 | 0.304293571 | −0.12841868 | 0.43271225 |
| CSDA | 0.174335429 | −0.25818643 | 0.43252186 |
| GATA3 | −0.15800157 | 0.27439999 | 0.43240156 |
| MGST1 | 0.260136214 | −0.171845 | 0.43198121 |
| CAMK4 | −0.21311271 | 0.21834766 | 0.43146038 |
| MCM8 | −0.16223123 | 0.26906441 | 0.43129564 |
| XLOC_014161 | 0.159774714 | −0.27144611 | 0.43122082 |
| COPG2 | 0.243141886 | −0.18801444 | 0.43115633 |
| TP53I3 | 0.221867429 | −0.20887982 | 0.43074725 |
| EMR2 | 0.165954643 | −0.26454793 | 0.43050257 |
| VNN1 | 0.249032214 | −0.1813005 | 0.43033271 |
| KIF11 | −0.15487474 | 0.27468888 | 0.42956362 |
| MSH2 | −0.117764 | 0.31093821 | 0.42870221 |
| WEE1 | −0.15173171 | 0.27648444 | 0.42821615 |
| P2RY13 | 0.317733 | −0.10982325 | 0.42755625 |
| HELB | −0.11619527 | 0.31125171 | 0.42744698 |
| TRANK1 | −0.12740821 | 0.29891346 | 0.42632168 |
| CCR1 | 0.300865143 | −0.12532832 | 0.42619346 |
| LOC100505921 | −0.15721229 | 0.26829006 | 0.42550234 |
| LHFPL2 | 0.341473714 | −0.08390814 | 0.42538186 |
| SLC24A4 | 0.199079071 | −0.22578986 | 0.42486893 |
| LTF | 0.763298829 | 0.33854093 | 0.4247579 |
| FCRL5 | 0.095907571 | 0.51954987 | 0.4236423 |
| PCDH9 | 0.471096286 | 0.89444049 | 0.42334421 |
| RBL1 | −0.1193045 | 0.30341787 | 0.42272237 |
| MAD2L1 | −0.1968512 | 0.22556866 | 0.42241986 |
| SHCBP1 | −0.18099769 | 0.24105691 | 0.4220546 |
| FOLR3 | 0.200585357 | −0.22105254 | 0.42163789 |
| ALDH2 | 0.138676143 | −0.28251946 | 0.42119561 |
| TGFBI | 0.218445357 | −0.20263325 | 0.42107861 |
| LY96 | 0.252913286 | −0.16756939 | 0.42048268 |
| GLB1L | 0.183340414 | −0.23602441 | 0.41936483 |
| NELL2 | −0.17102343 | 0.24754086 | 0.41856429 |
| SCPEP1 | 0.205820571 | −0.21247036 | 0.41829093 |
| MYBL2 | −0.24317244 | 0.17455584 | 0.41772829 |
| DYSF | 0.359312929 | −0.05823496 | 0.41754789 |
| NAIP | 0.3178735 | −0.09899714 | 0.41687064 |
| CD36 | 0.308184 | −0.10792732 | 0.41611132 |
| LY86 | 0.172096714 | −0.24375036 | 0.41584707 |
| PRAM1 | 0.206100857 | −0.20934879 | 0.41544964 |
| IL7R | −0.07469257 | 0.33924354 | 0.41393611 |
| PDLIM7 | 0.302697929 | −0.11055939 | 0.41325732 |
| MS4A4A | 0.334755214 | −0.07767843 | 0.41243364 |
| FCGR2A | 0.195130571 | −0.21716389 | 0.41229446 |
| DOCK5 | 0.142947243 | −0.26929281 | 0.41224005 |
| RAB34 | 0.203238143 | −0.20877086 | 0.412009 |
| CYBRD1 | 0.111162 | −0.30075943 | 0.41192143 |
| RNASE3 | 0.268917 | −0.14296371 | 0.41188071 |
| HPSE | 0.277266714 | −0.13327411 | 0.41054082 |
| ELANE | 0.113760757 | −0.29674864 | 0.41050939 |
| RNF24 | 0.273337871 | −0.13560884 | 0.40894671 |
| MYADM | 0.203808571 | −0.20445664 | 0.40826521 |
| SMARCD3 | 0.171004 | −0.23642889 | 0.40743289 |
| MS4A6A | 0.219724714 | −0.18766754 | 0.40739225 |
| LILRB3 | 0.219028571 | −0.18677839 | 0.40580696 |
| LPPR2 | 0.204843857 | −0.19941179 | 0.40425565 |
| FEN1 | −0.11046916 | 0.29375101 | 0.40422017 |
| TLR8 | 0.258494357 | −0.14398514 | 0.4024795 |
| GPR171 | 0.046738214 | 0.44807932 | 0.40134111 |
| PECAM1 | 0.166314929 | −0.23346854 | 0.39978346 |
| RENBP | 0.1793322 | −0.21994659 | 0.39927879 |
| IL18 | 0.094394 | −0.30436183 | 0.39875583 |
| CFD | 0.095564857 | −0.30247307 | 0.39803793 |
| ADAP2 | 0.191108 | −0.20639968 | 0.39750768 |
| PRRG4 | 0.234579614 | −0.16231581 | 0.39689543 |
| LMO2 | 0.316302143 | −0.08048718 | 0.39678932 |
| RPA2 | −0.15216443 | 0.24319079 | 0.39535521 |
| IMMP1L | −0.24381664 | 0.1514269 | 0.39524354 |
| RAB31 | 0.198269429 | −0.19655504 | 0.39482446 |
| MCTP1 | 0.189488857 | −0.20293439 | 0.39242325 |
| BLVRB | 0.186325571 | −0.20505411 | 0.39137968 |
| SLAMF1 | −0.05256957 | 0.33766054 | 0.39023011 |
| SRGN | 0.162264714 | −0.22679493 | 0.38905964 |
| KCTD12 | 0.256325714 | −0.13257457 | 0.38890029 |
| TIMP2 | 0.211432571 | −0.17675525 | 0.38818782 |
| EOMES | −0.15878157 | 0.22841004 | 0.38719161 |
| SLC7A7 | 0.159725571 | −0.22638171 | 0.38610729 |
| LILRA3 | 0.200592857 | −0.18353643 | 0.38412929 |
| SIGLEC15 | 0.251399786 | −0.13264211 | 0.38404189 |
| DRAM1 | 0.315899357 | −0.06714721 | 0.38304657 |
| CDCA3 | −0.09058453 | 0.29209835 | 0.38268288 |
| SULF2 | 0.153704714 | −0.22867929 | 0.382384 |
| LGALS3 | 0.190961214 | −0.19122154 | 0.38218275 |
| NLRP12 | 0.224418143 | −0.15755489 | 0.38197304 |
| CD33 | 0.155254 | −0.22667521 | 0.38192921 |
| ITPRIPL2 | 0.283875571 | −0.09768686 | 0.38156243 |
| CDC20 | −0.17666284 | 0.20478786 | 0.3814507 |
| TMEM127 | 0.144628571 | −0.23526511 | 0.37989368 |
| GIMAP7 | 0.036056571 | 0.41435014 | 0.37829357 |
| CPPED1 | 0.137228929 | −0.24017332 | 0.37740225 |
| FANCI | −0.16702414 | 0.21036312 | 0.37738726 |
| AKIRIN2 | 0.155994857 | −0.22050457 | 0.37649943 |
| S100A12 | 0.309248143 | −0.06721379 | 0.37646193 |
| STK39 | −0.11245929 | 0.26374314 | 0.37620243 |
| CLEC10A | 0.180977071 | −0.19424675 | 0.37522382 |
| CD68 | 0.146906 | −0.22801989 | 0.37492589 |
| E2F5 | 0.006467957 | 0.38078871 | 0.37432075 |
| CHST15 | 0.246691429 | −0.12753179 | 0.37422321 |
| ZNF668 | 0.118555714 | −0.25523732 | 0.37379304 |
| CLEC4A | 0.180520643 | −0.19312632 | 0.37364696 |
| OSCAR | 0.262092714 | −0.11077871 | 0.37287143 |
| CD28 | −0.21509714 | 0.15756741 | 0.37266456 |
| CSTA | 0.158350214 | −0.21139846 | 0.36974868 |
| TMEM64 | −0.2599964 | 0.10957631 | 0.36957271 |
| GLT25D1 | 0.16811 | −0.20127571 | 0.36938571 |
| LGALS2 | 0.183200857 | −0.18592343 | 0.36912429 |
| PFKFB4 | 0.160700929 | −0.20834925 | 0.36905018 |
| AMICA1 | 0.227419571 | −0.14141196 | 0.36883154 |
| STX10 | 0.202234786 | −0.16646264 | 0.36869743 |
| SYK | 0.196623286 | −0.1717975 | 0.36842079 |
| AGAP2 | −0.137432 | 0.23078254 | 0.36821454 |
| ACSL1 | 0.160613357 | −0.20691032 | 0.36752368 |
| RFC4 | −0.08400186 | 0.28309346 | 0.36709532 |
| IGSF6 | 0.225586 | −0.14143814 | 0.36702414 |
| CSF2RA | 0.2099765 | −0.15688386 | 0.36686036 |
| DAPK1 | 0.134109729 | −0.23158734 | 0.36569707 |
| CR2 | 0.116018943 | 0.48136556 | 0.36534662 |
| CARD9 | 0.140085929 | −0.22507043 | 0.36515636 |
| ZNF790 | −0.05887033 | 0.30589269 | 0.36476301 |
| NCF2 | 0.175950286 | −0.18565439 | 0.36160468 |
| MRFAP1L1 | −0.11274821 | 0.24734171 | 0.36008993 |
| AURKB | −0.3572037 | 0.00276418 | 0.35996788 |
| ANPEP | 0.270537786 | −0.08937875 | 0.35991654 |
| LTBR | 0.143410857 | −0.21426214 | 0.357673 |
| SLC15A3 | 0.143413786 | −0.21419907 | 0.35761286 |
| GNB4 | 0.182312143 | −0.17504436 | 0.3573565 |
| PRKAR1B | −0.13330653 | 0.2240327 | 0.35733923 |
| MEGF9 | 0.229724 | −0.1275625 | 0.3572865 |
| ABCD1 | 0.162001429 | −0.19449646 | 0.35649789 |
| S100A11 | 0.173434071 | −0.18187364 | 0.35530771 |
| PDGFC | 0.169980143 | −0.185277 | 0.35525714 |
| ATAD1 | −0.06876736 | 0.28555168 | 0.35431904 |
| SIGLEC1 | 0.323350029 | −0.03061627 | 0.3539663 |
| TNFSF13 | 0.118673786 | −0.2346925 | 0.35336629 |
| CD302 | 0.112564286 | −0.24046182 | 0.35302611 |
| ARHGEF10L | 0.140632286 | −0.211631 | 0.35226329 |
| HHEX | 0.216942429 | −0.1352445 | 0.35218693 |
| KRT23 | 0.182163071 | −0.16969303 | 0.3518561 |
| ESCO2 | 0.108076843 | 0.45909527 | 0.35101843 |
| RARRES3 | −0.19102486 | 0.15946211 | 0.35048696 |
| NCEH1 | 0.217908429 | −0.13163246 | 0.34954089 |
| ANO10 | 0.183057 | −0.16613929 | 0.34919629 |
| RAB32 | 0.160565929 | −0.18859543 | 0.34916136 |
| FCGRT | 0.128174071 | −0.22095846 | 0.34913254 |
| C3orf26 | −0.20833593 | 0.1404875 | 0.34882343 |
| ARAP3 | 0.172906214 | −0.17539179 | 0.348298 |
| TBXAS1 | 0.119282857 | −0.22890532 | 0.34818818 |
| FPR2 | 0.283766857 | −0.06392204 | 0.34768889 |
| AGTRAP | 0.174739214 | −0.17160118 | 0.34634039 |
| FGD4 | 0.153042571 | −0.1932215 | 0.34626407 |
| CCR7 | −0.11832114 | 0.22720743 | 0.34552857 |
| CSF1R | 0.110029357 | −0.23533893 | 0.34536829 |
| TTPAL | −0.09728186 | 0.24782729 | 0.34510914 |
| TLR7 | 0.252227071 | −0.09285861 | 0.34508568 |
| LILRA5 | 0.118514429 | −0.22476404 | 0.34327846 |
| KCNJ15 | 0.456193143 | 0.11300498 | 0.34318816 |
| HDDC2 | −0.19240571 | 0.15017121 | 0.34257693 |
| CENPE | −0.06459184 | 0.27777848 | 0.34237032 |
| DEPDC1B | 0.086165729 | 0.42821411 | 0.34204839 |
| F13A1 | 0.281931357 | −0.05990064 | 0.341832 |
| TKT | 0.178549571 | −0.16315625 | 0.34170582 |
| SIAE | 0.302025386 | −0.03965144 | 0.34167682 |
| PLA2G7 | 0.155596357 | −0.18373108 | 0.33932744 |
| HVCN1 | 0.196912286 | −0.142292 | 0.33920429 |
| VIM | 0.256285714 | −0.08262636 | 0.33891207 |
| SMYD3 | −0.23754843 | 0.10115771 | 0.33870614 |
| LILRB1 | 0.243770357 | −0.09430354 | 0.33807389 |
| SLC46A2 | 0.246823986 | −0.09115934 | 0.33798332 |
| CDC25A | −0.24464234 | 0.09325778 | 0.33790012 |
| LOC93622 | −0.19243486 | 0.14486786 | 0.33730271 |
| TFEC | 0.142966643 | −0.19367514 | 0.33664179 |
| BPI | 0.252416714 | −0.08360925 | 0.33602596 |
| MARCO | 0.262151 | −0.07320732 | 0.33535832 |
| PID1 | 0.163599786 | −0.17149768 | 0.33509746 |
| IKZF1 | −0.18795293 | 0.14708693 | 0.33503986 |
| GPATCH4 | −0.11773636 | 0.21681007 | 0.33454643 |
| BTN3A2 | −0.11306943 | 0.22099021 | 0.33405964 |
| LY9 | −0.14916079 | 0.18474139 | 0.33390218 |
| TTC7A | 0.158075857 | −0.17509714 | 0.333173 |
| SERPINA1 | 0.18943 | −0.14302168 | 0.33245168 |
| AKTIP | −0.10950264 | 0.22220111 | 0.33170375 |
| SPAG5 | −0.09952629 | 0.23141186 | 0.33093814 |
| IL1RN | 0.278950857 | −0.05174964 | 0.3307005 |
| ATG7 | 0.205422143 | −0.12514593 | 0.33056807 |
| PSAP | 0.121625143 | −0.20859004 | 0.33021518 |
| PGD | 0.230786857 | −0.09899668 | 0.32978354 |
| CFP | 0.128877286 | −0.20084218 | 0.32971946 |
| HK3 | 0.237489714 | −0.09144721 | 0.32893693 |
| SIGLEC7 | 0.126815571 | −0.20193039 | 0.32874596 |
| GRN | 0.207244143 | −0.120798 | 0.32804214 |
| QSOX1 | 0.183818214 | −0.14306671 | 0.32688493 |
| UBE2D1 | 0.176726357 | −0.14988214 | 0.3266085 |
| HEBP2 | 0.152510143 | −0.17333325 | 0.32584339 |
| BCL2A1 | 0.123345286 | −0.20224129 | 0.32558657 |
| BCKDK | 0.185639714 | −0.13980589 | 0.32544561 |
| MBOAT7 | 0.207825071 | −0.11713857 | 0.32496364 |
| ALOX5 | 0.187580429 | −0.13714918 | 0.32472961 |
| NLRC4 | 0.360432571 | 0.03675132 | 0.32368125 |
| PLD3 | 0.123464071 | −0.19904129 | 0.32250536 |
| HENMT1 | −0.11908886 | 0.20285339 | 0.32194225 |
| C20orf194 | 0.155664857 | −0.16459157 | 0.32025643 |
| NOD2 | 0.174258286 | −0.14543357 | 0.31969186 |
| QPCT | 0.17933 | −0.13977096 | 0.31910096 |
| MCM7 | −0.14002314 | 0.17880614 | 0.31882929 |
| PTPRK | 0.204003429 | 0.5226765 | 0.31867307 |
| RNASE2 | 0.272614786 | −0.04589457 | 0.31850936 |
| NPC2 | 0.116562286 | −0.20121646 | 0.31777875 |
| CD9 | 0.173752229 | −0.14344236 | 0.31719459 |
| BRI3 | 0.153947429 | −0.16324436 | 0.31719179 |
| TMEM55A | 0.1876985 | −0.12856771 | 0.31626621 |
| MPEG1 | 0.145838357 | −0.17023204 | 0.31607039 |
| TPCN1 | 0.124342714 | −0.19137939 | 0.31572211 |
| GAA | 0.162399571 | −0.15325996 | 0.31565954 |
| DICER1 | 0.234740429 | −0.08078946 | 0.31552989 |
| TTYH3 | 0.096881929 | −0.2184615 | 0.31534343 |
| RNF130 | 0.1476985 | −0.16759736 | 0.31529586 |
| SAMD13 | 0.469062286 | 0.1543228 | 0.31473949 |
| SLCO4C1 | −0.03990057 | 0.27477227 | 0.31467284 |
| CD14 | 0.206280286 | −0.10837093 | 0.31465121 |
| TTLL4 | 0.177558143 | −0.13611734 | 0.31367548 |
| FZD5 | 0.373442386 | 0.06099209 | 0.31245029 |
| MAP3K3 | 0.145047643 | −0.16736718 | 0.31241482 |
| CTSB | 0.134683429 | −0.17736096 | 0.31204439 |
| SMC2 | −0.12230134 | 0.18895004 | 0.31125139 |
| TMEM51 | 0.281504429 | −0.02969702 | 0.31120145 |
| IL21R | −0.10318271 | 0.20682311 | 0.31000582 |
| VNN2 | 0.238745286 | −0.07096643 | 0.30971171 |
| BST1 | 0.122712 | −0.18649204 | 0.30920404 |
| NCAPG2 | −0.1336442 | 0.17517352 | 0.30881772 |
| GPX1 | 0.194986 | −0.11377771 | 0.30876371 |
| KCNJ2 | 0.535975657 | 0.22722416 | 0.30875149 |
| FES | 0.202224429 | −0.10634068 | 0.30856511 |
| DPP4 | −0.09439186 | 0.21415932 | 0.30855118 |
| BTN3A1 | −0.11011443 | 0.19807043 | 0.30818486 |
| AZU1 | 0.223116043 | −0.0846731 | 0.30778914 |
| GMNN | −0.12069914 | 0.18704521 | 0.30774436 |
| GSN | 0.131577214 | −0.175545 | 0.30712221 |
| CPEB4 | 0.163479571 | −0.14039339 | 0.30387296 |
| IL12RB2 | −0.10310389 | 0.19992615 | 0.30303004 |
| AIF1 | 0.118723714 | −0.18273164 | 0.30145536 |
| APITD1 | −0.07477697 | 0.22439621 | 0.29917318 |
| BTK | 0.156705857 | −0.14215411 | 0.29885996 |
| NRM | 0.089331214 | −0.20934621 | 0.29867743 |
| MS4A1 | 0.079191429 | 0.37782723 | 0.2986358 |
| MFSD1 | 0.1568815 | −0.14147314 | 0.29835464 |
| REXO4 | −0.1100965 | 0.18783189 | 0.29792839 |
| SIGLEC5 | 0.191262986 | −0.10654275 | 0.29780574 |
| GRWD1 | −0.10044229 | 0.19704314 | 0.29748543 |
| TMEM176A | 0.119317543 | −0.17772454 | 0.29704208 |
| CCRL2 | 0.081377429 | −0.21558271 | 0.29696014 |
| EBF1 | 0.100721286 | 0.39671951 | 0.29599823 |
| BRWD3 | 0.1948784 | −0.1009656 | 0.295844 |
| ZWILCH | −0.10540566 | 0.18996072 | 0.29536638 |
| CDK2 | −0.12162653 | 0.1736169 | 0.29524343 |
| KLHL18 | 0.171266857 | −0.12381368 | 0.29508054 |
| ZNF367 | −0.00573039 | 0.28932663 | 0.29505701 |
| NFKBIA | 0.161213143 | −0.13376807 | 0.29498121 |
| DPYD | 0.156869357 | −0.138058 | 0.29492736 |
| ZFP36 | 0.175877571 | −0.11895879 | 0.29483636 |
| F5 | 0.250602129 | −0.04361346 | 0.29421559 |
| SMEK2 | −0.01661771 | 0.27744939 | 0.29406711 |
| GINS4 | −0.067823 | 0.22594522 | 0.29376822 |
| CD22 | 0.008552886 | 0.30209261 | 0.29353973 |
| SLC27A3 | 0.176830214 | −0.11502671 | 0.29185693 |
| CDC7 | 0.053590786 | 0.34504874 | 0.29145795 |
| GCA | 0.309009643 | 0.01764846 | 0.29136118 |
| SIRPB1 | 0.129623429 | −0.16148064 | 0.29110407 |
| CDC42BPB | 0.1311075 | −0.159489 | 0.2905965 |
| SPNS1 | 0.146933571 | −0.14342543 | 0.290359 |
| TSPAN15 | 0.066098514 | 0.35645661 | 0.29035809 |
| CST3 | 0.08667 | −0.20325982 | 0.28992982 |
| PLXNB2 | 0.120476429 | −0.16928714 | 0.28976357 |
| RASSF4 | 0.125179857 | −0.16421807 | 0.28939793 |
| LOC100288602 | −0.08719464 | 0.20071625 | 0.28791089 |
| PKIA | −0.07867879 | 0.20817289 | 0.28685168 |
| C9orf7 | 0.183859714 | −0.10165007 | 0.28550979 |
| TLR1 | 0.216556 | −0.06819975 | 0.28475575 |
| CEP76 | −0.05635327 | 0.22814923 | 0.2845025 |
| PRKCD | 0.195118357 | −0.08932918 | 0.28444754 |
| CD1A | 0.170681086 | −0.11343859 | 0.28411968 |
| PLEC | 0.205366286 | −0.07738589 | 0.28275218 |
| LILRA6 | 0.205201286 | −0.07564396 | 0.28084525 |
| RCCD1 | −0.128903 | 0.15155211 | 0.28045511 |
| DHFR | 0.008705214 | 0.28889754 | 0.28019233 |
| SLC31A2 | 0.201080214 | −0.07896014 | 0.28004036 |
| PRMT1 | −0.14745857 | 0.1323615 | 0.27982007 |
| NFAM1 | 0.149785286 | −0.12988161 | 0.27966689 |
| ADD3 | −0.10361586 | 0.17557793 | 0.27919379 |
| AOAH | 0.112956857 | −0.16581843 | 0.27877529 |
| TYROBP | 0.129111286 | −0.1495625 | 0.27867379 |
| LILRA2 | 0.083337857 | −0.19414179 | 0.27747964 |
| FGR | 0.139595571 | −0.13783404 | 0.27742961 |
| NCF1 | 0.188341643 | −0.08884914 | 0.27719079 |
| RACGAP1 | −0.04545993 | 0.23033114 | 0.27579106 |
| SGOL2 | −0.0736771 | 0.20052546 | 0.27420256 |
| GPR84 | 0.155422786 | −0.11842584 | 0.27384863 |
| FCRL1 | 0.0387364 | 0.31105258 | 0.27231618 |
| NINJ2 | 0.135472714 | −0.13671911 | 0.27219182 |
| MPP1 | 0.160403571 | −0.11111929 | 0.27152286 |
| FPR1 | 0.155853714 | −0.11522661 | 0.27108032 |
| PITPNB | −0.13456043 | 0.13234711 | 0.26690754 |
| RAD21 | −0.10346771 | 0.16341486 | 0.26688257 |
| GRINA | 0.075730143 | −0.18943504 | 0.26516518 |
| HDAC8 | 0.157113357 | −0.10713729 | 0.26425064 |
| GNG10 | 0.211676929 | −0.05257 | 0.26424693 |
| CD19 | 0.018627857 | 0.28259068 | 0.26396282 |
| EMILIN2 | 0.132286 | −0.13159482 | 0.26388082 |
| CCR2 | 0.4050525 | 0.14351464 | 0.26153786 |
| MTO1 | −0.03094193 | 0.23026036 | 0.26120229 |
| LILRB2 | 0.108566571 | −0.15243307 | 0.26099964 |
| DOK3 | 0.188393143 | −0.07252104 | 0.26091418 |
| ACTN1 | 0.223709071 | −0.03714046 | 0.26084954 |
| MKI67 | −0.02859244 | 0.23207986 | 0.2606723 |
| RANGAP1 | −0.12530014 | 0.13451096 | 0.25981111 |
| NPDC1 | −0.047562 | 0.21147393 | 0.25903593 |
| SPI1 | 0.122915286 | −0.13446354 | 0.25737882 |
| NEDD1 | −0.11064514 | 0.1462275 | 0.25687264 |
| PRIM2 | −0.03703164 | 0.21902236 | 0.256054 |
| HCK | 0.145662 | −0.11017818 | 0.25584018 |
| PRIM1 | −0.05954271 | 0.19627593 | 0.25581864 |
| FCN1 | 0.148124571 | −0.10727114 | 0.25539571 |
| TIMELESS | −0.05553264 | 0.1994745 | 0.25500714 |
| DSE | 0.174959714 | −0.07939089 | 0.25435061 |
| SAE1 | −0.14703571 | 0.10684029 | 0.253876 |
| S100A8 | 0.180040286 | −0.07339386 | 0.25343414 |
| TUBG1 | 0.043387857 | 0.29674596 | 0.25335811 |
| ATPAF2 | 0.141355 | −0.11012036 | 0.25147536 |
| ZNF131 | −0.14210886 | 0.10877468 | 0.25088354 |
| RPA3 | −0.04681671 | 0.20367254 | 0.25048925 |
| SIRPG | −0.07999457 | 0.17045821 | 0.25045279 |
| CKS1B | −0.17740757 | 0.07276836 | 0.25017593 |
| GNAQ | 0.160927429 | −0.08904675 | 0.24997418 |
| KIAA0125 | −0.01354736 | 0.23576654 | 0.24931389 |
| S100A9 | 0.215178714 | −0.03389743 | 0.24907614 |
| SP140 | −0.047052 | 0.20126089 | 0.24831289 |
| CDKN2A | −0.11248071 | 0.13441996 | 0.24690068 |
| HLA-DQB1 | 0.046151929 | −0.19956661 | 0.24571854 |
| BLK | −0.00402536 | 0.2414265 | 0.24545186 |
| PKMYT1 | −0.10169271 | 0.14326432 | 0.24495704 |
| TRAPPC5 | 0.121941143 | −0.12218889 | 0.24413004 |
| LAT2 | 0.145757571 | −0.09628264 | 0.24204021 |
| TMEM176B | 0.069299714 | −0.17272923 | 0.24202894 |
| ZYX | 0.168733429 | −0.07231025 | 0.24104368 |
| IFI30 | 0.144974 | −0.09603164 | 0.24100564 |
| CTSH | 0.080306286 | −0.16066829 | 0.24097457 |
| TNFSF13B | 0.105123786 | −0.1354275 | 0.24055129 |
| RDH14 | −0.09425121 | 0.14611864 | 0.24036986 |
| GBP3 | −0.06468743 | 0.17197089 | 0.23665832 |
| CEP72 | −0.05021723 | 0.18587307 | 0.2360903 |
| ECM1 | 0.243022929 | 0.00739127 | 0.23563166 |
| UMPS | −0.02724029 | 0.20625286 | 0.23349314 |
| P2RX5 | 0.043708071 | 0.27604918 | 0.23234111 |
| CTNNBIP1 | 0.055695071 | −0.17643986 | 0.23213493 |
| SMC4 | −0.0498615 | 0.18205457 | 0.23191607 |
| H2AFJ | 0.126527143 | −0.10458132 | 0.23110846 |
| ITGAM | 0.178884429 | −0.05205179 | 0.23093621 |
| RHOU | 0.091684 | −0.13749707 | 0.22918107 |
| LRRC8A | −0.15113114 | 0.07662004 | 0.22775118 |
| FAM105A | 0.051915571 | −0.17376321 | 0.22567879 |
| FAM185A | −0.1429 | 0.0825775 | 0.2254775 |
| RFC5 | −0.05649036 | 0.16896686 | 0.22545721 |
| TNFSF14 | 0.0590265 | 0.27745988 | 0.21843338 |
| CX3CR1 | 0.149541571 | 0.36685596 | 0.21731439 |
| PYCARD | 0.121423 | −0.09585457 | 0.21727757 |
| TMEM154 | 0.071312071 | −0.14592125 | 0.21723332 |
| SGOL1 | 0.177103671 | 0.3921782 | 0.21507453 |
| PARP9 | 0.024120429 | 0.23901993 | 0.2148995 |
| CTSD | 0.130801929 | −0.08273682 | 0.21353875 |
| DUSP22 | 0.117107714 | −0.09505114 | 0.21215886 |
| PTPN22 | −0.10790557 | 0.10260764 | 0.21051321 |
| AGPAT9 | 0.151721714 | −0.05847029 | 0.210192 |
| CLEC2D | −0.12874 | 0.080272 | 0.209012 |
| CDK14 | 0.174840186 | −0.03401502 | 0.20885521 |
| KNTC1 | −0.03739463 | 0.17103619 | 0.20843081 |
| PHKB | 0.134422429 | −0.07178939 | 0.20621182 |
| ESPL1 | −0.03134947 | 0.17278559 | 0.20413506 |
| HGSNAT | 0.083275071 | −0.12076807 | 0.20404314 |
| ERI2 | 0.238754243 | 0.03538946 | 0.20336479 |
| ENG | 0.069932214 | −0.13295171 | 0.20288393 |
| POLA1 | −0.0924215 | 0.10952729 | 0.20194879 |
| AP1S2 | 0.080564286 | −0.12097821 | 0.2015425 |
| ADAM12 | 0.063734771 | −0.13763672 | 0.20137149 |
| FCGR1B | 0.321847429 | 0.123407 | 0.19844043 |
| TIPIN | −0.073668 | 0.12286243 | 0.19653043 |
| NUSAP1 | −0.07885971 | 0.11708601 | 0.19594573 |
| TAF7 | −0.07773479 | 0.11804689 | 0.19578168 |
| GIMAP6 | 0.000750143 | 0.19616636 | 0.19541621 |
| DNAJA3 | −0.08410257 | 0.11076507 | 0.19486764 |
| PAK1 | 0.118213714 | −0.07653196 | 0.19474568 |
| ADH5 | −0.07254686 | 0.12209096 | 0.19463782 |
| CD79A | −0.04121671 | 0.15056154 | 0.19177825 |
| FCRL2 | 0.158976771 | 0.34783754 | 0.18886076 |
| UPP1 | 0.018764286 | −0.16593179 | 0.18469607 |
| SIGLEC16 | 0.136810429 | −0.04660093 | 0.18341136 |
| LOC729313 | −0.12535821 | 0.05648832 | 0.18184654 |
| TCL1A | 0.149912429 | 0.33119614 | 0.18128371 |
| RBL2 | −0.05727793 | 0.12376332 | 0.18104125 |
| RAN | −0.05334186 | 0.12015604 | 0.17349789 |
| ADRB2 | 0.045381429 | 0.21887268 | 0.17349125 |
| SLC16A3 | 0.166513429 | −0.00609168 | 0.17260511 |
| MYO1F | 0.139853143 | −0.03228971 | 0.17214286 |
| MYD88 | 0.127095 | −0.04501275 | 0.17210775 |
| SLC38A6 | 0.090774214 | −0.07870346 | 0.16947768 |
| POLA2 | −0.01999 | 0.14915151 | 0.16914151 |
| CYBB | 0.117763214 | −0.05079475 | 0.16855796 |
| WDR92 | 0.091065786 | 0.25956021 | 0.16849443 |
| SPIB | −0.00710193 | 0.16135964 | 0.16846157 |
| NUP88 | −0.05180564 | 0.11604057 | 0.16784621 |
| SERPING1 | 0.053220729 | −0.11385462 | 0.16707535 |
| SPINT2 | 0.075546857 | −0.09112346 | 0.16667032 |
| MSH6 | −0.06019686 | 0.1064115 | 0.16660836 |
| HESX1 | 0.287907157 | 0.12251361 | 0.16539355 |
| NCAPD3 | −0.06779679 | 0.09737246 | 0.16516925 |
| NCR1 | −0.1162164 | 0.04860299 | 0.16481939 |
| ATP8B4 | 0.183999614 | 0.02014505 | 0.16385456 |
| SFXN3 | 0.130900643 | −0.02609843 | 0.15699907 |
| VRK1 | −0.09116879 | 0.06370646 | 0.15487525 |
| GPR82 | 0.042014929 | 0.1932354 | 0.15122047 |
| NFE2 | 0.313747714 | 0.16882089 | 0.14492682 |
| PUS7 | −0.04881071 | 0.09542186 | 0.14423257 |
| E2F2 | 0.044565643 | 0.18640229 | 0.14183664 |
| TTF2 | 0.050887143 | 0.19187214 | 0.140985 |
| STIL | 0.087016229 | 0.22757289 | 0.14055666 |
| SAMD9L | 0.035826214 | 0.17281675 | 0.13699054 |
| NDUFA8 | −0.02098714 | 0.11569321 | 0.13668036 |
| POP5 | −0.08173779 | 0.05466404 | 0.13640182 |
| CEP152 | 0.105329543 | 0.23961047 | 0.13428093 |
| ANAPC10 | −0.04762379 | 0.08661393 | 0.13423771 |
| CD200 | 0.2610974 | 0.39524773 | 0.13415033 |
| LRFN4 | 0.0879955 | −0.04259586 | 0.13059136 |
| CDK4 | −0.07956357 | 0.05063243 | 0.130196 |
| SMC3 | −0.05600471 | 0.07299254 | 0.12899725 |
| RARA | 0.000606714 | −0.12788275 | 0.12848946 |
| CCR5 | 0.238368786 | 0.36464093 | 0.12627214 |
| SOD2 | 0.046425143 | −0.07868268 | 0.12510782 |
| IFIT1 | 0.165402214 | 0.04272914 | 0.12267307 |
| CKAP5 | 0.069255357 | 0.19171896 | 0.12246361 |
| MX2 | 0.111885786 | −0.00951561 | 0.12140139 |
| ARNTL2 | 0.217624257 | 0.09715989 | 0.12046437 |
| CCNF | 0.137269371 | 0.25590687 | 0.1186375 |
| CRTAM | 0.051424714 | 0.16918629 | 0.11776157 |
| SMARCAL1 | −0.02277143 | 0.09328157 | 0.116053 |
| PTGIR | 0.084466786 | −0.02833125 | 0.11279804 |
| MICB | −0.0206175 | 0.08935289 | 0.10997039 |
| CASP7 | −0.01341543 | 0.09496736 | 0.10838279 |
| ATR | 0.014273714 | 0.12143018 | 0.10715646 |
| TNFAIP6 | 0.250598143 | 0.14354166 | 0.10705648 |
| CXCR5 | −0.04794986 | 0.05755599 | 0.10550584 |
| EIF2B3 | −0.05847921 | 0.04699932 | 0.10547854 |
| TOPBP1 | 0.026353643 | 0.13146668 | 0.10511304 |
| JMJD4 | 0.000444143 | 0.10119504 | 0.10075089 |
| PLK1 | 0.4368879 | 0.34197855 | 0.09490935 |
| CSF2RB | −0.01162973 | −0.10319299 | 0.09156326 |
| AFF3 | 0.078227586 | −0.0107089 | 0.08893649 |
| HIBCH | 0.047619643 | 0.13565057 | 0.08803093 |
| SKP2 | 0.129403057 | 0.0431865 | 0.08621656 |
| MYO7B | 0.2198644 | 0.13441515 | 0.08544925 |
| CEBPE | 0.125281429 | 0.04241404 | 0.08286739 |
| RALGPS2 | 0.251390957 | 0.16886784 | 0.08252311 |
| MCM5 | −0.00196914 | 0.08003532 | 0.08200446 |
| IFIH1 | 0.13203 | 0.21265864 | 0.08062864 |
| PSEN2 | 0.025384786 | −0.05427521 | 0.07966 |
| SSBP1 | −0.02960171 | 0.04982696 | 0.07942868 |
| CHEK2 | −0.04968586 | 0.02740482 | 0.07709068 |
| TMPO | −0.008721 | 0.06823154 | 0.07695254 |
| CDC23 | 0.067964857 | 0.14406679 | 0.07610193 |
| TCHP | 0.135090829 | 0.20927789 | 0.07418706 |
| VPREB3 | 0.289311429 | 0.36304514 | 0.07373371 |
| CD72 | 0.013276714 | −0.05565957 | 0.06893629 |
| NPL | −0.06360071 | 0.004466 | 0.06806671 |
| DTYMK | 0.008803929 | 0.07686164 | 0.06805771 |
| TRAF3IP3 | 0.081071571 | 0.14877989 | 0.06770832 |
| CORO1A | −0.00652207 | 0.05883071 | 0.06535279 |
| GPR137B | 0.036305643 | −0.02776493 | 0.06407057 |
| POLD3 | 0.146014357 | 0.08214118 | 0.06387318 |
| FCRLA | 0.182630857 | 0.24622307 | 0.06359221 |
| ERCC6L | 0.302153757 | 0.24002681 | 0.06212694 |
| KIF23 | 0.091604929 | 0.03388473 | 0.0577202 |
| HLA-DOB | 0.097279429 | 0.15183752 | 0.05455809 |
| ALG8 | −0.02051879 | 0.03389046 | 0.05440925 |
| PSMA3 | −0.00942914 | 0.04492389 | 0.05435304 |
| CENPN | −0.15527079 | −0.10123914 | 0.05403164 |
| ACADM | 0.033363857 | 0.08598743 | 0.05262357 |
| RSAD2 | 0.133737286 | 0.1858205 | 0.05208321 |
| TSPAN13 | −0.05419761 | −0.00318473 | 0.05101289 |
| BRCA1 | 0.1411805 | 0.19119731 | 0.05001681 |
| ABCC3 | −0.07408826 | −0.12342445 | 0.04933619 |
| HLA-DOA | −0.06191443 | −0.014744 | 0.04717043 |
| VSIG10 | −0.03624206 | −0.07780171 | 0.04155966 |
| WHAMM | −0.05727821 | −0.01795696 | 0.03932125 |
| CEACAM1 | 0.031373857 | −0.00674968 | 0.03812354 |
| TFDP1 | 0.030716571 | 0.06752414 | 0.03680757 |
| KIF20B | 0.010597143 | 0.04736749 | 0.03677035 |
| FAM129C | 0.110318657 | 0.14503139 | 0.03471274 |
| MACROD2 | 0.0716402 | 0.10401612 | 0.03237592 |
| PPIL4 | 0.002354786 | 0.03106729 | 0.0287125 |
| CD24 | 0.428375214 | 0.40792088 | 0.02045434 |
| FAM129A | 0.113804357 | 0.09346804 | 0.02033632 |
| ZNF467 | −0.01551657 | 0.0041065 | 0.01962307 |
| ADAM28 | −0.0506592 | −0.07003481 | 0.01937561 |
| SAMHD1 | −0.0260325 | −0.01032382 | 0.01570868 |
| ECT2 | 0.099594771 | 0.08800196 | 0.01159281 |
| KMO | 0.078771714 | 0.09002297 | 0.01125126 |
| ASF1B | 0.069384957 | 0.0609988 | 0.00838616 |
| PSTPIP1 | 0.077608714 | 0.07232871 | 0.00528 |
| RMI1 | 0.229338371 | 0.23179671 | 0.00245834 |

### Study 2

The multi-antigenic CV9104 as described in WO2015024664 is an mRNA-based immunotherapeutic and a further development of CV9103 (Kübler H, et al. “Self-adjuvanted mRNA vaccination in advanced prostate cancer patients: a first-in-man phase I/IIa study.” J Immunother Cancer. 2015 Jun. 16; 3:26.). It encodes for six cancer antigens associated with prostate cancer: Muc-1, PSA, PSCA, PSMA, STEAP-1 and PAP. A total of 46 pre-operative prostate cancer patients were divided into three groups. Cohort A received up to four intradermal immunizations of 160 μg RNA for each antigen using the needle-free injection device Tropis. Patients in cohort B received 320 μg of RNA for each antigen by intradermal syringe-needle injection whereas cohort C patient remained untreated. Venous blood samples for gene expression profiling were taken at week 6 (1-2 weeks after the fourth mRNA treatment) and 8 weeks after prostatectomy. Blood was harvested into PAXgene® RNA tubes, frozen and stored at −80° C. until further processing. 14 subjects in cohort A, and 16 subjects each in cohorts B and C were evaluable.

Frozen PAXgene® samples were transferred to IMGM for isolation of RNA and microarray analysis. Blood samples were thawed and incubated at room temperature to ensure complete lysis of leukocytes before RNA isolation. Total RNA including miRNAs were isolated using the PAXgene® Blood miRNA Kit (Qiagen) according to the manufacturer's instructions including on-column DNAse digestion. Total RNA was eluted in 80 μl buffer.

RNA quantity and quality were assessed using the NanoDrop NK-1000 spectral photometer (peqlab) and integrity was analyzed on the 2100 Bioanalyzer using RNA 6000 Nano LabChip Kits (Agilent Technologies). Cyanine-3-labelling of cRNA and hybridization using the Agilent SurePrint G3 Human Gene Expression 8×60K v2 Microarray platform was performed by IMGM according to manufacturer's manuals (Agilent Technologies). Raw data was processed using following software tools: Feature Extraction 10.7.3.1 and GeneSpring GX 13.1.1 (both Agilent Techlogies). Quantile normalization was applied to the data set and log 2-transformed and filtered for probes detectable in at least one sample.

To determine the transcriptional changes in prostate cancer patients after CV9104 treatment we performed gene set enrichment analysis (GSEA) using blood transcriptional modules (BTMs) as gene sets (Li S et al. “Molecular signatures of antibody responses derived from a systems biology study of five human vaccines” Nat Immunol. 2014 February; 15(2):195-204). Gene lists were ranked based on paired t-test P values comparing the week 6 with baseline samples for each of the three patient cohorts. In addition, we also combined arms A and B (treated cohorts) to increase the statistical power and sensitivity of the GSEA approach. The GSEA results show that modules consistent with a T and NK cell profile were up-regulated at week 6 in subjects receiving CV9104 treatment but not in the control arm C (FIG. 1).

Gene Set Enrichment Analyses were performed comparing week 6 to week 1, and week 8 to week 1, for each of the two vaccine arms, and the control arm, individually, using the Blood Transcriptome Module gene sets as probes.

For each contrast, the 10 Blood Transcriptome Modules having the highest enrichment scores were selected, and the leading edge genes (the subset of genes that contribute most to the enrichment score) from each module were selected.

For this combined list of 725 unique leading edge genes, mean expression values for each experimental arm and time point were calculated. For transcripts that were represented by more than one probe in the microarray, the probe expression values of the probe having the highest mean expression in all samples was used.

Log 2 fold changes (differences in log 2 expression values) between week 6 and week 1, for each vaccine arm and the control arm were calculated.

To identify the genes that are regulated from week 1 to week 6 most differently in the vaccine recipients and the control group, the difference between the combined vaccine arm log 2 fold changes and the control arm log 2 fold changes was calculated (table 17). In table 17 the genes based on the absolute value of this difference, such that the genes at the top of the list are the most differently regulated between vaccine recipients and the control arm, whether those genes are up regulated to a greater extent over time, in vaccine recipients than in the control group, or down regulated to a greater extent over time, in vaccine recipients than in the control group.

### Overlapping Marker Genes of Study 1 and Study 2

Subsequently a subset of 402 of the 725 prostate cancer vaccine leading edge genes was selected, that were also found in the nsclc set of select genes (table 18).

This subset of select genes identified from analyses of both nsclc and prostate vaccine trials was ranked in the identical way as the total list of prostate vaccine leading edge genes, such that in table 18 the genes at the top of the list are the most differently regulated between prostate cancer vaccine recipients and the control arm, whether those genes are up regulated to a greater extent over time, in vaccine recipients than in the control group, or down regulated to a greater extent over time, in vaccine recipients than in the control group.

TABLE 17
difference between the combined vaccine arm log2 fold changes and the control
arm log2 fold changes; treatment with CV9104 (full name and accession number
as set out in Table 6).

| | | | | Differential | |
|---|---|---|---|---|---|
| | | | | Expression | Absolute value |
| Gene | | | | (armA + armB) − | ((armA + armB) − |
| Symbol | ArmA (A6v1) | ArmB (B6v1) | ArmC (C6v1) | armC | armC) |
| COX7B | −0.606408264 | −0.502341075 | 0.564692875 | −1.673442214 | 1.673442214 |
| LY96 | −0.665603021 | −0.467714725 | 0.450183881 | −1.583501628 | 1.583501628 |
| FIGNL1 | −0.720155553 | −0.143864925 | 0.655112416 | −1.519132894 | 1.519132894 |
| FOSL1 | 0.464989686 | 0.719168288 | −0.332139931 | 1.516297904 | 1.516297904 |
| S100A8 | −0.635815857 | −0.330874531 | 0.521352125 | −1.488042513 | 1.488042513 |
| GFRA3 | 0.44628215 | 0.135059706 | −0.759570281 | 1.340912138 | 1.340912138 |
| REEP1 | 0.665988507 | −0.0688409 | −0.742842819 | 1.339990426 | 1.339990426 |
| SUV39H2 | −0.106513621 | −0.968136388 | 0.264257213 | −1.338907221 | 1.338907221 |
| ZFYVE16 | −0.390657514 | −0.330864481 | 0.523502294 | −1.245024289 | 1.245024289 |
| NDUFA4 | −0.37000875 | −0.313184906 | 0.552544125 | −1.235737781 | 1.235737781 |
| FOXN1 | 0.558560786 | −0.034936269 | −0.643069656 | 1.166694173 | 1.166694173 |
| KIR2DL4 | 0.443792343 | 0.044371956 | −0.643219638 | 1.131383937 | 1.131383937 |
| GOLT1B | −0.282865464 | −0.210463281 | 0.6321674 | −1.125496146 | 1.125496146 |
| NLRC3 | 0.476656929 | 0.03283075 | −0.5611875 | 1.070675179 | 1.070675179 |
| ACTR6 | −0.310453271 | −0.124797413 | 0.624728813 | −1.059979496 | 1.059979496 |
| PLEKHF2 | −0.329232657 | −0.282916569 | 0.435252575 | −1.047401801 | 1.047401801 |
| TFEC | −0.318235514 | −0.242671238 | 0.483973406 | −1.044880158 | 1.044880158 |
| LGALS2 | −0.385623 | −0.130118344 | 0.527391187 | −1.043132531 | 1.043132531 |
| MSH6 | 0.261864307 | 0.025887787 | −0.732941019 | 1.020693113 | 1.020693113 |
| TARP | 0.378694321 | 0.076621125 | −0.561206219 | 1.016521665 | 1.016521665 |
| MND1 | −0.404708544 | −0.187105296 | 0.391868744 | −0.983682584 | 0.983682584 |
| S100A12 | −0.41744775 | −0.10775675 | 0.439791875 | −0.964996375 | 0.964996375 |
| AGL | 0.001249657 | −0.244289288 | 0.714159275 | −0.957198905 | 0.957198905 |
| NUPL2 | 0.145023043 | 0.174428356 | −0.636780513 | 0.956231912 | 0.956231912 |
| PKMYT1 | 0.142765721 | 0.224433575 | −0.588215069 | 0.955414365 | 0.955414365 |
| PTAFR | 0.379653786 | 0.146566969 | −0.422391625 | 0.948612379 | 0.948612379 |
| VSTM1 | −0.193381857 | −0.090299613 | 0.653519906 | −0.937201376 | 0.937201376 |
| SAMHD1 | 0.131552171 | −0.126445206 | −0.929604906 | 0.934711871 | 0.934711871 |
| ASPA | 0.5491103 | −0.275201025 | −0.646884206 | 0.920793481 | 0.920793481 |
| PPP1R12A | −0.234674457 | −0.430710625 | 0.246988487 | −0.91237357 | 0.91237357 |
| UQCRQ | −0.310938071 | −0.136146531 | 0.456713938 | −0.90379854 | 0.90379854 |
| OSMR | −0.321796071 | −0.129218388 | 0.450057575 | −0.901072034 | 0.901072034 |
| SP3 | −0.318489871 | −0.188125025 | 0.393770081 | −0.900384978 | 0.900384978 |
| JPH2 | 0.368767479 | 0.063154694 | −0.46770085 | 0.899623022 | 0.899623022 |
| APCDD1 | −0.273886843 | −0.087978469 | 0.529121788 | −0.890987099 | 0.890987099 |
| ABCG2 | 0.20306705 | 0.194013125 | −0.48904895 | 0.886129125 | 0.886129125 |
| TLR7 | −0.147615186 | −0.321601119 | 0.406793494 | −0.876009798 | 0.876009798 |
| ATP5C1 | −0.292455107 | −0.163626494 | 0.418519875 | −0.874601476 | 0.874601476 |
| GINS1 | −0.113533457 | −0.166327694 | 0.59447155 | −0.874332701 | 0.874332701 |
| CKS2 | −0.289032207 | −0.149659806 | 0.429285544 | −0.867977557 | 0.867977557 |
| NOC3L | −0.18972425 | −0.159537525 | 0.515942431 | −0.865204206 | 0.865204206 |
| DEK | −0.1939215 | −0.206601375 | 0.464482169 | −0.865005044 | 0.865005044 |
| HNMT | −0.295274607 | −0.333069056 | 0.23338665 | −0.861730313 | 0.861730313 |
| MS4A4A | −0.169447907 | −0.078548325 | 0.612627775 | −0.860624007 | 0.860624007 |
| TMEM55A | −0.253421071 | −0.191611125 | 0.41530585 | −0.860338046 | 0.860338046 |
| TMX1 | −0.122000464 | −0.269148656 | 0.468426 | −0.859575121 | 0.859575121 |
| USP1 | −0.251525429 | −0.143212106 | 0.460652213 | −0.855389747 | 0.855389747 |
| CCDC80 | 0.506134793 | −0.258483075 | −0.604203344 | 0.851855062 | 0.851855062 |
| PCNP | −0.236255293 | −0.19787625 | 0.412827844 | −0.846959387 | 0.846959387 |
| SHCBP1 | 0.042455016 | −0.179903435 | 0.709211203 | −0.846659622 | 0.846659622 |
| PANK3 | −0.165479279 | −0.210394325 | 0.466273875 | −0.842147479 | 0.842147479 |
| PAQR3 | 0.152645179 | −0.272095394 | 0.720825238 | −0.840275453 | 0.840275453 |
| RNF138 | −0.192648857 | −0.135220038 | 0.5105123 | −0.838381195 | 0.838381195 |
| CD302 | −0.218855893 | −0.262976719 | 0.352909463 | −0.834742074 | 0.834742074 |
| NUP35 | −0.173067586 | −0.047440819 | 0.608632838 | −0.829141242 | 0.829141242 |
| IMPA1 | −0.197582071 | −0.142449 | 0.484175444 | −0.824206515 | 0.824206515 |
| MS4A7 | −0.134199121 | −0.178824338 | 0.50987265 | −0.822896109 | 0.822896109 |
| SGMS1 | −0.136948443 | −0.118781288 | 0.559130738 | −0.814860468 | 0.814860468 |
| GALNT7 | −0.135803271 | −0.206088844 | 0.462692475 | −0.80458459 | 0.80458459 |
| CA14 | 0.260723136 | −0.096292 | −0.633484169 | 0.797915304 | 0.797915304 |
| RHOU | −0.231164307 | −0.186997156 | 0.375615037 | −0.793776501 | 0.793776501 |
| PHTF2 | −0.190424793 | −0.1227072 | 0.475077756 | −0.788209749 | 0.788209749 |
| S100A9 | −0.320247 | −0.153805438 | 0.307592 | −0.781644438 | 0.781644438 |
| FAM198B | −0.145417936 | −0.139593244 | 0.494443025 | −0.779454204 | 0.779454204 |
| ST3GAL6 | −0.237888643 | −0.076946094 | 0.457763138 | −0.772597874 | 0.772597874 |
| CLK4 | −0.141728729 | −0.188919619 | 0.441717688 | −0.772366035 | 0.772366035 |
| CLEC12A | −0.195821829 | −0.175847275 | 0.397432788 | −0.769101891 | 0.769101891 |
| GCA | −0.207991036 | −0.145952313 | 0.408222094 | −0.762165442 | 0.762165442 |
| VNN2 | −0.330711179 | −0.080868031 | 0.348413594 | −0.759992804 | 0.759992804 |
| C1GALT1C1 | −0.217019586 | −0.126876731 | 0.414085394 | −0.757981711 | 0.757981711 |
| DUSP6 | −0.247050464 | −0.126289281 | 0.379623406 | −0.752963152 | 0.752963152 |
| TNFAIP8 | −0.181705214 | −0.096119594 | 0.468258469 | −0.746083277 | 0.746083277 |
| TLR4 | −0.240381679 | −0.172451125 | 0.331507775 | −0.744340579 | 0.744340579 |
| FBLN1 | 0.1964213 | 0.051661288 | −0.49446335 | 0.742545938 | 0.742545938 |
| LRRK2 | −0.155146307 | −0.16335285 | 0.423976512 | −0.74247567 | 0.74247567 |
| SLC46A2 | −0.282207129 | −0.14557735 | 0.312664669 | −0.740449147 | 0.740449147 |
| KIAA1468 | −0.1790219 | −0.069215494 | 0.49012375 | −0.738361144 | 0.738361144 |
| AIF1 | −0.260561036 | 0.026826062 | 0.500316375 | −0.734051348 | 0.734051348 |
| HK3 | −0.234775807 | −0.097156719 | 0.401606381 | −0.733538907 | 0.733538907 |
| SFRP4 | −0.026588329 | −0.011432458 | 0.692197892 | −0.730218679 | 0.730218679 |
| ARFIP1 | −0.177901243 | −0.093895375 | 0.456699719 | −0.728496337 | 0.728496337 |
| COX6C | −0.219954821 | −0.05217725 | 0.449419019 | −0.72155109 | 0.72155109 |
| DMD | 0.217219014 | 0.187473034 | −0.314012238 | 0.718704286 | 0.718704286 |
| KYNU | −0.212530664 | −0.096688537 | 0.409357369 | −0.718576571 | 0.718576571 |
| PIK3C2A | −0.163213964 | −0.11146845 | 0.4358585 | −0.710540914 | 0.710540914 |
| NDUFB1 | −0.253689429 | −0.107063188 | 0.347131438 | −0.707884054 | 0.707884054 |
| VNN1 | −0.251333021 | −0.035195975 | 0.420730019 | −0.707259015 | 0.707259015 |
| MBNL1 | −0.15993675 | −0.120846844 | 0.424152594 | −0.704936188 | 0.704936188 |
| ABCA1 | −0.094451293 | −0.182310388 | 0.427960444 | −0.704722124 | 0.704722124 |
| PID1 | −0.2533088 | −0.008741875 | 0.435348662 | −0.697399337 | 0.697399337 |
| SRP9 | −0.242651436 | −0.045872794 | 0.405947088 | −0.694471317 | 0.694471317 |
| MAD2L1 | 0.115332107 | −0.108857088 | 0.699806038 | −0.693331018 | 0.693331018 |
| GLIPR1 | −0.2337439 | −0.054035794 | 0.403475625 | −0.691255319 | 0.691255319 |
| NUP107 | −0.134396293 | −0.066036206 | 0.488358206 | −0.688790705 | 0.688790705 |
| GZMK | −0.174657143 | −0.005958313 | 0.505133219 | −0.685748674 | 0.685748674 |
| STAG2 | −0.239123807 | −0.229557394 | 0.215309431 | −0.683990632 | 0.683990632 |
| NDUFB3 | −0.343863679 | −0.035564125 | 0.302523406 | −0.68195121 | 0.68195121 |
| CYP1B1 | −0.265192214 | −0.256149313 | 0.157784531 | −0.679126058 | 0.679126058 |
| MYBL2 | 0.270149364 | 0.164981162 | −0.243983306 | 0.679113833 | 0.679113833 |
| KLF1 | 0.363923679 | 0.009731187 | −0.305278563 | 0.678933429 | 0.678933429 |
| IFI30 | −0.188249714 | −0.067891938 | 0.422337406 | −0.678479058 | 0.678479058 |
| ICOS | −0.195708336 | −0.1017867 | 0.379751075 | −0.677246111 | 0.677246111 |
| TNFSF11 | 0.142212907 | 0.4536486 | −0.079978531 | 0.675840038 | 0.675840038 |
| NDUFB2 | −0.171665107 | −0.084994719 | 0.418124344 | −0.67478417 | 0.67478417 |
| TGM2 | 0.535738693 | 0.164614425 | 0.026084025 | 0.674269093 | 0.674269093 |
| CUL5 | −0.104067314 | −0.157484131 | 0.408924988 | −0.670476433 | 0.670476433 |
| CD28 | −0.208329314 | −0.103989013 | 0.357281481 | −0.669599808 | 0.669599808 |
| SGK1 | −0.211915821 | −0.061046906 | 0.394467688 | −0.667430415 | 0.667430415 |
| PLA2G7 | −0.180527121 | −0.099741525 | 0.381482469 | −0.661751115 | 0.661751115 |
| RMI1 | −0.199934986 | −0.04289555 | 0.413915156 | −0.656745692 | 0.656745692 |
| GPSM2 | −0.309434029 | 0.137679244 | 0.481535488 | −0.653290272 | 0.653290272 |
| ZDHHC17 | −0.0802091 | −0.095862544 | 0.471754831 | −0.647826475 | 0.647826475 |
| CCR2 | −0.152288 | −0.171936281 | 0.319978406 | −0.644202687 | 0.644202687 |
| FCN1 | −0.153257 | −0.084273781 | 0.405351719 | −0.6428825 | 0.6428825 |
| ZWINT | −0.07826045 | −0.311652 | 0.25231555 | −0.642228 | 0.642228 |
| AMICA1 | −0.193698571 | −0.108480063 | 0.336439188 | −0.638617821 | 0.638617821 |
| CLEC7A | −0.342173264 | −0.138714325 | 0.15760535 | −0.638492939 | 0.638492939 |
| ERBB2IP | −0.08476425 | −0.0988105 | 0.4519725 | −0.63554725 | 0.63554725 |
| MSH2 | −0.07557825 | −0.037042687 | 0.521162963 | −0.6337839 | 0.6337839 |
| RIN2 | −0.200246857 | −0.019231919 | 0.412999538 | −0.632478313 | 0.632478313 |
| MORC3 | −0.139277757 | −0.150871294 | 0.339561112 | −0.629710163 | 0.629710163 |
| MFSD1 | −0.141775179 | −0.013945156 | 0.467410156 | −0.623130491 | 0.623130491 |
| KCNJ2 | −0.114933607 | −0.185105 | 0.322908125 | −0.622946732 | 0.622946732 |
| LMNB1 | −0.200508029 | −0.200736762 | 0.220277363 | −0.621522154 | 0.621522154 |
| CCT2 | −0.125265893 | −0.03601025 | 0.457937938 | −0.61921408 | 0.61921408 |
| IFNG | −0.149614136 | −0.045758312 | 0.4236398 | −0.619012248 | 0.619012248 |
| EEF1G | −0.114996857 | −0.112516 | 0.390455125 | −0.617967982 | 0.617967982 |
| CSTA | −0.44455025 | 0.042660713 | 0.212461756 | −0.614351294 | 0.614351294 |
| MYBL1 | −0.114428693 | −0.076983169 | 0.422538056 | −0.613949918 | 0.613949918 |
| MNDA | −0.026950107 | 0.015162656 | 0.600877938 | −0.612665388 | 0.612665388 |
| PAQR9 | 0.64432315 | −0.0259158 | 0.006397187 | 0.612010163 | 0.612010163 |
| ZBTB11 | −0.167650657 | −0.232606775 | 0.211601269 | −0.611858701 | 0.611858701 |
| RBP7 | −0.21202525 | −0.083316812 | 0.315361469 | −0.610703531 | 0.610703531 |
| ZNF700 | −0.135721093 | 0.039511994 | 0.513713188 | −0.609922287 | 0.609922287 |
| BST1 | −0.140568821 | −0.056846906 | 0.4086275 | −0.606043228 | 0.606043228 |
| ATP5O | −0.170172643 | 0.013251563 | 0.446425125 | −0.603346205 | 0.603346205 |
| ATAD2 | −0.182130779 | −0.024369794 | 0.396607131 | −0.603107704 | 0.603107704 |
| DLGAP5 | −0.426307121 | 0.209794425 | 0.383806556 | −0.600319253 | 0.600319253 |
| NOD2 | −0.102332229 | −0.045682 | 0.451739 | −0.599753229 | 0.599753229 |
| LILRA3 | −0.15567315 | −0.014400075 | 0.423342813 | −0.593416037 | 0.593416037 |
| CLEC4A | −0.173481786 | −0.021148656 | 0.396113531 | −0.590743973 | 0.590743973 |
| TNFSF13B | −0.169967071 | −0.160481406 | 0.258797906 | −0.589246384 | 0.589246384 |
| CSF2RA | −0.132171093 | −0.0562483 | 0.400639056 | −0.589058449 | 0.589058449 |
| BRI3 | −0.103865857 | −0.058436312 | 0.425253469 | −0.587555638 | 0.587555638 |
| RNF6 | −0.109517936 | −0.087212163 | 0.390214419 | −0.586944517 | 0.586944517 |
| PTTG3P | 0.00596525 | −0.251395406 | 0.338894319 | −0.584324475 | 0.584324475 |
| HSPD1 | −0.147717536 | −0.001791687 | 0.434780906 | −0.584290129 | 0.584290129 |
| GTF2H1 | −0.187885957 | 0.078702969 | 0.472787963 | −0.581970951 | 0.581970951 |
| LMO2 | −0.169893857 | −0.058435719 | 0.351577969 | −0.579907545 | 0.579907545 |
| KIAA0101 | −0.041091293 | −0.16191575 | 0.375725275 | −0.578732318 | 0.578732318 |
| RFC5 | −0.098490279 | −0.093795681 | 0.385063719 | −0.577349679 | 0.577349679 |
| RAD50 | −0.140694379 | 0.0111879 | 0.447346706 | −0.576853185 | 0.576853185 |
| FEN1 | 0.084685657 | −0.025203925 | 0.636262238 | −0.576780505 | 0.576780505 |
| NUP54 | −0.11629505 | 0.019289113 | 0.479259756 | −0.576265694 | 0.576265694 |
| NDUFS4 | −0.264559636 | 0.068452419 | 0.377871181 | −0.573978398 | 0.573978398 |
| PTPN22 | −0.2530378 | −0.011789731 | 0.308368238 | −0.573195769 | 0.573195769 |
| CDKN3 | −0.444022514 | −0.092208262 | 0.035993937 | −0.572224714 | 0.572224714 |
| MAGEA12 | 0.289114343 | 0.171728456 | −0.109750269 | 0.570593068 | 0.570593068 |
| VPS4B | −0.048524464 | −0.153786 | 0.364946938 | −0.567257402 | 0.567257402 |
| EIF3E | −0.072937214 | 0.03722425 | 0.529808094 | −0.565521058 | 0.565521058 |
| HLA-DPB1 | −0.070855679 | −0.032144094 | 0.460644375 | −0.563644147 | 0.563644147 |
| NCAPG | 0.197792818 | 0.125748924 | −0.239994872 | 0.563536614 | 0.563536614 |
| CREBZF | −0.096819921 | 0.0781624 | 0.544589869 | −0.56324739 | 0.56324739 |
| ITSN2 | 0.132176686 | −0.003463494 | −0.434241769 | 0.562954961 | 0.562954961 |
| QPCT | −0.154081286 | 0.022183281 | 0.42981575 | −0.561713754 | 0.561713754 |
| BZW1 | −0.136054786 | −0.134614969 | 0.288626881 | −0.559296636 | 0.559296636 |
| NDC80 | −0.188322236 | 0.161280275 | 0.530858019 | −0.557899979 | 0.557899979 |
| ZFAND6 | −0.163411964 | −0.011106687 | 0.381657531 | −0.556176183 | 0.556176183 |
| KIR2DS2 | 0.164195243 | 0.152554619 | −0.23917585 | 0.555925712 | 0.555925712 |
| CCNC | −0.106666914 | −0.121181388 | 0.326487469 | −0.554335771 | 0.554335771 |
| EIF3H | −0.058134393 | −0.069187344 | 0.426115156 | −0.553436893 | 0.553436893 |
| FPR1 | −0.162620214 | 0.013841 | 0.404412125 | −0.553191339 | 0.553191339 |
| SLC40A1 | −0.174765179 | −0.084959656 | 0.2901025 | −0.549827335 | 0.549827335 |
| NUP160 | −0.023343321 | −0.093974831 | 0.432388269 | −0.549706421 | 0.549706421 |
| ENPP5 | 0.035325664 | 0.1957733 | 0.778261925 | −0.547162961 | 0.547162961 |
| MARCH1 | −0.175800229 | 0.011747531 | 0.381939919 | −0.545992616 | 0.545992616 |
| DPYD | −0.177738393 | −0.109984838 | 0.256353806 | −0.544077037 | 0.544077037 |
| TGFBI | −0.106554929 | −0.098723625 | 0.337612906 | −0.54289146 | 0.54289146 |
| FBXO5 | 0.010836921 | −0.242645981 | 0.310992263 | −0.542801322 | 0.542801322 |
| ATP5L | −0.144960714 | −0.094053063 | 0.301385187 | −0.540398964 | 0.540398964 |
| G3BP2 | −0.11234 | −0.005620688 | 0.419481844 | −0.537442531 | 0.537442531 |
| MARCKS | −0.104609857 | −0.143516469 | 0.287992844 | −0.53611917 | 0.53611917 |
| SEC63 | −0.137641714 | −0.015913837 | 0.381527888 | −0.535083439 | 0.535083439 |
| COX6A1 | −0.101929893 | −0.099744875 | 0.331444625 | −0.533119393 | 0.533119393 |
| PDGFA | 0.138137329 | 0.066844713 | −0.327700538 | 0.532682579 | 0.532682579 |
| CDT1 | 0.192584229 | 0.285041244 | −0.050867625 | 0.528493097 | 0.528493097 |
| KLRB1 | −0.158156529 | 0.037751856 | 0.40706125 | −0.527465922 | 0.527465922 |
| SMC2 | −0.257334636 | 0.049506637 | 0.315906338 | −0.523734336 | 0.523734336 |
| HLA-DMB | −0.1101265 | −0.04071075 | 0.3716505 | −0.52248775 | 0.52248775 |
| TYROBP | −0.094629357 | −0.082295813 | 0.345559625 | −0.522484795 | 0.522484795 |
| GLT8D2 | 0.019518921 | −0.049303425 | 0.491399144 | −0.521183647 | 0.521183647 |
| GMNN | −0.091846936 | −0.034380125 | 0.394157213 | −0.520384273 | 0.520384273 |
| FGR | −0.145875929 | −0.074496219 | 0.298854313 | −0.51922646 | 0.51922646 |
| COX5B | −0.0485035 | −0.072662125 | 0.396214906 | −0.517380531 | 0.517380531 |
| KIF18A | 0.356767059 | 0.233752233 | 0.074428434 | 0.516090859 | 0.516090859 |
| KIF20B | −0.096148593 | −0.117388763 | 0.300891063 | −0.514428418 | 0.514428418 |
| HOXD10 | 0.632420936 | 0.239668344 | 0.359192931 | 0.512896348 | 0.512896348 |
| RAD21 | −0.094918893 | −0.085931906 | 0.331705812 | −0.512556612 | 0.512556612 |
| MATR3 | −0.104278193 | −0.021778331 | 0.386447731 | −0.512504255 | 0.512504255 |
| APOBEC3B | −0.205192143 | −0.102170781 | 0.203892688 | −0.511255612 | 0.511255612 |
| HMOX1 | −0.128383179 | −0.123476031 | 0.258965875 | −0.510825085 | 0.510825085 |
| SLC31A2 | −0.181080679 | −0.067661719 | 0.261799219 | −0.510541616 | 0.510541616 |
| VNN3 | −0.206968286 | −0.096329569 | 0.201148394 | −0.504446248 | 0.504446248 |
| TSPAN5 | 0.244892929 | −0.06738175 | −0.323473044 | 0.500984222 | 0.500984222 |
| GATA1 | 0.292226893 | 0.104003775 | −0.102690375 | 0.498921043 | 0.498921043 |
| MARCO | −0.1259919 | 0.027786294 | 0.3990929 | −0.497298506 | 0.497298506 |
| SIRPB2 | −0.196183571 | 0.061583687 | 0.358098994 | −0.492698878 | 0.492698878 |
| LST1 | −0.149602786 | −0.06008475 | 0.281335125 | −0.491022661 | 0.491022661 |
| CEACAM7 | 0.306448264 | −0.125145288 | −0.309436544 | 0.490739521 | 0.490739521 |
| KIF20A | 0.233543341 | 0.129709219 | −0.127108786 | 0.490361346 | 0.490361346 |
| EED | −0.114456714 | 0.0091522 | 0.382570156 | −0.487874671 | 0.487874671 |
| RAB31 | −0.149398929 | −0.094196406 | 0.244100281 | −0.487695616 | 0.487695616 |
| NDUFB6 | −0.110910071 | −0.048859 | 0.327792125 | −0.487561196 | 0.487561196 |
| HSPE1 | −0.0697995 | −0.012676531 | 0.404518219 | −0.48699425 | 0.48699425 |
| TMEM154 | −0.173941464 | −0.048407781 | 0.262545125 | −0.484894371 | 0.484894371 |
| TLR1 | −0.097821071 | −0.081679344 | 0.305166437 | −0.484666853 | 0.484666853 |
| CD14 | −0.208618036 | −0.051658563 | 0.223004937 | −0.483281536 | 0.483281536 |
| SPC25 | 0.062161971 | 0.0350892 | 0.575205913 | −0.477954741 | 0.477954741 |
| ASGR1 | −0.1607095 | −0.008193156 | 0.307480156 | −0.476382812 | 0.476382812 |
| STK26 | −0.13457105 | −0.217412675 | 0.124148462 | −0.476132187 | 0.476132187 |
| EVI5 | −0.132586436 | 0.043848456 | 0.387096088 | −0.475834067 | 0.475834067 |
| SLC35B3 | 0.020614057 | −0.0244136 | 0.469842775 | −0.473642318 | 0.473642318 |
| FBXW2 | −0.132826129 | −0.051475106 | 0.2891366 | −0.473437835 | 0.473437835 |
| TLR6 | −0.060246393 | −0.074889556 | 0.33679665 | −0.471932599 | 0.471932599 |
| EMILIN2 | −0.184897107 | −0.021353875 | 0.264784219 | −0.471035201 | 0.471035201 |
| CCNB1 | −0.00534135 | 0.045066525 | −0.424402231 | 0.464127406 | 0.464127406 |
| HLA-DRB1 | −0.030511964 | −0.084263563 | 0.347531469 | −0.462306996 | 0.462306996 |
| GAS1 | 0.706006857 | −0.370432569 | −0.126699725 | 0.462274013 | 0.462274013 |
| RNASE2 | −0.126518993 | 0.061956337 | 0.396981 | −0.461543655 | 0.461543655 |
| CTSH | −0.080287786 | −0.022522656 | 0.357824813 | −0.460635254 | 0.460635254 |
| PILRA | −0.144449464 | −0.023444219 | 0.292670375 | −0.460564058 | 0.460564058 |
| S1PR3 | −0.013708679 | −0.113372613 | 0.332896331 | −0.459977622 | 0.459977622 |
| TNF | −0.09332625 | −0.146601688 | 0.219674388 | −0.459602325 | 0.459602325 |
| IL1RN | −0.148671 | −0.016940094 | 0.293804406 | −0.4594155 | 0.4594155 |
| CEP55 | −0.117062229 | 0.007737219 | 0.349900525 | −0.459225535 | 0.459225535 |
| ECT2 | 0.122074414 | −0.210264431 | 0.368810144 | −0.457000161 | 0.457000161 |
| AQP9 | −0.145872286 | −0.093673875 | 0.216232344 | −0.455778504 | 0.455778504 |
| NDUFAB1 | −0.040424964 | −0.031471563 | 0.382994375 | −0.454890902 | 0.454890902 |
| SLA | −0.000813893 | −0.049338625 | 0.404489031 | −0.454641549 | 0.454641549 |
| FES | −0.125205321 | −0.048358844 | 0.280610906 | −0.454175071 | 0.454175071 |
| HSPA6 | −0.096868036 | −0.0902495 | 0.266483469 | −0.453601004 | 0.453601004 |
| RNF130 | −0.043926929 | −0.130700938 | 0.278100375 | −0.452728241 | 0.452728241 |
| CEP120 | −0.070382471 | −0.424163256 | −0.041858012 | −0.452687715 | 0.452687715 |
| PNPLA8 | −0.131805071 | −0.098537 | 0.221471156 | −0.451813228 | 0.451813228 |
| HLA-DRB4 | −0.101285143 | −0.062042563 | 0.287547938 | −0.450875643 | 0.450875643 |
| ZBED5 | 0.160821464 | −0.1594155 | 0.450142688 | −0.448736723 | 0.448736723 |
| RANBP1 | −0.1626735 | 0.143935363 | 0.428623312 | −0.44736145 | 0.44736145 |
| MORF4L2 | −0.106883443 | −0.055327206 | 0.284950756 | −0.447161405 | 0.447161405 |
| PADI2 | −0.143229757 | −0.038802994 | 0.264994838 | −0.447027588 | 0.447027588 |
| TPX2 | 0.0290026 | 0.380640269 | −0.036550481 | 0.44619335 | 0.44619335 |
| NEK2 | 0.103340043 | 0.318715881 | −0.023429244 | 0.445485168 | 0.445485168 |
| EPB42 | 0.275517036 | 0.066022594 | −0.103539594 | 0.445079223 | 0.445079223 |
| ARMC1 | −0.10337225 | −0.046463075 | 0.290383863 | −0.440219188 | 0.440219188 |
| SMARCA5 | 0.07529565 | 0.081833119 | −0.281553706 | 0.438682475 | 0.438682475 |
| CSF1R | −0.024051321 | −0.119220813 | 0.2941795 | −0.437451634 | 0.437451634 |
| ATP5J2 | −0.151913893 | −0.044919719 | 0.239247969 | −0.43608158 | 0.43608158 |
| RPA3 | −0.212202643 | 0.118521156 | 0.341430438 | −0.435111924 | 0.435111924 |
| SMARCD3 | −0.160738571 | −0.034559813 | 0.237403562 | −0.432701946 | 0.432701946 |
| EIF3K | 0.006367607 | −0.050250937 | 0.384884031 | −0.428767362 | 0.428767362 |
| GPR56 | 0.248091393 | 0.253490812 | 0.073507031 | 0.428075174 | 0.428075174 |
| ATP5I | −0.226922214 | 0.147404594 | 0.348038938 | −0.427556558 | 0.427556558 |
| NIF3L1 | −0.026661943 | −0.056488794 | 0.341900894 | −0.42505163 | 0.42505163 |
| CD53 | −0.074409643 | 0.015283625 | 0.365545125 | −0.424671143 | 0.424671143 |
| MGST1 | −0.077665114 | 0.044218675 | 0.38925555 | −0.422701989 | 0.422701989 |
| COX7A2L | −0.025108571 | −0.088414375 | 0.308798 | −0.422320946 | 0.422320946 |
| FCGR1B | −0.244800429 | −0.064144188 | 0.112213438 | −0.421158054 | 0.421158054 |
| KCNE3 | −0.131369571 | −0.086003438 | 0.203527313 | −0.420900321 | 0.420900321 |
| XPO1 | −0.081257921 | 0.018815275 | 0.35681695 | −0.419259596 | 0.419259596 |
| PDE6D | −0.006646264 | −0.010540344 | 0.401125363 | −0.418311971 | 0.418311971 |
| MS4A6A | −0.125765393 | −0.039039156 | 0.253117937 | −0.417922487 | 0.417922487 |
| FBL | 0.014052786 | −0.040557875 | 0.3913615 | −0.417866589 | 0.417866589 |
| SIRPB1 | 0.001492071 | 0.268654588 | −0.145668094 | 0.415814753 | 0.415814753 |
| RAD51 | 0.091396443 | 0.185171756 | −0.138751738 | 0.415319937 | 0.415319937 |
| PLBD1 | −0.148141286 | −0.029220937 | 0.237610156 | −0.414972379 | 0.414972379 |
| LAT2 | −0.091685357 | −0.050819656 | 0.271164531 | −0.413669545 | 0.413669545 |
| LILRB3 | −0.114279071 | −0.103150719 | 0.19605325 | −0.41348304 | 0.41348304 |
| LILRA5 | −0.214061843 | −0.042955475 | 0.156297888 | −0.413315205 | 0.413315205 |
| PRIM1 | −0.063817571 | −0.033363275 | 0.315208994 | −0.41238984 | 0.41238984 |
| CFD | 0.093822357 | −0.059507156 | 0.446115375 | −0.411800174 | 0.411800174 |
| NCF2 | −0.095531464 | 0.057808937 | 0.373561219 | −0.411283746 | 0.411283746 |
| NDUFA2 | −0.068903929 | −0.064259906 | 0.278068969 | −0.411232804 | 0.411232804 |
| SMPDL3A | −0.042925271 | −0.039885175 | 0.32809285 | −0.410903296 | 0.410903296 |
| AGTRAP | −0.154270893 | 0.011986469 | 0.267882563 | −0.410166987 | 0.410166987 |
| GIMAP7 | −0.106066571 | 0.085459281 | 0.389390281 | −0.409997571 | 0.409997571 |
| IRAK3 | −0.055851193 | −0.049636381 | 0.302656369 | −0.408143943 | 0.408143943 |
| ASGR2 | −0.228676107 | 0.05324825 | 0.232312313 | −0.40774017 | 0.40774017 |
| UQCRC1 | −0.094910286 | −0.061424375 | 0.250938906 | −0.407273567 | 0.407273567 |
| CD36 | −0.087890679 | −0.026308156 | 0.29306775 | −0.407266585 | 0.407266585 |
| CD33 | −0.069894214 | −0.020831219 | 0.315814469 | −0.406539902 | 0.406539902 |
| PSAP | −0.107558786 | −0.003443781 | 0.294580281 | −0.405582848 | 0.405582848 |
| RAD51C | −0.068298621 | 0.081866631 | 0.418556406 | −0.404988396 | 0.404988396 |
| LCP1 | −0.095719357 | 0.037039969 | 0.345936844 | −0.404616232 | 0.404616232 |
| BCL6 | −0.116291893 | −0.126068313 | 0.162234313 | −0.404594518 | 0.404594518 |
| LILRA6 | −0.082732143 | −0.056291844 | 0.262668219 | −0.401692205 | 0.401692205 |
| CCT8 | −0.056735286 | 0.029709437 | 0.373685594 | −0.400711442 | 0.400711442 |
| ACSL1 | −0.072008321 | −0.141371812 | 0.18713075 | −0.400510884 | 0.400510884 |
| HCLS1 | −0.05346175 | −0.018238 | 0.328341781 | −0.400041531 | 0.400041531 |
| ITGA4 | −0.021362636 | 0.017255644 | 0.395887519 | −0.399994511 | 0.399994511 |
| SULF2 | −0.175644286 | 0.007419937 | 0.231142375 | −0.399366723 | 0.399366723 |
| CRTAM | 0.005827757 | 0.005227244 | 0.409946113 | −0.398891112 | 0.398891112 |
| CCT4 | 0.048661071 | 0.030773688 | 0.475969125 | −0.396534366 | 0.396534366 |
| CHRDL1 | −0.187312629 | 0.15926425 | −0.420098525 | 0.392050146 | 0.392050146 |
| GZMA | −0.119411321 | 0.071365812 | 0.343343812 | −0.391389321 | 0.391389321 |
| PGD | −0.075605786 | −0.079848531 | 0.233971188 | −0.389425504 | 0.389425504 |
| ARID4B | −0.098521307 | −0.038940956 | 0.251950206 | −0.38941247 | 0.38941247 |
| RFC4 | −0.064330557 | 0.042476944 | 0.367389475 | −0.389243088 | 0.389243088 |
| TNFRSF10B | −0.099273321 | −0.096856187 | 0.192978219 | −0.389107728 | 0.389107728 |
| POLD3 | −0.097467114 | 0.016328319 | 0.307658844 | −0.388797639 | 0.388797639 |
| UQCRFS1 | −0.020328964 | −0.001193125 | 0.366491781 | −0.388013871 | 0.388013871 |
| ROCK1 | −0.015296636 | −0.130577269 | 0.241015525 | −0.386889429 | 0.386889429 |
| NDUFS3 | −0.05276875 | 0.028971156 | 0.361374563 | −0.385172156 | 0.385172156 |
| CCT3 | 0.022924643 | 0.030373906 | 0.434754688 | −0.381456138 | 0.381456138 |
| HLA-DMA | −0.064727036 | −0.095813 | 0.220344813 | −0.380884848 | 0.380884848 |
| GP9 | 0.098050357 | 0.230333563 | −0.051893531 | 0.380277451 | 0.380277451 |
| MYD88 | −0.113476607 | 0.062618813 | 0.329212625 | −0.38007042 | 0.38007042 |
| SH2D1A | −0.150883521 | −0.100137519 | 0.128488456 | −0.379509496 | 0.379509496 |
| NCF1 | −0.124598929 | −0.031149125 | 0.22353825 | −0.379286304 | 0.379286304 |
| CD27 | 0.142986429 | −0.015922438 | −0.251951625 | 0.379015616 | 0.379015616 |
| SPRY4 | 0.37953555 | −0.069613044 | −0.067842113 | 0.377764619 | 0.377764619 |
| NLGN1 | −0.135739121 | −0.440554081 | −0.199182031 | −0.377111171 | 0.377111171 |
| HPSE | −0.276072121 | −0.044384475 | 0.056582837 | −0.377039434 | 0.377039434 |
| MED23 | −0.081364179 | 0.045862488 | 0.341019063 | −0.376520754 | 0.376520754 |
| UBE2T | −0.096130436 | −0.064383438 | 0.214767288 | −0.375281161 | 0.375281161 |
| LILRA2 | −0.109144071 | −0.061932719 | 0.201981906 | −0.373058696 | 0.373058696 |
| LEF1 | 0.05173875 | 0.016449531 | 0.440937813 | −0.372749531 | 0.372749531 |
| KNTC1 | 0.03019865 | 0.021303213 | 0.422944694 | −0.371442831 | 0.371442831 |
| ACPP | −0.052862964 | −0.038900388 | 0.278133725 | −0.369897077 | 0.369897077 |
| APOBEC3A | −0.091132643 | −0.059349375 | 0.218231219 | −0.368713237 | 0.368713237 |
| GOLPH3L | −0.116393657 | −0.058737531 | 0.193511413 | −0.368642601 | 0.368642601 |
| UQCRH | −0.174240321 | 0.067663719 | 0.261680406 | −0.368257009 | 0.368257009 |
| SLC7A7 | −0.075728964 | −0.03531975 | 0.256691094 | −0.367739808 | 0.367739808 |
| FGFBP2 | −0.026483621 | 0.044735331 | 0.385422 | −0.36717029 | 0.36717029 |
| TMPO | 0.056282186 | −0.05915685 | 0.362219844 | −0.365094508 | 0.365094508 |
| CEP76 | 0.057769386 | −0.11883195 | 0.304023344 | −0.365085908 | 0.365085908 |
| CPVL | −0.121587907 | −0.057256063 | 0.185360906 | −0.364204876 | 0.364204876 |
| IL10RA | −0.093252321 | 0.061976562 | 0.331903094 | −0.363178853 | 0.363178853 |
| TYMS | 0.215262371 | 0.229346038 | 0.081789275 | 0.362819134 | 0.362819134 |
| TSN | −0.051023429 | 0.004856975 | 0.315315175 | −0.361481629 | 0.361481629 |
| CDC25A | 0.203142321 | −0.116647119 | −0.274576838 | 0.36107204 | 0.36107204 |
| CKS1B | −0.063159979 | −0.033092825 | 0.264611125 | −0.360863929 | 0.360863929 |
| GPR171 | −0.129736571 | 0.101393125 | 0.331161438 | −0.359504884 | 0.359504884 |
| POLA2 | 0.030734921 | −0.042516512 | 0.345878394 | −0.357659985 | 0.357659985 |
| CLK1 | −0.101373336 | −0.065989581 | 0.190234075 | −0.357596992 | 0.357596992 |
| RAN | 0.0305255 | 0.0161995 | 0.403059781 | −0.356334781 | 0.356334781 |
| F5 | −0.191259607 | −0.063712219 | 0.100646831 | −0.355618657 | 0.355618657 |
| CD2 | 0.01264925 | 0.033470375 | 0.401702594 | −0.355582969 | 0.355582969 |
| NDUFA8 | −0.019786071 | 0.068549906 | 0.404028281 | −0.355264446 | 0.355264446 |
| RBM47 | −0.069904107 | 0.003304031 | 0.288030781 | −0.354630857 | 0.354630857 |
| CST3 | −0.126935536 | −0.025867938 | 0.201113344 | −0.353916817 | 0.353916817 |
| NDUFA11 | −0.016871929 | 0.005966313 | 0.342687969 | −0.353593585 | 0.353593585 |
| SKA3 | 0.100919436 | 0.177433125 | −0.073734256 | 0.352086817 | 0.352086817 |
| EIF3D | 0.020153786 | −0.065306187 | 0.306289719 | −0.351442121 | 0.351442121 |
| FHL2 | 0.000630921 | 0.214066981 | −0.135850413 | 0.350548315 | 0.350548315 |
| MAFB | 0.001780571 | −0.078602094 | 0.272882656 | −0.349704179 | 0.349704179 |
| TLK1 | −0.081011579 | 0.047498619 | 0.31585145 | −0.34936441 | 0.34936441 |
| CHAC2 | −0.109671707 | −0.180782856 | 0.058605019 | −0.349059582 | 0.349059582 |
| RAB21 | −0.080717893 | −0.034387687 | 0.233636156 | −0.348741737 | 0.348741737 |
| COX6B1 | −0.05439875 | −0.012977531 | 0.280276125 | −0.347652406 | 0.347652406 |
| GPR18 | −0.047635314 | −0.086936481 | 0.210504875 | −0.345076671 | 0.345076671 |
| SLAMF1 | −0.138963829 | −0.005397137 | 0.197378794 | −0.34173976 | 0.34173976 |
| NUP88 | −0.016508186 | −0.058129887 | 0.266949519 | −0.341587592 | 0.341587592 |
| UQCRC2 | −0.057371893 | 0.025039281 | 0.303439531 | −0.335772143 | 0.335772143 |
| BRCA1 | −0.155206629 | −0.018376169 | 0.161965494 | −0.335548291 | 0.335548291 |
| CYFIP1 | −0.060388986 | −0.015578656 | 0.258815819 | −0.334783461 | 0.334783461 |
| TMOD1 | 0.194032093 | 0.014683806 | −0.124742444 | 0.333458343 | 0.333458343 |
| TIPIN | 0.180150893 | −0.032065738 | 0.481311544 | −0.333226388 | 0.333226388 |
| COX8A | −0.017822929 | −0.005128219 | 0.309650406 | −0.332601554 | 0.332601554 |
| LILRB2 | −0.082981571 | −0.058156344 | 0.191195625 | −0.33233354 | 0.33233354 |
| RFC2 | −0.028812529 | −0.014747981 | 0.287979225 | −0.331539735 | 0.331539735 |
| IL13RA1 | −0.090803679 | 0.109964344 | 0.350655125 | −0.33149446 | 0.33149446 |
| CD300LF | −0.05083975 | −0.062153219 | 0.218185469 | −0.331178438 | 0.331178438 |
| NCF4 | −0.07311125 | −0.034869719 | 0.222047906 | −0.330028875 | 0.330028875 |
| RRM1 | −0.05435685 | −0.034887119 | 0.240450581 | −0.32969455 | 0.32969455 |
| ELAVL3 | 0.373095546 | −0.024622615 | 0.01926285 | 0.329210081 | 0.329210081 |
| LTBR | −0.160517679 | −0.091051437 | 0.0769038 | −0.328472916 | 0.328472916 |
| ALOX5 | −0.097675857 | −0.025537 | 0.203811688 | −0.327024545 | 0.327024545 |
| PLAUR | −0.143859321 | 0.005075062 | 0.186813438 | −0.325597696 | 0.325597696 |
| CRISPLD2 | −0.2434387 | −0.018830487 | 0.062991538 | −0.325260725 | 0.325260725 |
| PYGL | −0.116781321 | −0.027644562 | 0.180310781 | −0.324736665 | 0.324736665 |
| MDFIC | −0.222482193 | 0.115050188 | 0.217266475 | −0.32469848 | 0.32469848 |
| CFP | −0.121672286 | −0.045736219 | 0.156494219 | −0.323902723 | 0.323902723 |
| KIF2C | 0.215050936 | −0.030802981 | −0.138030888 | 0.322278842 | 0.322278842 |
| HCK | −0.058467464 | −0.040651875 | 0.223122531 | −0.322241871 | 0.322241871 |
| THEMIS | −0.030008079 | 0.077853244 | 0.369118469 | −0.321273304 | 0.321273304 |
| KIAA1598 | 0.226799843 | −0.125207913 | 0.422753275 | −0.321161345 | 0.321161345 |
| IL1R2 | −0.077493407 | 0.041097838 | 0.284015131 | −0.320410701 | 0.320410701 |
| FAM83D | −0.013678003 | −0.252206619 | 0.053864803 | −0.319749425 | 0.319749425 |
| CXCR2 | −0.097071286 | 0.001763562 | 0.224435781 | −0.319743504 | 0.319743504 |
| RTN1 | 0.086391936 | −0.013562106 | 0.391497244 | −0.318667414 | 0.318667414 |
| TIMP2 | −0.066331929 | 0.056842875 | 0.307963156 | −0.31745221 | 0.31745221 |
| GATA3 | −0.077782507 | 0.018922819 | 0.257456344 | −0.316316032 | 0.316316032 |
| UBA3 | −0.116247871 | 0.034844356 | 0.234209469 | −0.315612984 | 0.315612984 |
| PRRX1 | 0.21504705 | 0.1026264 | 0.002564387 | 0.315109063 | 0.315109063 |
| NLRP3 | −0.099970607 | 0.025493031 | 0.240302719 | −0.314780295 | 0.314780295 |
| UHRF1 | −0.065325229 | −0.332139738 | −0.711249206 | 0.31378424 | 0.31378424 |
| OIP5 | 0.082459536 | −0.051354256 | 0.344000944 | −0.312895664 | 0.312895664 |
| PAK1 | −0.078231214 | 0.004895375 | 0.239366437 | −0.312702277 | 0.312702277 |
| LY86 | −0.091854214 | −0.00419325 | 0.216384781 | −0.312432246 | 0.312432246 |
| KLRD1 | 0.136897079 | 0.334659263 | 0.159358556 | 0.312197785 | 0.312197785 |
| DTL | 0.002756621 | 0.090291919 | 0.404853569 | −0.311805029 | 0.311805029 |
| SIRPA | −0.091310143 | 0.054190688 | 0.274285937 | −0.311405393 | 0.311405393 |
| TRAT1 | −0.162242243 | 0.083363331 | 0.232039775 | −0.310918687 | 0.310918687 |
| RASSF4 | −0.075619321 | −0.01065925 | 0.222700469 | −0.30897904 | 0.30897904 |
| PECAM1 | −0.07363625 | −0.060431938 | 0.174908031 | −0.308976219 | 0.308976219 |
| RFC3 | 0.123554629 | 0.092185369 | −0.092420456 | 0.308160454 | 0.308160454 |
| ACTL6A | 0.052985686 | −0.039293506 | 0.321694169 | −0.308001989 | 0.308001989 |
| CHEK1 | −0.038283775 | 0.139021444 | 0.408487894 | −0.307750225 | 0.307750225 |
| BUB1 | 0.132609543 | 0.251248256 | 0.691503475 | −0.307645676 | 0.307645676 |
| PYCARD | −0.023505893 | 0.005845094 | 0.289019406 | −0.306680205 | 0.306680205 |
| EIF3F | −0.055520607 | 0.026444656 | 0.276166344 | −0.305242295 | 0.305242295 |
| ARHGEF10L | −0.103017607 | −0.023003438 | 0.178905688 | −0.304926732 | 0.304926732 |
| ITM2A | −0.040229429 | −0.003932375 | 0.260308 | −0.304469804 | 0.304469804 |
| LILRB1 | −0.039777321 | −0.074463563 | 0.187777313 | −0.302018196 | 0.302018196 |
| NUP37 | 0.023116864 | 0.00830125 | 0.333344706 | −0.301926592 | 0.301926592 |
| GIMAP6 | 0.04174575 | 0.040583437 | 0.383533563 | −0.301204375 | 0.301204375 |
| EVI2B | −0.095254571 | 0.010024438 | 0.215455906 | −0.30068604 | 0.30068604 |
| C1S | 0.258450671 | −0.256533794 | −0.298766269 | 0.300683146 | 0.300683146 |
| RASGRP1 | −0.014854864 | −0.03644825 | 0.248511125 | −0.299814239 | 0.299814239 |
| PRKAR2B | 0.073416286 | 0.265515313 | 0.039988094 | 0.298943504 | 0.298943504 |
| AGPAT9 | −0.1247355 | −0.055078937 | 0.11891075 | −0.298725187 | 0.298725187 |
| FPR2 | 0.1502825 | 0.290507219 | 0.147100688 | 0.293689031 | 0.293689031 |
| AURKB | −0.055779007 | −0.010595813 | 0.227200356 | −0.293575176 | 0.293575176 |
| TNFAIP2 | −0.040341571 | −0.026401312 | 0.226633688 | −0.293376571 | 0.293376571 |
| NUP62 | −0.053117464 | 0.056916312 | 0.297069625 | −0.293270777 | 0.293270777 |
| EIF3G | −0.042194 | 0.005646562 | 0.254368094 | −0.290915531 | 0.290915531 |
| GOPC | 0.317457793 | −0.152528806 | 0.455231825 | −0.290302838 | 0.290302838 |
| CCT7 | −0.003881321 | 0.017706781 | 0.3038845 | −0.29005904 | 0.29005904 |
| IMPA2 | 0.018998714 | 0.001092125 | 0.3094655 | −0.289374661 | 0.289374661 |
| UPF3B | −0.0048737 | 0.106697188 | 0.390858644 | −0.289035156 | 0.289035156 |
| SACM1L | −0.037368293 | 0.019169344 | 0.270208994 | −0.288407943 | 0.288407943 |
| CSF3R | −0.107332786 | −0.055135406 | 0.12043925 | −0.282907442 | 0.282907442 |
| PVRIG | 0.1293885 | 0.192957313 | 0.040053344 | 0.282292469 | 0.282292469 |
| RAD54L | 0.044659614 | 0.076583669 | 0.400908406 | −0.279665123 | 0.279665123 |
| ATP9A | −0.018721557 | 0.295013075 | −0.002296063 | 0.27858758 | 0.27858758 |
| NDUFB4 | 0.022882536 | 0.037768063 | 0.337617125 | −0.276966527 | 0.276966527 |
| HOMER3 | −0.161564357 | 0.025836844 | 0.139398125 | −0.275125638 | 0.275125638 |
| TNS3 | −0.056496 | 0.038417688 | 0.256140831 | −0.274219144 | 0.274219144 |
| NLRP12 | −0.12088475 | −0.031573812 | 0.121329781 | −0.273788344 | 0.273788344 |
| ADRB2 | 0.123512536 | 0.071676156 | 0.468501 | −0.273312308 | 0.273312308 |
| NUP43 | 0.016272214 | 0.011833656 | 0.300534375 | −0.272428504 | 0.272428504 |
| CCT6A | 0.028902071 | 0.000999344 | 0.301071531 | −0.271170116 | 0.271170116 |
| TLR2 | 0.061149464 | 0.135409938 | 0.466517625 | −0.269958223 | 0.269958223 |
| FAM72A | −0.163401771 | 0.030480644 | 0.135066594 | −0.267987721 | 0.267987721 |
| SERPINA1 | −0.140922143 | −0.065372844 | 0.061537 | −0.267831987 | 0.267831987 |
| SLC24A4 | −0.081812479 | −0.068289906 | 0.11610685 | −0.266209235 | 0.266209235 |
| GLT1D1 | −0.113676429 | −0.0532025 | 0.098683344 | −0.265562272 | 0.265562272 |
| HSD17B4 | −0.048726514 | 0.014324044 | 0.231119369 | −0.265521839 | 0.265521839 |
| UXT | 0.008390679 | −0.049655906 | 0.223751813 | −0.26501704 | 0.26501704 |
| CEBPA | −0.063082393 | −0.011602812 | 0.188959594 | −0.263644799 | 0.263644799 |
| FCGR2C | −0.031273143 | −0.036346781 | 0.195244844 | −0.262864768 | 0.262864768 |
| PTPRC | −0.122061857 | 0.008952469 | 0.149388813 | −0.262498201 | 0.262498201 |
| KLRK1 | −0.053411821 | 0.059479675 | 0.267907138 | −0.261839284 | 0.261839284 |
| CX3CR1 | 0.086424036 | 0.090047375 | 0.437198031 | −0.260726621 | 0.260726621 |
| LYL1 | 0.182472857 | 0.003204219 | −0.07453525 | 0.260212326 | 0.260212326 |
| C7 | 0.248340043 | −0.171803606 | −0.183461375 | 0.259997812 | 0.259997812 |
| BCL2 | −0.045814221 | 0.171313662 | 0.3841322 | −0.258632759 | 0.258632759 |
| MBOAT7 | −0.073752321 | −0.061278969 | 0.122161094 | −0.257192384 | 0.257192384 |
| KIR2DL5A | 0.050876864 | 0.149283706 | −0.0565809 | 0.256741471 | 0.256741471 |
| MELK | 0.164654093 | 0.035156056 | −0.055374975 | 0.255185124 | 0.255185124 |
| CTNNA1 | −0.021998464 | 0.054949294 | 0.287663938 | −0.254713108 | 0.254713108 |
| PLXNB2 | −0.080407464 | −0.076382 | 0.097792812 | −0.254582277 | 0.254582277 |
| SELL | −0.09905125 | −0.052091125 | 0.102181406 | −0.253323781 | 0.253323781 |
| CRYBB3 | 0.064264571 | 0.18436605 | −0.004553106 | 0.253183728 | 0.253183728 |
| KLRF1 | 0.035960571 | 0.04410145 | 0.332430812 | −0.252368791 | 0.252368791 |
| ANLN | 0.00399203 | −0.056580606 | 0.199374681 | −0.251963258 | 0.251963258 |
| NDUFC2 | 0.065413107 | 0.014681656 | 0.329968812 | −0.249874049 | 0.249874049 |
| PLK4 | 0.449380134 | −0.000561887 | 0.69820218 | −0.249383934 | 0.249383934 |
| DOK2 | 0.016806536 | −0.011930906 | 0.252665938 | −0.247790308 | 0.247790308 |
| DOK3 | −0.115201607 | −0.041282688 | 0.091301563 | −0.247785857 | 0.247785857 |
| TBCK | −0.014807386 | 0.130582063 | 0.362802656 | −0.247027979 | 0.247027979 |
| PRKCH | 0.071830929 | 0.062598125 | 0.381239719 | −0.246810665 | 0.246810665 |
| CYC1 | 0.019239321 | −0.069918187 | 0.195557469 | −0.246236335 | 0.246236335 |
| ABLIM1 | 0.060434464 | 0.064552875 | 0.370007188 | −0.245019848 | 0.245019848 |
| EMR2 | −0.054620571 | −0.0337915 | 0.154048406 | −0.242460478 | 0.242460478 |
| P2RY13 | −0.074662014 | −0.08374915 | 0.082303819 | −0.240714983 | 0.240714983 |
| KIR3DL1 | 0.118526179 | 0.170095588 | 0.049008837 | 0.239612929 | 0.239612929 |
| CD163 | −0.021365143 | 0.019498188 | 0.236402875 | −0.23826983 | 0.23826983 |
| CDCA7 | 0.040049543 | 0.049382125 | −0.148590044 | 0.238021712 | 0.238021712 |
| PDGFRA | 0.217143546 | 0.071091616 | 0.051219727 | 0.237015435 | 0.237015435 |
| CCL4L2 | 0.060749536 | 0.175954719 | 0.473478094 | −0.236773839 | 0.236773839 |
| HHEX | 0.027009136 | −0.042128188 | 0.221468875 | −0.236587927 | 0.236587927 |
| KLRC3 | 0.031305064 | 0.134056913 | 0.401069388 | −0.235707411 | 0.235707411 |
| NDUFS7 | −0.046897464 | 0.06665025 | 0.25466375 | −0.234910964 | 0.234910964 |
| GINS4 | 0.055271907 | 0.121248963 | 0.410527975 | −0.234007105 | 0.234007105 |
| PRAM1 | 0.024903857 | −0.060584344 | 0.198265156 | −0.233945643 | 0.233945643 |
| IFNGR2 | −0.123364643 | −0.045524969 | 0.063991406 | −0.232881018 | 0.232881018 |
| KIAA0513 | 0.000826321 | −0.049439281 | 0.184025656 | −0.232638616 | 0.232638616 |
| SPI1 | −0.095116929 | −0.09664825 | 0.040667094 | −0.232432272 | 0.232432272 |
| POC1A | −0.019389429 | −0.095124331 | 0.117207925 | −0.231721685 | 0.231721685 |
| DONSON | 0.338211479 | 0.065411256 | 0.171937125 | 0.23168561 | 0.23168561 |
| MEGF9 | −0.039111643 | −0.021554844 | 0.170999562 | −0.231666049 | 0.231666049 |
| CDC25C | 0.112939829 | 0.075938763 | −0.042544388 | 0.231422979 | 0.231422979 |
| PTGER2 | −0.042991157 | 0.128184388 | 0.315065313 | −0.229872082 | 0.229872082 |
| CDCA8 | −0.000913593 | 0.044746369 | 0.273661531 | −0.229828755 | 0.229828755 |
| UQCR10 | −0.03205 | 0.080985719 | 0.278651313 | −0.229715594 | 0.229715594 |
| COPB2 | 0.023108679 | 0.011450375 | 0.264066375 | −0.229507321 | 0.229507321 |
| ALDH2 | −0.079059607 | −0.023502094 | 0.126694219 | −0.22925592 | 0.22925592 |
| RNASE6 | −0.171393643 | 0.151863531 | 0.209186938 | −0.228717049 | 0.228717049 |
| STAT4 | 0.046057929 | 0.0061195 | 0.279347488 | −0.227170059 | 0.227170059 |
| SLC15A3 | −0.076069929 | −0.055751 | 0.094933844 | −0.226754772 | 0.226754772 |
| SLC35A5 | −0.041571021 | 0.049835756 | 0.234523494 | −0.226258759 | 0.226258759 |
| PLK1 | 1.73E−05 | 0.21058035 | −0.01565185 | 0.2262495 | 0.2262495 |
| SMC1A | 0.012533143 | 0.117303819 | −0.096245612 | 0.226082574 | 0.226082574 |
| CDK1 | −0.135575529 | 0.039025156 | 0.129455113 | −0.226005485 | 0.226005485 |
| FBP1 | −0.059616036 | −0.000195656 | 0.166176312 | −0.225988004 | 0.225988004 |
| CLIC3 | 0.165462929 | 0.149849875 | 0.089348594 | 0.22596421 | 0.22596421 |
| ETV1 | 0.012184231 | −0.240764519 | −0.451045356 | 0.222465069 | 0.222465069 |
| MYO1F | −0.059316857 | −0.018704438 | 0.144439406 | −0.222460701 | 0.222460701 |
| S100A11 | −0.133485 | −0.047393 | 0.040467813 | −0.221345813 | 0.221345813 |
| PIKFYVE | 0.104854321 | 0.100655869 | −0.015508438 | 0.221018628 | 0.221018628 |
| MYOF | 0.01329225 | 0.085496481 | 0.318671794 | −0.219883063 | 0.219883063 |
| AOAH | −0.039758964 | −0.016910688 | 0.162713031 | −0.219382683 | 0.219382683 |
| CD3D | −0.019660714 | −0.079698687 | 0.119301125 | −0.218660527 | 0.218660527 |
| TMTC2 | −0.056239136 | 0.078163744 | 0.238933619 | −0.217009011 | 0.217009011 |
| SUCLG1 | −0.065113321 | 0.069687812 | 0.221268938 | −0.216694446 | 0.216694446 |
| PTGDR | 0.163682371 | 0.1285211 | 0.075578575 | 0.216624896 | 0.216624896 |
| TREM1 | −0.2144935 | 0.009985344 | 0.011707031 | −0.216215188 | 0.216215188 |
| FCGRT | −0.071927429 | −0.021679125 | 0.122436219 | −0.216042772 | 0.216042772 |
| NUP133 | −0.077487443 | 0.048664469 | 0.185974988 | −0.214797962 | 0.214797962 |
| ACBD3 | −0.048152843 | 0.164027606 | 0.329671319 | −0.213796555 | 0.213796555 |
| CD8B | 0.062121 | 0.077815406 | 0.353468062 | −0.213531656 | 0.213531656 |
| HELLS | 0.086903171 | 0.019568225 | −0.1070155 | 0.213486896 | 0.213486896 |
| NFIA | 0.371265486 | −0.0424143 | 0.115813775 | 0.213037411 | 0.213037411 |
| PTPN11 | −0.093143579 | 0.070667075 | 0.189791044 | −0.212267547 | 0.212267547 |
| PRF1 | 0.142380979 | 0.187776544 | 0.118929719 | 0.211227804 | 0.211227804 |
| NCAM1 | 0.186510829 | 0.170383288 | 0.146273838 | 0.210620279 | 0.210620279 |
| CYBB | −0.091119571 | −0.020434094 | −0.322162281 | 0.210608616 | 0.210608616 |
| SYK | −0.06395725 | 0.002388875 | 0.148010469 | −0.209578844 | 0.209578844 |
| TBXAS1 | −0.073986893 | −0.048228219 | 0.086518344 | −0.208733455 | 0.208733455 |
| ZAP70 | −0.017364357 | 0.013869219 | 0.203887688 | −0.207382826 | 0.207382826 |
| SEMA4A | −0.197552929 | 0.03085925 | 0.040039219 | −0.206732897 | 0.206732897 |
| LYN | −0.036844786 | −0.003103938 | 0.165543937 | −0.205492661 | 0.205492661 |
| TRIM10 | 0.153861014 | 0.004640925 | −0.046917244 | 0.205419183 | 0.205419183 |
| HJURP | −0.013993607 | 0.179469288 | 0.039795713 | 0.205271393 | 0.205271393 |
| PRKCD | −0.007103429 | −0.019443625 | 0.17715875 | −0.203705804 | 0.203705804 |
| KIF11 | 0.3559893 | −0.363408594 | 0.195700206 | −0.2031195 | 0.2031195 |
| RHOH | 0.038042607 | 0.058467281 | 0.29882175 | −0.202311862 | 0.202311862 |
| GRN | 0.1997915 | 0.269006406 | 0.266844781 | 0.201953125 | 0.201953125 |
| TTC1 | −0.045822357 | 0.04050645 | 0.196106169 | −0.201422076 | 0.201422076 |
| ITGAM | −0.034672786 | 0.009557375 | 0.174503438 | −0.199618848 | 0.199618848 |
| RXRA | −0.034103821 | −0.026557688 | 0.138651594 | −0.199313103 | 0.199313103 |
| NUP85 | 0.030187679 | 0.021720875 | 0.250158788 | −0.198250234 | 0.198250234 |
| XCL1 | −0.040837821 | −0.030924194 | 0.12604125 | −0.197803265 | 0.197803265 |
| BTK | −0.246700964 | 0.115514375 | 0.066183563 | −0.197370152 | 0.197370152 |
| KIR2DS4 | 0.109764193 | 0.151970006 | 0.065144675 | 0.196589524 | 0.196589524 |
| GINS2 | 0.086710507 | 0.03973685 | 0.321476863 | −0.195029505 | 0.195029505 |
| CARD11 | 0.086571436 | 0.038937688 | 0.320536975 | −0.195027852 | 0.195027852 |
| NUP205 | 0.007000286 | 0.043720156 | 0.244397563 | −0.193677121 | 0.193677121 |
| COX5A | 0.001266714 | 0.073535125 | 0.267407969 | −0.192606129 | 0.192606129 |
| IL12RB2 | 0.116243957 | 0.168973447 | 0.475805088 | −0.190587683 | 0.190587683 |
| ITGB2 | −0.092971857 | 0.032449906 | 0.128161219 | −0.18868317 | 0.18868317 |
| FXYD6 | −0.132427886 | −0.032194075 | 0.020960069 | −0.185582029 | 0.185582029 |
| NKG7 | 0.100092 | 0.177019062 | 0.091530313 | 0.18558075 | 0.18558075 |
| CLEC12B | 0.126618243 | −0.216706594 | −0.274877219 | 0.184788868 | 0.184788868 |
| TBX21 | 0.147042607 | 0.171793313 | 0.134367281 | 0.184468638 | 0.184468638 |
| TMEM106C | −0.072996871 | 0.096723306 | 0.207120563 | −0.183394128 | 0.183394128 |
| EOMES | 0.031210564 | 0.180122613 | 0.028667006 | 0.182666171 | 0.182666171 |
| CPPED1 | −0.075764786 | −0.006007437 | 0.097081875 | −0.178854098 | 0.178854098 |
| HEPACAM2 | 0.30810525 | −0.328715588 | 0.15575645 | −0.176366788 | 0.176366788 |
| BMI1 | −0.03228375 | 0.041806344 | 0.185530888 | −0.176008294 | 0.176008294 |
| CST7 | 0.042171821 | −0.157493094 | 0.059774281 | −0.175095554 | 0.175095554 |
| KIR2DL2 | 0.150170293 | 0.048071569 | 0.023736862 | 0.174504999 | 0.174504999 |
| PROS1 | −0.141067664 | −0.0105115 | 0.0210835 | −0.172662664 | 0.172662664 |
| SLC28A1 | 0.059450621 | 0.037232388 | 0.268637725 | −0.171954716 | 0.171954716 |
| STEAP4 | −0.024713186 | −0.025752356 | 0.119735125 | −0.170200667 | 0.170200667 |
| RORA | −0.005687036 | 0.04953275 | 0.213705344 | −0.169859629 | 0.169859629 |
| MATK | 0.128941 | −0.057783469 | −0.097076062 | 0.168233594 | 0.168233594 |
| TCF7 | 0.015533071 | 0.119273375 | 0.302479313 | −0.167672866 | 0.167672866 |
| MCTP1 | −0.064457314 | 0.02050475 | 0.121421475 | −0.165374039 | 0.165374039 |
| CCNB2 | 0.11113635 | 0.160589037 | 0.106655813 | 0.165069575 | 0.165069575 |
| CCL5 | 0.082592786 | 0.179442438 | 0.097006 | 0.165029223 | 0.165029223 |
| TRIP13 | −0.151740693 | 0.029123638 | −0.287642194 | 0.165025138 | 0.165025138 |
| TNFSF12 | −0.048036679 | 0.070770719 | 0.186750219 | −0.164016179 | 0.164016179 |
| PDGFRB | 0.171366921 | 0.026544106 | 0.034163281 | 0.163747746 | 0.163747746 |
| NDUFB11 | −0.040507464 | −0.013961031 | 0.107839563 | −0.162308058 | 0.162308058 |
| ELP4 | 0.000880343 | 0.100837619 | 0.263235831 | −0.16151787 | 0.16151787 |
| KLRC1 | 0.000687164 | 0.099160981 | 0.261111831 | −0.161263686 | 0.161263686 |
| PMS1 | 0.09187805 | 0.017723269 | 0.269440381 | −0.159839063 | 0.159839063 |
| SCPEP1 | −0.0993085 | 0.080756812 | 0.139985219 | −0.158536906 | 0.158536906 |
| SP140 | −0.097582571 | 0.103564562 | 0.164278719 | −0.158296728 | 0.158296728 |
| POLA1 | 0.052668236 | 0.040824187 | 0.251305694 | −0.157813271 | 0.157813271 |
| FASLG | 0.085012064 | 0.136574256 | 0.378932025 | −0.157345704 | 0.157345704 |
| SLC35A3 | 0.324811771 | 0.145015194 | 0.626809575 | −0.15698261 | 0.15698261 |
| SORT1 | −0.009813957 | 0.002481187 | 0.147676013 | −0.155008782 | 0.155008782 |
| ABCA8 | 0.031660065 | 0.053897269 | 0.240461319 | −0.154903985 | 0.154903985 |
| DTYMK | −0.04659875 | 0.132572344 | 0.239921625 | −0.153948031 | 0.153948031 |
| NOP56 | −0.000568964 | 0.084934719 | 0.237542219 | −0.153176464 | 0.153176464 |
| SMARCAL1 | −0.086243036 | 0.100209938 | 0.166715688 | −0.152748786 | 0.152748786 |
| PLEK | 0.019009071 | −0.065560094 | 0.10602225 | −0.152573272 | 0.152573272 |
| SEH1L | 0.03765995 | 0.080064425 | 0.269638244 | −0.151913869 | 0.151913869 |
| ITK | −0.015928207 | 0.14679825 | 0.281890887 | −0.151020845 | 0.151020845 |
| NFAM1 | −0.027323071 | −0.0392185 | 0.084381437 | −0.150923009 | 0.150923009 |
| ITPR3 | 0.374672979 | −0.044524775 | 0.179360838 | 0.150787366 | 0.150787366 |
| TNFRSF18 | 0.028010107 | 0.041735181 | 0.218599825 | −0.148854537 | 0.148854537 |
| FCAR | −0.066841164 | −0.027840525 | 0.053792012 | −0.148473702 | 0.148473702 |
| E2F7 | −0.405322936 | 0.215555531 | −0.041365931 | −0.148401473 | 0.148401473 |
| KCNQ1 | −0.048365643 | 0.064700375 | 0.162571312 | −0.14623658 | 0.14623658 |
| CENPW | −0.164889786 | 0.230038812 | 0.210302344 | −0.145153317 | 0.145153317 |
| SIGLEC7 | −0.024430871 | −0.088715794 | 0.031245875 | −0.14439254 | 0.14439254 |
| PCNA | 0.035651979 | 0.039530006 | 0.219408269 | −0.144226284 | 0.144226284 |
| ANO10 | −0.039469393 | 0.011942625 | 0.11532075 | −0.142847518 | 0.142847518 |
| PTPRE | −0.076416679 | 0.046165188 | 0.110726156 | −0.140977647 | 0.140977647 |
| ABCC13 | 0.198030307 | −0.048670069 | 0.008429625 | 0.140930613 | 0.140930613 |
| TSNAX | −0.041252929 | 0.119107719 | 0.215539006 | −0.137684216 | 0.137684216 |
| SSBP1 | 0.043657036 | 0.076313656 | 0.256911875 | −0.136941183 | 0.136941183 |
| TGFBR3 | 0.057640943 | 0.225603875 | 0.146874275 | 0.136370543 | 0.136370543 |
| CENPE | 0.094657979 | 0.11519455 | 0.345300175 | −0.135447646 | 0.135447646 |
| NDUFS8 | −0.009986071 | 0.024954469 | 0.149320406 | −0.134352009 | 0.134352009 |
| CARD9 | 0.000456214 | −0.024868219 | 0.109741531 | −0.134153536 | 0.134153536 |
| CHAF1B | 0.015375293 | 0.113135369 | 0.260426356 | −0.131915695 | 0.131915695 |
| IL6R | −0.0899715 | 0.080235 | −0.141129344 | 0.131392844 | 0.131392844 |
| CD8A | 0.062538893 | 0.115027688 | 0.307421594 | −0.129855013 | 0.129855013 |
| CDCA5 | 0.141994621 | 0.0153348 | 0.0285846 | 0.128744821 | 0.128744821 |
| CEP72 | 0.121346421 | 0.049939025 | 0.299817094 | −0.128531647 | 0.128531647 |
| KIAA1109 | −0.036829514 | 0.057112206 | 0.148373894 | −0.128091202 | 0.128091202 |
| H2AFX | 0.016909343 | 0.107348406 | −0.003697669 | 0.127955418 | 0.127955418 |
| HOPX | 0.112690921 | 0.061422119 | 0.301787156 | −0.127674116 | 0.127674116 |
| ARL4C | 0.072803357 | 0.103496344 | 0.302879469 | −0.126579768 | 0.126579768 |
| TLR8 | −0.045178079 | −0.045833031 | 0.032884969 | −0.123896079 | 0.123896079 |
| SLC16A3 | −0.165960286 | −0.097487344 | −0.140923594 | −0.122524036 | 0.122524036 |
| CD96 | −0.016845807 | 0.130848437 | 0.234244275 | −0.120241645 | 0.120241645 |
| FMR1 | −0.016440529 | −0.004763262 | 0.098062581 | −0.119266372 | 0.119266372 |
| CAMK4 | 0.17398735 | −0.051962625 | 0.241167213 | −0.119142488 | 0.119142488 |
| CENPF | 0.071971921 | −0.075413106 | 0.115432769 | −0.118873954 | 0.118873954 |
| ANP32E | −0.073683321 | 0.110316563 | 0.1554933 | −0.118860059 | 0.118860059 |
| LCK | 0.079214964 | 0.091696875 | 0.287152063 | −0.116240223 | 0.116240223 |
| KCTD12 | −0.10831425 | −0.100223344 | −0.093382125 | −0.115155469 | 0.115155469 |
| CASC5 | −0.194317893 | 0.338253019 | 0.258946669 | −0.115011543 | 0.115011543 |
| SGOL2 | 0.218390882 | −0.199590273 | 0.133709203 | −0.114908593 | 0.114908593 |
| TNFRSF1B | −0.074531214 | 0.014785844 | 0.054734187 | −0.114479558 | 0.114479558 |
| GZMM | 0.085528179 | 0.056466125 | 0.027774781 | 0.114219522 | 0.114219522 |
| IL7R | 0.072157107 | 0.148358125 | 0.334539719 | −0.114024487 | 0.114024487 |
| MCM6 | 0.042624929 | 0.199344263 | 0.35289755 | −0.110928359 | 0.110928359 |
| CD1D | −0.013206666 | 0.246479216 | 0.343240125 | −0.109967576 | 0.109967576 |
| ASPM | −0.312100279 | 0.056704519 | −0.147088063 | −0.108307697 | 0.108307697 |
| PAX2 | 0.161487071 | −0.055965913 | −0.001837237 | 0.107358396 | 0.107358396 |
| ZMYM2 | 0.129444679 | 0.026701812 | 0.263450963 | −0.107304471 | 0.107304471 |
| IL2RB | 0.247864286 | 0.082890125 | 0.22417725 | 0.106577161 | 0.106577161 |
| PTTG1 | 0.098365936 | 0.086615375 | 0.079258663 | 0.105722648 | 0.105722648 |
| FANCI | 0.073934264 | 0.125870263 | 0.096870381 | 0.102934146 | 0.102934146 |
| PCSK6 | 0.0278519 | 0.096634169 | 0.022053987 | 0.102432081 | 0.102432081 |
| GZMH | 0.072301857 | 0.187933406 | 0.36239375 | −0.102158487 | 0.102158487 |
| SLC11A1 | −0.089359857 | −0.104323438 | −0.092454281 | −0.101229013 | 0.101229013 |
| ETS1 | 0.015150521 | 0.148691106 | 0.063176088 | 0.10066554 | 0.10066554 |
| CKAP5 | 0.047345043 | 0.056420319 | 0.203781756 | −0.100016395 | 0.100016395 |
| WDFY3 | −0.003150579 | 0.035841919 | 0.132396669 | −0.099705329 | 0.099705329 |
| IL18R1 | 0.052565443 | 0.131353344 | 0.282953375 | −0.099034588 | 0.099034588 |
| HAVCR2 | −0.009178479 | 0.108920556 | 0.194893488 | −0.09515141 | 0.09515141 |
| SH3BGRL2 | −0.050841007 | 0.158029269 | 0.201938813 | −0.094750551 | 0.094750551 |
| CENPN | −0.16359285 | 0.087772819 | 0.017766275 | −0.093586306 | 0.093586306 |
| ATF2 | 0.010376893 | 0.0675732 | 0.170737869 | −0.092787776 | 0.092787776 |
| EXOC1 | 0.294340857 | 0.212963188 | 0.414737406 | 0.092566638 | 0.092566638 |
| KIR2DS3 | 0.170303736 | −0.057887187 | 0.204776925 | −0.092360377 | 0.092360377 |
| TUBG1 | 0.1275628 | 0.057753981 | 0.094358969 | 0.090957812 | 0.090957812 |
| NUF2 | 0.079239486 | 0.052017034 | 0.220492578 | −0.089236059 | 0.089236059 |
| SMC4 | −0.049519036 | 0.068988038 | 0.106945031 | −0.087476029 | 0.087476029 |
| NUSAP1 | −0.207199657 | 0.195487413 | 0.07446825 | −0.086180495 | 0.086180495 |
| CHST15 | 0.035716286 | −0.081956625 | 0.03941675 | −0.085657089 | 0.085657089 |
| PTPN4 | 0.183461529 | 0.179189413 | 0.277530538 | 0.085120404 | 0.085120404 |
| TACC3 | −0.083857321 | 0.0461285 | 0.044973144 | −0.082701965 | 0.082701965 |
| FGD4 | −0.131588607 | 0.087883312 | 0.03755975 | −0.081265045 | 0.081265045 |
| PLXND1 | −0.0189865 | 0.11060225 | 0.171687562 | −0.080071813 | 0.080071813 |
| FCGR2A | −0.032769571 | −0.020548844 | 0.025650156 | −0.078968571 | 0.078968571 |
| CCR7 | 0.115547179 | 0.108481656 | 0.145899094 | 0.078129741 | 0.078129741 |
| FANCD2 | 0.037001443 | 0.003119969 | 0.117773888 | −0.077652476 | 0.077652476 |
| S1PR5 | 0.087563907 | 0.209525425 | 0.221772069 | 0.075317263 | 0.075317263 |
| HMBS | 0.231740036 | −0.055886406 | 0.101409062 | 0.074444567 | 0.074444567 |
| UBE2S | 0.01034575 | 0.041629813 | −0.020495281 | 0.072470844 | 0.072470844 |
| CD247 | 0.114385179 | 0.142090938 | 0.18457025 | 0.071905866 | 0.071905866 |
| FOXM1 | 0.204105021 | −0.232078488 | −0.098508462 | 0.070534996 | 0.070534996 |
| NUP155 | 0.144700671 | −0.031175944 | 0.182827806 | −0.069303079 | 0.069303079 |
| CD7 | 0.012400536 | −0.038204844 | −0.093956906 | 0.068152598 | 0.068152598 |
| RAB32 | −0.064779993 | 0.057404756 | 0.060534719 | −0.067909955 | 0.067909955 |
| PLEKHF1 | 0.147747779 | 0.083882938 | 0.164267781 | 0.067362935 | 0.067362935 |
| EXO1 | 0.12917555 | −0.015257425 | 0.178355519 | −0.064437394 | 0.064437394 |
| NNMT | 0.233886154 | −0.064754751 | 0.233446688 | −0.064315284 | 0.064315284 |
| CTSW | 0.086388214 | 0.086623606 | 0.109152531 | 0.063859289 | 0.063859289 |
| TNFSF13 | −0.104992364 | 0.204233313 | 0.162872469 | −0.063631521 | 0.063631521 |
| SKA1 | 0.172363579 | −0.101240878 | 0.007584162 | 0.063538538 | 0.063538538 |
| TXK | 0.167692714 | 0.13788895 | 0.24493265 | 0.060649014 | 0.060649014 |
| CDCA2 | 0.131150257 | 0.08530005 | 0.276015065 | −0.059564758 | 0.059564758 |
| DHFR | 0.0808114 | −0.078742712 | 0.061597675 | −0.059528988 | 0.059528988 |
| MCM4 | 0.310664586 | −0.021865731 | 0.229669113 | 0.059129742 | 0.059129742 |
| NPL | −0.028528464 | 0.0650085 | −0.022373125 | 0.058853161 | 0.058853161 |
| PRC1 | −0.00396665 | −0.0044553 | −0.066523444 | 0.058101494 | 0.058101494 |
| ATIC | 0.18636285 | 0.088420625 | 0.217445369 | 0.057338106 | 0.057338106 |
| AFF2 | −0.089054274 | −0.124852861 | −0.271109782 | 0.057202648 | 0.057202648 |
| SLA2 | −0.007006543 | 0.103122831 | 0.152447188 | −0.056330899 | 0.056330899 |
| SH2D2A | 0.053755679 | 0.084245688 | 0.081803031 | 0.056198335 | 0.056198335 |
| NCAPD3 | 0.15018295 | 0.019467175 | 0.113947944 | 0.055702181 | 0.055702181 |
| GPR114 | 0.048830007 | 0.178102206 | 0.172434275 | 0.054497938 | 0.054497938 |
| DPP4 | 0.010671021 | 0.171490388 | 0.235689063 | −0.053527654 | 0.053527654 |
| LAT | −0.009395143 | 0.149325594 | 0.191682219 | −0.051751768 | 0.051751768 |
| CLINT1 | 0.024371071 | 0.09246525 | 0.16695325 | −0.050116929 | 0.050116929 |
| SPOCK2 | 0.0540895 | 0.116730031 | 0.220384125 | −0.049564594 | 0.049564594 |
| TIGIT | −0.002881429 | 0.110435856 | 0.154252106 | −0.046697679 | 0.046697679 |
| CXCR3 | 0.054416679 | 0.173273125 | 0.181010313 | 0.046679491 | 0.046679491 |
| CYBRD1 | 0.040529114 | −0.119726244 | −0.03273975 | −0.046457379 | 0.046457379 |
| TMEM71 | 0.093268964 | −0.009283281 | 0.130187875 | −0.046202192 | 0.046202192 |
| MCM5 | 0.015791714 | −0.022235375 | 0.038842312 | −0.045285973 | 0.045285973 |
| TCHP | 0.03122065 | 0.102604531 | 0.17764235 | −0.043817169 | 0.043817169 |
| SIRT1 | 0.072246879 | 0.282646475 | 0.396445619 | −0.041552265 | 0.041552265 |
| ECM2 | 0.06183995 | −0.034499769 | −0.013579256 | 0.040919438 | 0.040919438 |
| NACC2 | −0.040991 | 0.051050375 | 0.050223031 | −0.040163656 | 0.040163656 |
| CD5 | 0.013059036 | 0.051298562 | 0.101524563 | −0.037166964 | 0.037166964 |
| PBX1 | −0.146203364 | 0.1381656 | −0.044659887 | 0.036622123 | 0.036622123 |
| MPEG1 | 0.228906821 | 0.122075031 | 0.317887406 | 0.033094446 | 0.033094446 |
| STMN1 | 0.152387964 | 0.054988531 | 0.175320975 | 0.032055521 | 0.032055521 |
| SLAMF7 | 0.09940525 | 0.078786469 | 0.208776125 | −0.030584406 | 0.030584406 |
| CLEC4E | 0.03307095 | 0.250178863 | 0.253125025 | 0.030124787 | 0.030124787 |
| ITGAX | −0.003191536 | 0.03580425 | 0.058384406 | −0.025771692 | 0.025771692 |
| UBASH3A | 0.078104014 | 0.0524895 | 0.155564163 | −0.024970648 | 0.024970648 |
| TTC7B | −0.042112171 | 0.233417081 | 0.167371531 | 0.023933379 | 0.023933379 |
| SPAG5 | 0.113789793 | 0.087098931 | 0.177345094 | 0.02354363 | 0.02354363 |
| IL21R | 0.074056021 | 0.027803787 | 0.120988831 | −0.019129022 | 0.019129022 |
| GNLY | 0.021276571 | 0.16815425 | 0.205857406 | −0.016426585 | 0.016426585 |
| PRKCQ | 0.009845486 | 0.135340319 | 0.161210463 | −0.016024658 | 0.016024658 |
| KIF23 | −0.096498793 | 0.083153288 | −0.028673256 | 0.015327751 | 0.015327751 |
| NDUFA3 | 0.079680821 | 0.028160906 | 0.0970595 | 0.010782228 | 0.010782228 |
| F13A1 | −0.047338571 | 0.205989906 | 0.148638469 | 0.010012866 | 0.010012866 |
| SAMD3 | 0.161056436 | 0.096928381 | 0.266791975 | −0.008807158 | 0.008807158 |
| IKZF1 | 0.065568071 | 0.061111063 | 0.133848906 | −0.007169772 | 0.007169772 |
| RARRES3 | 0.097648143 | 0.035314469 | 0.128298781 | 0.00466383 | 0.00466383 |
| GZMB | 0.0733865 | 0.237917812 | 0.315513938 | −0.004209625 | 0.004209625 |
| EZH2 | 0.039164079 | 0.015619287 | 0.055791712 | −0.001008346 | 0.001008346 |

TABLE 18
Subset of select genes identified from analyses
of both nsclc and prostate vaccine trials

| | Week5_0 | Week5_0 | |
|---|---|---|---|
| | GE difference_ | GE difference_ | absolute value |
| Gene | mean_pat_ | mean_pat_ | ((armA + |
| symbol | clust1 | clust2 | armB) − armC) |
| LY96 | 0.25291329 | −0.1675694 | 1.58350163 |
| FIGNL1 | −0.3911252 | 0.07060053 | 1.51913289 |
| S100A8 | 0.18004029 | −0.0733939 | 1.48804251 |
| NLRC3 | −0.3230981 | 0.37479546 | 1.07067518 |
| TFEC | 0.14296664 | −0.1936751 | 1.04488016 |
| LGALS2 | 0.18320086 | −0.1859234 | 1.04313253 |
| MSH6 | −0.0601969 | 0.1064115 | 1.02069311 |
| TARP | −0.3253611 | 0.43529539 | 1.01652167 |
| MND1 | −0.239129 | 0.51650061 | 0.98368258 |
| S100A12 | 0.30924814 | −0.0672138 | 0.96499638 |
| PKMYT1 | −0.1016927 | 0.14326432 | 0.95541437 |
| PTAFR | 0.36795829 | −0.0962296 | 0.94861238 |
| VSTM1 | 0.22415614 | −0.292179 | 0.93720138 |
| SAMHD1 | −0.0260325 | −0.0103238 | 0.93471187 |
| APCDD1 | 0.5716404 | −0.0894154 | 0.8909871 |
| TLR7 | 0.25222707 | −0.0928586 | 0.8760098 |
| HNMT | 0.23255914 | −0.3267737 | 0.86173031 |
| MS4A4A | 0.33475521 | −0.0776784 | 0.86062401 |
| TMEM55A | 0.1876985 | −0.1285677 | 0.86033805 |
| SHCBP1 | −0.1809977 | 0.24105691 | 0.84665962 |
| CD302 | 0.11256429 | −0.2404618 | 0.83474207 |
| RHOU | 0.091684 | −0.1374971 | 0.7937765 |
| S100A9 | 0.21517871 | −0.0338974 | 0.78164444 |
| FAM198B | 0.39951821 | −0.1879804 | 0.7794542 |
| ST3GAL6 | 0.454685 | −0.2018707 | 0.77259787 |
| CLEC12A | 0.20369814 | −0.2397915 | 0.76910189 |
| GCA | 0.30900964 | 0.01764846 | 0.76216544 |
| VNN2 | 0.23874529 | −0.0709664 | 0.7599928 |
| DUSP6 | 0.40522829 | −0.0708414 | 0.75296315 |
| TLR4 | 0.283682 | −0.2026975 | 0.74434058 |
| LRRK2 | 0.17933043 | −0.2534506 | 0.74247567 |
| SLC46A2 | 0.24682399 | −0.0911593 | 0.74044915 |
| AIF1 | 0.11872371 | −0.1827316 | 0.73405135 |
| HK3 | 0.23748971 | −0.0914472 | 0.73353891 |
| VNN1 | 0.24903221 | −0.1813005 | 0.70725902 |
| PID1 | 0.16359979 | −0.1714977 | 0.69739934 |
| MAD2L1 | −0.1968512 | 0.22556866 | 0.69333102 |
| GZMK | −0.2942172 | 0.25558446 | 0.68574867 |
| CYP1B1 | 0.32722971 | −0.2386703 | 0.67912606 |
| MYBL2 | −0.2431724 | 0.17455584 | 0.67911383 |
| IFI30 | 0.144974 | −0.0960316 | 0.67847906 |
| ICOS | −0.1726607 | 0.33378096 | 0.67724611 |
| CD28 | −0.2150971 | 0.15756741 | 0.66959981 |
| SGK1 | 0.31619786 | −0.4520401 | 0.66743042 |
| PLA2G7 | 0.15559636 | −0.1837311 | 0.66175112 |
| RMI1 | 0.22933837 | 0.23179671 | 0.65674569 |
| CCR2 | 0.4050525 | 0.14351464 | 0.64420269 |
| FCN1 | 0.14812457 | −0.1072711 | 0.6428825 |
| ZWINT | −0.2281671 | 0.37430662 | 0.642228 |
| AMICA1 | 0.22741957 | −0.141412 | 0.63861782 |
| CLEC7A | 0.17440871 | −0.3200379 | 0.63849294 |
| MSH2 | −0.117764 | 0.31093821 | 0.6337839 |
| MFSD1 | 0.1568815 | −0.1414731 | 0.62313049 |
| KCNJ2 | 0.53597566 | 0.22722416 | 0.62294673 |
| IFNG | −0.4260917 | 0.47086009 | 0.61901225 |
| CSTA | 0.15835021 | −0.2113985 | 0.61435129 |
| MYBL1 | −0.3237831 | 0.35550657 | 0.61394992 |
| MNDA | 0.30412057 | −0.1542547 | 0.61266539 |
| RBP7 | 0.23331714 | −0.3036785 | 0.61070353 |
| BST1 | 0.122712 | −0.186492 | 0.60604323 |
| DLGAP5 | −0.4315828 | 0.63624947 | 0.60031925 |
| NOD2 | 0.17425829 | −0.1454336 | 0.59975323 |
| LILRA3 | 0.20059286 | −0.1835364 | 0.59341604 |
| CLEC4A | 0.18052064 | −0.1931263 | 0.59074397 |
| TNFSF13B | 0.10512379 | −0.1354275 | 0.58924638 |
| CSF2RA | 0.2099765 | −0.1568839 | 0.58905845 |
| BRI3 | 0.15394743 | −0.1632444 | 0.58755564 |
| LMO2 | 0.31630214 | −0.0804872 | 0.57990754 |
| KIAA0101 | −0.4763506 | 0.48525963 | 0.57873232 |
| RFC5 | −0.0564904 | 0.16896686 | 0.57734968 |
| FEN1 | −0.1104692 | 0.29375101 | 0.57678051 |
| PTPN22 | −0.1079056 | 0.10260764 | 0.57319577 |
| CDKN3 | −0.2396239 | 0.37401346 | 0.57222471 |
| QPCT | 0.17933 | −0.139771 | 0.56171375 |
| NDC80 | −0.2696579 | 0.34558996 | 0.55789998 |
| KIR2DS2 | −0.512763 | 0.28105043 | 0.55592571 |
| FPR1 | 0.15585371 | −0.1152266 | 0.55319134 |
| ENPP5 | −0.1980916 | 0.55005255 | 0.54716296 |
| DPYD | 0.15686936 | −0.138058 | 0.54407704 |
| TGFBI | 0.21844536 | −0.2026333 | 0.54289146 |
| CDT1 | −0.3920278 | 0.50043445 | 0.5284931 |
| KLRB1 | −0.3829834 | 0.23405564 | 0.52746592 |
| SMC2 | −0.1223013 | 0.18895004 | 0.52373434 |
| TYROBP | 0.12911129 | −0.1495625 | 0.52248479 |
| GMNN | −0.1206991 | 0.18704521 | 0.52038427 |
| FGR | 0.13959557 | −0.137834 | 0.51922646 |
| KIF18A | −0.4757864 | 0.79021046 | 0.51609086 |
| KIF20B | 0.01059714 | 0.04736749 | 0.51442842 |
| RAD21 | −0.1034677 | 0.16341486 | 0.51255661 |
| SLC31A2 | 0.20108021 | −0.0789601 | 0.51054162 |
| VNN3 | 0.08985123 | −0.3932456 | 0.50444625 |
| MARCO | 0.262151 | −0.0732073 | 0.49729851 |
| KIF20A | −0.2128 | 0.41019729 | 0.49036135 |
| RAB31 | 0.19826943 | −0.196555 | 0.48769562 |
| TMEM154 | 0.07131207 | −0.1459213 | 0.48489437 |
| TLR1 | 0.216556 | −0.0681997 | 0.48466685 |
| CD14 | 0.20628029 | −0.1083709 | 0.48328154 |
| SPC25 | −0.3826298 | 0.44521026 | 0.47795474 |
| ASGR1 | 0.3071775 | −0.2405057 | 0.47638281 |
| EVI5 | 0.304583 | −0.3472783 | 0.47583407 |
| TLR6 | 0.32942427 | −0.1833656 | 0.4719326 |
| EMILIN2 | 0.132286 | −0.1315948 | 0.4710352 |
| CCNB1 | −0.2205839 | 0.48166149 | 0.46412741 |
| RNASE2 | 0.27261479 | −0.0458946 | 0.46154366 |
| CTSH | 0.08030629 | −0.1606683 | 0.46063525 |
| S1PR3 | 0.39169693 | −0.1508594 | 0.45997762 |
| IL1RN | 0.27895086 | −0.0517496 | 0.4594155 |
| CEP55 | −0.2470836 | 0.61351535 | 0.45922553 |
| ECT2 | 0.09959477 | 0.08800196 | 0.45700016 |
| AQP9 | 0.45163329 | −0.1484292 | 0.4557785 |
| FES | 0.20222443 | −0.1063407 | 0.45417507 |
| RNF130 | 0.1476985 | −0.1675974 | 0.45272824 |
| PADI2 | 0.24145129 | −0.273758 | 0.44702759 |
| TPX2 | −0.1368961 | 0.39044902 | 0.44619335 |
| NEK2 | −0.4797225 | 0.28285472 | 0.44548517 |
| CSF1R | 0.11002936 | −0.2353389 | 0.43745163 |
| RPA3 | −0.0468167 | 0.20367254 | 0.43511192 |
| SMARCD3 | 0.171004 | −0.2364289 | 0.43270195 |
| GPR56 | −0.3941556 | 0.42937814 | 0.42807517 |
| MGST1 | 0.26013621 | −0.171845 | 0.42270199 |
| FCGR1B | 0.32184743 | 0.123407 | 0.42115805 |
| KCNE3 | 0.28663807 | −0.1760301 | 0.42090032 |
| MS4A6A | 0.21972471 | −0.1876675 | 0.41792249 |
| SIRPB1 | 0.12962343 | −0.1614806 | 0.41581475 |
| RAD51 | −0.3247114 | 0.60031006 | 0.41531994 |
| PLBD1 | 0.30856714 | −0.1336086 | 0.41497238 |
| LAT2 | 0.14575757 | −0.0962826 | 0.41366954 |
| LILRB3 | 0.21902857 | −0.1867784 | 0.41348304 |
| LILRA5 | 0.11851443 | −0.224764 | 0.41331521 |
| PRIM1 | −0.0595427 | 0.19627593 | 0.41238984 |
| CFD | 0.09556486 | −0.3024731 | 0.41180017 |
| NCF2 | 0.17595029 | −0.1856544 | 0.41128375 |
| AGTRAP | 0.17473921 | −0.1716012 | 0.41016699 |
| GIMAP7 | 0.03605657 | 0.41435014 | 0.40999757 |
| IRAK3 | 0.17726571 | −0.3114963 | 0.40814394 |
| ASGR2 | 0.39454371 | −0.2091958 | 0.40774017 |
| CD36 | 0.308184 | −0.1079273 | 0.40726658 |
| CD33 | 0.155254 | −0.2266752 | 0.4065399 |
| PSAP | 0.12162514 | −0.20859 | 0.40558285 |
| BCL6 | 0.2903715 | −0.2510326 | 0.40459452 |
| LILRA6 | 0.20520129 | −0.075644 | 0.40169221 |
| ACSL1 | 0.16061336 | −0.2069103 | 0.40051088 |
| SULF2 | 0.15370471 | −0.2286793 | 0.39936672 |
| CRTAM | 0.05142471 | 0.16918629 | 0.39889111 |
| GZMA | −0.3233462 | 0.44860282 | 0.39138932 |
| PGD | 0.23078686 | −0.0989967 | 0.3894255 |
| RFC4 | −0.0840019 | 0.28309346 | 0.38924309 |
| POLD3 | 0.14601436 | 0.08214118 | 0.38879764 |
| MYD88 | 0.127095 | −0.0450128 | 0.38007042 |
| SH2D1A | −0.2182584 | 0.37141254 | 0.3795095 |
| NCF1 | 0.18834164 | −0.0888491 | 0.3792863 |
| CD27 | −0.1918529 | 0.347574 | 0.37901562 |
| HPSE | 0.27726671 | −0.1332741 | 0.37703943 |
| UBE2T | −0.1152586 | 0.50473483 | 0.37528116 |
| LILRA2 | 0.08333786 | −0.1941418 | 0.3730587 |
| LEF1 | −0.2306776 | 0.27287407 | 0.37274953 |
| KNTC1 | −0.0373946 | 0.17103619 | 0.37144283 |
| ACPP | 0.33864257 | −0.2515963 | 0.36989708 |
| SLC7A7 | 0.15972557 | −0.2263817 | 0.36773981 |
| FGFBP2 | −0.5226531 | 0.47477268 | 0.36717029 |
| TMPO | −0.008721 | 0.06823154 | 0.36509451 |
| CEP76 | −0.0563533 | 0.22814923 | 0.36508591 |
| CPVL | 0.19077136 | −0.265568 | 0.36420488 |
| TYMS | −0.437593 | 0.48260443 | 0.36281913 |
| CDC25A | −0.2446423 | 0.09325778 | 0.36107204 |
| CKS1B | −0.1774076 | 0.07276836 | 0.36086393 |
| GPR171 | 0.04673821 | 0.44807932 | 0.35950488 |
| POLA2 | −0.01999 | 0.14915151 | 0.35765998 |
| RAN | −0.0533419 | 0.12015604 | 0.35633478 |
| F5 | 0.25060213 | −0.0436135 | 0.35561866 |
| CD2 | −0.2164654 | 0.38027496 | 0.35558297 |
| NDUFA8 | −0.0209871 | 0.11569321 | 0.35526445 |
| RBM47 | 0.34911643 | −0.2308224 | 0.35463086 |
| CST3 | 0.08667 | −0.2032598 | 0.35391682 |
| SKA3 | −0.3279785 | 0.82917226 | 0.35208682 |
| SLAMF1 | −0.0525696 | 0.33766054 | 0.34173976 |
| NUP88 | −0.0518056 | 0.11604057 | 0.34158759 |
| BRCA1 | 0.1411805 | 0.19119731 | 0.33554829 |
| CYFIP1 | 0.27867043 | −0.1617847 | 0.33478346 |
| TIPIN | −0.073668 | 0.12286243 | 0.33322639 |
| LILRB2 | 0.10856657 | −0.1524331 | 0.33233354 |
| IL13RA1 | 0.22220743 | −0.3724449 | 0.33149446 |
| NCF4 | 0.28713014 | −0.1936098 | 0.33002888 |
| LTBR | 0.14341086 | −0.2142621 | 0.32847292 |
| ALOX5 | 0.18758043 | −0.1371492 | 0.32702454 |
| CRISPLD2 | 0.32555891 | −0.3586666 | 0.32526073 |
| PYGL | 0.29134307 | −0.2516874 | 0.32473667 |
| CFP | 0.12887729 | −0.2008422 | 0.32390272 |
| KIF2C | −0.1844746 | 0.36618417 | 0.32227884 |
| HCK | 0.145662 | −0.1101782 | 0.32224187 |
| THEMIS | −0.1614729 | 0.303302 | 0.3212733 |
| KIAA1598 | 0.19605307 | −0.2428218 | 0.32116134 |
| TIMP2 | 0.21143257 | −0.1767553 | 0.31745221 |
| GATA3 | −0.1580016 | 0.27439999 | 0.31631603 |
| NLRP3 | 0.19856679 | −0.4459587 | 0.31478029 |
| UHRF1 | −0.2013698 | 0.38829452 | 0.31378424 |
| OIP5 | −0.2158287 | 0.49771236 | 0.31289566 |
| PAK1 | 0.11821371 | −0.076532 | 0.31270228 |
| LY86 | 0.17209671 | −0.2437504 | 0.31243225 |
| KLRD1 | −0.3996624 | 0.33700039 | 0.31219778 |
| DTL | −0.320412 | 0.56387945 | 0.31180503 |
| SIRPA | 0.30036914 | −0.1727997 | 0.31140539 |
| TRAT1 | −0.1332071 | 0.33377432 | 0.31091869 |
| RASSF4 | 0.12517986 | −0.1642181 | 0.30897904 |
| PECAM1 | 0.16631493 | −0.2334685 | 0.30897622 |
| BUB1 | −0.2520322 | 0.5736033 | 0.30764568 |
| PYCARD | 0.121423 | −0.0958546 | 0.30668021 |
| ARHGEF10L | 0.14063229 | −0.211631 | 0.30492673 |
| ITM2A | −0.2660534 | 0.35634646 | 0.3044698 |
| LILRB1 | 0.24377036 | −0.0943035 | 0.3020182 |
| GIMAP6 | 0.00075014 | 0.19616636 | 0.30120438 |
| RASGRP1 | −0.2903379 | 0.30770064 | 0.29981424 |
| AGPAT9 | 0.15172171 | −0.0584703 | 0.29872519 |
| FPR2 | 0.28376686 | −0.063922 | 0.29368903 |
| AURKB | −0.3572037 | 0.00276418 | 0.29357518 |
| IMPA2 | 0.30031436 | −0.1785237 | 0.28937466 |
| CSF3R | 0.31417671 | −0.245967 | 0.28290744 |
| PVRIG | −0.3300437 | 0.43754479 | 0.28229247 |
| RAD54L | −0.1882477 | 0.31971504 | 0.27966512 |
| ATP9A | −0.1948771 | 0.29154147 | 0.27858758 |
| HOMER3 | 0.26818543 | −0.2048115 | 0.27512564 |
| TNS3 | 0.27859793 | −0.1568785 | 0.27421914 |
| NLRP12 | 0.22441814 | −0.1575549 | 0.27378834 |
| ADRB2 | 0.04538143 | 0.21887268 | 0.27331231 |
| TLR2 | 0.44489771 | −0.1334344 | 0.26995822 |
| SERPINA1 | 0.18943 | −0.1430217 | 0.26783199 |
| SLC24A4 | 0.19907907 | −0.2257899 | 0.26620923 |
| GLT1D1 | 0.29008614 | −0.1784594 | 0.26556227 |
| KLRK1 | −0.2942924 | 0.34556071 | 0.26183928 |
| CX3CR1 | 0.14954157 | 0.36685596 | 0.26072662 |
| BCL2 | −0.1933056 | 0.25353029 | 0.25863276 |
| MBOAT7 | 0.20782507 | −0.1171386 | 0.25719238 |
| KIR2DL5A | −0.3670154 | 0.33725142 | 0.25674147 |
| MELK | −0.4531195 | 0.54402785 | 0.25518512 |
| PLXNB2 | 0.12047643 | −0.1692871 | 0.25458228 |
| KLRF1 | −0.4228596 | 0.32693082 | 0.25236879 |
| ANLN | −0.5264763 | 0.72441581 | 0.25196326 |
| DOK3 | 0.18839314 | −0.072521 | 0.24778586 |
| PRKCH | −0.3584819 | 0.26582529 | 0.24681067 |
| ABLIM1 | −0.1923061 | 0.3216015 | 0.24501985 |
| EMR2 | 0.16595464 | −0.2645479 | 0.24246048 |
| P2RY13 | 0.317733 | −0.1098233 | 0.24071498 |
| KIR3DL1 | −0.4576109 | 0.25522369 | 0.23961293 |
| CD163 | 0.28459529 | −0.2914575 | 0.23826983 |
| CDCA7 | −0.21515 | 0.40727161 | 0.23802171 |
| HHEX | 0.21694243 | −0.1352445 | 0.23658793 |
| KLRC3 | −0.4404443 | 0.27508348 | 0.23570741 |
| GINS4 | −0.067823 | 0.22594522 | 0.23400711 |
| PRAM1 | 0.20610086 | −0.2093488 | 0.23394564 |
| IFNGR2 | 0.22150264 | −0.2453426 | 0.23288102 |
| SPI1 | 0.12291529 | −0.1344635 | 0.23243227 |
| MEGF9 | 0.229724 | −0.1275625 | 0.23166605 |
| CDCA8 | −0.2282616 | 0.2586116 | 0.22982876 |
| ALDH2 | 0.13867614 | −0.2825195 | 0.22925592 |
| RNASE6 | 0.24234229 | −0.2412168 | 0.22871705 |
| STAT4 | −0.3668694 | 0.33649532 | 0.22717006 |
| SLC15A3 | 0.14341379 | −0.2141991 | 0.22675477 |
| PLK1 | 0.4368879 | 0.34197855 | 0.2262495 |
| CDK1 | −0.2976619 | 0.35394821 | 0.22600548 |
| FBP1 | 0.24521021 | −0.2207796 | 0.225988 |
| CLIC3 | −0.4618959 | 0.20322614 | 0.22596421 |
| MYO1F | 0.13985314 | −0.0322897 | 0.2224607 |
| S100A11 | 0.17343407 | −0.1818736 | 0.22134581 |
| AOAH | 0.11295686 | −0.1658184 | 0.21938268 |
| CD3D | −0.2334819 | 0.39212054 | 0.21866053 |
| TMTC2 | 0.38385421 | −0.1644296 | 0.21700901 |
| PTGDR | −0.2460782 | 0.42568543 | 0.2166249 |
| TREM1 | 0.31924157 | −0.3498546 | 0.21621519 |
| FCGRT | 0.12817407 | −0.2209585 | 0.21604277 |
| CD8B | −0.1882037 | 0.34228961 | 0.21353166 |
| PRF1 | −0.3665207 | 0.42110561 | 0.2112278 |
| NCAM1 | −0.3655757 | 0.18317471 | 0.21062028 |
| CYBB | 0.11776321 | −0.0507948 | 0.21060862 |
| SYK | 0.19662329 | −0.1717975 | 0.20957884 |
| TBXAS1 | 0.11928286 | −0.2289053 | 0.20873346 |
| ZAP70 | −0.3733384 | 0.35043057 | 0.20738283 |
| HJURP | −0.3412081 | 0.49581671 | 0.20527139 |
| PRKCD | 0.19511836 | −0.0893292 | 0.2037058 |
| KIF11 | −0.1548747 | 0.27468888 | 0.2031195 |
| RHOH | −0.2636339 | 0.24160936 | 0.20231186 |
| GRN | 0.20724414 | −0.120798 | 0.20195312 |
| ITGAM | 0.17888443 | −0.0520518 | 0.19961885 |
| XCL1 | −0.4685001 | 0.17072114 | 0.19780327 |
| BTK | 0.15670586 | −0.1421541 | 0.19737015 |
| KIR2DS4 | −0.4630044 | 0.33428446 | 0.19658952 |
| GINS2 | −0.3195001 | 0.46183131 | 0.19502951 |
| IL12RB2 | −0.1031039 | 0.19992615 | 0.19058768 |
| FXYD6 | 0.2127035 | −0.3337615 | 0.18558203 |
| NKG7 | −0.4003281 | 0.36869686 | 0.18558075 |
| TBX21 | −0.3874794 | 0.3888075 | 0.18446864 |
| EOMES | −0.1587816 | 0.22841004 | 0.18266617 |
| CPPED1 | 0.13722893 | −0.2401733 | 0.1788541 |
| CST7 | −0.4068891 | 0.35034489 | 0.17509555 |
| KIR2DL2 | −0.5014236 | 0.33998994 | 0.174505 |
| STEAP4 | 0.45599867 | −0.0182688 | 0.17020067 |
| RORA | −0.3176324 | 0.13335904 | 0.16985963 |
| TCF7 | −0.2205655 | 0.27400575 | 0.16767287 |
| MCTP1 | 0.18948886 | −0.2029344 | 0.16537404 |
| CCNB2 | −0.1946636 | 0.39873935 | 0.16506957 |
| CCL5 | −0.3745718 | 0.36271357 | 0.16502922 |
| TRIP13 | −0.1144978 | 0.32300382 | 0.16502514 |
| PDGFRB | −0.3998149 | 0.51231926 | 0.16374775 |
| KLRC1 | −0.4014783 | 0.32408429 | 0.16126369 |
| SCPEP1 | 0.20582057 | −0.2124704 | 0.15853691 |
| SP140 | −0.047052 | 0.20126089 | 0.15829673 |
| POLA1 | −0.0924215 | 0.10952729 | 0.15781327 |
| FASLG | −0.4090754 | 0.63064534 | 0.1573457 |
| SORT1 | 0.32733014 | −0.233492 | 0.15500878 |
| DTYMK | 0.00880393 | 0.07686164 | 0.15394803 |
| SMARCAL1 | −0.0227714 | 0.09328157 | 0.15274879 |
| ITK | −0.294615 | 0.26540107 | 0.15102084 |
| NFAM1 | 0.14978529 | −0.1298816 | 0.15092301 |
| ITPR3 | −0.1481427 | 0.30532818 | 0.15078737 |
| E2F7 | −0.1723229 | 0.35509695 | 0.14840147 |
| SIGLEC7 | 0.12681557 | −0.2019304 | 0.14439254 |
| PCNA | −0.2071294 | 0.26508754 | 0.14422628 |
| ANO10 | 0.183057 | −0.1661393 | 0.14284752 |
| SSBP1 | −0.0296017 | 0.04982696 | 0.13694118 |
| TGFBR3 | −0.5264719 | 0.3242523 | 0.13637054 |
| CENPE | −0.0645918 | 0.27777848 | 0.13544765 |
| CARD9 | 0.14008593 | −0.2250704 | 0.13415354 |
| CHAF1B | −0.2801058 | 0.34651381 | 0.13191569 |
| CD8A | −0.2015009 | 0.32647193 | 0.12985501 |
| CDCA5 | −0.3264995 | 0.35916156 | 0.12874482 |
| CEP72 | −0.0502172 | 0.18587307 | 0.12853165 |
| HOPX | −0.4079511 | 0.31981293 | 0.12767412 |
| TLR8 | 0.25849436 | −0.1439851 | 0.12389608 |
| SLC16A3 | 0.16651343 | −0.0060917 | 0.12252404 |
| CD96 | −0.2954874 | 0.27057925 | 0.12024164 |
| CAMK4 | −0.2131127 | 0.21834766 | 0.11914249 |
| CENPF | −0.2633705 | 0.45853571 | 0.11887395 |
| LCK | −0.1946184 | 0.24340629 | 0.11624022 |
| KCTD12 | 0.25632571 | −0.1325746 | 0.11515547 |
| CASC5 | −0.0853667 | 0.3705584 | 0.11501154 |
| SGOL2 | −0.0736771 | 0.20052546 | 0.11490859 |
| GZMM | −0.5277151 | 0.29230996 | 0.11421952 |
| IL7R | −0.0746926 | 0.33924354 | 0.11402449 |
| MCM6 | −0.2673995 | 0.30639182 | 0.11092836 |
| CD1D | 0.33226726 | −0.3583073 | 0.10996758 |
| ASPM | −0.3264533 | 0.53236356 | 0.1083077 |
| IL2RB | −0.4383737 | 0.31879714 | 0.10657716 |
| PTTG1 | −0.1689252 | 0.35232025 | 0.10572265 |
| FANCI | −0.1670241 | 0.21036312 | 0.10293415 |
| GZMH | −0.3635874 | 0.43763271 | 0.10215849 |
| SLC11A1 | 0.25345971 | −0.2653527 | 0.10122901 |
| ETS1 | −0.3095553 | 0.270924 | 0.10066554 |
| CKAP5 | 0.06925536 | 0.19171896 | 0.10001639 |
| WDFY3 | 0.227156 | −0.3627252 | 0.09970533 |
| IL18R1 | −0.2915981 | 0.23804614 | 0.09903459 |
| CENPN | −0.1552708 | −0.1012391 | 0.09358631 |
| KIR2DS3 | −0.4409509 | 0.13590208 | 0.09236038 |
| TUBG1 | 0.04338786 | 0.29674596 | 0.09095781 |
| NUF2 | −0.1199278 | 0.38920616 | 0.08923606 |
| SMC4 | −0.0498615 | 0.18205457 | 0.08747603 |
| NUSAP1 | −0.0788597 | 0.11708601 | 0.08618049 |
| CHST15 | 0.24669143 | −0.1275318 | 0.08565709 |
| PTPN4 | −0.3326756 | 0.29473275 | 0.0851204 |
| FGD4 | 0.15304257 | −0.1932215 | 0.08126504 |
| FCGR2A | 0.19513057 | −0.2171639 | 0.07896857 |
| CCR7 | −0.1183211 | 0.22720743 | 0.07812974 |
| S1PR5 | −0.4823231 | 0.27190594 | 0.07531726 |
| CD247 | −0.4255066 | 0.30918132 | 0.07190587 |
| CD7 | −0.4413583 | 0.228816 | 0.0681526 |
| RAB32 | 0.16056593 | −0.1885954 | 0.06790996 |
| PLEKHF1 | −0.2791689 | 0.33819318 | 0.06736293 |
| EXO1 | −0.4247823 | 0.59886942 | 0.06443739 |
| CTSW | −0.487019 | 0.32969107 | 0.06385929 |
| TNFSF13 | 0.11867379 | −0.2346925 | 0.06363152 |
| TXK | −0.3989911 | 0.28206181 | 0.06064901 |
| CDCA2 | −0.3801736 | 0.42248241 | 0.05956476 |
| DHFR | 0.00870521 | 0.28889754 | 0.05952899 |
| MCM4 | −0.2320073 | 0.36402665 | 0.05912974 |
| NPL | −0.0636007 | 0.004466 | 0.05885316 |
| SLA2 | −0.3782789 | 0.24943868 | 0.0563309 |
| SH2D2A | −0.3770402 | 0.36344946 | 0.05619833 |
| NCAPD3 | −0.0677968 | 0.09737246 | 0.05570218 |
| GPR114 | −0.2936257 | 0.30667602 | 0.05449794 |
| DPP4 | −0.0943919 | 0.21415932 | 0.05352765 |
| LAT | −0.2510211 | 0.318139 | 0.05175177 |
| SPOCK2 | −0.3122131 | 0.25077596 | 0.04956459 |
| TIGIT | −0.2286996 | 0.43677026 | 0.04669768 |
| CXCR3 | −0.3350559 | 0.40025993 | 0.04667949 |
| CYBRD1 | 0.111162 | −0.3007594 | 0.04645738 |
| MCM5 | −0.0019691 | 0.08003532 | 0.04528597 |
| TCHP | 0.13509083 | 0.20927789 | 0.04381717 |
| NACC2 | 0.18404114 | −0.26109 | 0.04016366 |
| CD5 | −0.2471322 | 0.27680911 | 0.03716696 |
| MPEG1 | 0.14583836 | −0.170232 | 0.03309445 |
| STMN1 | −0.3249196 | 0.34691546 | 0.03205552 |
| SLAMF7 | −0.2300298 | 0.34091768 | 0.03058441 |
| CLEC4E | 0.2837066 | −0.3068958 | 0.03012479 |
| UBASH3A | −0.2202034 | 0.35947714 | 0.02497065 |
| SPAG5 | −0.0995263 | 0.23141186 | 0.02354363 |
| IL21R | −0.1031827 | 0.20682311 | 0.01912902 |
| GNLY | −0.4441947 | 0.35938757 | 0.01642658 |
| PRKCQ | −0.3047643 | 0.28771886 | 0.01602466 |
| KIF23 | 0.09160493 | 0.03388473 | 0.01532775 |
| F13A1 | 0.28193136 | −0.0599006 | 0.01001287 |
| SAMD3 | −0.372721 | 0.44468854 | 0.00880716 |
| IKZF1 | −0.1879529 | 0.14708693 | 0.00716977 |
| RARRES3 | −0.1910249 | 0.15946211 | 0.00466383 |
| GZMB | −0.3850094 | 0.24324889 | 0.00420963 |

The invention further comprises the following embodiments:

### Embodiment 1

A method of determining the prognosis of a patient comprising the steps of
(a) determining the expression level of at least one marker gene selected from the group consisting of the marker genes set out in table 1 in a sample of the patient to obtain a gene expression profile;
(b) determining the prognosis of the patient based on the gene expression profile obtained in step (a).

### Embodiment 2

Method according to embodiment 1, wherein the patient is a cancer or tumor patient.

### Embodiment 3

Method according to embodiment 1 or 2, wherein the at least one marker gene is selected from the group consisting of marker genes set out in table 2.

### Embodiment 4

Method according to embodiment 3, wherein the at least one marker gene is selected from the group consisting of marker genes set out in table 3.

### Embodiment 5

Method according to embodiment 4, wherein the at least one marker gene is selected from group consisting of marker genes set out in table 4.

### Embodiment 6

Method according to embodiment 5, wherein the at least one marker gene is selected from group consisting of marker genes set out in table 5.

### Embodiment 7

Method according to embodiment 1, wherein at least 10 marker genes, preferably at least 30 marker genes, more preferably at least 50 marker genes, most preferably at least 100 marker genes are selected from table 1.

### Embodiment 8

Method according to embodiment 3, wherein at least 10 marker genes, preferably at least 30 marker genes, more preferably at least 50 marker genes, most preferably at least 100 marker genes are selected from table 2.

### Embodiment 9

Method according to embodiment 4, wherein at least 10 marker genes, preferably at least 30 marker genes, more preferably at least 50 marker genes are selected from table 3.

### Embodiment 10

Method according embodiment 5, wherein at least 10 marker genes, preferably at least 20 marker genes, more preferably at least 30 marker genes a selected from the group consisting of from table 4.

### Embodiment 11

Method according to any one of the preceding embodiments, wherein the expression level of at least one marker gene is measured before administration and/or after administration of at least one dose of a therapeutic agent.

### Embodiment 12

Method according to embodiment 11, wherein the gene expression profile of step (a) is obtained by determining the difference of the expression level of the at least one marker gene measured before administration of the therapeutic agent and after administration of at least one dose of the therapeutic agent.

### Embodiment 13

Method according to embodiment 11 or 12, wherein the therapeutic agent is an immunostimulatory composition and/or a vaccine and/or an immunotherapeutic agent.

### Embodiment 14

Method according to any one of embodiments 11 to 13, wherein the immunostimulatory composition and/or vaccine comprises at least one antigen selected from the group consisting of MAGE-C1, MAGE-C2, NY-ESO-1, Survivin and 5T4 or fragments or variants thereof.

### Embodiment 15

Method according to any one of embodiments 11 to 14, wherein the immunostimulatory composition and/or vaccine comprises the antigens MAGE-C1, MAGE-C2, NY-ESO-1, Survivin and 5T4 or fragments or variants thereof.

### Embodiment 16

Method according to embodiment 11 to 15, wherein the antigen(s) are present as peptides or proteins and/or are encoded by at least one nucleotide sequence.

### Embodiment 17

Method according to any one of embodiments 11 to 16, wherein the antigen(s) are encoded by at least one mRNA molecule.

### Embodiment 18

Method according to any one of embodiments 11 to 17, wherein the cancer is selected from the group consisting of Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood′, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland′ Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

### Embodiment 19

Method according to embodiment 18, wherein the cancer is lung cancer, preferably non-small cell lung cancer (NSCLC).

### Embodiment 20

Method according to any one of the preceding embodiments, wherein the sample of the patient comprises peripheral blood mono-nuclear cells (PBMCs).

### Embodiment 21

Method according to any one of the preceding embodiments, wherein in step (b) a hierarchical clustering algorithm is applied.

### Embodiment 22

Kit, diagnostic composition or device for the analysis of at least one marker gene set out in table 1 comprising at least one primer and/or probe selective for determining the expression level of at least one marker gene set out in table 1.

### Embodiment 23

Kit, diagnostic composition or device of embodiment 22, further comprising an enzyme for primer elongation, nucleotides and/or labeling agents.

### Embodiment 24

Microarray, comprising at least one probe selective for determining the expression level of at least one marker gene set out in table 1.

### Embodiment 25

Use of a microarray according to embodiment 24 for determining the prognosis of a cancer patient.

### Embodiment 26

Use of a kit, diagnostic composition or device according to embodiment 22 or 23 for determining the prognosis of a cancer patient.

1. A method of determining the prognosis of a patient comprising the steps of
(a) determining the expression level of at least 30 marker genes selected from the group consisting of the marker genes set out in table 4 in a sample of the patient to obtain a gene expression profile;
(b) determining the prognosis of the patient based on the gene expression profile obtained in step (a).

2. Method according to claim 1, wherein the patient is a cancer or tumor patient.

3. Method according to claim 1, wherein at least 10 additional marker genes, preferably at least 20 additional marker genes, more preferably at least 70 additional marker genes are selected from table 1.

4. Method according to any one of the preceding claims, wherein at least 10 additional marker genes, preferably at least 20 additional marker genes, more preferably at least 70 additional marker genes are selected from table 2.

5. Method according to any one of the preceding claims, wherein at least 10 additional marker genes, preferably at least 20 additional marker genes are selected from table 3.

6. Method according to any one of the preceding claims, wherein the expression level of at least one marker gene is measured before administration and/or after administration of at least one dose of a therapeutic agent.

7. Method according to claim 6, wherein the gene expression profile of step (a) is obtained by determining the difference of the expression level of the at least one marker gene measured before administration of the therapeutic agent and after administration of at least one dose of the therapeutic agent.

8. Method according to claim 6 or 7, wherein the therapeutic agent is an immunostimulatory composition and/or a vaccine and/or an immunotherapeutic agent.

9. Method according to any one of claims 6 to 8, wherein the immunostimulatory composition and/or vaccine comprises at least one antigen selected from the group consisting of MAGE-C1, MAGE-C2, NY-ESO-1, Survivin and 5T4 or fragments or variants thereof.

10. Method according to any one of claims 6 to 9, wherein the immunostimulatory composition and/or vaccine comprises the antigens MAGE-C1, MAGE-C2, NY-ESO-1, Survivin and 5T4 or fragments or variants thereof.

11. Method according to claim 6 to 10, wherein the antigen(s) are present as peptides or proteins and/or are encoded by at least one nucleotide sequence.

12. Method according to any one of claims 6 to 11, wherein the antigen(s) are encoded by at least one mRNA molecule.

13. Method according to any one of claims 6 to 12, wherein the cancer is selected from the group consisting of Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland’ Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

14. Method according to claim 13, wherein the cancer is lung cancer, preferably non-small cell lung cancer (NSCLC).

15. Method according to any one of the preceding claims, wherein the sample of the patient comprises peripheral blood mono-nuclear cells (PBMCs).

16. Method according to any one of the preceding claims, wherein in step (b) a hierarchical clustering algorithm is applied.

17. Kit, diagnostic composition or device for the analysis of at least one marker gene set out in table 1 comprising at least one primer and/or probe selective for determining the expression level of at least one marker gene set out in table 1.

18. Kit, diagnostic composition or device of claim 17, further comprising an enzyme for primer elongation, nucleotides and/or labeling agents.

19. Microarray, comprising at least one probe selective for determining the expression level of at least one marker gene set out in table 1.

20. Use of a microarray according to claim 19 for determining the prognosis of a cancer patient.

21. Use of a kit, diagnostic composition or device according to claim 17 or 18 for determining the prognosis of a cancer patient.

22. A method of determining the prognosis of a patient comprising the steps of
(a) determining the expression level of at least one marker gene selected from the group consisting of the marker genes set out in table 11 in a sample of the patient to obtain a gene expression profile;
(b) determining the prognosis of the patient based on the gene expression profile obtained in step (a).

23. Method according to claim 22, wherein the patient is a cancer or tumor patient.

24. Method according to claim 22 or 23, wherein the at least one marker genes is selected from the group consisting of marker genes set out in table 12.

25. Method according to claim 24, wherein the at least one marker gene is selected from the group consisting of marker genes set out in table 13.

26. Method according to claim 25, wherein the at least one marker gene is selected from group consisting of marker genes set out in table 14.

27. Method according to claim 26, wherein the at least one marker gene is selected from group consisting of marker genes set out in table 15.

28. Method according to claim 22, wherein at least 10 marker genes, preferably at least 30 marker genes, more preferably at least 50 marker genes, most preferably at least 100 marker genes are selected from table 11.

29. Method according to claim 24, wherein at least 10 marker genes, preferably at least 30 marker genes, more preferably at least 50 marker genes, most preferably at least 100 marker genes are selected from table 12.

30. Method according to claim 25, wherein at least 10 marker genes, preferably at least 30 marker genes, more preferably at least 50 marker genes are selected from table 13.

31. Method according claim 26, wherein at least 10 marker genes, preferably at least 20 marker genes, more preferably at least 30 marker genes a selected from the group consisting of from table 14.

32. Method according to any one of claims 22 to 31, wherein the expression level of at least one marker gene is measured before administration and/or after administration of at least one dose of a therapeutic agent.

33. Method according to claim 32, wherein the gene expression profile of step (a) is obtained by determining the difference of the expression level of the at least one marker gene measured before administration of the therapeutic agent and after administration of at least one dose of the therapeutic agent.

34. Method according to claim 32 or 33, wherein the therapeutic agent is an immunostimulatory composition and/or a vaccine and/or an immunotherapeutic agent.

35. Method according to any one of claims 32 to 34, wherein the immunostimulatory composition and/or vaccine comprises at least one antigen selected from the group consisting of MAGE-C1, MAGE-C2, NY-ESO-1, Survivin, 5T4, PSA, PSMA, PSCA, STEAP, PAP and MUC1 or fragments or variants thereof.

36. Method according to any one of claims 32 to 35, wherein the immunostimulatory composition and/or vaccine comprises the antigens
(i) MAGE-C1, MAGE-C2, NY-ESO-1, Survivin and 5T4 or fragments or variants thereof; or
(ii) PSA, PSMA, PSCA, STEAP, PAP and MUC1 or fragments or variants thereof.

37. Method according to claim 32 to 36, wherein the antigen(s) are present as peptides or proteins and/or are encoded by at least one nucleotide sequence.

38. Method according to any one of claims 32 to 37, wherein the antigen(s) are encoded by at least one mRNA molecule.

39. Method according to any one of claims 22 to 38, wherein the cancer is selected from the group consisting of Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland’ Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

40. Method according to claim 39, wherein the cancer is lung cancer or prostate cancer.

41. Method according to claim 40, wherein the lung cancer is preferably non-small cell lung cancer (NSCLC).

42. Method according to claims 22 to 41, wherein the sample of the patient comprises peripheral blood mono-nuclear cells (PBMCs).

43. Method according to claims 22 to 42, wherein in step (b) a hierarchical clustering algorithm is applied.

44. Kit, diagnostic composition or device for the analysis of at least one marker gene set out in table 11 comprising at least one primer and/or probe selective for determining the expression level of at least one marker gene set out in table 11.

45. Kit, diagnostic composition or device of claim 44, further comprising an enzyme for primer elongation, nucleotides and/or labeling agents.

46. Microarray, comprising at least one probe selective for determining the expression level of at least one marker gene set out in table 11.

47. Use of a microarray according to claim 46 for determining the prognosis of a cancer patient.

48. Use of a kit, diagnostic composition or device according to claim 44 or 45 for determining the prognosis of a cancer patient.

49. A method of determining the prognosis of a patient comprising the steps of
(a) determining the expression level of at least one marker gene selected from the group consisting of the marker genes set out in table 6 in a sample of the patient to obtain a gene expression profile;
(b) determining the prognosis of the patient based on the gene expression profile obtained in step (a).

50. Method according to claim 49, wherein the patient is a cancer or tumor patient.

51. Method according to claim 49 or 50, wherein the at least one marker genes is selected from the group consisting of marker genes set out in table 7.

52. Method according to claim 51, wherein the at least one marker gene is selected from the group consisting of marker genes set out in table 8.

53. Method according to claim 51, wherein the at least one marker gene is selected from group consisting of marker genes set out in table 9.

54. Method according to claim 51, wherein the at least one marker gene is selected from group consisting of marker genes set out in table 10.

55. Method according to claim 49, wherein at least 10 marker genes, preferably at least 30 marker genes, more preferably at least 50 marker genes, most preferably at least 100 marker genes are selected from table 6.

56. Method according to claim 51, wherein at least 10 marker genes, preferably at least 30 marker genes, more preferably at least 50 marker genes, most preferably at least 100 marker genes are selected from table 7.

57. Method according to claim 52, wherein at least 10 marker genes, preferably at least 30 marker genes, more preferably at least 50 marker genes are selected from table 8.

58. Method according claim 53, wherein at least 10 marker genes, preferably at least 20 marker genes, more preferably at least 30 marker genes a selected from the group consisting of from table 9.

59. Method according to any one of claims 49 to 58, wherein the expression level of at least one marker gene is measured before administration and/or after administration of at least one dose of a therapeutic agent.

60. Method according to claim 59, wherein the gene expression profile of step (a) is obtained by determining the difference of the expression level of the at least one marker gene measured before administration of the therapeutic agent and after administration of at least one dose of the therapeutic agent.

61. Method according to claim 59 or 60, wherein the therapeutic agent is an immunostimulatory composition and/or a vaccine and/or an immunotherapeutic agent.

62. Method according to any one of claims 59 to 61, wherein the immunostimulatory composition and/or vaccine comprises at least one antigen selected from the group consisting of PSA, PSMA, PSCA, STEAP, PAP and MUC1 or fragments or variants thereof.

63. Method according to any one of claims 59 to 62, wherein the immunostimulatory composition and/or vaccine comprises the antigens PSA, PSMA, PSCA, STEAP, PAP and MUC1 or fragments or variants thereof.

64. Method according to claim 62 or 63, wherein the antigen(s) are present as peptides or proteins and/or are encoded by at least one nucleotide sequence.

65. Method according to any one of claims 59 to 64, wherein the antigen(s) are encoded by at least one mRNA molecule.

66. Method according to any one of claims 50 to 65, wherein the cancer is selected from the group consisting of Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood’, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland’ Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

67. Method according to claim 66, wherein the cancer is prostate cancer.

68. Method according to any one of claims 49 to 67, wherein the sample of the patient comprises peripheral blood mono-nuclear cells (PBMCs).

69. Method according to any one of claims 49 to 68, wherein in step (b) a hierarchical clustering algorithm is applied.

70. Kit, diagnostic composition or device for the analysis of at least one marker gene set out in table 6 comprising at least one primer and/or probe selective for determining the expression level of at least one marker gene set out in table 6.

71. Kit, diagnostic composition or device of claim 70, further comprising an enzyme for primer elongation, nucleotides and/or labeling agents.

72. Microarray, comprising at least one probe selective for determining the expression level of at least one marker gene set out in table 6.

73. Use of a microarray according to claim 72 for determining the prognosis of a cancer patient.

74. Use of a kit, diagnostic composition or device according to claim 70 or 71 for determining the prognosis of a cancer patient.
